(19) United States
(12) Patent Application Publication
(10) Pub. No.: US 2024/0316209 A1
(43) Pub. Date: Sep. 26, 2024

(54) ANTIBODIES CONTAINING UNNATURAL AMINO ACIDS AND METHODS OF MAKING AND USING THE SAME

(71) Applicant: BrickBio, Inc., Woburn, MA (US)

(72) Inventors: James Sebastian Italia, Charlestown, MA (US); Nikolaos Biris, Westborough, MA (US)

(21) Appl. No.: 18/044,943

(22) PCT Filed: Sep. 10, 2021
(86) PCT No.: PCT/US2021/049953

§ 371 Date: Mar. 10, 2023

Related U.S. Application Data
(60) Provisional application No. 63/144,628, filed on Feb. 2, 2021.
(60) Provisional application No. 63/076,814, filed on Sep. 10, 2020.

(51) Int. Cl. A61K 47/68; C07K 16/32; C12N 9/00

(52) U.S. Cl. CPC A61K 47/68031; A61K 47/6889; C07K 16/32; C12N 9/93; C12Y 601/01001; C12Y 601/01026

(57) ABSTRACT

The invention relates generally to engineered antibodies containing unnatural amino acids (UAAs) and methods of making and using such antibodies.

### CROSS-REFERENCE TO RELATED APPLICATIONS

This application claims the benefit of and priority to U.S. Provisional Patent Application Nos. 63/076,814, filed Sep. 10, 2020, and 63/144,628, filed Feb. 2, 2021, which are incorporated herein by reference in their entirety for all purposes.

### FIELD OF THE INVENTION

The present disclosure relates, in general, to the field of engineered antibodies where orthogonal tRNA/aminoacyl-tRNA synthetase pairs are used to incorporate unnatural amino acids (UAAs) into an antibody of interest.

### BACKGROUND

In nature, proteins are produced in cells via processes known as transcription and translation. During transcription, a gene comprising a series of codons that collectively encode a protein of interest is transcribed into messenger RNA (mRNA). During translation, a ribosome attaches to and moves along the mRNA and incorporates specific amino acids into a polypeptide chain being synthesized (translated) from the mRNA at positions corresponding to the codons to produce the protein. During translation, naturally occurring amino acids coupled to transfer RNAs (tRNAs) enter the ribosome. The tRNAs, which contain an anti-codon sequence, hybridize to their respective codon sequences in mRNA and transfer the amino acid they are carrying into the nascent protein chain at the appropriate position as the protein is synthesized.

Over the last few decades, significant efforts have been made to produce homogenous preparations of site-specifically modified proteins, e.g., mammalian proteins, on commercial scale quantities for use in a variety of applications, including, for example, therapeutics and diagnostics. Furthermore, efforts have been made to produce these modified mammalian proteins in eukaryotic cells (e.g., mammalian cells) because the proteins may be more readily produced in a properly folded and fully active form and/or post-translationally modified in a manner similar to the native protein naturally produced in a mammalian cell.

One approach for producing proteins that contain site-specific modifications involves the site-specific incorporation of one or more unnatural amino acids (UAAs) into a protein of interest. The ability to site-specifically incorporate UAAs into proteins in vivo has become a powerful tool to augment protein function or introduce new chemical functionalities not found in nature. The core elements required for this technology include: an engineered tRNA, an engineered aminoacyl-tRNA synthetase (aaRS) that charges the tRNA with a UAA, and a unique codon, e.g., a stop codon, directing the incorporation of the UAA into the protein as it is being synthesized.

Central to this approach is the use of an engineered tRNA/aaRS pair in which the aaRS charges the tRNA with the UAA of interest without cross-reacting with the tRNAs and amino acids normally present in the expression host cell. This has been accomplished by using an engineered tRNA/aaRS pair derived from an organism in different domain of life as the expression host cell so as to maximize the orthogonality between the engineered tRNA/aaRS pair (e.g., an engineered bacterial tRNA/aaRS pair) and the tRNA/aaRS pairs naturally found in the expression host cell (e.g., mammalian cell). The engineered tRNA, which is charged with the UAA via the aaRS, binds or hybridizes to the unique codon, such as a premature stop codon (UAG, UGA, UAA) present in the mRNA encoding the protein to be expressed. See, for example, FIG. 1A, which shows the synthesis of a protein using an endogenous tRNA and an endogenous aaRS from the expression host cell and an engineered orthogonal tRNA and an orthogonal aaRS introduced into the host cell so as to facilitate the incorporation of a UAA into the protein as it is synthesized via the ribosome. To date, a variety of orthogonal tRNA/aaRS pairs have been produced for certain of the naturally occurring amino acids (see, e.g., U.S. Patent Publication US2017/0349891, and Zheng et al. (2018) BIOCHEM. 57:441-445). The approach facilitates the expression of proteins containing site specific modifications such as bioconjugation handles and photoactivatable crosslinkers, which can be used as therapeutics (e.g., antibody drug conjugates (ADCs), bi-specific antibodies (e.g., bispecific monoclonal antibodies), nanobodies, chemokines, vaccines, coagulation factors, hormones, and enzymes).

Conjugation of drugs, payloads, oligonucleotides, half-life extenders, and other molecules to antibodies has been explored as a method to improve therapeutic activity. However, the site of conjugation can impact biophysical properties, PK/PD, efficacy, manufacturing yield, etc. Accordingly, despite the efforts made to date, there remains a need for the identification of suitable conjugation sites in antibodies, expression platforms that allow for the incorporation of unnatural amino acids into antibodies with high efficiency, and methods for generating antibody conjugates with high efficiency.

### SUMMARY OF THE INVENTION

The invention is based, in part, on the discovery of specific locations in antibodies that allow for efficient incorporation of one, or more, unnatural amino acids (UAAs) into the antibodies. The invention is further based, in part, on the discovery of combinations of specified positions in antibodies, UAAs for incorporation at those positions, and molecules for conjugation at those UAAs. Among other things, the combinations of position, UAA, and molecule allow for the efficient generation of antibody conjugates with desirable properties, including, for example, expression yield, drug to antibody ratio (DAR), lack of aggregation, stability, and binding affinity.

Accordingly, in one aspect, the invention provides an antibody comprising (e.g., in a heavy chain or a fragment thereof):
(i) the amino acid sequence of SEQ ID NO: 118, with an unnatural amino acid (UAA) at a position corresponding to P6 of SEQ ID NO: 118; the amino acid sequence of SEQ ID NO: 119, with an unnatural amino acid (UAA) at a position corresponding to P14 of SEQ ID NO: 119; the amino acid sequence of SEQ ID NO: 120, with an unnatural amino acid (UAA) at a position corresponding to P14 of SEQ ID NO: 120; or the amino acid sequence of SEQ ID NO: 121, with an unnatural amino acid (UAA) at a position corresponding to P14 of SEQ ID NO: 121;
(ii) the amino acid sequence of SEQ ID NO: 122, with an unnatural amino acid (UAA) at a position corresponding to G6 of SEQ ID NO: 122; the amino acid sequence of SEQ ID NO: 123, with an unnatural amino acid (UAA) at a position corresponding to G17 of SEQ ID NO: 123; the amino acid sequence of SEQ ID NO: 120, with an unnatural amino acid (UAA) at a position corresponding to G66 of SEQ ID NO: 120; or the amino acid sequence of SEQ ID NO: 121, with an unnatural amino acid (UAA) at a position corresponding to G66 of SEQ ID NO: 121;
(iii) the amino acid sequence of SEQ ID NO: 124, with an unnatural amino acid (UAA) at a position corresponding to D6 of SEQ ID NO: 124; the amino acid sequence of SEQ ID NO: 125, with an unnatural amino acid (UAA) at a position corresponding to D7 of SEQ ID NO: 125; the amino acid sequence of SEQ ID NO: 120, with an unnatural amino acid (UAA) at a position corresponding to D73 of SEQ ID NO: 120; or the amino acid sequence of SEQ ID NO: 121, with an unnatural amino acid (UAA) at a position corresponding to D73 of SEQ ID NO: 121;
(iv) the amino acid sequence of SEQ ID NO: 126, with an unnatural amino acid (UAA) at a position corresponding to L6 of SEQ ID NO: 126; the amino acid sequence of SEQ ID NO: 127, with an unnatural amino acid (UAA) at a position corresponding to L6 of SEQ ID NO: 127; the amino acid sequence of SEQ ID NO: 120, with an unnatural amino acid (UAA) at a position corresponding to L115 of SEQ ID NO: 120; or the amino acid sequence of SEQ ID NO: 121, with an unnatural amino acid (UAA) at a position corresponding to L115 of SEQ ID NO: 121;
(v) the amino acid sequence of SEQ ID NO: 128, with an unnatural amino acid (UAA) at a position corresponding to A6 of SEQ ID NO: 128; the amino acid sequence of SEQ ID NO: 129, with an unnatural amino acid (UAA) at a position corresponding to A1 of SEQ ID NO: 129; the amino acid sequence of SEQ ID NO: 130, with an unnatural amino acid (UAA) at a position corresponding to A1 of SEQ ID NO: 130; or the amino acid sequence of SEQ ID NO: 121, with an unnatural amino acid (UAA) at a position corresponding to A121 of SEQ ID NO: 121;
(vi) the amino acid sequence of SEQ ID NO: 131, with an unnatural amino acid (UAA) at a position corresponding to K6 of SEQ ID NO: 131; the amino acid sequence of SEQ ID NO: 129, with an unnatural amino acid (UAA) at a position corresponding to K3 of SEQ ID NO: 129; the amino acid sequence of SEQ ID NO: 130, with an unnatural amino acid (UAA) at a position corresponding to K3 of SEQ ID NO: 130; or the amino acid sequence of SEQ ID NO: 121, with an unnatural amino acid (UAA) at a position corresponding to K124 of SEQ ID NO: 121;
(vii) the amino acid sequence of SEQ ID NO: 132, with an unnatural amino acid (UAA) at a position corresponding to T6 of SEQ ID NO: 132; the amino acid sequence of SEQ ID NO: 129, with an unnatural amino acid (UAA) at a position corresponding to T18 of SEQ ID NO: 129; the amino acid sequence of SEQ ID NO: 130, with an unnatural amino acid (UAA) at a position corresponding to T18 of SEQ ID NO: 130; or the amino acid sequence of SEQ ID NO: 121, with an unnatural amino acid (UAA) at a position corresponding to T138 of SEQ ID NO: 121;
(viii) the amino acid sequence of SEQ ID NO: 133, with an unnatural amino acid (UAA) at a position corresponding to A6 of SEQ ID NO: 133; the amino acid sequence of SEQ ID NO: 129, with an unnatural amino acid (UAA) at a position corresponding to A23 of SEQ ID NO: 129; the amino acid sequence of SEQ ID NO: 130, with an unnatural amino acid (UAA) at a position corresponding to A23 of SEQ ID NO: 130; or the amino acid sequence of SEQ ID NO: 121, with an unnatural amino acid (UAA) at a position corresponding to A143 of SEQ ID NO: 121;
(ix) the amino acid sequence of SEQ ID NO: 134, with an unnatural amino acid (UAA) at a position corresponding to V6 of SEQ ID NO: 134; the amino acid sequence of SEQ ID NO: 129, with an unnatural amino acid (UAA) at a position corresponding to V37 of SEQ ID NO: 129; the amino acid sequence of SEQ ID NO: 130, with an unnatural amino acid (UAA) at a position corresponding to V37 of SEQ ID NO: 130; or the amino acid sequence of SEQ ID NO: 121, with an unnatural amino acid (UAA) at a position corresponding to V157 of SEQ ID NO: 121;
(x) the amino acid sequence of SEQ ID NO: 174, with an unnatural amino acid (UAA) at a position corresponding to T6 of SEQ ID NO: 174; the amino acid sequence of SEQ ID NO: 129, with an unnatural amino acid (UAA) at a position corresponding to T38 of SEQ ID NO: 129; the amino acid sequence of SEQ ID NO: 130, with an unnatural amino acid (UAA) at a position corresponding to T38 of SEQ ID NO: 130; or the amino acid sequence of SEQ ID NO: 121, with an unnatural amino acid (UAA) at a position corresponding to T158 of SEQ ID NO: 121;
(xi) the amino acid sequence of SEQ ID NO: 135, with an unnatural amino acid (UAA) at a position corresponding to S6 of SEQ ID NO: 135; the amino acid sequence of SEQ ID NO: 129, with an unnatural amino acid (UAA) at a position corresponding to S40 of SEQ ID NO: 129; the amino acid sequence of SEQ ID NO: 130, with an unnatural amino acid (UAA) at a position corresponding to S40 of SEQ ID NO: 130; or the amino acid sequence of SEQ ID NO: 121, with an unnatural amino acid (UAA) at a position corresponding to S160 of SEQ ID NO: 121;
(xii) the amino acid sequence of SEQ ID NO: 136, with an unnatural amino acid (UAA) at a position corresponding to T6 of SEQ ID NO: 136; the amino acid sequence of SEQ ID NO: 129, with an unnatural amino acid (UAA) at a position corresponding to T47 of SEQ ID NO: 129; the amino acid sequence of SEQ ID NO: 130, with an unnatural amino acid (UAA) at a position corresponding to T47 of SEQ ID NO: 130; or the amino acid sequence of SEQ ID NO: 121, with an unnatural amino acid (UAA) at a position corresponding to T167 of SEQ ID NO: 121;
(xiii) the amino acid sequence of SEQ ID NO: 137, with an unnatural amino acid (UAA) at a position corresponding to T6 of SEQ ID NO: 137; the amino acid sequence of SEQ ID NO: 129, with an unnatural amino acid (UAA) at a position corresponding to T78 of SEQ ID NO: 129; the amino acid sequence of SEQ ID NO: 130, with an unnatural amino acid (UAA) at a position corresponding to T78 of SEQ ID NO: 130; or the amino acid sequence of SEQ ID NO: 121, with an unnatural amino acid (UAA) at a position corresponding to T198 of SEQ ID NO: 121;
(xiv) the amino acid sequence of SEQ ID NO: 138, with an unnatural amino acid (UAA) at a position corresponding to N6 of SEQ ID NO: 138; the amino acid sequence of SEQ ID NO: 129, with an unnatural amino acid (UAA) at a position corresponding to N84 of SEQ ID NO: 129; the amino acid sequence of SEQ ID NO: 130, with an unnatural amino acid (UAA) at a position corresponding to N84 of SEQ ID NO: 130; or the amino acid sequence of SEQ ID NO: 121, with an unnatural amino acid (UAA) at a position corresponding to N204 of SEQ ID NO: 121;
(xv) the amino acid sequence of SEQ ID NO: 139, with an unnatural amino acid (UAA) at a position corresponding to V6 of SEQ ID NO: 139; the amino acid sequence of SEQ ID NO: 129, with an unnatural amino acid (UAA) at a position corresponding to V85 of SEQ ID NO: 129; the amino acid sequence of SEQ ID NO: 130, with an unnatural amino acid (UAA) at a position corresponding to V85 of SEQ ID NO: 130; or the amino acid sequence of SEQ ID NO: 121, with an unnatural amino acid (UAA) at a position corresponding to V205 of SEQ ID NO: 121;
(xvi) the amino acid sequence of SEQ ID NO: 140, with an unnatural amino acid (UAA) at a position corresponding to N6 of SEQ ID NO: 140; the amino acid sequence of SEQ ID NO: 129, with an unnatural amino acid (UAA) at a position corresponding to N86 of SEQ ID NO: 129; the amino acid sequence of SEQ ID NO: 130, with an unnatural amino acid (UAA) at a position corresponding to N86 of SEQ ID NO: 130; or the amino acid sequence of SEQ ID NO: 121, with an unnatural amino acid (UAA) at a position corresponding to N206 of SEQ ID NO: 121;
(xvii) the amino acid sequence of SEQ ID NO: 141, with an unnatural amino acid (UAA) at a position corresponding to K6 of SEQ ID NO: 141; the amino acid sequence of SEQ ID NO: 129, with an unnatural amino acid (UAA) at a position corresponding to K93 of SEQ ID NO: 129; the amino acid sequence of SEQ ID NO: 130, with an unnatural amino acid (UAA) at a position corresponding to K93 of SEQ ID NO: 130; or the amino acid sequence of SEQ ID NO: 121, with an unnatural amino acid (UAA) at a position corresponding to K213 of SEQ ID NO: 121;
(xviii) the amino acid sequence of SEQ ID NO: 142, with an unnatural amino acid (UAA) at a position corresponding to D6 of SEQ ID NO: 142; the amino acid sequence of SEQ ID NO: 129, with an unnatural amino acid (UAA) at a position corresponding to D95 of SEQ ID NO: 129; the amino acid sequence of SEQ ID NO: 130, with an unnatural amino acid (UAA) at a position corresponding to D95 of SEQ ID NO: 130; or the amino acid sequence of SEQ ID NO: 121, with an unnatural amino acid (UAA) at a position corresponding to D215 of SEQ ID NO: 121;
(xix) the amino acid sequence of SEQ ID NO: 143, with an unnatural amino acid (UAA) at a position corresponding to 16 of SEQ ID NO: 143; the amino acid sequence of SEQ ID NO: 144, with an unnatural amino acid (UAA) at a position corresponding to 123 of SEQ ID NO: 144; the amino acid sequence of SEQ ID NO: 130, with an unnatural amino acid (UAA) at a position corresponding to 1136 of SEQ ID NO: 130; or the amino acid sequence of SEQ ID NO: 121, with an unnatural amino acid (UAA) at a position corresponding to 1256 of SEQ ID NO: 121;
(xx) the amino acid sequence of SEQ ID NO: 145, with an unnatural amino acid (UAA) at a position corresponding to K6 of SEQ ID NO: 145; the amino acid sequence of SEQ ID NO: 144, with an unnatural amino acid (UAA) at a position corresponding to K44 of SEQ ID NO: 144; the amino acid sequence of SEQ ID NO: 130, with an unnatural amino acid (UAA) at a position corresponding to K157 of SEQ ID NO: 130; or the amino acid sequence of SEQ ID NO: 121, with an unnatural amino acid (UAA) at a position corresponding to K277 of SEQ ID NO: 121;
(xxi) the amino acid sequence of SEQ ID NO: 146, with an unnatural amino acid (UAA) at a position corresponding to Y6 of SEQ ID NO: 146; the amino acid sequence of SEQ ID NO: 144, with an unnatural amino acid (UAA) at a position corresponding to Y48 of SEQ ID NO: 144; the amino acid sequence of SEQ ID NO: 130, with an unnatural amino acid (UAA) at a position corresponding to Y161 of SEQ ID NO: 130; or the amino acid sequence of SEQ ID NO: 121, with an unnatural amino acid (UAA) at a position corresponding to Y281 of SEQ ID NO: 121;
(xxii) the amino acid sequence of SEQ ID NO: 147, with an unnatural amino acid (UAA) at a position corresponding to K6 of SEQ ID NO: 147; the amino acid sequence of SEQ ID NO: 144, with an unnatural amino acid (UAA) at a position corresponding to K58 of SEQ ID NO: 144; the amino acid sequence of SEQ ID NO: 130, with an unnatural amino acid (UAA) at a position corresponding to K171 of SEQ ID NO: 130; or the amino acid sequence of SEQ ID NO: 121, with an unnatural amino acid (UAA) at a position corresponding to K291 of SEQ ID NO: 121;
(xxiii) the amino acid sequence of SEQ ID NO: 148, with an unnatural amino acid (UAA) at a position corresponding to K6 of SEQ ID NO: 148; the amino acid sequence of SEQ ID NO: 144, with an unnatural amino acid (UAA) at a position corresponding to K60 of SEQ ID NO: 144; the amino acid sequence of SEQ ID NO: 130, with an unnatural amino acid (UAA) at a position corresponding to K173 of SEQ ID NO: 130; or the amino acid sequence of SEQ ID NO: 121, with an unnatural amino acid (UAA) at a position corresponding to K293 of SEQ ID NO: 121;
(xxiv) the amino acid sequence of SEQ ID NO: 149, with an unnatural amino acid (UAA) at a position corresponding to N6 of SEQ ID NO: 149; the amino acid sequence of SEQ ID NO: 144, with an unnatural amino acid (UAA) at a position corresponding to N67 of SEQ ID NO: 144; the amino acid sequence of SEQ ID NO: 130, with an unnatural amino acid (UAA) at a position corresponding to N180 of SEQ ID NO: 130; or the amino acid sequence of SEQ ID NO: 121, with an unnatural amino acid (UAA) at a position corresponding to N300 of SEQ ID NO: 121;
(xxv) the amino acid sequence of SEQ ID NO: 150, with an unnatural amino acid (UAA) at a position corresponding to F6 of SEQ ID NO: 150; the amino acid sequence of SEQ ID NO: 151, with an unnatural amino acid (UAA) at a position corresponding to F64 of SEQ ID NO: 151; the amino acid sequence of SEQ ID NO: 130, with an unnatural amino acid (UAA) at a position corresponding to F287 of SEQ ID NO: 130; or the amino acid sequence of SEQ ID NO: 121, with an unnatural amino acid (UAA) at a position corresponding to F407 of SEQ ID NO: 121; and/or
(xxvi) the amino acid sequence of SEQ ID NO: 191, with an unnatural amino acid (UAA) at a position corresponding to K6 of SEQ ID NO: 191; the amino acid sequence of SEQ ID NO: 129, with an unnatural amino acid (UAA) at a position corresponding to K16 of SEQ ID NO: 129; the amino acid sequence of SEQ ID NO: 130, with an unnatural amino acid (UAA) at a position corresponding to K16 of SEQ ID NO: 130; or the amino acid sequence of SEQ ID NO: 121, with an unnatural amino acid (UAA) at a position corresponding to K136 of SEQ ID NO: 121.

In another aspect, the invention provides an antibody comprising (e.g., in a light chain or a fragment thereof):
(i) the amino acid sequence of SEQ ID NO: 152, with an unnatural amino acid (UAA) at a position corresponding to V6 of SEQ ID NO: 152; the amino acid sequence of SEQ ID NO: 153, with an unnatural amino acid (UAA) at a position corresponding to V15 of SEQ ID NO: 153; the amino acid sequence of SEQ ID NO: 154, with an unnatural amino acid (UAA) at a position corresponding to V15 of SEQ ID NO: 154; or the amino acid sequence of SEQ ID NO: 155, with an unnatural amino acid (UAA) at a position corresponding to V15 of SEQ ID NO: 155;
(ii) the amino acid sequence of SEQ ID NO: 156, with an unnatural amino acid (UAA) at a position corresponding to T6 of SEQ ID NO: 156; the amino acid sequence of SEQ ID NO: 153, with an unnatural amino acid (UAA) at a position corresponding to T20 of SEQ ID NO: 153; the amino acid sequence of SEQ ID NO: 154, with an unnatural amino acid (UAA) at a position corresponding to T20 of SEQ ID NO: 154; or the amino acid sequence of SEQ ID NO: 155, with an unnatural amino acid (UAA) at a position corresponding to T20 of SEQ ID NO: 155;
(iii) the amino acid sequence of SEQ ID NO: 157, with an unnatural amino acid (UAA) at a position corresponding to R6 of SEQ ID NO: 157; the amino acid sequence of SEQ ID NO: 158, with an unnatural amino acid (UAA) at a position corresponding to R1 of SEQ ID NO: 158; the amino acid sequence of SEQ ID NO: 154, with an unnatural amino acid (UAA) at a position corresponding to R24 of SEQ ID NO: 154; or the amino acid sequence of SEQ ID NO: 155, with an unnatural amino acid (UAA) at a position corresponding to R24 of SEQ ID NO: 155;
(iv) the amino acid sequence of SEQ ID NO: 159, with an unnatural amino acid (UAA) at a position corresponding to S6 of SEQ ID NO: 159; the amino acid sequence of SEQ ID NO: 160, with an unnatural amino acid (UAA) at a position corresponding to S4 of SEQ ID NO: 160; the amino acid sequence of SEQ ID NO: 154, with an unnatural amino acid (UAA) at a position corresponding to S60 of SEQ ID NO: 154; or the amino acid sequence of SEQ ID NO: 155, with an unnatural amino acid (UAA) at a position corresponding to S60 of SEQ ID NO: 155;
(v) the amino acid sequence of SEQ ID NO: 161, with an unnatural amino acid (UAA) at a position corresponding to K6 of SEQ ID NO: 161; the amino acid sequence of SEQ ID NO: 162, with an unnatural amino acid (UAA) at a position corresponding to K10 of SEQ ID NO: 162; the amino acid sequence of SEQ ID NO: 154, with an unnatural amino acid (UAA) at a position corresponding to K107 of SEQ ID NO: 154; or the amino acid sequence of SEQ ID NO: 155, with an unnatural amino acid (UAA) at a position corresponding to K107 of SEQ ID NO: 155;
(vi) the amino acid sequence of SEQ ID NO: 163, with an unnatural amino acid (UAA) at a position corresponding to T6 of SEQ ID NO: 163; the amino acid sequence of SEQ ID NO: 162, with an unnatural amino acid (UAA) at a position corresponding to T12 of SEQ ID NO: 162; the amino acid sequence of SEQ ID NO: 154, with an unnatural amino acid (UAA) at a position corresponding to T109 of SEQ ID NO: 154; or the amino acid sequence of SEQ ID NO: 155, with an unnatural amino acid (UAA) at a position corresponding to T109 of SEQ ID NO: 155;
(vii) the amino acid sequence of SEQ ID NO: 164, with an unnatural amino acid (UAA) at a position corresponding to V6 of SEQ ID NO: 164; the amino acid sequence of SEQ ID NO: 162, with an unnatural amino acid (UAA) at a position corresponding to V13 of SEQ ID NO: 162; the amino acid sequence of SEQ ID NO: 154, with an unnatural amino acid (UAA) at a position corresponding to V110 of SEQ ID NO: 154; or the amino acid sequence of SEQ ID NO: 155, with an unnatural amino acid (UAA) at a position corresponding to V110 of SEQ ID NO: 155;
(viii) the amino acid sequence of SEQ ID NO: 165, with an unnatural amino acid (UAA) at a position corresponding to A6 of SEQ ID NO: 165; the amino acid sequence of SEQ ID NO: 166, with an unnatural amino acid (UAA) at a position corresponding to A1 of SEQ ID NO: 166; or the amino acid sequence of SEQ ID NO: 155, with an unnatural amino acid (UAA) at a position corresponding to A111 of SEQ ID NO: 155;
(ix) the amino acid sequence of SEQ ID NO: 167, with an unnatural amino acid (UAA) at a position corresponding to Q6 of SEQ ID NO: 167; the amino acid sequence of SEQ ID NO: 166, with an unnatural amino acid (UAA) at a position corresponding to Q37 of SEQ ID NO: 166; or the amino acid sequence of SEQ ID NO: 155, with an unnatural amino acid (UAA) at a position corresponding to Q147 of SEQ ID NO: 155;
(x) the amino acid sequence of SEQ ID NO: 168, with an unnatural amino acid (UAA) at a position corresponding to L6 of SEQ ID NO: 168; the amino acid sequence of SEQ ID NO: 166, with an unnatural amino acid (UAA) at a position corresponding to L44 of SEQ ID NO: 166; or the amino acid sequence of SEQ ID NO: 155, with an unnatural amino acid (UAA) at a position corresponding to L154 of SEQ ID NO: 155;
(xi) the amino acid sequence of SEQ ID NO: 169, with an unnatural amino acid (UAA) at a position corresponding to G6 of SEQ ID NO: 169; the amino acid sequence of SEQ ID NO: 166, with an unnatural amino acid (UAA) at a position corresponding to G47 of SEQ ID NO: 166; or the amino acid sequence of SEQ ID NO: 155, with an unnatural amino acid (UAA) at a position corresponding to G157 of SEQ ID NO: 155;
(xii) the amino acid sequence of SEQ ID NO: 175, with an unnatural amino acid (UAA) at a position corresponding to K6 of SEQ ID NO: 175; the amino acid sequence of SEQ ID NO: 166, with an unnatural amino acid (UAA) at a position corresponding to K59 of SEQ ID NO: 166; or the amino acid sequence of SEQ ID NO: 155, with an unnatural amino acid (UAA) at a position corresponding to K169 of SEQ ID NO: 155;
(xiii) the amino acid sequence of SEQ ID NO: 170, with an unnatural amino acid (UAA) at a position corresponding to A6 of SEQ ID NO: 170; the amino acid sequence of SEQ ID NO: 166, with an unnatural amino acid (UAA) at a position corresponding to A83 of SEQ ID NO: 166; or the amino acid sequence of SEQ ID NO: 155, with an unnatural amino acid (UAA) at a position corresponding to A193 of SEQ ID NO: 155;
(xiv) the amino acid sequence of SEQ ID NO: 171, with an unnatural amino acid (UAA) at a position corresponding to V6 of SEQ ID NO: 171; the amino acid sequence of SEQ ID NO: 166, with an unnatural amino acid (UAA) at a position corresponding to V95 of SEQ ID NO: 166; or the amino acid sequence of SEQ ID NO: 155, with an unnatural amino acid (UAA) at a position corresponding to V205 of SEQ ID NO: 155;
(xv) the amino acid sequence of SEQ ID NO: 172, with an unnatural amino acid (UAA) at a position corresponding to T6 of SEQ ID NO: 172; the amino acid sequence of SEQ ID NO: 166, with an unnatural amino acid (UAA) at a position corresponding to T96 of SEQ ID NO: 166; or the amino acid sequence of SEQ ID NO: 155, with an unnatural amino acid (UAA) at a position corresponding to T206 of SEQ ID NO: 155; and/or
(xvi) the amino acid sequence of SEQ ID NO: 173, with an unnatural amino acid (UAA) at a position corresponding to S6 of SEQ ID NO: 173; the amino acid sequence of SEQ ID NO: 166, with an unnatural amino acid (UAA) at a position corresponding to S98 of SEQ ID NO: 166; or the amino acid sequence of SEQ ID NO: 155, with an unnatural amino acid (UAA) at a position corresponding to S208 of SEQ ID NO: 155

In certain embodiments, the antibody comprises one, two, three, four, or more than four unnatural amino acids (UAAs).

In certain embodiments, the UAA is: (i) a tryptophan analog (e.g., 5-HTP and 5-AzW); (ii) a leucine analog (e.g., LCA and Cys-5-N3); (iii) a tyrosine analog (e.g., OmeY, AzF, and OpropY); or (iv) a pyrrolysine analog (e.g., Bock, CpK, and AzK).

In certain embodiments, the antibody further comprises a chemical modification of the unnatural amino acid (UAA), e.g., a conjugation to a molecule, e.g., a detectable label or a drug (e.g., a small molecule drug). In certain embodiments, the molecule is: AEB, AEVB, AFP, an amatoxin, an auristatin (e.g., auristatin E), a calicheamicin, CC-1065 or a CC-1065 analog, chalicheamicin, combretastatin, DM1, DM4, docetaxel, dolastatin-10, DUBA, a duocarmycin, echinomycin, FAM, maytansine, a maytansinoid, MMAD, MMAE, MMAF, a morpholino-doxorubicin (e.g., cyanomorpholino-doxorubicin), netropsin, an oligonucleotide (e.g., a DNA, RNA, or LNA oligonucleotide), paclitaxel, PBD, a peptide (e.g., a therapeutic peptide), rhizoxin, a small molecule (e.g., a therapeutic small molecule) SN-38, topotecan, a topoisomerase inhibitor, or a toxoid. In certain embodiments, the molecule is conjugated to the UAA by a linker, e.g., a cleavable linker, a non-cleavable linker, a peptide-based linker, or a PEG-based linker.

In certain embodiments, the antibody has an average drug antibody ratio (DAR) of at least 0.5, 0.6, 0.7, 0.8, 0.9, 1.0, 1.5, 1.6, 1.7, 1.8, 1.9, 2.0, 3.5, 3.6, 3.7, 3.8, 3.9, or 4.0, as measured by hydrophobic interaction chromatography (HIC). In certain embodiments, the antibody has an average drug antibody ratio (DAR) that is within 5%, 10%, 15%, 20%, 25%, 30%, 35%, or 40% of the number of UAAs in the antibody.

In certain embodiments, at least 40%, 50%, 60%, 70%, 80%, or 90% of the antibody remains following incubation in human plasma for 72 hours at 37° C. In certain embodiments, at least 40%, 50%, 60%, 70%, 80%, or 90% of the antibody remains following incubation with Cathepsin B for 240 minutes at 37° C.

In certain embodiments, the antibody has a binding affinity for a target antigen of 20 nM, 15 nM, 10 nM, 9 nM, 8 nM, 7 nM, 6 nM, 5 nM, 4 nM, 3 nM, 2 nM, 1 nM, 0.75 nM, 0.5 nM, 0.1 nM, 0.075 nM, or 0.05 nM or lower, as measured by enzyme-linked immunosorbent assay (ELISA). In certain embodiments, the antibody has a binding affinity for a target antigen that is within 0.1 fold, 0.2 fold, 0.3 fold, 0.4 fold, 0.5 fold, 1.0 fold, 1.5 fold, 2.0 fold, 3.0 fold, 4.0 fold, 5.0 fold, 6.0 fold, 8.0 fold, or 10.0 fold of the binding affinity for the target antigen of a reference antibody, wherein the reference antibody is an otherwise identical antibody that does not comprise the UAA, as measured by ELISA.

In certain embodiments, the antibody is an IgG1, IgG2, IgG3, or IgG4 antibody.

In another aspect, the invention provides a method of producing any of the foregoing antibodies. The method comprises culturing a cell with: (i) a nucleic acid comprising a nucleotide sequence encoding a tRNA comprising an anticodon that hybridizes to a codon selected from UAG, UGA, and UAA, and is capable of being charged with the unnatural amino acid (UAA); (ii) a nucleic acid comprising a nucleotide sequence encoding an aminoacyl-tRNA synthetase capable of charging the tRNA with the unnatural amino acid (UAA); and (iii) a nucleic acid comprising a nucleotide sequence encoding a heavy chain, a light chain, or a combination of a heavy chain and light chain of the antibody and comprising the codon selected from UAG, UGA, and UAA; under conditions that permit the tRNA, when expressed in the cell and charged with the unnatural amino acid (UAA), to hybridize to the codon and direct the incorporation of the unnatural amino acid (UAA) into the antibody.

In certain embodiments, the amount of the antibody comprising the unnatural amino acid (UAA) expressed by the cell is at least 10%, 20%, 30%, 40%, 50%, 60%, 70%, 80%, or 90% of the amount of a reference antibody expressed by the same cell or a similar cell. In certain embodiments, the reference antibody is an otherwise identical antibody that does not comprise the UAA, for example, the reference antibody comprises a wild-type amino acid residue at the position corresponding to the unnatural amino acid (UAA).

In certain embodiments, following protein G purification (e.g., following only protein G purification) less than 10%, 9%, 8%, 7%, 6%, 5%, 4%, 3%, 2%, or 1% of the antibody is aggregated, as measured by size exclusion chromatography (SEC).

In certain embodiments, the tRNA is an analog or derivative of a prokaryotic tryptophanyl-tRNA, e.g., an E. coli tryptophanyl-tRNA. For example, the tRNA may comprise a nucleotide sequence selected from any one of SEQ ID NOs: 49-54 or 108-113. In certain embodiments, the aminoacyl-tRNA synthetase is an analog or derivative of a prokaryotic tryptophanyl-tRNA synthetase, e.g., an E. coli tryptophanyl-tRNA synthetase. For example, the aminoacyl-tRNA synthetase may comprise an amino acid sequence selected from any one of SEQ ID NOs: 44-48. In certain embodiments, the codon is UGA. In certain embodiments, the UAA is a tryptophan analog, e.g., a non-naturally occurring tryptophan analog. In certain embodiments, the UAA is 5-HTP or 5-AzW.

In certain embodiments, the tRNA is an analog or derivative of a prokaryotic leucyl-tRNA, e.g., an E. coli leucyl-tRNA. For example, the tRNA may comprise a nucleotide sequence selected from any one of SEQ ID NOs: 16-43. In certain embodiments, the aminoacyl-tRNA synthetase is an analog or derivative of a prokaryotic leucyl-tRNA synthetase, e.g., an E. coli leucyl-tRNA synthetase. For example, the aminoacyl-tRNA synthetase may comprise an amino acid sequence selected from any one of SEQ ID NOs: 1-15. In certain embodiments, the codon is UAG. In certain embodiments, the UAA is a leucine analog, e.g., a non-naturally occurring leucine analog. In certain embodiments, the UAA is LCA or Cys-5-N3.

In certain embodiments, the tRNA is an analog or derivative of a prokaryotic tyrosyl-tRNA, e.g., an E. coli tyrosyl-tRNA. For example, the tRNA may comprise a nucleotide sequence selected from any one of SEQ ID NOs: 68-69 or 104-105. In certain embodiments, the aminoacyl-tRNA synthetase is an analog or derivative of a prokaryotic tyrosyl-tRNA synthetase, e.g., an E. coli tyrosyl-tRNA synthetase. For example, the aminoacyl-tRNA synthetase may comprise the amino acid sequence of SEQ ID NO: 70. In certain embodiments, the codon is UAG. In certain embodiments, the UAA is a tyrosine analog, e.g., a non-naturally occurring tyrosine analog. In certain embodiments, the UAA is OmeY, AzF, or OpropY.

In certain embodiments, the tRNA is an analog or derivative of an archael pyrrolysyl-tRNA, e.g., an M. barkeri pyrrolysyl-tRNA. For example, the tRNA may comprise a nucleotide sequence selected from any one of SEQ ID NOs: 72-100 or 106-107. In certain embodiments, the aminoacyl-tRNA synthetase is an analog or derivative of an archael pyrrolysyl-tRNA synthetase, e.g., an M. barkeri pyrrolysyl-tRNA synthetase. For example, the aminoacyl-tRNA synthetase may comprise the amino acid sequence of SEQ ID NO: 101. In certain embodiments, the codon is UAG. In certain embodiments, the UAA is a pyrrolysine analog, e.g., a non-naturally occurring pyrrolysine analog. In certain embodiments, the UAA is BocK, CpK, or AzK.

In certain embodiments, the cell is a human cell, e.g., a human embryonic kidney (HEK) or a Chinese hamster ovary (CHO) cell.

In certain embodiments, the method further comprises contacting the cell with the UAA. In certain embodiments, the method further comprises purifying the antibody. In certain embodiments, the method further comprises chemically modifying the UAA, for example, conjugating the UAA to a conjugation to a molecule, e.g., a detectable label or a drug (e.g., a small molecule drug). In certain embodiments, the molecule is: AEB, AEVB, AFP, an amatoxin, an auristatin (e.g., auristatin E), a calicheamicin, CC-1065 or a CC-1065 analog, chalicheamicin, combretastatin, DM1, DM4, docetaxel, dolastatin-10, DUBA, a duocarmycin, echinomycin, FAM, maytansine, a maytansinoid, MMAD, MMAE, MMAF, a morpholino-doxorubicin (e.g., cyanomorpholino-doxorubicin), netropsin, an oligonucleotide (e.g., a DNA, RNA, or LNA oligonucleotide), paclitaxel, PBD, a peptide (e.g., a therapeutic peptide), rhizoxin, a small molecule (e.g., a therapeutic small molecule) SN-38, topotecan, a topoisomerase inhibitor, or a toxoid. In certain embodiments, the molecule is conjugated to the UAA by a linker, e.g., a cleavable linker, a non-cleavable linker, a peptide-based linker, or a PEG-based linker.

These and other aspects and features of the invention are described in the following detailed description and claims.

### DESCRIPTION OF THE DRAWINGS

The invention can be more completely understood with reference to the following drawings.

FIG. 1A depicts a schematic overview of genetic code expansion using unnatural amino acids (UAAs). FIG. 1B depicts a schematic overview of the antibody-drug conjugation methodologies described herein.

FIG. 2 depicts the heavy chain amino acid sequence and light chain sequence of Trastuzumab. Amino acid residues described in the present disclosure are bolded and underlined.

FIG. 3 depicts an enzyme-linked immunosorbent assay (ELISA) for various antibody-drug conjugates (ADCs) conjugated to the UAA LCA.

FIG. 4 depicts:

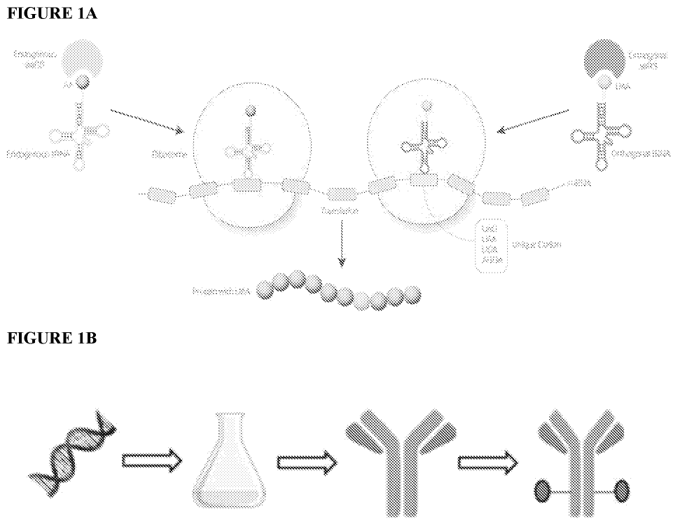

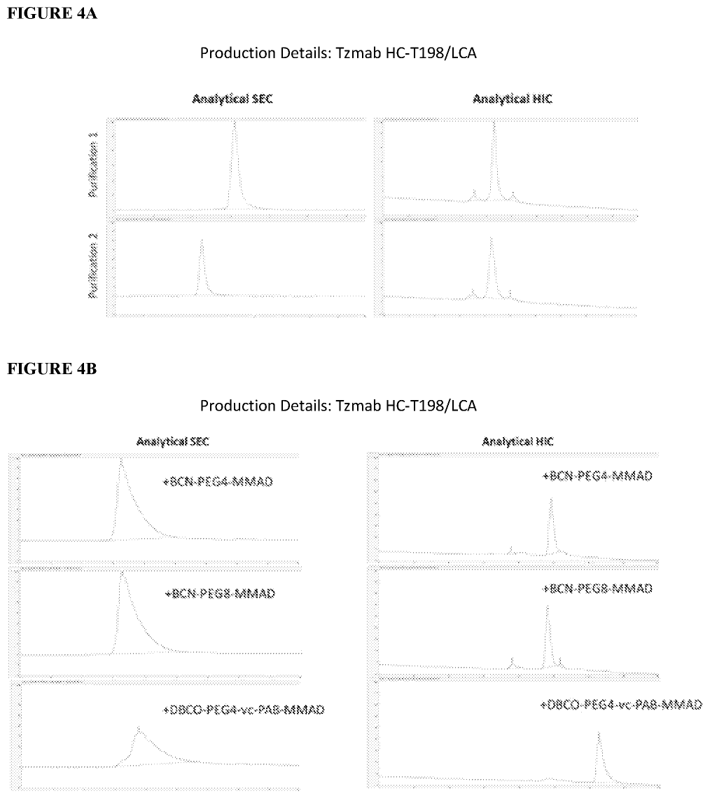

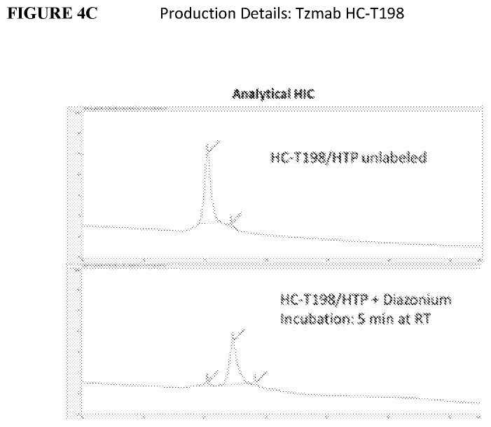

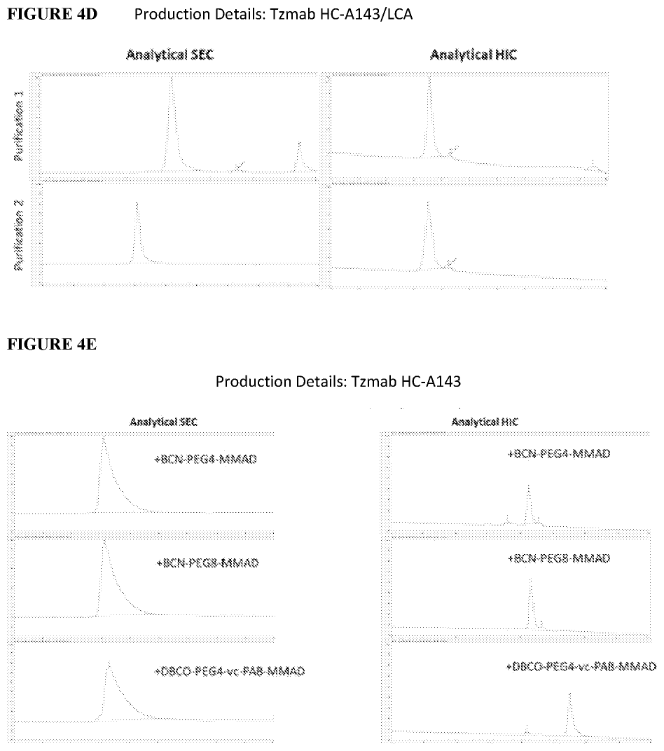

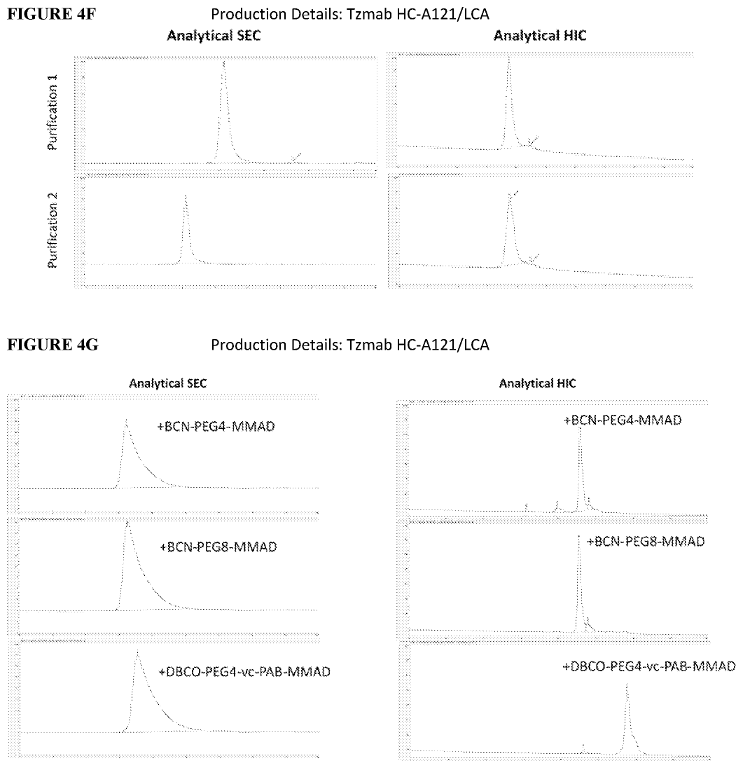

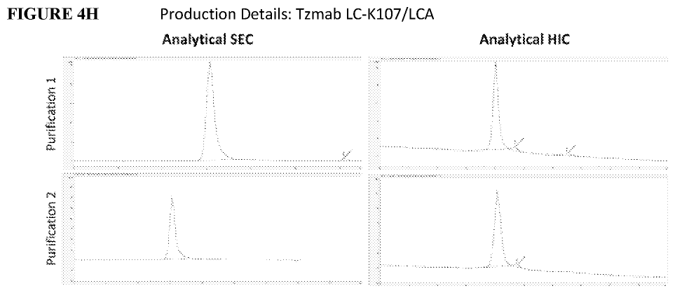

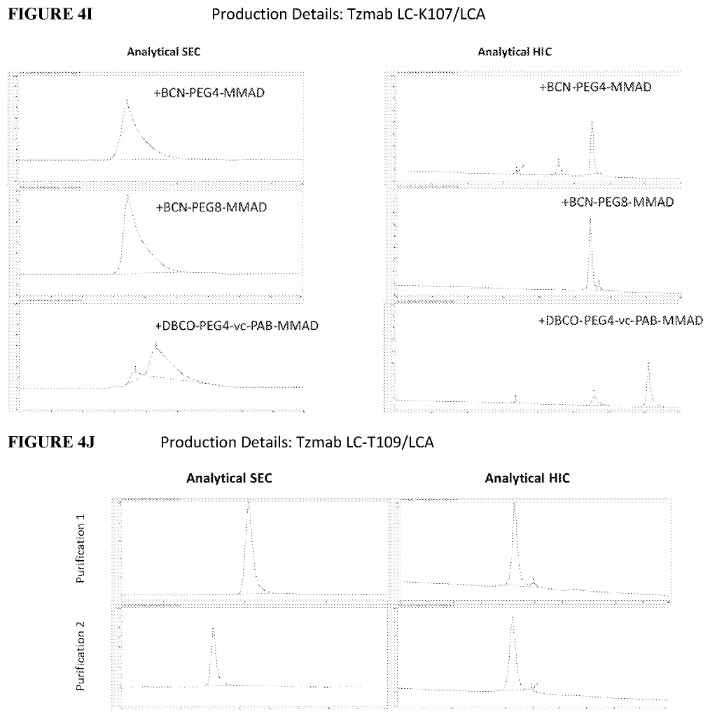

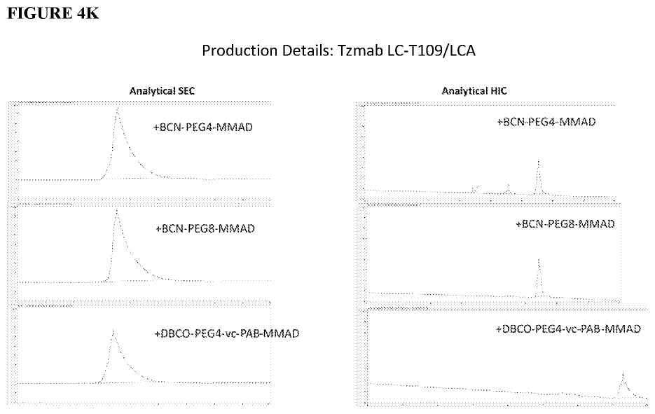

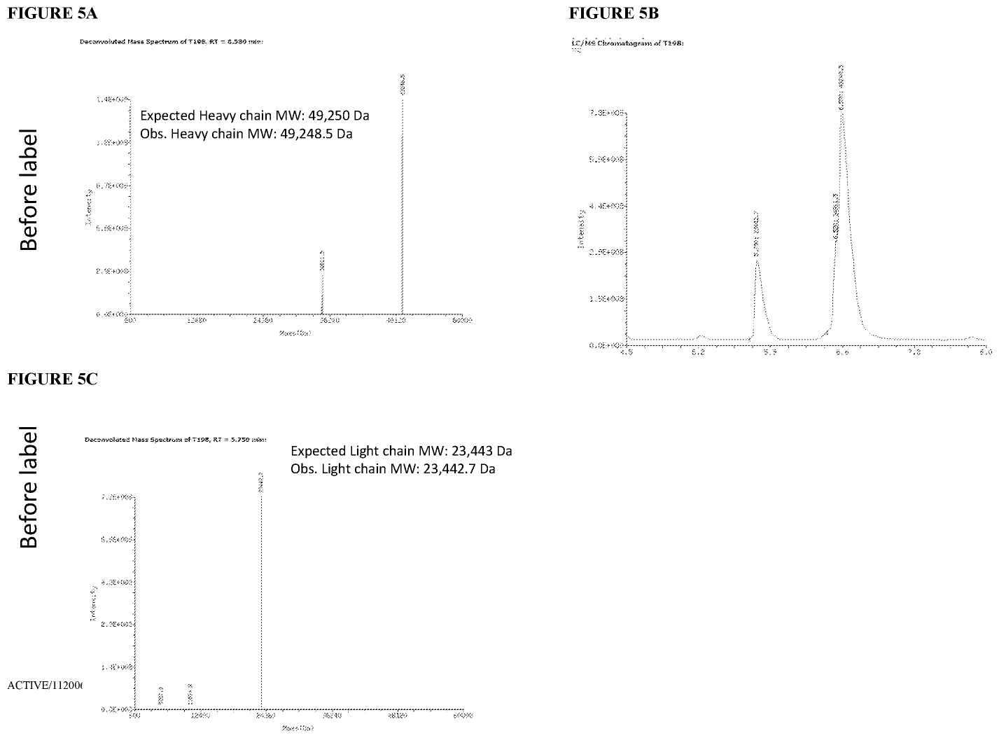

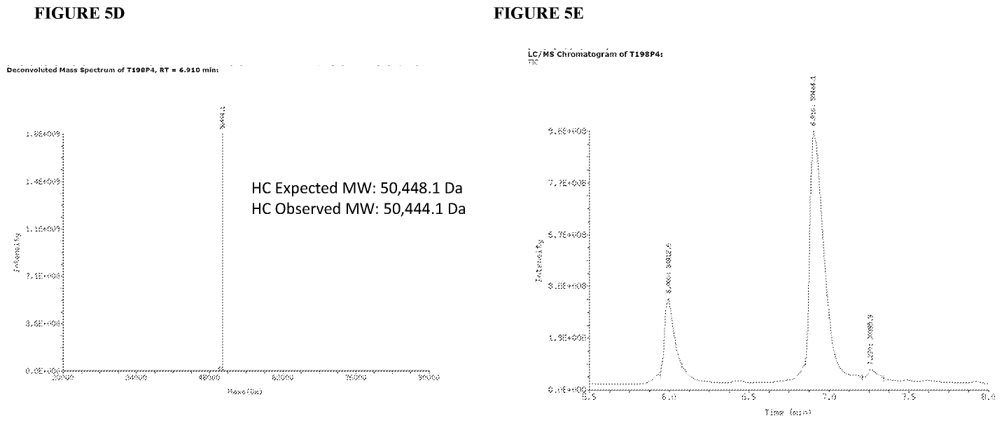

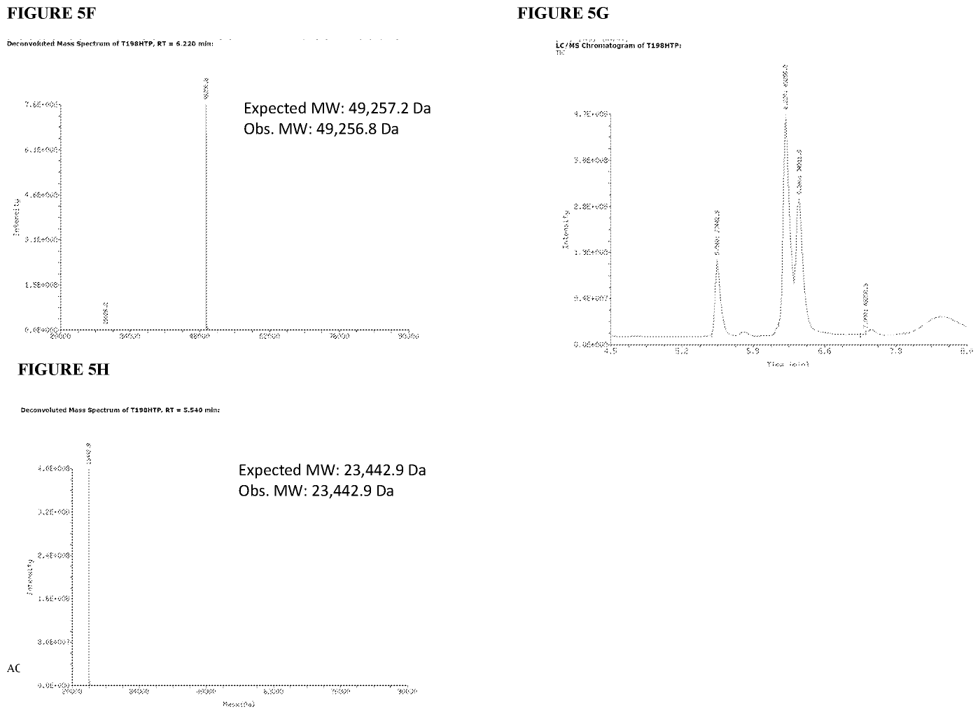

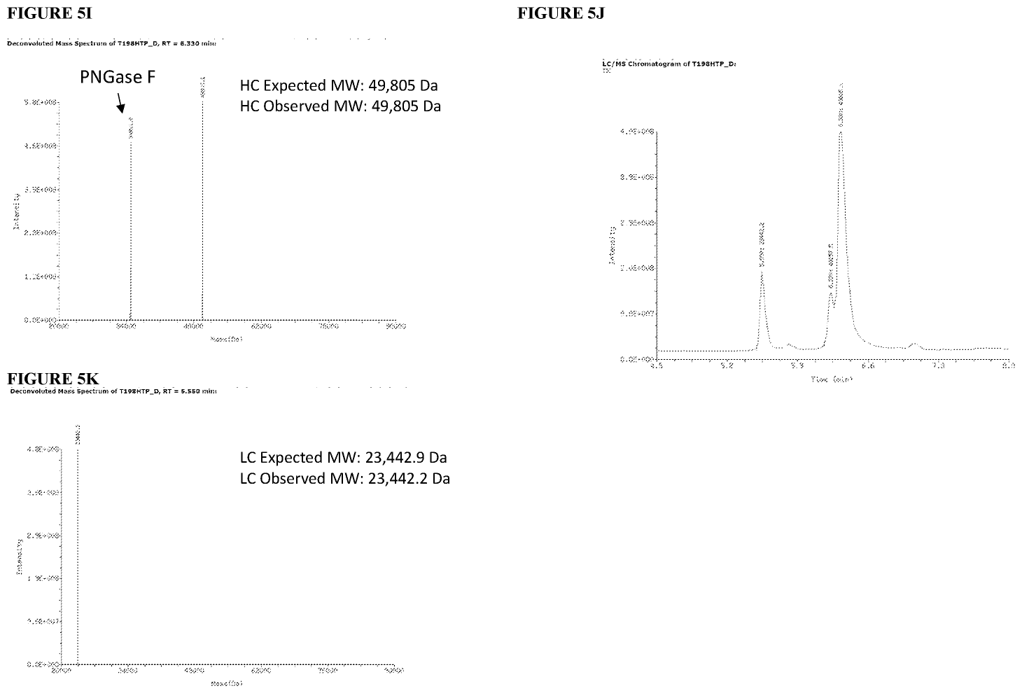

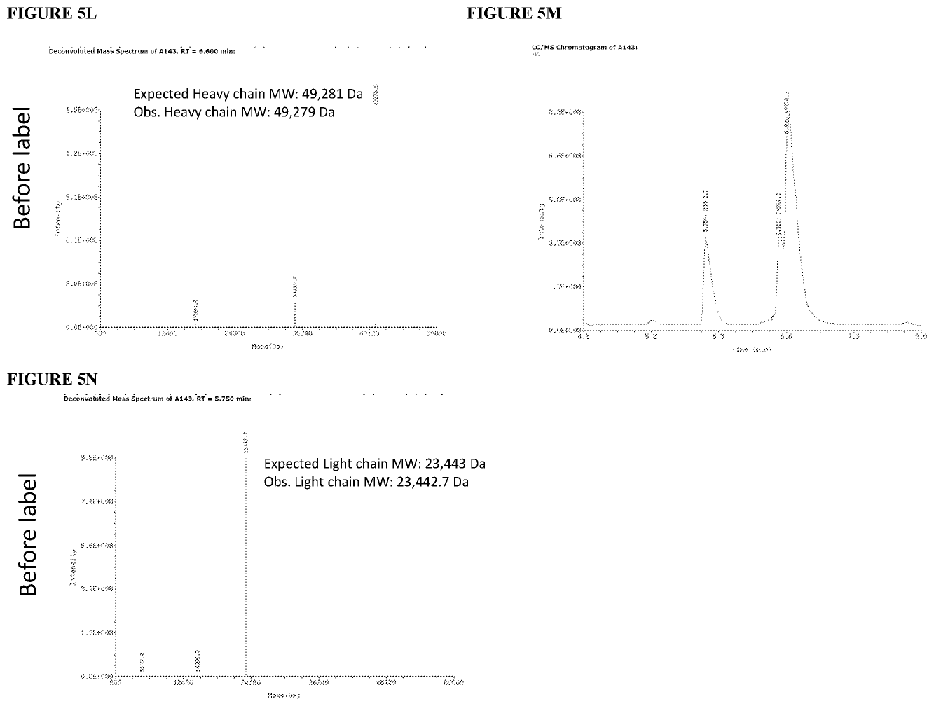

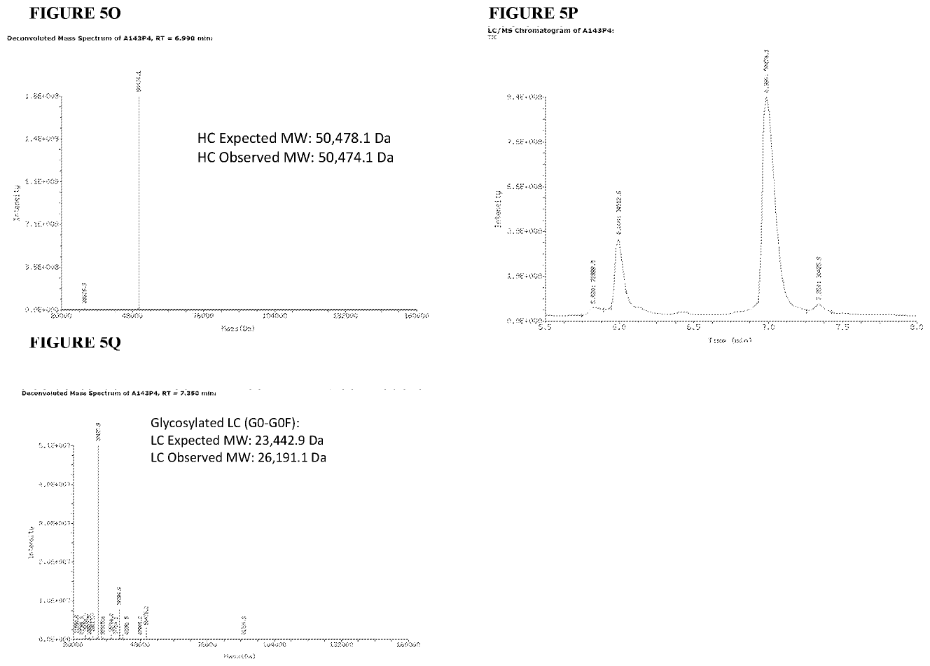

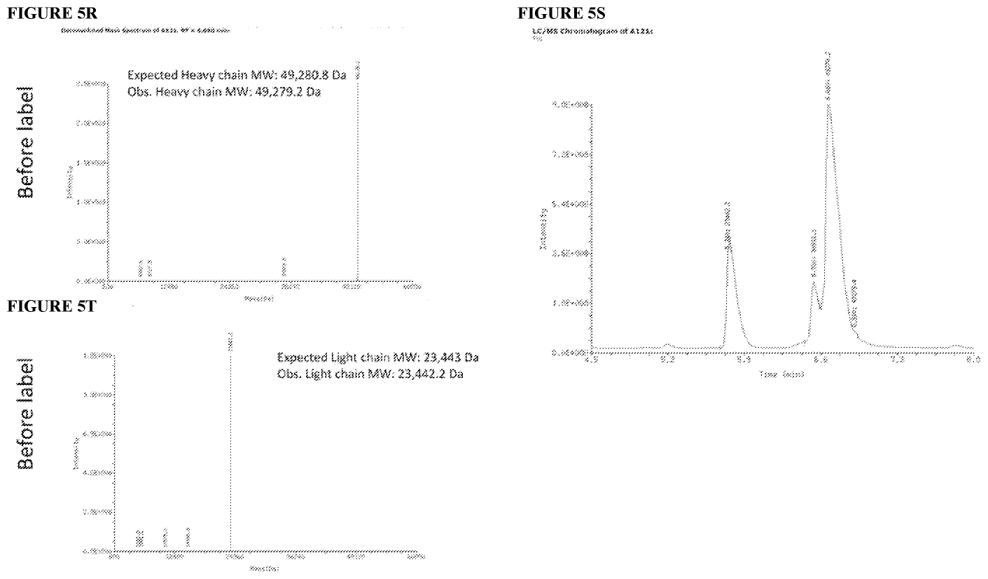

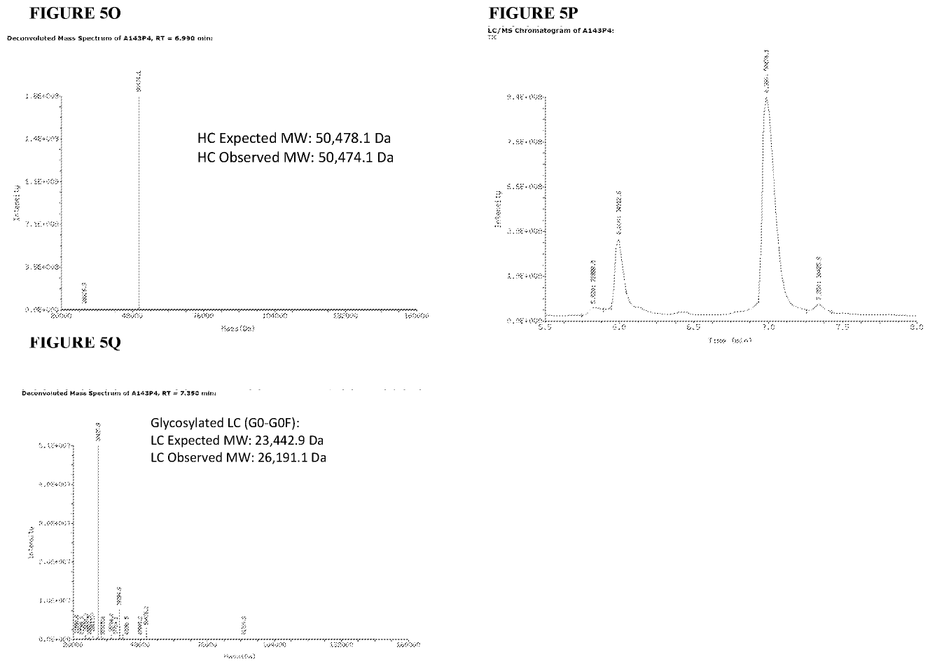

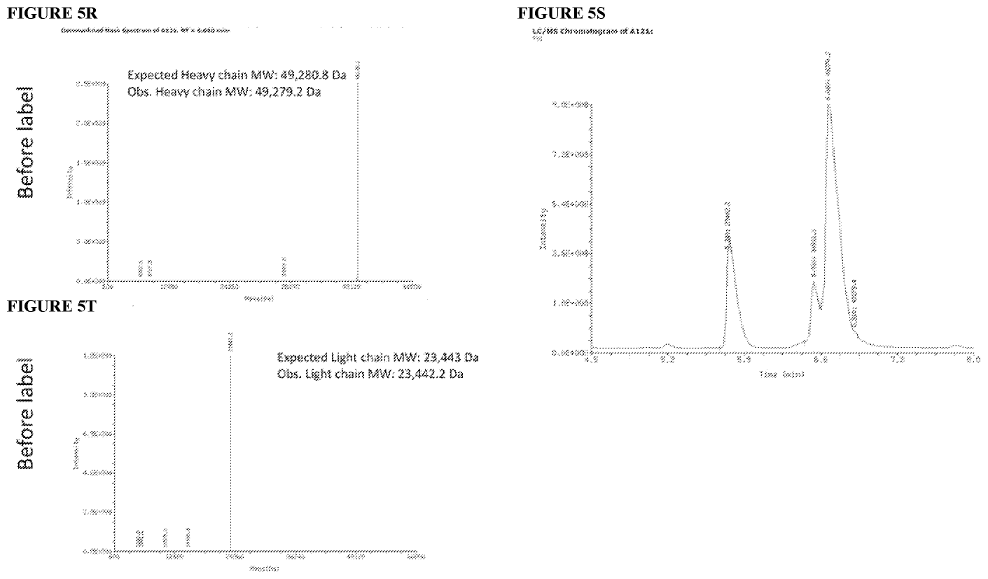

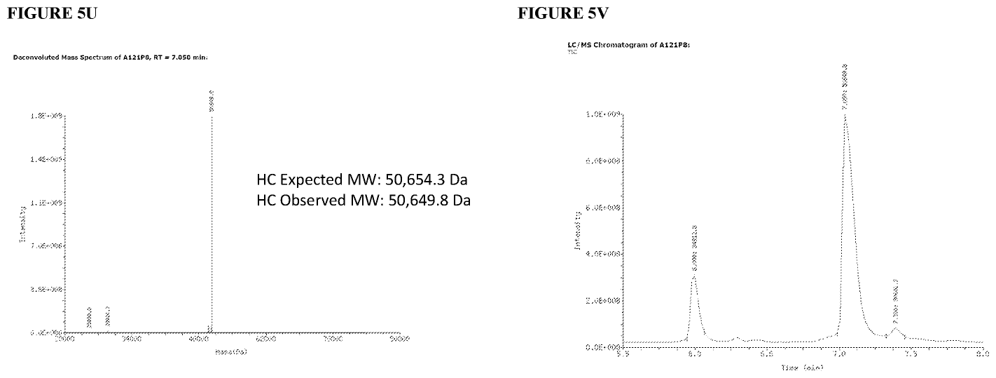

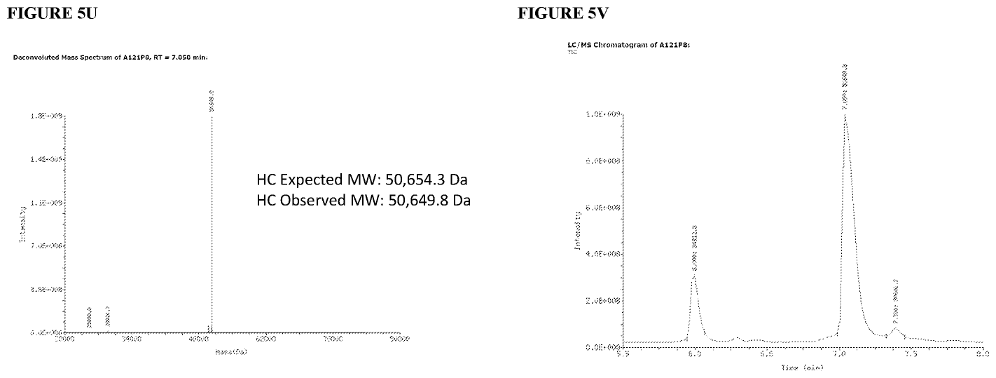

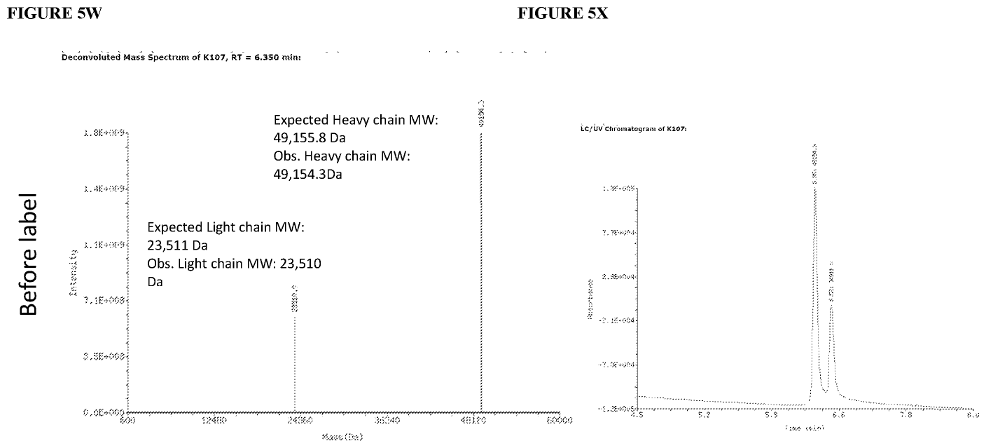

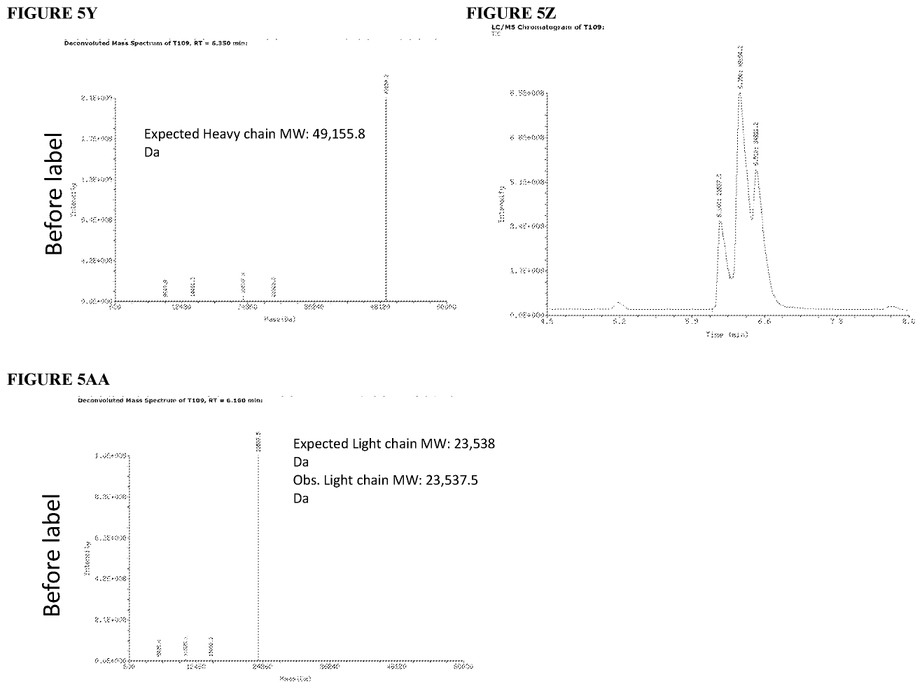

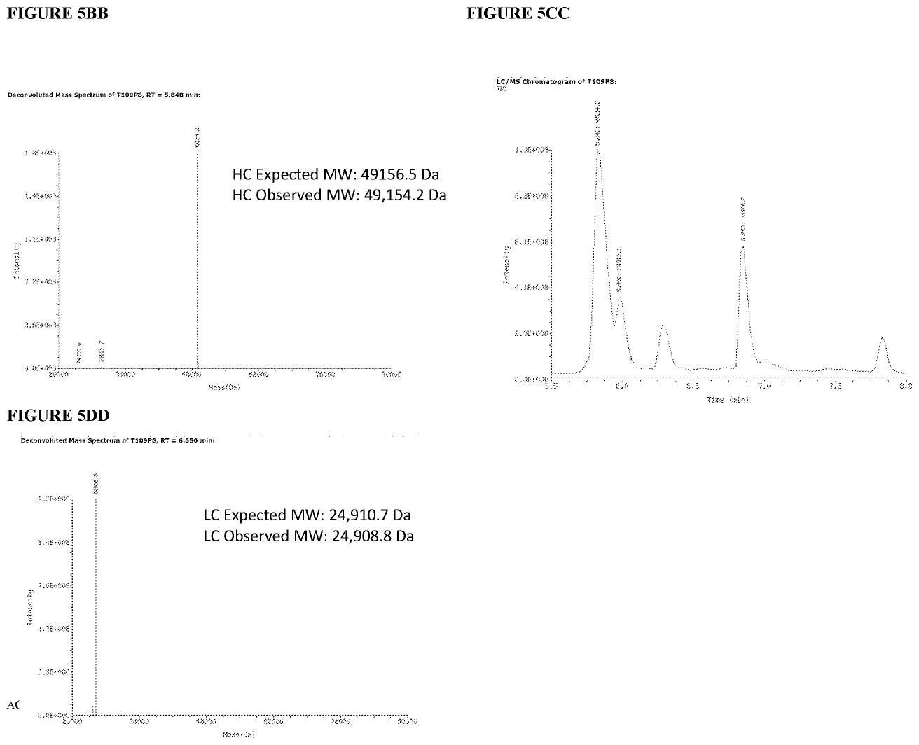

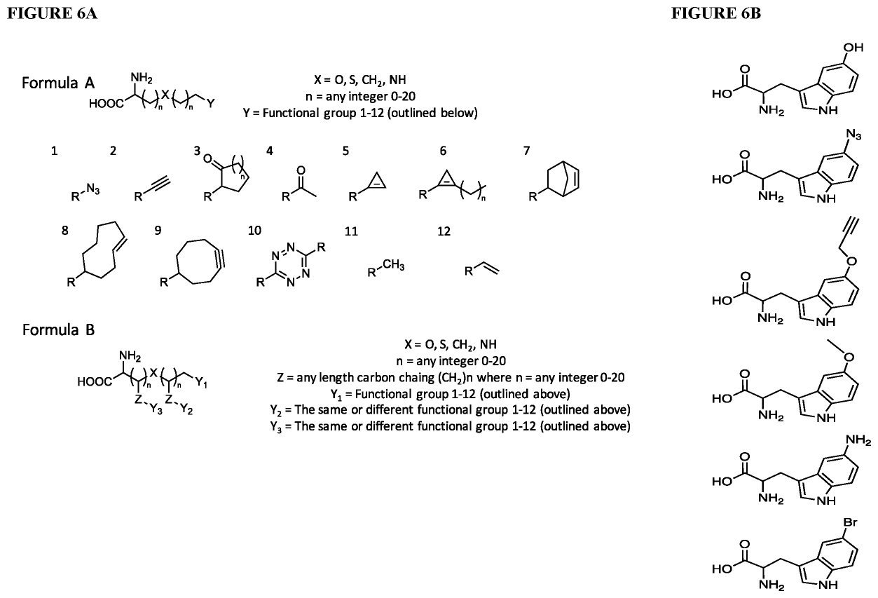

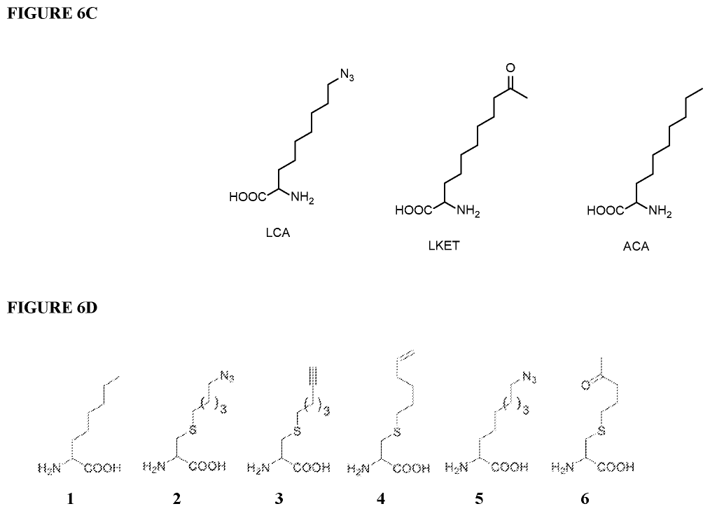

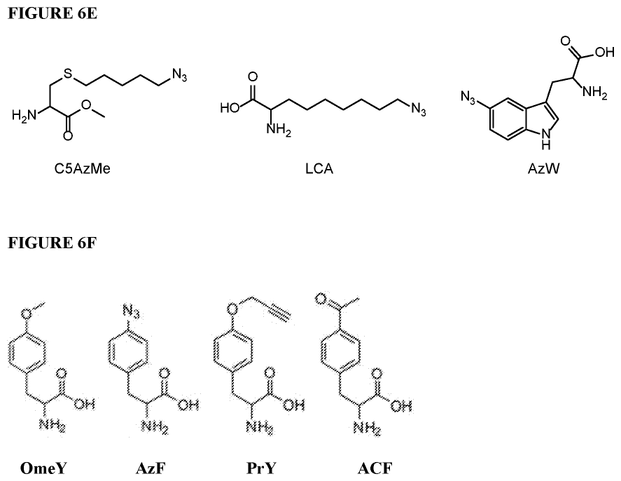

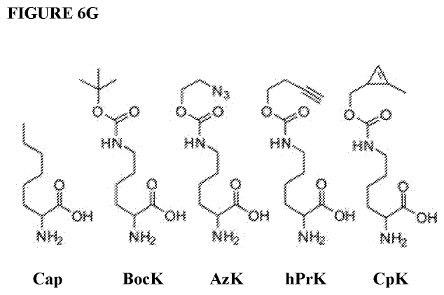

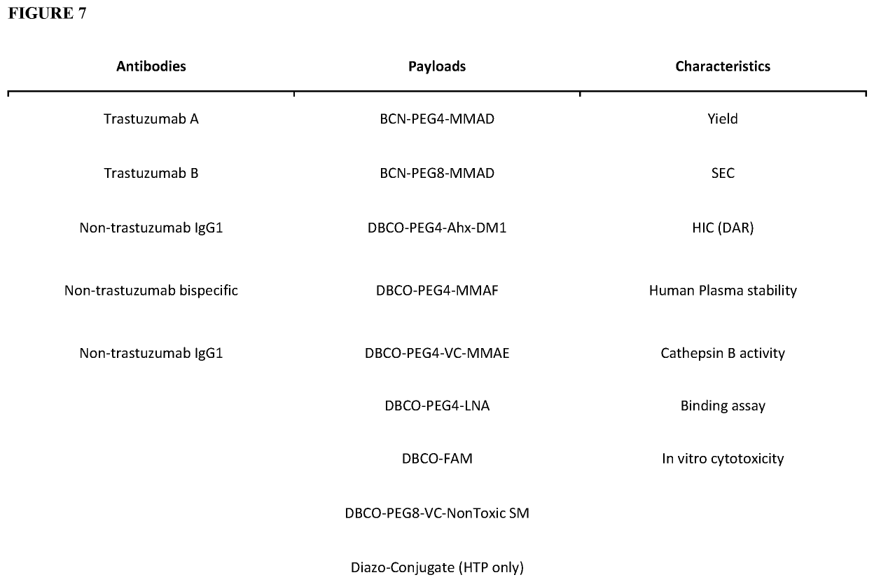

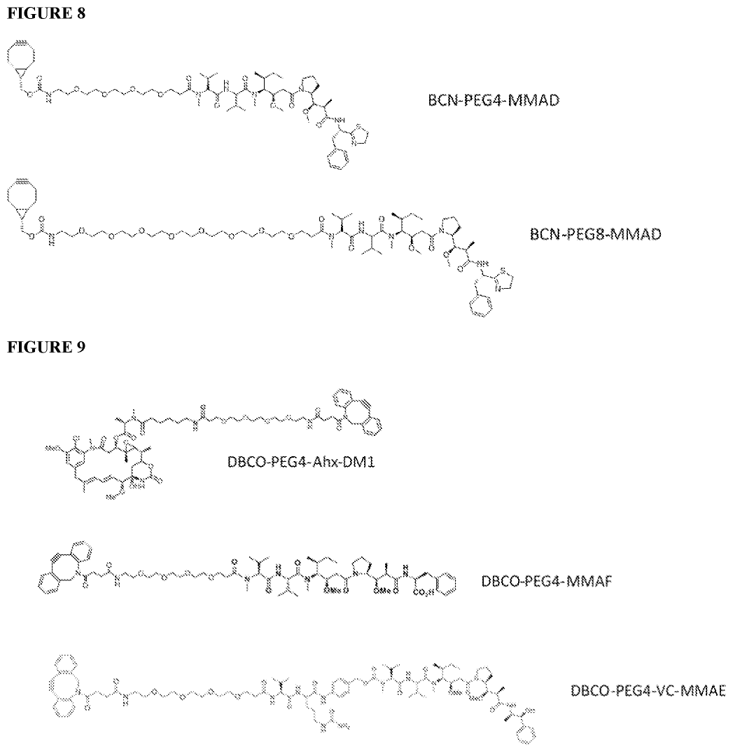

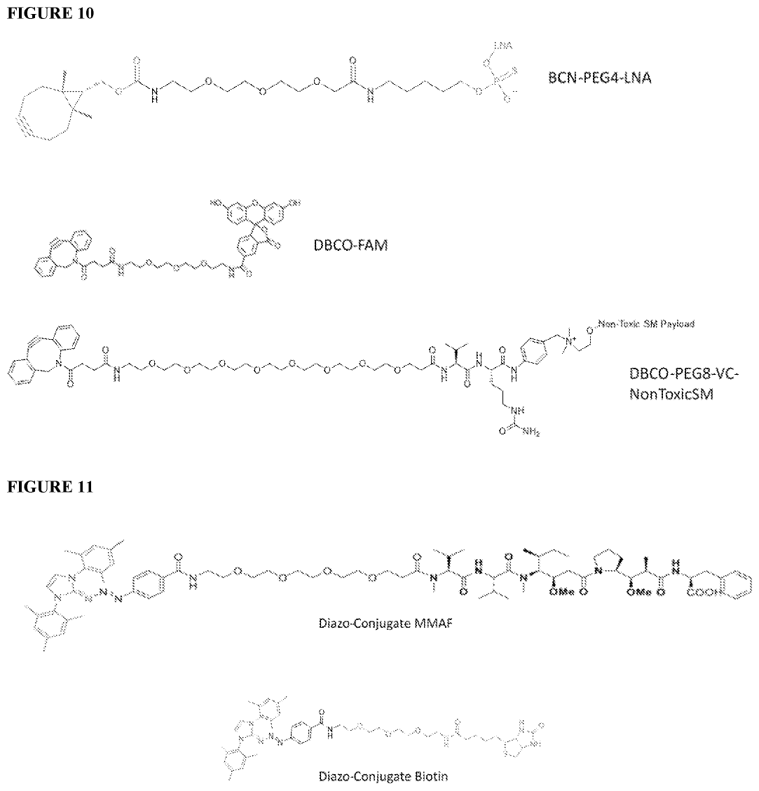

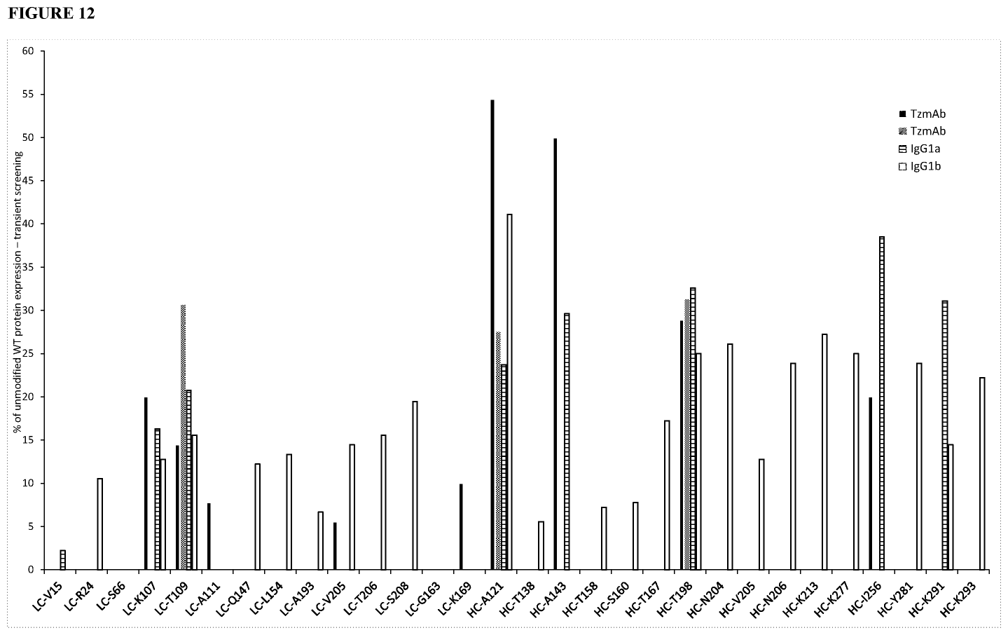

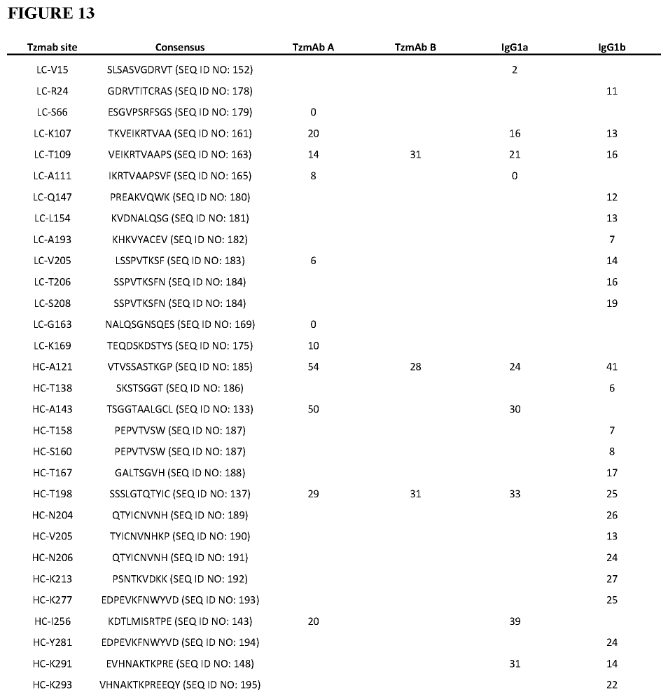

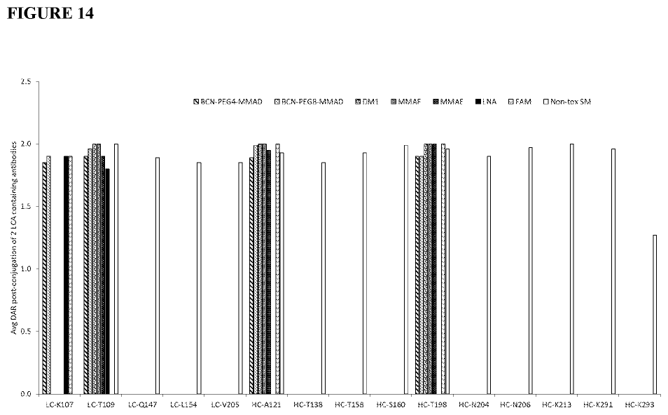

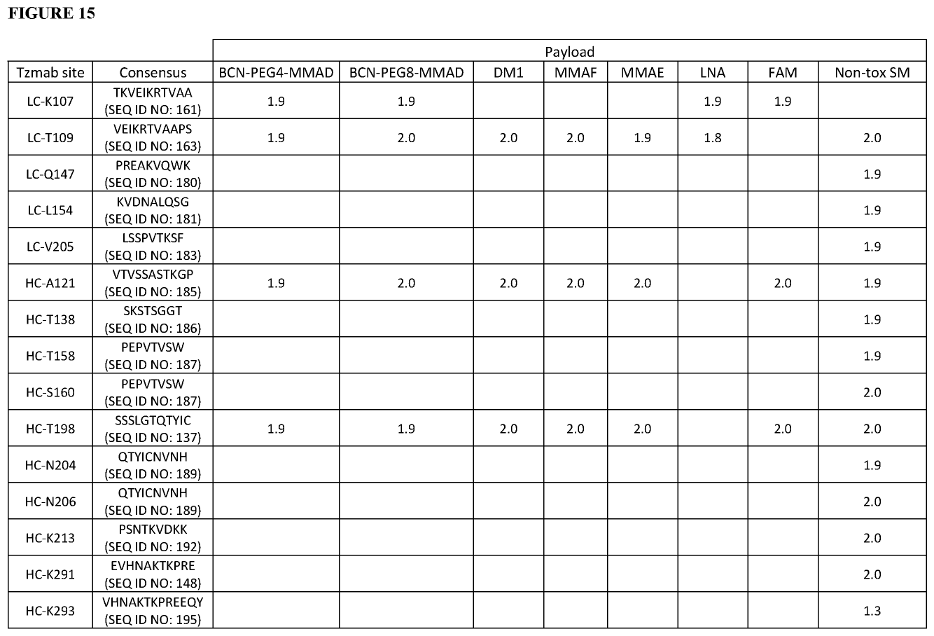

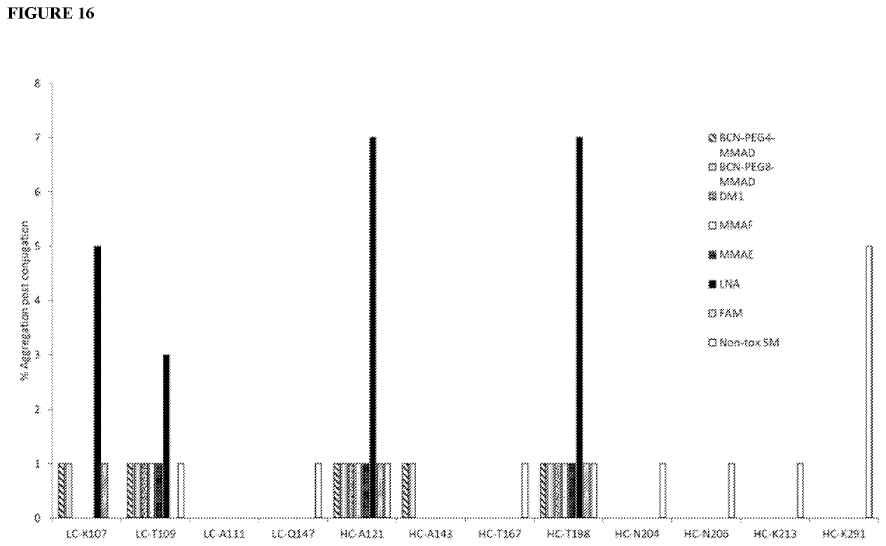

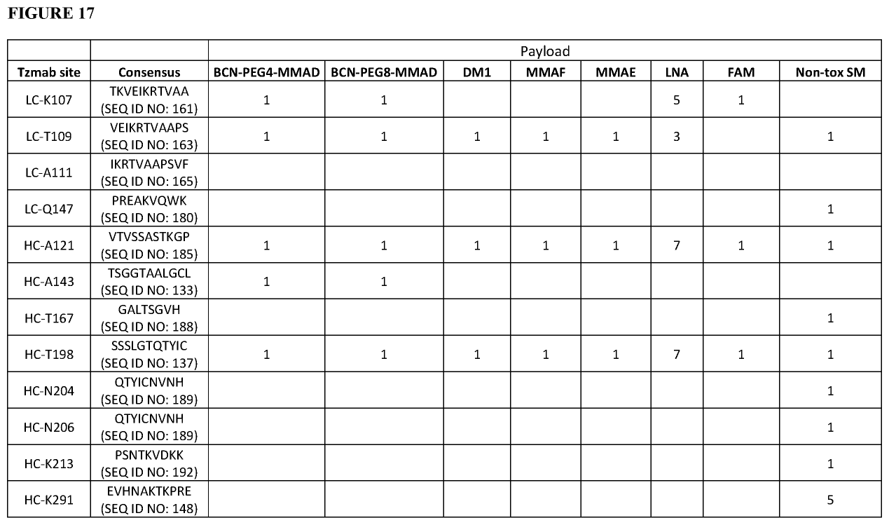

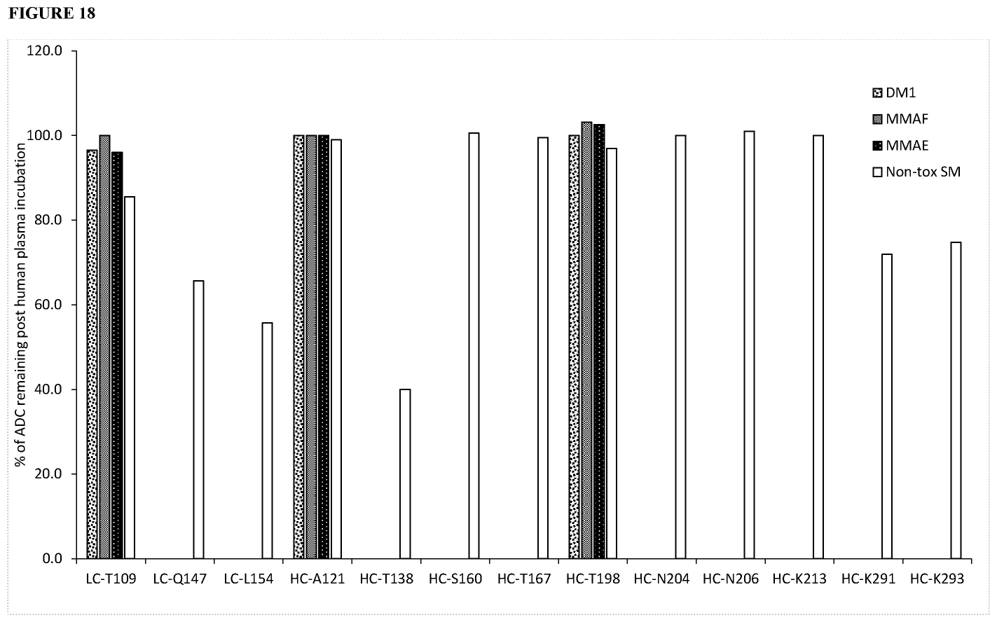

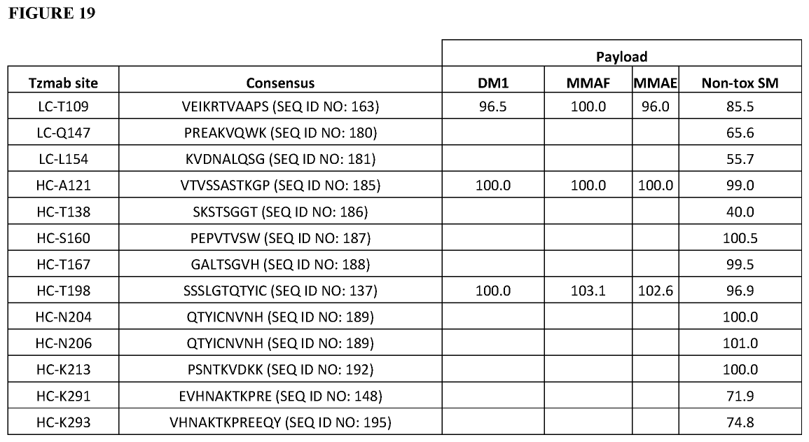

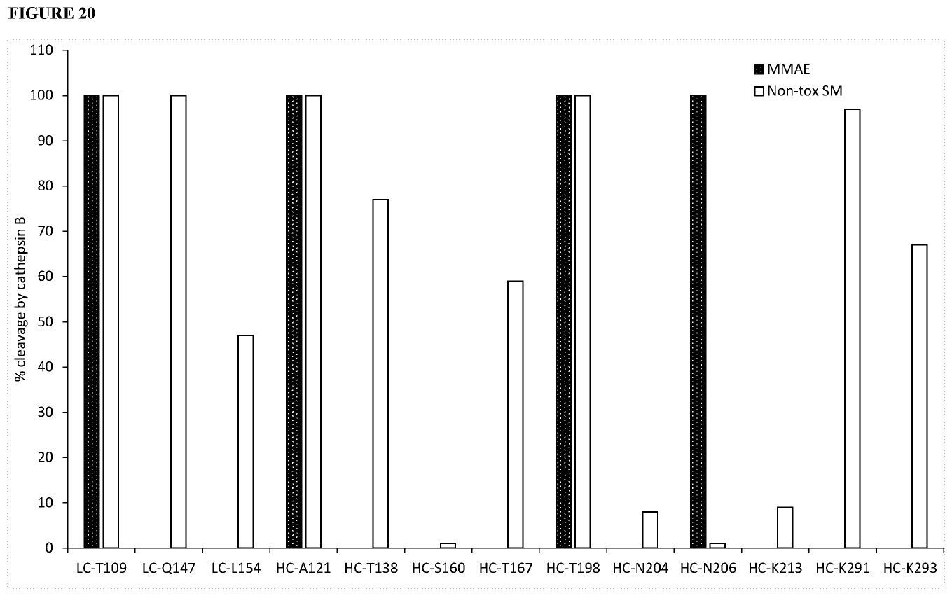

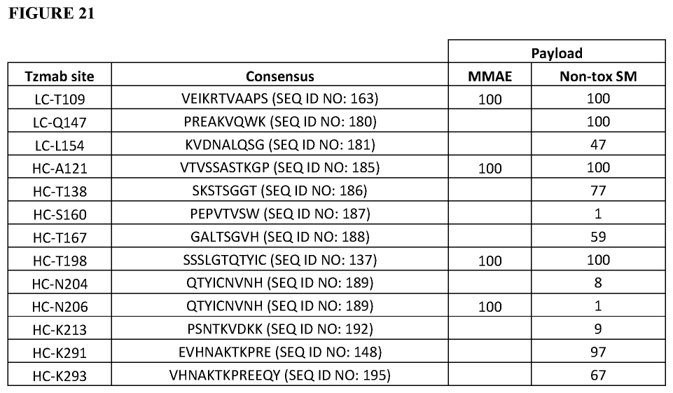

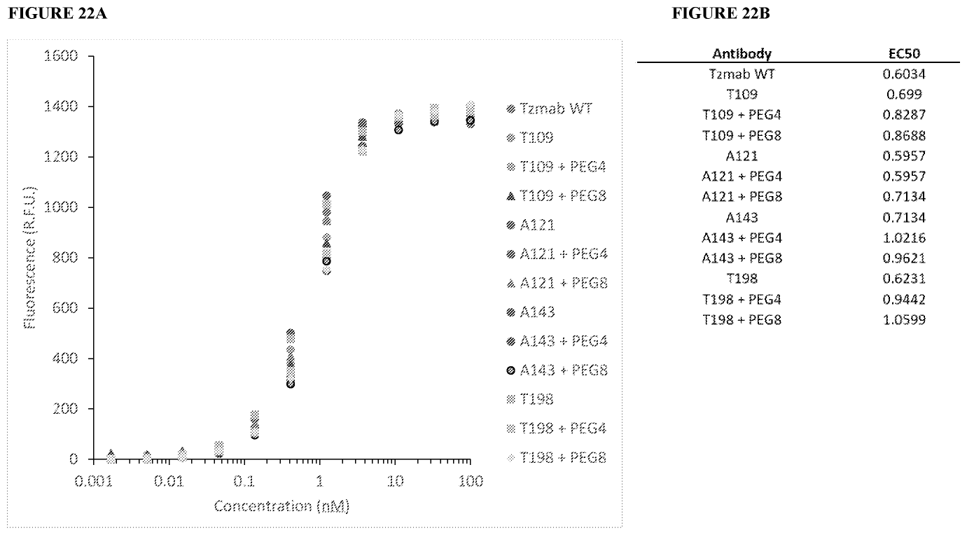

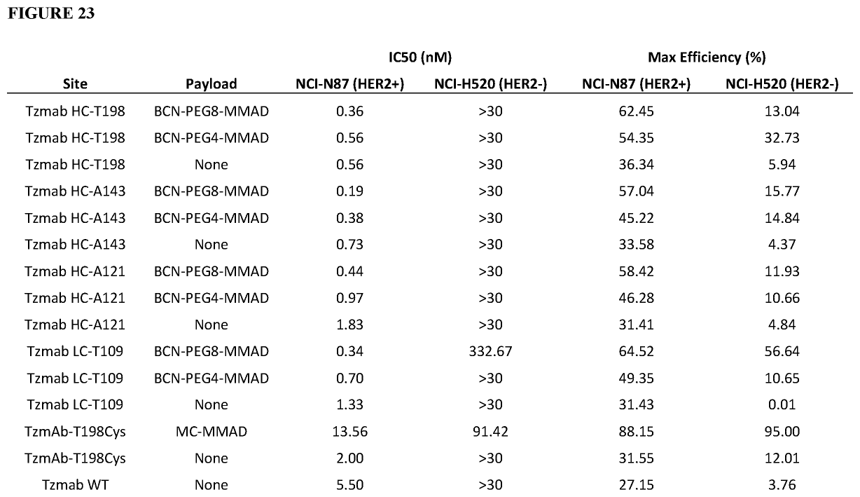

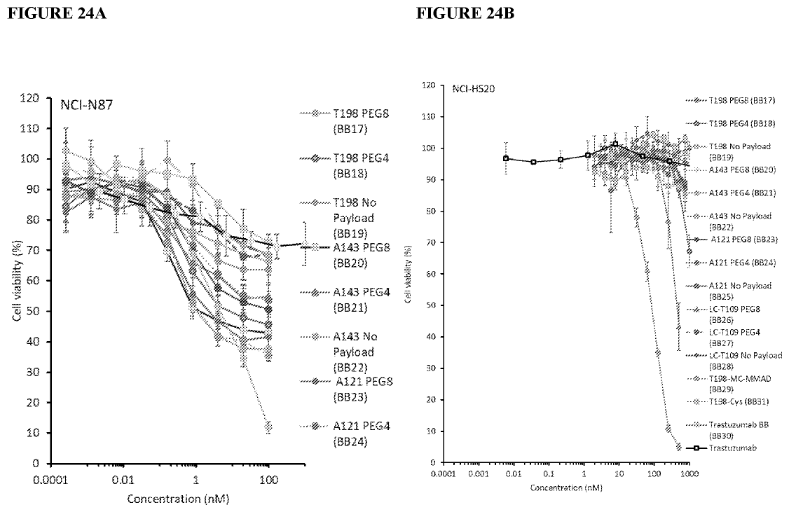

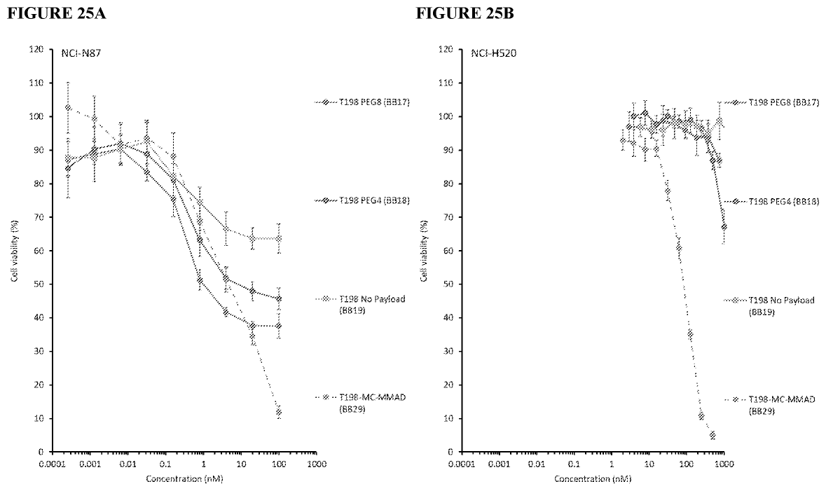

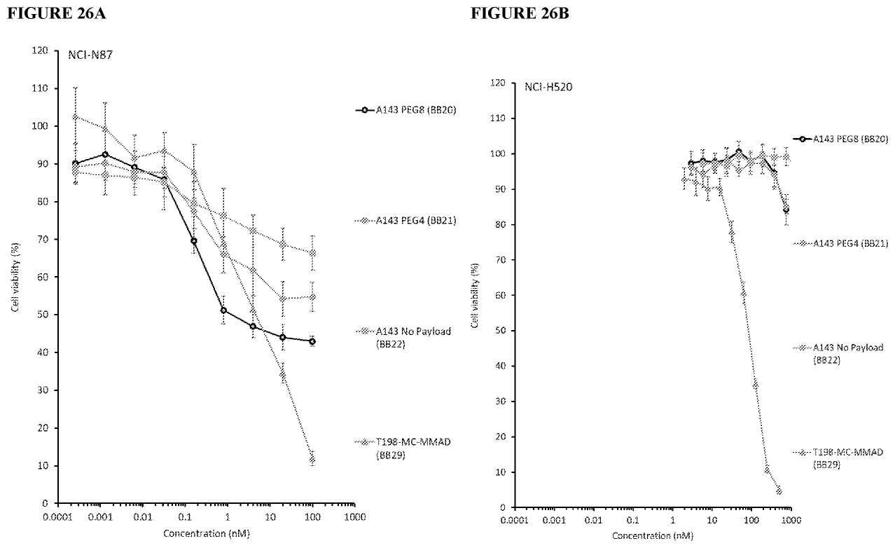

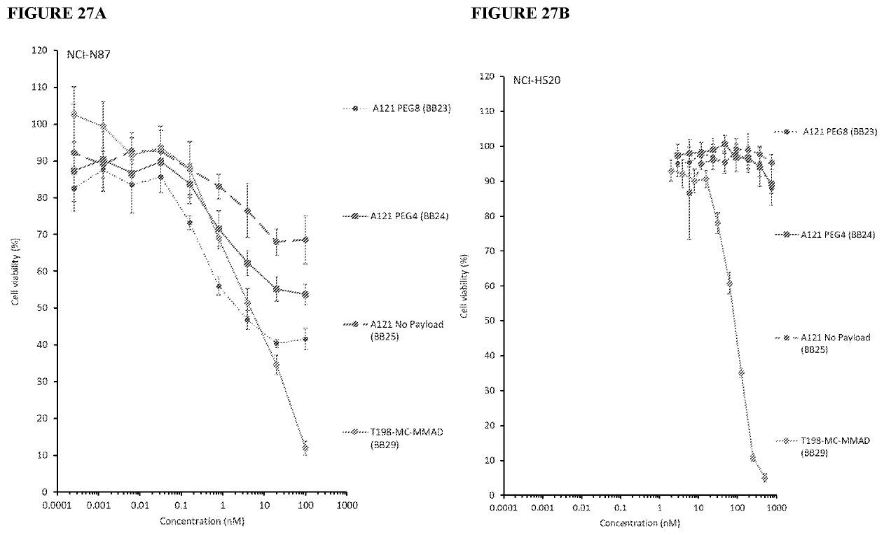

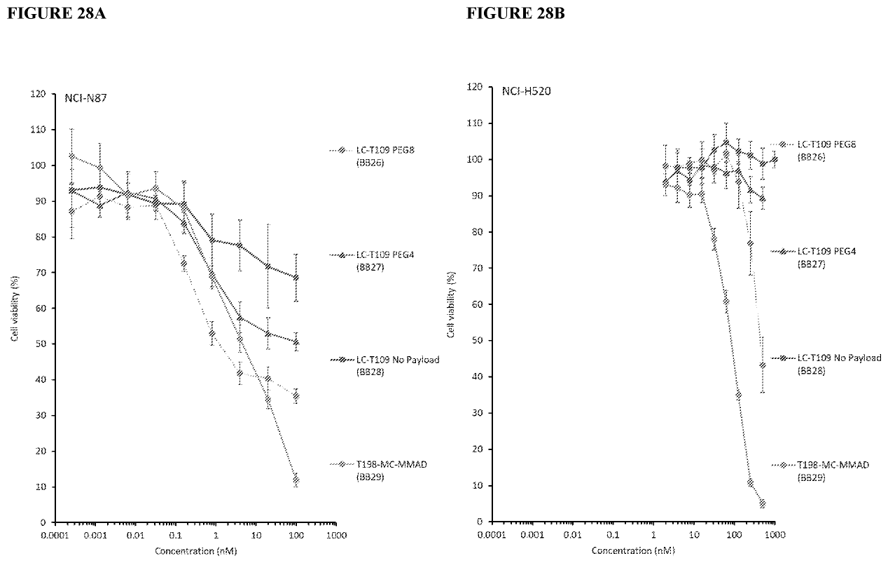

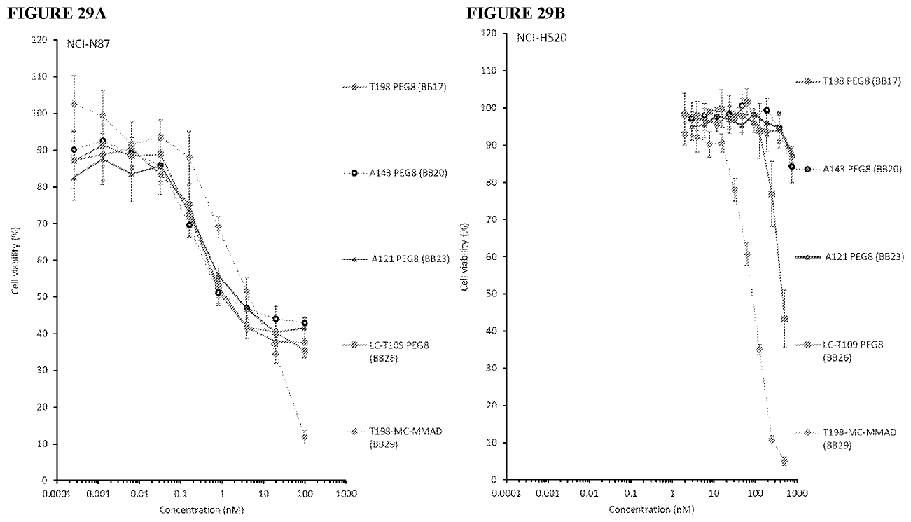

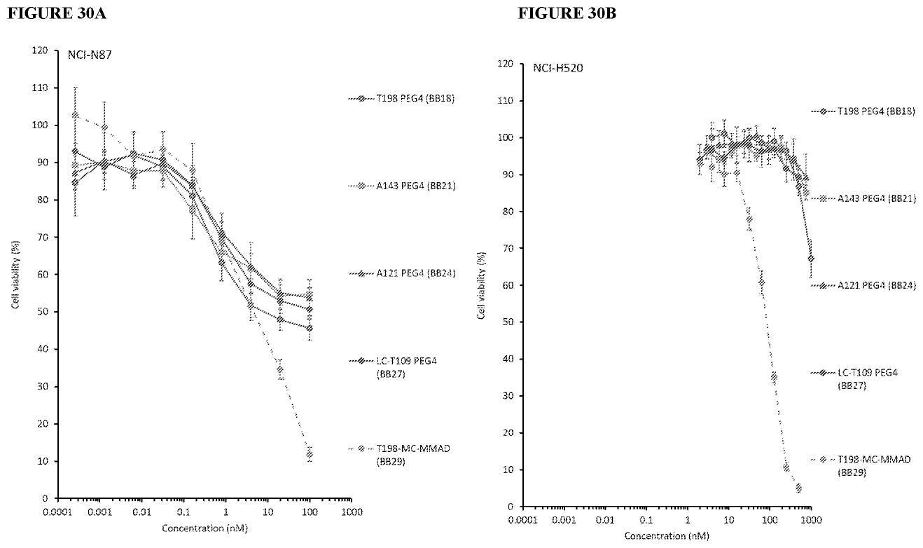

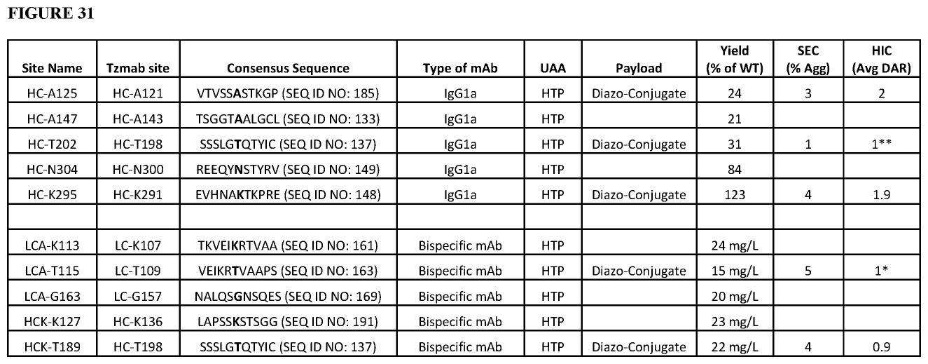

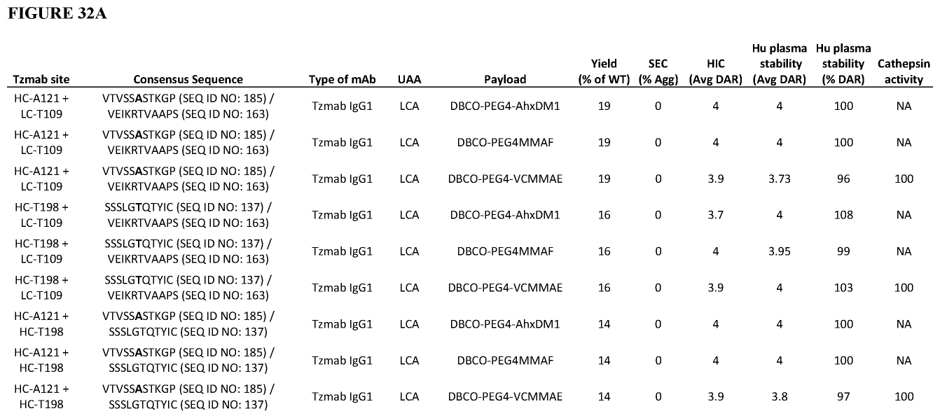

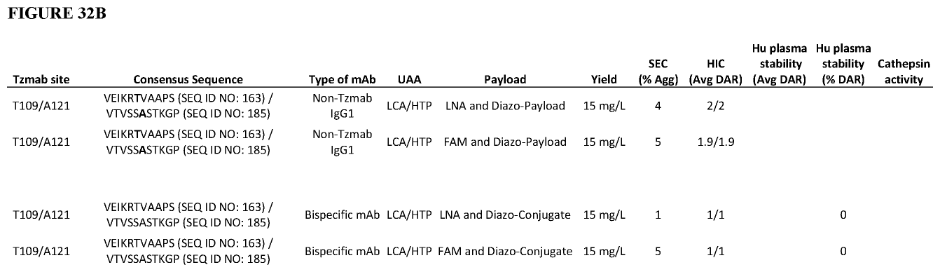

Trastuzumab HC-T198-LCA antibody drug conjugate production characterization by size exclusion chromatography (SEC) and hydrophobic interaction chromatography (HIC) (FIG. 4A), SEC and HIC with payload conjugated (FIG. 4B and FIG. 4C).

Trastuzumab HC-A143-LCA antibody drug conjugate production characterization by size exclusion chromatography (SEC) and hydrophobic interaction chromatography (HIC) (FIG. 4D), SEC and HIC with payload conjugated (FIG. 4E).

Trastuzumab HC-A121-LCA antibody drug conjugate production characterization by size exclusion chromatography (SEC) and hydrophobic interaction chromatography (HIC) (FIG. 4F), SEC and HIC with payload conjugated (FIG. 4G).

Trastuzumab LC-K107-LCA antibody drug conjugate production by size exclusion chromatography (SEC) and hydrophobic interaction chromatography (HIC) (FIG. 4H), SEC and HIC with payload conjugated (FIG. 4I).

Trastuzumab LC-T109-LCA antibody drug conjugate production characterization by size exclusion chromatography (SEC) and hydrophobic interaction chromatography (HIC) (FIG. 4J), SEC and HIC with payload conjugated (FIG. 4K).

FIG. 5 depicts:

Liquid chromatography-mass spectrometry (LC-MS) for Trastuzumab HC-T198-LCA, deconvoluted values shown in FIG. 5A and FIG. 5C, and an LC-MS chromatogram in FIG. 5B.

LC-MS for Trastuzumab HC-T198-BCN-PEG4-MMAD, deconvoluted values shown in FIG. 5D and an LC-MS chromatogram in FIG. 5E.

LC-MS for Trastuzumab HC-T198-HTP, deconvoluted values shown in FIG. 5F and FIG. 5H, and an LC-MS chromatogram in FIG. 5G.

LC-MS for Trastuzumab HC-T198-HTP+Diazonium-Biotin, deconvoluted values shown in FIG. 5I and FIG. 5K, and an LC-MS chromatogram in FIG. 5J.

LC-MS for Trastuzumab HC-A143-LCA, deconvoluted values shown in FIG. 5L and FIG. 5N, and an LC-MS chromatogram in FIG. 5M.

LC-MS for Trastuzumab HC-A143-BCN-PEG4-MMAD, deconvoluted values shown in FIG. 5O and FIG. 5Q, and an LC-MS chromatogram in FIG. 5P.

LC-MS for Trastuzumab HC-A121/LCA, deconvoluted values shown in FIG. 5R and FIG. 5T, and an LC-MS chromatogram in FIG. 5S.

LC-MS for Trastuzumab HC-A121/BCN-PEG8-MMAD deconvoluted values shown in FIG. 5U, and an LC-MS chromatogram in FIG. 5V.

LC-MS for Trastuzumab LC-K107/LCA deconvoluted values shown in FIG. 5W and an LC-MS chromatogram in FIG. 5X.

LC-MS for Trastuzumab LC-T109/LCA deconvoluted values shown in FIG. 5Y and FIG. 5AA, and an LC-MS chromatogram in FIG. 5Z.

LC-MS for Trastuzumab LC-T109/BCN-PEG8-MMAD deconvoluted values shown in FIG. 5BB and FIG. 5DD, and an LC-MS chromatogram in FIG. 5CC.

FIG. 6A depicts a subset of UAAs that are exemplary substrates for a leucyl tRNA-synthetase.

FIG. 6B depicts a subset of UAAs that are exemplary substrates for a tryptophanyl tRNA-synthetase.

FIG. 6C depicts a subset of UAAs that are exemplary substrates for a leucyl tRNA-synthetase.

FIG. 6D depicts a subset of UAAs that are exemplary substrates for a leucyl tRNA-synthetase.

FIG. 6E depicts UAAs C5Az, LCA, and AzW.

FIG. 6F depicts a subset of UAAs that are exemplary substrates for a tyrosyl tRNA-synthetase.

FIG. 6G depicts a subset of UAAs that are exemplary substrates for a pyrrolysyl tRNA-synthetase.

FIG. 7 presents a summary of antibody studies of the present disclosure, including ADC details.

FIG. 8 depicts the chemical structures of example payloads BCN-PEG4-MMAD and BCN-PEG8-MMAD.

FIG. 9 depicts the chemical structures of example payloads DBCO-PEG4-Ahx-DM1, DBCO-PEG4-MMAF, and DBCO-PEG4-VC-MMAE.

FIG. 10 depicts the chemical structures of example payloads DBCO-PEG4-LNA, DBCO-FAM, and DBCO-PEG8-VC-Nontoxic-small molecule (NonToxic SM).

FIG. 11 depicts the chemical structures of example payloads Diazo-Conjugate MMAF and Diazo-Conjugate biotin.

FIG. 12 depicts expression levels of LCA-containing antibodies at single sites of UAA incorporation.

FIG. 13 presents a summary of expression levels of antibodies with single sites of LCA incorporation as described herein.

FIG. 14 depicts the average drug-antibody ratio (DAR) of LCA-containing antibodies with a variety of payloads.

FIG. 15 presents a summary of the average drug-antibody ratio (DAR) of LCA-containing antibodies with a variety of payloads.

FIG. 16 depicts the size-exclusion chromatography (SEC) aggregation data for the LCA-based ADCs.

FIG. 17 presents a summary of the size-exclusion chromatography (SEC) aggregation data for the LCA-based ADCs.

FIG. 18 depicts stability in human plasma of LCA-based ADCs.

FIG. 19 presents a summary of stability in human plasma for LCA-based ADCs.

FIG. 20 depicts percentage cleavage of payload following incubation with cathepsin B for LCA-based ADCs.

FIG. 21 presents a summary of percentage cleavage of payload following incubation with cathepsin B for LCA-based ADCs.

FIG. 22A depicts binding of ADCs to HER2 as determined by ELISA.

FIG. 22B presents EC50 values for the ELISA, demonstrating that sub-nanomolar affinity is maintained with ADCs of the present disclosure.

FIG. 23 depicts a table of the IC50 values for ADCs containing DBCO-PEG4/PEG8-MMAD payload as determined by cytotoxicity studies using Cell-Titer Glo®.

FIG. 24A and FIG. 24B depicts cytotoxicity traces for the indicated ADCs.

FIG. 25A and FIG. 25B depict T198 cytotoxicity curves in comparison to a T198 MC-MMAD cysteine site-specific mutant.

FIG. 26A and FIG. 26B depict A143 cytotoxicity curves in comparison to a T198 MC-MMAD cysteine site-specific mutant.

FIG. 27A and FIG. 27B depict A121 curves in comparison to a T198 MC-MMAD cysteine site-specific mutant.

FIG. 28A and FIG. 28B depict T109 curves in comparison to a T198 MC-MMAD cysteine site-specific mutant

FIG. 29A and FIG. 29B show cytotoxicity comparison for PEG4 and PEG8 payloads across tested sites.

FIG. 30A and FIG. 30B show cytotoxicity comparison for PEG4 and PEG8 payloads across tested sites.

FIG. 31 presents a summary of yield, aggregation, and hydrophobicity data obtained using HTP ADCs.

FIG. 32 presents a summary of yield, aggregation, and hydrophobicity data obtained using ADCs with multiple UAAs (LCA and HTP) incorporated. Values for trastuzumab are presented in FIG. 32A, and values for a non-Trastuzumab IgG1 and a bispecific antibody are presented in FIG. 32B.

### DETAILED DESCRIPTION

The invention is based, in part, on the discovery of specified positions in antibodies that allow for efficient incorporation of one, or more, unnatural amino acids (UAAs) into the antibodies. The invention is further based, in part, on the discovery of combinations of specified positions in antibodies, UAAs for incorporation at those positions, and molecules for conjugation at those UAAs. Among other things, the combinations of position, UAA, and molecule allow for the efficient generation of antibody conjugates with desirable properties, including, for example, drug to antibody ratio (DAR), lack of aggregation, stability, and binding affinity.

Accordingly, in one aspect, the invention provides an antibody including a UAA at one or more positions corresponding to P14, G66, D73, L155, A121, K124, T138, A143, V157, T158, S160, T167, T198, N204, V205, N206, K213, D215, I256, K277, Y281, K291, K293, N300, or F407 of an antibody heavy chain or heavy chain fragment (e.g., at the corresponding positions in SEQ ID NO: 121). In another aspect, the invention provides an antibody including a UAA at one or more positions corresponding to V15, T20, R24, S60, S66, K107, T109, V110, A111, Q147, L154, G157, K169 A193, V205, T206, or S208 of an antibody light chain or light chain fragment (e.g., at the corresponding positions in SEQ ID NO: 155).

In another aspect, the invention provides a method of producing any of the foregoing antibodies. The method comprises culturing a cell with: (i) a nucleic acid comprising a nucleotide sequence encoding a tRNA comprising an anticodon that hybridizes to a codon selected from UAG, UGA, and UAA, and is capable of being charged with the unnatural amino acid (UAA); (ii) a nucleic acid comprising a nucleotide sequence encoding an aminoacyl-tRNA synthetase capable of charging the tRNA with the unnatural amino acid (UAA); and (iii) a nucleic acid comprising a nucleotide sequence encoding a heavy chain, a light chain, or a combination of a heavy chain and light chain of the antibody and comprising the codon selected from UAG, UGA, and UAA; under conditions that permit the tRNA, when expressed in the cell and charged with the unnatural amino acid (UAA), to hybridize to the codon and direct the incorporation of the unnatural amino acid (UAA) into the antibody.

As used herein, the term “orthogonal” refers to a molecule (e.g., an orthogonal tRNA or an orthogonal aminoacyl-tRNA synthetase) that is used with reduced efficiency by an expression system of interest (e.g., an endogenous cellular translation system). For example, an orthogonal tRNA in a translation system of interest is aminoacylated by any endogenous aminoacyl-tRNA synthetase of the translation system of interest with reduced or even zero efficiency, when compared to aminoacylation of an endogenous tRNA by an endogenous aminoacyl-tRNA synthetase. In another example, an orthogonal aminoacyl-tRNA synthetase aminoacylates any endogenous tRNA in the translation system of interest with reduced or even zero efficiency, as compared to aminoacylation of an endogenous tRNA by an endogenous aminoacyl-tRNA synthetase.

Various features and aspects of the invention are discussed in more detail below.

### I. Antibodies

Encompassed by the invention are antibodies including unnatural amino acids (UAAs) and methods of making the same.

As used herein, unless otherwise indicated, the term “antibody” is understood to mean an intact antibody (e.g., an intact monoclonal antibody), or a fragment thereof, such as a Fc fragment of an antibody (e.g., an Fc fragment of a monoclonal antibody), or an antigen-binding fragment of an antibody (e.g., an antigen-binding fragment of a monoclonal antibody), including an intact antibody, antigen-binding fragment, or Fc fragment that has been modified, engineered, or chemically conjugated. Examples of antigen-binding fragments include Fab, Fab′, (Fab′)₂, Fv, single chain antibodies (e.g., scFv), minibodies, and diabodies. Examples of antibodies that have been modified or engineered include chimeric antibodies, humanized antibodies, and multispecific antibodies (e.g., bispecific antibodies). An example of a chemically conjugated antibody is an antibody conjugated to a toxin moiety.

Typically, antibodies are multimeric proteins that contain four polypeptide chains. Two of the polypeptide chains are called immunoglobulin heavy chains (H chains), and two of the polypeptide chains are called immunoglobulin light chains (L chains). The immunoglobulin heavy and light chains are connected by an interchain disulfide bond. The immunoglobulin heavy chains are connected by interchain disulfide bonds. A light chain consists of one variable region (V_{L}) and one constant region (C_{L}). The heavy chain consists of one variable region (V_{H}) and at least three constant regions (CH₁, CH₂ and CH₃). The variable regions determine the binding specificity of the antibody.

The variable heavy (VH) and variable light (VL) regions can be further subdivided into regions of hypervariability, termed “complementarity determining regions” (“CDR”), interspersed with regions that are more conserved, termed “framework regions” (FR). Human antibodies have three VH CDRs and three VL CDRs, separated by framework regions FR1-FR4. The extent of the FRs and CDRs has been defined (Kabat, E. A., et al. (1991) SEQUENCES OF PROTEINS OF IMMUNOLOGICAL INTEREST, FIFTH EDITION, U.S. Department of Health and Human Services, NIH Publication No. 91-3242; and Chothia, C. et al. (1987) J. MOL. BIOL. 196:901-917). Each VH and VL is typically composed of three CDRs and four FRs, arranged from amino-terminus to carboxyl-terminus in the following order: FR1, CDR1, FR2, CDR2, FR3, CDR3, and FR4.

An antibody molecule may have (i) a heavy chain constant region chosen from, e.g., the heavy chain constant regions of IgG1, IgG2, IgG3, IgG4, IgM, IgA1, IgA2, IgD, and IgE; particularly, chosen from, e.g., the (e.g., human) heavy chain constant regions of IgG1, IgG2, IgG3, and IgG4, and/or (ii) a light chain constant region chosen from, e.g., the (e.g., human) light chain constant regions of kappa or lambda.

The incorporation of an unnatural amino acid can be done for a variety of purposes, including tailoring changes in antibody structure and/or function, changing size, acidity, nucleophilicity, hydrogen bonding, hydrophobicity, accessibility of protease target sites, targeting to a moiety (e.g., for a protein array), adding a biologically active molecule, attaching a polymer, attaching a radionuclide, modulating serum half-life, modulating tissue penetration (e.g., tumors), modulating active transport, modulating tissue, cell or organ specificity or distribution, modulating immunogenicity, modulating protease resistance, etc. Antibodies that include an unnatural amino acid can have enhanced or even entirely new biophysical properties. For example, the following properties are optionally modified by inclusion of an unnatural amino acid into antibody: toxicity, biodistribution, structural properties, spectroscopic properties, chemical and/or photochemical properties, catalytic ability, half-life (including but not limited to, serum half-life), ability to react with other molecules, including but not limited to, covalently or noncovalently, and the like. The compositions including antibodies that include at least one unnatural amino acid are useful for, including but not limited to, novel therapeutics, diagnostics, and binding proteins.

An antibody may have at least one, for example, at least two, at least three, at least four, at least five, at least six, at least seven, at least eight, at least nine, or at least ten or more UAAs. The UAAs can be the same or different. For example, there can be 1, 2, 3, 4, 5, 6, 7, 8, 9, or 10 or more different sites in the antibody that comprise 1, 2, 3, 4, 5, 6, 7, 8, 9, or 10 or more different UAAs. An antibody may have at least one, but fewer than all, of a particular amino acid present in the antibody substituted with the UAA. For a given antibody with more than one UAA, the UAA can be identical or different (for example, the antibody can include two or more different types of UAAs, or can include two of the same UAA). For a given antibody with more than two UAAs, the UAAs can be the same, different or a combination of a multiple unnatural amino acid of the same kind with at least one different UAA.

Antibodies contemplated herein may comprise a UAA in a heavy chain or a fragment thereof, for example, in one or more of a heavy chain FR1, CDR1, FR2, CDR2, FR3, CDR3, FR4, or constant region (e.g., an IgG1 constant region). Alternatively or in addition, antibodies contemplated herein may comprise a UAA in a light chain or a fragment thereof, for example, in a light chain FR1, CDR1, FR2, CDR2, FR3, CDR3, FR4, or constant region (e.g., a kappa constant region).

In certain embodiments, a UAA is located in a heavy chain FR1 region of an antibody. For example, in certain embodiments, the antibody comprises: the amino acid sequence of SEQ ID NO: 118, or an amino acid sequence having at least 85%, 90%, 95%, 96%, 97%, 98%, or 99% sequence identity to SEQ ID NO: 118, with an unnatural amino acid (UAA) at a position corresponding to P6 of SEQ ID NO: 118; the amino acid sequence of SEQ ID NO: 119, or an amino acid sequence having at least 85%, 90%, 95%, 96%, 97%, 98%, or 99% sequence identity to SEQ ID NO: 119, with an unnatural amino acid (UAA) at a position corresponding to P14 of SEQ ID NO: 119; the amino acid sequence of SEQ ID NO: 120, or an amino acid sequence having at least 85%, 90%, 95%, 96%, 97%, 98%, or 99% sequence identity to SEQ ID NO: 120, with an unnatural amino acid (UAA) at a position corresponding to P14 of SEQ ID NO: 120; or the amino acid sequence of SEQ ID NO: 121, or an amino acid sequence having at least 85%, 90%, 95%, 96%, 97%, 98%, or 99% sequence identity to SEQ ID NO: 121 with an unnatural amino acid (UAA) at a position corresponding to P14 of SEQ ID NO: 121.

In certain embodiments, a UAA is located in a heavy chain CDRH2 region of an antibody. For example, in certain embodiments, the antibody comprises: the amino acid sequence of SEQ ID NO: 122, or an amino acid sequence having at least 85%, 90%, 95%, 96%, 97%, 98%, or 99% sequence identity to SEQ ID NO: 122, with an unnatural amino acid (UAA) at a position corresponding to G6 of SEQ ID NO: 122; the amino acid sequence of SEQ ID NO: 123, or an amino acid sequence having at least 85%, 90%, 95%, 96%, 97%, 98%, or 99% sequence identity to SEQ ID NO: 123, with an unnatural amino acid (UAA) at a position corresponding to G17 of SEQ ID NO: 123; the amino acid sequence of SEQ ID NO: 120, or an amino acid sequence having at least 85%, 90%, 95%, 96%, 97%, 98%, or 99% sequence identity to SEQ ID NO: 120, with an unnatural amino acid (UAA) at a position corresponding to G66 of SEQ ID NO: 120; or the amino acid sequence of SEQ ID NO: 121, or an amino acid sequence having at least 85%, 90%, 95%, 96%, 97%, 98%, or 99% sequence identity to SEQ ID NO: 121, with an unnatural amino acid (UAA) at a position corresponding to G66 of SEQ ID NO: 121.

In certain embodiments, a UAA is located in a heavy chain FR3 region of an antibody. For example, in certain embodiments, the antibody comprises: the amino acid sequence of SEQ ID NO: 124, or an amino acid sequence having at least 85%, 90%, 95%, 96%, 97%, 98%, or 99% sequence identity to SEQ ID NO: 124, with an unnatural amino acid (UAA) at a position corresponding to D6 of SEQ ID NO: 124; the amino acid sequence of SEQ ID NO: 125, or an amino acid sequence having at least 85%, 90%, 95%, 96%, 97%, 98%, or 99% sequence identity to SEQ ID NO: 125, with an unnatural amino acid (UAA) at a position corresponding to D7 of SEQ ID NO: 125; the amino acid sequence of SEQ ID NO: 120, or an amino acid sequence having at least 85%, 90%, 95%, 96%, 97%, 98%, or 99% sequence identity to SEQ ID NO: 120, with an unnatural amino acid (UAA) at a position corresponding to D73 of SEQ ID NO: 120; or the amino acid sequence of SEQ ID NO: 121, or an amino acid sequence having at least 85%, 90%, 95%, 96%, 97%, 98%, or 99% sequence identity to SEQ ID NO: 121, with an unnatural amino acid (UAA) at a position corresponding to D73 of SEQ ID NO: 121.

In certain embodiments, a UAA is located in a heavy chain FR4 region of an antibody. For example, in certain embodiments, the antibody comprises: the amino acid sequence of SEQ ID NO: 126, or an amino acid sequence having at least 85%, 90%, 95%, 96%, 97%, 98%, or 99% sequence identity to SEQ ID NO: 126, with an unnatural amino acid (UAA) at a position corresponding to L6 of SEQ ID NO: 126; the amino acid sequence of SEQ ID NO: 127, or an amino acid sequence having at least 85%, 90%, 95%, 96%, 97%, 98%, or 99% sequence identity to SEQ ID NO: 127, with an unnatural amino acid (UAA) at a position corresponding to L6 of SEQ ID NO: 127; the amino acid sequence of SEQ ID NO: 120, or an amino acid sequence having at least 85%, 90%, 95%, 96%, 97%, 98%, or 99% sequence identity to SEQ ID NO: 120, with an unnatural amino acid (UAA) at a position corresponding to L115 of SEQ ID NO: 120; or the amino acid sequence of SEQ ID NO: 121, or an amino acid sequence having at least 85%, 90%, 95%, 96%, 97%, 98%, or 99% sequence identity to SEQ ID NO: 121, with an unnatural amino acid (UAA) at a position corresponding to L115 of SEQ ID NO: 121.

In certain embodiments, a UAA is located in a heavy chain CH1 region of an antibody. For example, in certain embodiments, the antibody comprises: the amino acid sequence of SEQ ID NO: 128, or an amino acid sequence having at least 85%, 90%, 95%, 96%, 97%, 98%, or 99% sequence identity to SEQ ID NO: 128, with an unnatural amino acid (UAA) at a position corresponding to A6 of SEQ ID NO: 128; the amino acid sequence of SEQ ID NO: 185, or an amino acid sequence having at least 85%, 90%, 95%, 96%, 97%, 98%, or 99% sequence identity to SEQ ID NO: 185, with an unnatural amino acid (UAA) at a position corresponding to A6 of SEQ ID NO: 185; the amino acid sequence of SEQ ID NO: 129, or an amino acid sequence having at least 85%, 90%, 95%, 96%, 97%, 98%, or 99% sequence identity to SEQ ID NO: 129, with an unnatural amino acid (UAA) at a position corresponding to A1 of SEQ ID NO: 129; the amino acid sequence of SEQ ID NO: 130, or an amino acid sequence having at least 85%, 90%, 95%, 96%, 97%, 98%, or 99% sequence identity to SEQ ID NO: 130, with an unnatural amino acid (UAA) at a position corresponding to A1 of SEQ ID NO: 130; or the amino acid sequence of SEQ ID NO: 121, or an amino acid sequence having at least 85%, 90%, 95%, 96%, 97%, 98%, or 99% sequence identity to SEQ ID NO: 121, with an unnatural amino acid (UAA) at a position corresponding to A121 of SEQ ID NO: 121. In certain embodiments, the antibody comprises: the amino acid sequence of SEQ ID NO: 131, or an amino acid sequence having at least 85%, 90%, 95%, 96%, 97%, 98%, or 99% sequence identity to SEQ ID NO: 131, with an unnatural amino acid (UAA) at a position corresponding to K6 of SEQ ID NO: 131; the amino acid sequence of SEQ ID NO: 129, or an amino acid sequence having at least 85%, 90%, 95%, 96%, 97%, 98%, or 99% sequence identity to SEQ ID NO: 129, with an unnatural amino acid (UAA) at a position corresponding to K3 of SEQ ID NO: 129; the amino acid sequence of SEQ ID NO: 130, or an amino acid sequence having at least 85%, 90%, 95%, 96%, 97%, 98%, or 99% sequence identity to SEQ ID NO: 130, with an unnatural amino acid (UAA) at a position corresponding to K3 of SEQ ID NO: 130; or the amino acid sequence of SEQ ID NO: 121, or an amino acid sequence having at least 85%, 90%, 95%, 96%, 97%, 98%, or 99% sequence identity to SEQ ID NO: 121, with an unnatural amino acid (UAA) at a position corresponding to K124 of SEQ ID NO: 121. In certain embodiments, the antibody comprises: the amino acid sequence of SEQ ID NO: 191, or an amino acid sequence having at least 85%, 90%, 95%, 96%, 97%, 98%, or 99% sequence identity to SEQ ID NO: 191, with an unnatural amino acid (UAA) at a position corresponding to K6 of SEQ ID NO: 191; the amino acid sequence of SEQ ID NO: 129, or an amino acid sequence having at least 85%, 90%, 95%, 96%, 97%, 98%, or 99% sequence identity to SEQ ID NO: 129, with an unnatural amino acid (UAA) at a position corresponding to K16 of SEQ ID NO: 129; the amino acid sequence of SEQ ID NO: 130, or an amino acid sequence having at least 85%, 90%, 95%, 96%, 97%, 98%, or 99% sequence identity to SEQ ID NO: 130, with an unnatural amino acid (UAA) at a position corresponding to K16 of SEQ ID NO: 130; or the amino acid sequence of SEQ ID NO: 121, or an amino acid sequence having at least 85%, 90%, 95%, 96%, 97%, 98%, or 99% sequence identity to SEQ ID NO: 121, with an unnatural amino acid (UAA) at a position corresponding to K136 of SEQ ID NO: 121. In certain embodiments, the antibody comprises: the amino acid sequence of SEQ ID NO: 132, or an amino acid sequence having at least 85%, 90%, 95%, 96%, 97%, 98%, or 99% sequence identity to SEQ ID NO: 132, with an unnatural amino acid (UAA) at a position corresponding to T6 of SEQ ID NO: 132; the amino acid sequence of SEQ ID NO: 186, or an amino acid sequence having at least 85%, 90%, 95%, 96%, 97%, 98%, or 99% sequence identity to SEQ ID NO: 186, with an unnatural amino acid (UAA) at a position corresponding to T4 of SEQ ID NO: 186; the amino acid sequence of SEQ ID NO: 129, or an amino acid sequence having at least 85%, 90%, 95%, 96%, 97%, 98%, or 99% sequence identity to SEQ ID NO: 129, with an unnatural amino acid (UAA) at a position corresponding to T18 of SEQ ID NO: 129; the amino acid sequence of SEQ ID NO: 130, or an amino acid sequence having at least 85%, 90%, 95%, 96%, 97%, 98%, or 99% sequence identity to SEQ ID NO: 130, with an unnatural amino acid (UAA) at a position corresponding to T18 of SEQ ID NO: 130; or the amino acid sequence of SEQ ID NO: 121, or an amino acid sequence having at least 85%, 90%, 95%, 96%, 97%, 98%, or 99% sequence identity to SEQ ID NO: 121, with an unnatural amino acid (UAA) at a position corresponding to T138 of SEQ ID NO: 121. In certain embodiments, the antibody comprises: the amino acid sequence of SEQ ID NO: 133, or an amino acid sequence having at least 85%, 90%, 95%, 96%, 97%, 98%, or 99% sequence identity to SEQ ID NO: 133, with an unnatural amino acid (UAA) at a position corresponding to A6 of SEQ ID NO: 133; the amino acid sequence of SEQ ID NO: 129, or an amino acid sequence having at least 85%, 90%, 95%, 96%, 97%, 98%, or 99% sequence identity to SEQ ID NO: 129, with an unnatural amino acid (UAA) at a position corresponding to A23 of SEQ ID NO: 129; the amino acid sequence of SEQ ID NO: 130, or an amino acid sequence having at least 85%, 90%, 95%, 96%, 97%, 98%, or 99% sequence identity to SEQ ID NO: 130, with an unnatural amino acid (UAA) at a position corresponding to A23 of SEQ ID NO: 130; or the amino acid sequence of SEQ ID NO: 121, or an amino acid sequence having at least 85%, 90%, 95%, 96%, 97%, 98%, or 99% sequence identity to SEQ ID NO: 121, with an unnatural amino acid (UAA) at a position corresponding to A143 of SEQ ID NO: 121. In certain embodiments, the antibody comprises: the amino acid sequence of SEQ ID NO: 134, or an amino acid sequence having at least 85%, 90%, 95%, 96%, 97%, 98%, or 99% sequence identity to SEQ ID NO: 134, with an unnatural amino acid (UAA) at a position corresponding to V6 of SEQ ID NO: 134; the amino acid sequence of SEQ ID NO: 129, or an amino acid sequence having at least 85%, 90%, 95%, 96%, 97%, 98%, or 99% sequence identity to SEQ ID NO: 129, with an unnatural amino acid (UAA) at a position corresponding to V37 of SEQ ID NO: 129; the amino acid sequence of SEQ ID NO: 130, or an amino acid sequence having at least 85%, 90%, 95%, 96%, 97%, 98%, or 99% sequence identity to SEQ ID NO: 130, with an unnatural amino acid (UAA) at a position corresponding to V37 of SEQ ID NO: 130; or the amino acid sequence of SEQ ID NO: 121, or an amino acid sequence having at least 85%, 90%, 95%, 96%, 97%, 98%, or 99% sequence identity to SEQ ID NO: 121, with an unnatural amino acid (UAA) at a position corresponding to V157 of SEQ ID NO: 121. In certain embodiments, the antibody comprises: the amino acid sequence of SEQ ID NO: 174, or an amino acid sequence having at least 85%, 90%, 95%, 96%, 97%, 98%, or 99% sequence identity to SEQ ID NO: 174, with an unnatural amino acid (UAA) at a position corresponding to T6 of SEQ ID NO: 174; the amino acid sequence of SEQ ID NO: 187, or an amino acid sequence having at least 85%, 90%, 95%, 96%, 97%, 98%, or 99% sequence identity to SEQ ID NO: 187, with an unnatural amino acid (UAA) at a position corresponding to T5 of SEQ ID NO: 187; the amino acid sequence of SEQ ID NO: 129, or an amino acid sequence having at least 85%, 90%, 95%, 96%, 97%, 98%, or 99% sequence identity to SEQ ID NO: 129, with an unnatural amino acid (UAA) at a position corresponding to T38 of SEQ ID NO: 129; the amino acid sequence of SEQ ID NO: 130, or an amino acid sequence having at least 85%, 90%, 95%, 96%, 97%, 98%, or 99% sequence identity to SEQ ID NO: 130, with an unnatural amino acid (UAA) at a position corresponding to T38 of SEQ ID NO: 130; or the amino acid sequence of SEQ ID NO: 121, or an amino acid sequence having at least 85%, 90%, 95%, 96%, 97%, 98%, or 99% sequence identity to SEQ ID NO: 121, with an unnatural amino acid (UAA) at a position corresponding to T158 of SEQ ID NO: 121. In certain embodiments, the antibody comprises: the amino acid sequence of SEQ ID NO: 135, or an amino acid sequence having at least 85%, 90%, 95%, 96%, 97%, 98%, or 99% sequence identity to SEQ ID NO: 135, with an unnatural amino acid (UAA) at a position corresponding to S6 of SEQ ID NO: 135; the amino acid sequence of SEQ ID NO: 187, or an amino acid sequence having at least 85%, 90%, 95%, 96%, 97%, 98%, or 99% sequence identity to SEQ ID NO: 187, with an unnatural amino acid (UAA) at a position corresponding to S7 of SEQ ID NO: 187; the amino acid sequence of SEQ ID NO: 129, or an amino acid sequence having at least 85%, 90%, 95%, 96%, 97%, 98%, or 99% sequence identity to SEQ ID NO: 129, with an unnatural amino acid (UAA) at a position corresponding to S40 of SEQ ID NO: 129; the amino acid sequence of SEQ ID NO: 130, or an amino acid sequence having at least 85%, 90%, 95%, 96%, 97%, 98%, or 99% sequence identity to SEQ ID NO: 130, with an unnatural amino acid (UAA) at a position corresponding to S40 of SEQ ID NO: 130; or the amino acid sequence of SEQ ID NO: 121, or an amino acid sequence having at least 85%, 90%, 95%, 96%, 97%, 98%, or 99% sequence identity to SEQ ID NO: 121, with an unnatural amino acid (UAA) at a position corresponding to S160 of SEQ ID NO: 121. In certain embodiments, the antibody comprises: the amino acid sequence of SEQ ID NO: 136, or an amino acid sequence having at least 85%, 90%, 95%, 96%, 97%, 98%, or 99% sequence identity to SEQ ID NO: 136, with an unnatural amino acid (UAA) at a position corresponding to T6 of SEQ ID NO: 136; the amino acid sequence of SEQ ID NO: 188, or an amino acid sequence having at least 85%, 90%, 95%, 96%, 97%, 98%, or 99% sequence identity to SEQ ID NO: 188, with an unnatural amino acid (UAA) at a position corresponding to T4 of SEQ ID NO: 188; the amino acid sequence of SEQ ID NO: 129, or an amino acid sequence having at least 85%, 90%, 95%, 96%, 97%, 98%, or 99% sequence identity to SEQ ID NO: 129, with an unnatural amino acid (UAA) at a position corresponding to T47 of SEQ ID NO: 129; the amino acid sequence of SEQ ID NO: 130, or an amino acid sequence having at least 85%, 90%, 95%, 96%, 97%, 98%, or 99% sequence identity to SEQ ID NO: 130, with an unnatural amino acid (UAA) at a position corresponding to T47 of SEQ ID NO: 130; or the amino acid sequence of SEQ ID NO: 121, or an amino acid sequence having at least 85%, 90%, 95%, 96%, 97%, 98%, or 99% sequence identity to SEQ ID NO: 121, with an unnatural amino acid (UAA) at a position corresponding to T167 of SEQ ID NO: 121. In certain embodiments, the antibody comprises: the amino acid sequence of SEQ ID NO: 137, or an amino acid sequence having at least 85%, 90%, 95%, 96%, 97%, 98%, or 99% sequence identity to SEQ ID NO: 137, with an unnatural amino acid (UAA) at a position corresponding to T6 of SEQ ID NO: 137; the amino acid sequence of SEQ ID NO: 129, or an amino acid sequence having at least 85%, 90%, 95%, 96%, 97%, 98%, or 99% sequence identity to SEQ ID NO: 129, with an unnatural amino acid (UAA) at a position corresponding to T78 of SEQ ID NO: 129; the amino acid sequence of SEQ ID NO: 130, or an amino acid sequence having at least 85%, 90%, 95%, 96%, 97%, 98%, or 99% sequence identity to SEQ ID NO: 130, with an unnatural amino acid (UAA) at a position corresponding to T78 of SEQ ID NO: 130; or the amino acid sequence of SEQ ID NO: 121, or an amino acid sequence having at least 85%, 90%, 95%, 96%, 97%, 98%, or 99% sequence identity to SEQ ID NO: 121, with an unnatural amino acid (UAA) at a position corresponding to T198 of SEQ ID NO: 121. In certain embodiments, the antibody comprises: the amino acid sequence of SEQ ID NO: 138, or an amino acid sequence having at least 85%, 90%, 95%, 96%, 97%, 98%, or 99% sequence identity to SEQ ID NO: 138, with an unnatural amino acid (UAA) at a position corresponding to N6 of SEQ ID NO: 138; the amino acid sequence of SEQ ID NO: 189, or an amino acid sequence having at least 85%, 90%, 95%, 96%, 97%, 98%, or 99% sequence identity to SEQ ID NO: 189, with an unnatural amino acid (UAA) at a position corresponding to N6 of SEQ ID NO: 189; the amino acid sequence of SEQ ID NO: 129, or an amino acid sequence having at least 85%, 90%, 95%, 96%, 97%, 98%, or 99% sequence identity to SEQ ID NO: 129, with an unnatural amino acid (UAA) at a position corresponding to N84 of SEQ ID NO: 129; the amino acid sequence of SEQ ID NO: 130, or an amino acid sequence having at least 85%, 90%, 95%, 96%, 97%, 98%, or 99% sequence identity to SEQ ID NO: 130, with an unnatural amino acid (UAA) at a position corresponding to N84 of SEQ ID NO: 130; or the amino acid sequence of SEQ ID NO: 121, or an amino acid sequence having at least 85%, 90%, 95%, 96%, 97%, 98%, or 99% sequence identity to SEQ ID NO: 121, with an unnatural amino acid (UAA) at a position corresponding to N204 of SEQ ID NO: 121. In certain embodiments, the antibody comprises: the amino acid sequence of SEQ ID NO: 139, or an amino acid sequence having at least 85%, 90%, 95%, 96%, 97%, 98%, or 99% sequence identity to SEQ ID NO: 139, with an unnatural amino acid (UAA) at a position corresponding to V6 of SEQ ID NO: 139; the amino acid sequence of SEQ ID NO: 190, or an amino acid sequence having at least 85%, 90%, 95%, 96%, 97%, 98%, or 99% sequence identity to SEQ ID NO: 190, with an unnatural amino acid (UAA) at a position corresponding to V6 of SEQ ID NO: 190; the amino acid sequence of SEQ ID NO: 129, or an amino acid sequence having at least 85%, 90%, 95%, 96%, 97%, 98%, or 99% sequence identity to SEQ ID NO: 129, with an unnatural amino acid (UAA) at a position corresponding to V85 of SEQ ID NO: 129; the amino acid sequence of SEQ ID NO: 130, or an amino acid sequence having at least 85%, 90%, 95%, 96%, 97%, 98%, or 99% sequence identity to SEQ ID NO: 130, with an unnatural amino acid (UAA) at a position corresponding to V85 of SEQ ID NO: 130; or the amino acid sequence of SEQ ID NO: 121, or an amino acid sequence having at least 85%, 90%, 95%, 96%, 97%, 98%, or 99% sequence identity to SEQ ID NO: 121, with an unnatural amino acid (UAA) at a position corresponding to V205 of SEQ ID NO: 121. In certain embodiments, the antibody comprises: the amino acid sequence of SEQ ID NO: 140, or an amino acid sequence having at least 85%, 90%, 95%, 96%, 97%, 98%, or 99% sequence identity to SEQ ID NO: 140, with an unnatural amino acid (UAA) at a position corresponding to N6 of SEQ ID NO: 140; the amino acid sequence of SEQ ID NO: 189, or an amino acid sequence having at least 85%, 90%, 95%, 96%, 97%, 98%, or 99% sequence identity to SEQ ID NO: 189, with an unnatural amino acid (UAA) at a position corresponding to N8 of SEQ ID NO: 189; the amino acid sequence of SEQ ID NO: 129, or an amino acid sequence having at least 85%, 90%, 95%, 96%, 97%, 98%, or 99% sequence identity to SEQ ID NO: 129, with an unnatural amino acid (UAA) at a position corresponding to N86 of SEQ ID NO: 129; the amino acid sequence of SEQ ID NO: 130, or an amino acid sequence having at least 85%, 90%, 95%, 96%, 97%, 98%, or 99% sequence identity to SEQ ID NO: 130, with an unnatural amino acid (UAA) at a position corresponding to N86 of SEQ ID NO: 130; or the amino acid sequence of SEQ ID NO: 121, or an amino acid sequence having at least 85%, 90%, 95%, 96%, 97%, 98%, or 99% sequence identity to SEQ ID NO: 121, with an unnatural amino acid (UAA) at a position corresponding to N206 of SEQ ID NO: 121. In certain embodiments, the antibody comprises: the amino acid sequence of SEQ ID NO: 141, or an amino acid sequence having at least 85%, 90%, 95%, 96%, 97%, 98%, or 99% sequence identity to SEQ ID NO: 141, with an unnatural amino acid (UAA) at a position corresponding to K6 of SEQ ID NO: 141; the amino acid sequence of SEQ ID NO: 192, or an amino acid sequence having at least 85%, 90%, 95%, 96%, 97%, 98%, or 99% sequence identity to SEQ ID NO: 192, with an unnatural amino acid (UAA) at a position corresponding to K5 of SEQ ID NO: 192; the amino acid sequence of SEQ ID NO: 129, or an amino acid sequence having at least 85%, 90%, 95%, 96%, 97%, 98%, or 99% sequence identity to SEQ ID NO: 129, with an unnatural amino acid (UAA) at a position corresponding to K93 of SEQ ID NO: 129; the amino acid sequence of SEQ ID NO: 130, or an amino acid sequence having at least 85%, 90%, 95%, 96%, 97%, 98%, or 99% sequence identity to SEQ ID NO: 130, with an unnatural amino acid (UAA) at a position corresponding to K93 of SEQ ID NO: 130; or the amino acid sequence of SEQ ID NO: 121, or an amino acid sequence having at least 85%, 90%, 95%, 96%, 97%, 98%, or 99% sequence identity to SEQ ID NO: 121, with an unnatural amino acid (UAA) at a position corresponding to K213 of SEQ ID NO: 121. In certain embodiments, the antibody comprises: the amino acid sequence of SEQ ID NO: 142, or an amino acid sequence having at least 85%, 90%, 95%, 96%, 97%, 98%, or 99% sequence identity to SEQ ID NO: 142, with an unnatural amino acid (UAA) at a position corresponding to D6 of SEQ ID NO: 142; the amino acid sequence of SEQ ID NO: 129, or an amino acid sequence having at least 85%, 90%, 95%, 96%, 97%, 98%, or 99% sequence identity to SEQ ID NO: 129, with an unnatural amino acid (UAA) at a position corresponding to D95 of SEQ ID NO: 129; the amino acid sequence of SEQ ID NO: 130, or an amino acid sequence having at least 85%, 90%, 95%, 96%, 97%, 98%, or 99% sequence identity to SEQ ID NO: 130, with an unnatural amino acid (UAA) at a position corresponding to D95 of SEQ ID NO: 130; or the amino acid sequence of SEQ ID NO: 121, or an amino acid sequence having at least 85%, 90%, 95%, 96%, 97%, 98%, or 99% sequence identity to SEQ ID NO: 121, with an unnatural amino acid (UAA) at a position corresponding to D215 of SEQ ID NO: 121.

In certain embodiments, a UAA is located in a heavy chain CH2 region of an antibody. For example, in certain embodiments, the antibody comprises: the amino acid sequence of SEQ ID NO: 143, or an amino acid sequence having at least 85%, 90%, 95%, 96%, 97%, 98%, or 99% sequence identity to SEQ ID NO: 143, with an unnatural amino acid (UAA) at a position corresponding to 16 of SEQ ID NO: 143; the amino acid sequence of SEQ ID NO: 144, or an amino acid sequence having at least 85%, 90%, 95%, 96%, 97%, 98%, or 99% sequence identity to SEQ ID NO: 144, with an unnatural amino acid (UAA) at a position corresponding to 123 of SEQ ID NO: 144; the amino acid sequence of SEQ ID NO: 130, or an amino acid sequence having at least 85%, 90%, 95%, 96%, 97%, 98%, or 99% sequence identity to SEQ ID NO: 130, with an unnatural amino acid (UAA) at a position corresponding to 1136 of SEQ ID NO: 130; or the amino acid sequence of SEQ ID NO: 121, or an amino acid sequence having at least 85%, 90%, 95%, 96%, 97%, 98%, or 99% sequence identity to SEQ ID NO: 121, with an unnatural amino acid (UAA) at a position corresponding to 1256 of SEQ ID NO: 121. In certain embodiments, the antibody comprises: the amino acid sequence of SEQ ID NO: 145, or an amino acid sequence having at least 85%, 90%, 95%, 96%, 97%, 98%, or 99% sequence identity to SEQ ID NO: 145, with an unnatural amino acid (UAA) at a position corresponding to K6 of SEQ ID NO: 145; the amino acid sequence of SEQ ID NO: 193, or an amino acid sequence having at least 85%, 90%, 95%, 96%, 97%, 98%, or 99% sequence identity to SEQ ID NO: 193, with an unnatural amino acid (UAA) at a position corresponding to K6 of SEQ ID NO: 193; the amino acid sequence of SEQ ID NO: 144, or an amino acid sequence having at least 85%, 90%, 95%, 96%, 97%, 98%, or 99% sequence identity to SEQ ID NO: 144, with an unnatural amino acid (UAA) at a position corresponding to K44 of SEQ ID NO: 144; the amino acid sequence of SEQ ID NO: 130, or an amino acid sequence having at least 85%, 90%, 95%, 96%, 97%, 98%, or 99% sequence identity to SEQ ID NO: 130, with an unnatural amino acid (UAA) at a position corresponding to K157 of SEQ ID NO: 130; or the amino acid sequence of SEQ ID NO: 121, or an amino acid sequence having at least 85%, 90%, 95%, 96%, 97%, 98%, or 99% sequence identity to SEQ ID NO: 121, with an unnatural amino acid (UAA) at a position corresponding to K277 of SEQ ID NO: 121. In certain embodiments, the antibody comprises: the amino acid sequence of SEQ ID NO: 146, or an amino acid sequence having at least 85%, 90%, 95%, 96%, 97%, 98%, or 99% sequence identity to SEQ ID NO: 146, with an unnatural amino acid (UAA) at a position corresponding to Y6 of SEQ ID NO: 146; the amino acid sequence of SEQ ID NO: 193, or an amino acid sequence having at least 85%, 90%, 95%, 96%, 97%, 98%, or 99% sequence identity to SEQ ID NO: 193, with an unnatural amino acid (UAA) at a position corresponding to Y10 of SEQ ID NO: 193; the amino acid sequence of SEQ ID NO: 144, or an amino acid sequence having at least 85%, 90%, 95%, 96%, 97%, 98%, or 99% sequence identity to SEQ ID NO: 144, with an unnatural amino acid (UAA) at a position corresponding to Y48 of SEQ ID NO: 144; the amino acid sequence of SEQ ID NO: 130, or an amino acid sequence having at least 85%, 90%, 95%, 96%, 97%, 98%, or 99% sequence identity to SEQ ID NO: 130, with an unnatural amino acid (UAA) at a position corresponding to Y161 of SEQ ID NO: 130; or the amino acid sequence of SEQ ID NO: 121, or an amino acid sequence having at least 85%, 90%, 95%, 96%, 97%, 98%, or 99% sequence identity to SEQ ID NO: 121, with an unnatural amino acid (UAA) at a position corresponding to Y281 of SEQ ID NO: 121. In certain embodiments, the antibody comprises: the amino acid sequence of SEQ ID NO: 147, or an amino acid sequence having at least 85%, 90%, 95%, 96%, 97%, 98%, or 99% sequence identity to SEQ ID NO: 147, with an unnatural amino acid (UAA) at a position corresponding to K6 of SEQ ID NO: 147; the amino acid sequence of SEQ ID NO: 144, or an amino acid sequence having at least 85%, 90%, 95%, 96%, 97%, 98%, or 99% sequence identity to SEQ ID NO: 144, with an unnatural amino acid (UAA) at a position corresponding to K58 of SEQ ID NO: 144; the amino acid sequence of SEQ ID NO: 130, or an amino acid sequence having at least 85%, 90%, 95%, 96%, 97%, 98%, or 99% sequence identity to SEQ ID NO: 130, with an unnatural amino acid (UAA) at a position corresponding to K171 of SEQ ID NO: 130; or the amino acid sequence of SEQ ID NO: 121, or an amino acid sequence having at least 85%, 90%, 95%, 96%, 97%, 98%, or 99% sequence identity to SEQ ID NO: 121, with an unnatural amino acid (UAA) at a position corresponding to K291 of SEQ ID NO: 121. In certain embodiments, the antibody comprises: the amino acid sequence of SEQ ID NO: 148, or an amino acid sequence having at least 85%, 90%, 95%, 96%, 97%, 98%, or 99% sequence identity to SEQ ID NO: 148, with an unnatural amino acid (UAA) at a position corresponding to K6 of SEQ ID NO: 148; the amino acid sequence of SEQ ID NO: 194, or an amino acid sequence having at least 85%, 90%, 95%, 96%, 97%, 98%, or 99% sequence identity to SEQ ID NO: 194, with an unnatural amino acid (UAA) at a position corresponding to K7 of SEQ ID NO: 194; the amino acid sequence of SEQ ID NO: 144, or an amino acid sequence having at least 85%, 90%, 95%, 96%, 97%, 98%, or 99% sequence identity to SEQ ID NO: 144, with an unnatural amino acid (UAA) at a position corresponding to K60 of SEQ ID NO: 144; the amino acid sequence of SEQ ID NO: 130, or an amino acid sequence having at least 85%, 90%, 95%, 96%, 97%, 98%, or 99% sequence identity to SEQ ID NO: 130, with an unnatural amino acid (UAA) at a position corresponding to K173 of SEQ ID NO: 130; or the amino acid sequence of SEQ ID NO: 121, or an amino acid sequence having at least 85%, 90%, 95%, 96%, 97%, 98%, or 99% sequence identity to SEQ ID NO: 121, with an unnatural amino acid (UAA) at a position corresponding to K293 of SEQ ID NO: 121. In certain embodiments, the antibody comprises: the amino acid sequence of SEQ ID NO: 149, or an amino acid sequence having at least 85%, 90%, 95%, 96%, 97%, 98%, or 99% sequence identity to SEQ ID NO: 149, with an unnatural amino acid (UAA) at a position corresponding to N6 of SEQ ID NO: 149; the amino acid sequence of SEQ ID NO: 144, or an amino acid sequence having at least 85%, 90%, 95%, 96%, 97%, 98%, or 99% sequence identity to SEQ ID NO: 144, with an unnatural amino acid (UAA) at a position corresponding to N67 of SEQ ID NO: 144; the amino acid sequence of SEQ ID NO: 130, or an amino acid sequence having at least 85%, 90%, 95%, 96%, 97%, 98%, or 99% sequence identity to SEQ ID NO: 130, with an unnatural amino acid (UAA) at a position corresponding to N180 of SEQ ID NO: 130; or the amino acid sequence of SEQ ID NO: 121, or an amino acid sequence having at least 85%, 90%, 95%, 96%, 97%, 98%, or 99% sequence identity to SEQ ID NO: 121, with an unnatural amino acid (UAA) at a position corresponding to N300 of SEQ ID NO: 121.

In certain embodiments, a UAA is located in a heavy chain CH3 region of an antibody. For example, in certain embodiments, the antibody comprises: the amino acid sequence of SEQ ID NO: 150, or an amino acid sequence having at least 85%, 90%, 95%, 96%, 97%, 98%, or 99% sequence identity to SEQ ID NO: 150, with an unnatural amino acid (UAA) at a position corresponding to F6 of SEQ ID NO: 150; the amino acid sequence of SEQ ID NO: 151, or an amino acid sequence having at least 85%, 90%, 95%, 96%, 97%, 98%, or 99% sequence identity to SEQ ID NO: 151, with an unnatural amino acid (UAA) at a position corresponding to F64 of SEQ ID NO: 151; the amino acid sequence of SEQ ID NO: 130, or an amino acid sequence having at least 85%, 90%, 95%, 96%, 97%, 98%, or 99% sequence identity to SEQ ID NO: 130, with an unnatural amino acid (UAA) at a position corresponding to F287 of SEQ ID NO: 130; or the amino acid sequence of SEQ ID NO: 121, or an amino acid sequence having at least 85%, 90%, 95%, 96%, 97%, 98%, or 99% sequence identity to SEQ ID NO: 121, with an unnatural amino acid (UAA) at a position corresponding to F407 of SEQ ID NO: 121.

In certain embodiments, a UAA is located in a light chain FR1 region of an antibody. For example, in certain embodiments, the antibody comprises: the amino acid sequence of SEQ ID NO: 152, or an amino acid sequence having at least 85%, 90%, 95%, 96%, 97%, 98%, or 99% sequence identity to SEQ ID NO: 152, with an unnatural amino acid (UAA) at a position corresponding to V6 of SEQ ID NO: 152; the amino acid sequence of SEQ ID NO: 153, or an amino acid sequence having at least 85%, 90%, 95%, 96%, 97%, 98%, or 99% sequence identity to SEQ ID NO: 153, with an unnatural amino acid (UAA) at a position corresponding to V15 of SEQ ID NO: 153; the amino acid sequence of SEQ ID NO: 154, or an amino acid sequence having at least 85%, 90%, 95%, 96%, 97%, 98%, or 99% sequence identity to SEQ ID NO: 154, with an unnatural amino acid (UAA) at a position corresponding to V15 of SEQ ID NO: 154; or the amino acid sequence of SEQ ID NO: 155, or an amino acid sequence having at least 85%, 90%, 95%, 96%, 97%, 98%, or 99% sequence identity to SEQ ID NO: 155, with an unnatural amino acid (UAA) at a position corresponding to V15 of SEQ ID NO: 155. In certain embodiments, the antibody comprises: the amino acid sequence of SEQ ID NO: 156, or an amino acid sequence having at least 85%, 90%, 95%, 96%, 97%, 98%, or 99% sequence identity to SEQ ID NO: 156, with an unnatural amino acid (UAA) at a position corresponding to T6 of SEQ ID NO: 156; the amino acid sequence of SEQ ID NO: 153, or an amino acid sequence having at least 85%, 90%, 95%, 96%, 97%, 98%, or 99% sequence identity to SEQ ID NO: 153, with an unnatural amino acid (UAA) at a position corresponding to T20 of SEQ ID NO: 153; the amino acid sequence of SEQ ID NO: 154, or an amino acid sequence having at least 85%, 90%, 95%, 96%, 97%, 98%, or 99% sequence identity to SEQ ID NO: 154, with an unnatural amino acid (UAA) at a position corresponding to T20 of SEQ ID NO: 154; or the amino acid sequence of SEQ ID NO: 155, or an amino acid sequence having at least 85%, 90%, 95%, 96%, 97%, 98%, or 99% sequence identity to SEQ ID NO: 155, with an unnatural amino acid (UAA) at a position corresponding to T20 of SEQ ID NO: 155.

In certain embodiments, a UAA is located in a light chain CDR1 region of an antibody. For example, in certain embodiments, the antibody comprises: the amino acid sequence of SEQ ID NO: 157, or an amino acid sequence having at least 85%, 90%, 95%, 96%, 97%, 98%, or 99% sequence identity to SEQ ID NO: 157, with an unnatural amino acid (UAA) at a position corresponding to R6 of SEQ ID NO: 157; the amino acid sequence of SEQ ID NO: 178, or an amino acid sequence having at least 85%, 90%, 95%, 96%, 97%, 98%, or 99% sequence identity to SEQ ID NO: 178, with an unnatural amino acid (UAA) at a position corresponding to R9 of SEQ ID NO: 178; the amino acid sequence of SEQ ID NO: 158, or an amino acid sequence having at least 85%, 90%, 95%, 96%, 97%, 98%, or 99% sequence identity to SEQ ID NO: 158, with an unnatural amino acid (UAA) at a position corresponding to R1 of SEQ ID NO: 158; the amino acid sequence of SEQ ID NO: 154, or an amino acid sequence having at least 85%, 90%, 95%, 96%, 97%, 98%, or 99% sequence identity to SEQ ID NO: 154, with an unnatural amino acid (UAA) at a position corresponding to R24 of SEQ ID NO: 154; or the amino acid sequence of SEQ ID NO: 155, or an amino acid sequence having at least 85%, 90%, 95%, 96%, 97%, 98%, or 99% sequence identity to SEQ ID NO: 155, with an unnatural amino acid (UAA) at a position corresponding to R24 of SEQ ID NO: 155.

In certain embodiments, a UAA is located in a light chain FR3 region of an antibody. For example, in certain embodiments, the antibody comprises: the amino acid sequence of SEQ ID NO: 159, or an amino acid sequence having at least 85%, 90%, 95%, 96%, 97%, 98%, or 99% sequence identity to SEQ ID NO: 159, with an unnatural amino acid (UAA) at a position corresponding to S6 of SEQ ID NO: 159; the amino acid sequence of SEQ ID NO: 159, or an amino acid sequence having at least 85%, 90%, 95%, 96%, 97%, 98%, or 99% sequence identity to SEQ ID NO: 159, with an unnatural amino acid (UAA) at a position corresponding to S11 of SEQ ID NO: 159; the amino acid sequence of SEQ ID NO: 179, or an amino acid sequence having at least 85%, 90%, 95%, 96%, 97%, 98%, or 99% sequence identity to SEQ ID NO: 179, with an unnatural amino acid (UAA) at a position corresponding to S6 of SEQ ID NO: 179; the amino acid sequence of SEQ ID NO: 160, or an amino acid sequence having at least 85%, 90%, 95%, 96%, 97%, 98%, or 99% sequence identity to SEQ ID NO: 160, with an unnatural amino acid (UAA) at a position corresponding to S4 of SEQ ID NO: 160; the amino acid sequence of SEQ ID NO: 154, or an amino acid sequence having at least 85%, 90%, 95%, 96%, 97%, 98%, or 99% sequence identity to SEQ ID NO: 154, with an unnatural amino acid (UAA) at a position corresponding to S60 of SEQ ID NO: 154; or the amino acid sequence of SEQ ID NO: 155, or an amino acid sequence having at least 85%, 90%, 95%, 96%, 97%, 98%, or 99% sequence identity to SEQ ID NO: 155, with an unnatural amino acid (UAA) at a position corresponding to S60 of SEQ ID NO: 155.

In certain embodiments, a UAA is located in a light chain FR4 region of an antibody. For example, in certain embodiments, the antibody comprises: the amino acid sequence of SEQ ID NO: 161, or an amino acid sequence having at least 85%, 90%, 95%, 96%, 97%, 98%, or 99% sequence identity to SEQ ID NO: 161, with an unnatural amino acid (UAA) at a position corresponding to K6 of SEQ ID NO: 161; the amino acid sequence of SEQ ID NO: 162, or an amino acid sequence having at least 85%, 90%, 95%, 96%, 97%, 98%, or 99% sequence identity to SEQ ID NO: 162, with an unnatural amino acid (UAA) at a position corresponding to K10 of SEQ ID NO: 162; the amino acid sequence of SEQ ID NO: 154, or an amino acid sequence having at least 85%, 90%, 95%, 96%, 97%, 98%, or 99% sequence identity to SEQ ID NO: 154, with an unnatural amino acid (UAA) at a position corresponding to K107 of SEQ ID NO: 154; or the amino acid sequence of SEQ ID NO: 155, or an amino acid sequence having at least 85%, 90%, 95%, 96%, 97%, 98%, or 99% sequence identity to SEQ ID NO: 155, with an unnatural amino acid (UAA) at a position corresponding to K107 of SEQ ID NO: 155. In certain embodiments, the antibody comprises: the amino acid sequence of SEQ ID NO: 163, or an amino acid sequence having at least 85%, 90%, 95%, 96%, 97%, 98%, or 99% sequence identity to SEQ ID NO: 163, with an unnatural amino acid (UAA) at a position corresponding to T6 of SEQ ID NO: 163; the amino acid sequence of SEQ ID NO: 162, or an amino acid sequence having at least 85%, 90%, 95%, 96%, 97%, 98%, or 99% sequence identity to SEQ ID NO: 162, with an unnatural amino acid (UAA) at a position corresponding to T12 of SEQ ID NO: 162; the amino acid sequence of SEQ ID NO: 154, or an amino acid sequence having at least 85%, 90%, 95%, 96%, 97%, 98%, or 99% sequence identity to SEQ ID NO: 154, with an unnatural amino acid (UAA) at a position corresponding to T109 of SEQ ID NO: 154; or the amino acid sequence of SEQ ID NO: 155, or an amino acid sequence having at least 85%, 90%, 95%, 96%, 97%, 98%, or 99% sequence identity to SEQ ID NO: 155, with an unnatural amino acid (UAA) at a position corresponding to T109 of SEQ ID NO: 155. In certain embodiments, the antibody comprises: the amino acid sequence of SEQ ID NO: 164, or an amino acid sequence having at least 85%, 90%, 95%, 96%, 97%, 98%, or 99% sequence identity to SEQ ID NO: 164, with an unnatural amino acid (UAA) at a position corresponding to V6 of SEQ ID NO: 164; the amino acid sequence of SEQ ID NO: 162, or an amino acid sequence having at least 85%, 90%, 95%, 96%, 97%, 98%, or 99% sequence identity to SEQ ID NO: 162, with an unnatural amino acid (UAA) at a position corresponding to V13 of SEQ ID NO: 162; the amino acid sequence of SEQ ID NO: 154, or an amino acid sequence having at least 85%, 90%, 95%, 96%, 97%, 98%, or 99% sequence identity to SEQ ID NO: 154, with an unnatural amino acid (UAA) at a position corresponding to V110 of SEQ ID NO: 154; or the amino acid sequence of SEQ ID NO: 155, or an amino acid sequence having at least 85%, 90%, 95%, 96%, 97%, 98%, or 99% sequence identity to SEQ ID NO: 155, with an unnatural amino acid (UAA) at a position corresponding to V110 of SEQ ID NO: 155.

In certain embodiments, a UAA is located in a light chain constant region of an antibody. For example, in certain embodiments, the antibody comprises: the amino acid sequence of SEQ ID NO: 165, or an amino acid sequence having at least 85%, 90%, 95%, 96%, 97%, 98%, or 99% sequence identity to SEQ ID NO: 165, with an unnatural amino acid (UAA) at a position corresponding to A6 of SEQ ID NO: 165; the amino acid sequence of SEQ ID NO: 166, or an amino acid sequence having at least 85%, 90%, 95%, 96%, 97%, 98%, or 99% sequence identity to SEQ ID NO: 165, with an unnatural amino acid (UAA) at a position corresponding to A1 of SEQ ID NO: 166; or the amino acid sequence of SEQ ID NO: 155, or an amino acid sequence having at least 85%, 90%, 95%, 96%, 97%, 98%, or 99% sequence identity to SEQ ID NO: 155, with an unnatural amino acid (UAA) at a position corresponding to A111 of SEQ ID NO: 155. In certain embodiments, the antibody comprises: the amino acid sequence of SEQ ID NO: 167, or an amino acid sequence having at least 85%, 90%, 95%, 96%, 97%, 98%, or 99% sequence identity to SEQ ID NO: 167, with an unnatural amino acid (UAA) at a position corresponding to Q6 of SEQ ID NO: 167; the amino acid sequence of SEQ ID NO: 180, or an amino acid sequence having at least 85%, 90%, 95%, 96%, 97%, 98%, or 99% sequence identity to SEQ ID NO: 180, with an unnatural amino acid (UAA) at a position corresponding to Q7 of SEQ ID NO: 180; the amino acid sequence of SEQ ID NO: 166, or an amino acid sequence having at least 85%, 90%, 95%, 96%, 97%, 98%, or 99% sequence identity to SEQ ID NO: 166, with an unnatural amino acid (UAA) at a position corresponding to Q37 of SEQ ID NO: 166; or the amino acid sequence of SEQ ID NO: 155, or an amino acid sequence having at least 85%, 90%, 95%, 96%, 97%, 98%, or 99% sequence identity to SEQ ID NO: 155, with an unnatural amino acid (UAA) at a position corresponding to Q147 of SEQ ID NO: 155. In certain embodiments, the antibody comprises: the amino acid sequence of SEQ ID NO: 168, or an amino acid sequence having at least 85%, 90%, 95%, 96%, 97%, 98%, or 99% sequence identity to SEQ ID NO: 168, with an unnatural amino acid (UAA) at a position corresponding to L6 of SEQ ID NO: 168; the amino acid sequence of SEQ ID NO: 181, or an amino acid sequence having at least 85%, 90%, 95%, 96%, 97%, 98%, or 99% sequence identity to SEQ ID NO: 181, with an unnatural amino acid (UAA) at a position corresponding to L6 of SEQ ID NO: 181; the amino acid sequence of SEQ ID NO: 166, or an amino acid sequence having at least 85%, 90%, 95%, 96%, 97%, 98%, or 99% sequence identity to SEQ ID NO: 166, with an unnatural amino acid (UAA) at a position corresponding to L44 of SEQ ID NO: 166; or the amino acid sequence of SEQ ID NO: 155, or an amino acid sequence having at least 85%, 90%, 95%, 96%, 97%, 98%, or 99% sequence identity to SEQ ID NO: 155, with an unnatural amino acid (UAA) at a position corresponding to L154 of SEQ ID NO: 155. In certain embodiments, the antibody comprises: the amino acid sequence of SEQ ID NO: 169, or an amino acid sequence having at least 85%, 90%, 95%, 96%, 97%, 98%, or 99% sequence identity to SEQ ID NO: 169, with an unnatural amino acid (UAA) at a position corresponding to G6 of SEQ ID NO: 169; the amino acid sequence of SEQ ID NO: 166, or an amino acid sequence having at least 85%, 90%, 95%, 96%, 97%, 98%, or 99% sequence identity to SEQ ID NO: 166, with an unnatural amino acid (UAA) at a position corresponding to G47 of SEQ ID NO: 166; or the amino acid sequence of SEQ ID NO: 155, or an amino acid sequence having at least 85%, 90%, 95%, 96%, 97%, 98%, or 99% sequence identity to SEQ ID NO: 155, with an unnatural amino acid (UAA) at a position corresponding to G157 of SEQ ID NO: 155. In certain embodiments, the antibody comprises: the amino acid sequence of SEQ ID NO: 175, or an amino acid sequence having at least 85%, 90%, 95%, 96%, 97%, 98%, or 99% sequence identity to SEQ ID NO: 175, with an unnatural amino acid (UAA) at a position corresponding to K6 of SEQ ID NO: 175; the amino acid sequence of SEQ ID NO: 166, or an amino acid sequence having at least 85%, 90%, 95%, 96%, 97%, 98%, or 99% sequence identity to SEQ ID NO: 166, with an unnatural amino acid (UAA) at a position corresponding to K59 of SEQ ID NO: 166; or the amino acid sequence of SEQ ID NO: 155, or an amino acid sequence having at least 85%, 90%, 95%, 96%, 97%, 98%, or 99% sequence identity to SEQ ID NO: 155, with an unnatural amino acid (UAA) at a position corresponding to K169 of SEQ ID NO: 155. In certain embodiments, the antibody comprises: the amino acid sequence of SEQ ID NO: 170, or an amino acid sequence having at least 85%, 90%, 95%, 96%, 97%, 98%, or 99% sequence identity to SEQ ID NO: 170, with an unnatural amino acid (UAA) at a position corresponding to A6 of SEQ ID NO: 170; the amino acid sequence of SEQ ID NO: 182, or an amino acid sequence having at least 85%, 90%, 95%, 96%, 97%, 98%, or 99% sequence identity to SEQ ID NO: 182, with an unnatural amino acid (UAA) at a position corresponding to A6 of SEQ ID NO: 182; the amino acid sequence of SEQ ID NO: 166, or an amino acid sequence having at least 85%, 90%, 95%, 96%, 97%, 98%, or 99% sequence identity to SEQ ID NO: 166, with an unnatural amino acid (UAA) at a position corresponding to A83 of SEQ ID NO: 166; or the amino acid sequence of SEQ ID NO: 155, or an amino acid sequence having at least 85%, 90%, 95%, 96%, 97%, 98%, or 99% sequence identity to SEQ ID NO: 155, with an unnatural amino acid (UAA) at a position corresponding to A193 of SEQ ID NO: 155. In certain embodiments, the antibody comprises: the amino acid sequence of SEQ ID NO: 171, or an amino acid sequence having at least 85%, 90%, 95%, 96%, 97%, 98%, or 99% sequence identity to SEQ ID NO: 171, with an unnatural amino acid (UAA) at a position corresponding to V6 of SEQ ID NO: 171; the amino acid sequence of SEQ ID NO: 183, or an amino acid sequence having at least 85%, 90%, 95%, 96%, 97%, 98%, or 99% sequence identity to SEQ ID NO: 183, with an unnatural amino acid (UAA) at a position corresponding to V5 of SEQ ID NO: 183; the amino acid sequence of SEQ ID NO: 166, or an amino acid sequence having at least 85%, 90%, 95%, 96%, 97%, 98%, or 99% sequence identity to SEQ ID NO: 166, with an unnatural amino acid (UAA) at a position corresponding to V95 of SEQ ID NO: 166; or the amino acid sequence of SEQ ID NO: 155, or an amino acid sequence having at least 85%, 90%, 95%, 96%, 97%, 98%, or 99% sequence identity to SEQ ID NO: 155, with an unnatural amino acid (UAA) at a position corresponding to V205 of SEQ ID NO: 155. In certain embodiments, the antibody comprises: the amino acid sequence of SEQ ID NO: 172, or an amino acid sequence having at least 85%, 90%, 95%, 96%, 97%, 98%, or 99% sequence identity to SEQ ID NO: 172, with an unnatural amino acid (UAA) at a position corresponding to T6 of SEQ ID NO: 172; the amino acid sequence of SEQ ID NO: 184, or an amino acid sequence having at least 85%, 90%, 95%, 96%, 97%, 98%, or 99% sequence identity to SEQ ID NO: 184, with an unnatural amino acid (UAA) at a position corresponding to T5 of SEQ ID NO: 184; the amino acid sequence of SEQ ID NO: 166, or an amino acid sequence having at least 85%, 90%, 95%, 96%, 97%, 98%, or 99% sequence identity to SEQ ID NO: 166, with an unnatural amino acid (UAA) at a position corresponding to T96 of SEQ ID NO: 166; or the amino acid sequence of SEQ ID NO: 155, or an amino acid sequence having at least 85%, 90%, 95%, 96%, 97%, 98%, or 99% sequence identity to SEQ ID NO: 155, with an unnatural amino acid (UAA) at a position corresponding to T206 of SEQ ID NO: 155. In certain embodiments, the antibody comprises: the amino acid sequence of SEQ ID NO: 173, or an amino acid sequence having at least 85%, 90%, 95%, 96%, 97%, 98%, or 99% sequence identity to SEQ ID NO: 173, with an unnatural amino acid (UAA) at a position corresponding to S6 of SEQ ID NO: 173; the amino acid sequence of SEQ ID NO: 184, or an amino acid sequence having at least 85%, 90%, 95%, 96%, 97%, 98%, or 99% sequence identity to SEQ ID NO: 184, with an unnatural amino acid (UAA) at a position corresponding to S7 of SEQ ID NO: 184; the amino acid sequence of SEQ ID NO: 166, or an amino acid sequence having at least 85%, 90%, 95%, 96%, 97%, 98%, or 99% sequence identity to SEQ ID NO: 166, with an unnatural amino acid (UAA) at a position corresponding to S98 of SEQ ID NO: 166; or the amino acid sequence of SEQ ID NO: 155, or an amino acid sequence having at least 85%, 90%, 95%, 96%, 97%, 98%, or 99% sequence identity to SEQ ID NO: 155, with an unnatural amino acid (UAA) at a position corresponding to S208 of SEQ ID NO: 155.

Throughout the specification, a first position in a first antibody, antibody fragment, or amino acid sequence is considered to “correspond” with a second position in a second, different antibody, antibody fragment, or amino acid sequence, if a person of skill in the art would understand the first and second positions to correspond to the same position in the primary, secondary, or tertiary structure of their respective antibody, antibody fragment, or amino acid sequence. It is understood that the first and second positions may correspond to each other even if they have a different numbered position relative to N-terminus of their respective antibody, antibody fragment, or amino acid sequence, or if a different amino acid is present at the first and second positions. Primary, secondary, or tertiary structure analysis of antibodies, antibody fragments, or amino acid sequences may be performed using any method known in the art, including, for example, sequence analysis software such as BLAST.

Sequence identity may be determined in various ways that are within the skill of a person skilled in the art, e.g., using publicly available computer software such as BLAST, BLAST-2, ALIGN or Megalign (DNASTAR) software. BLAST (Basic Local Alignment Search Tool) analysis using the algorithm employed by the programs blastp, blastn, blastx, tblastn and tblastx (Karlin et al., (1990) PROC. NATL. ACAD. SCI. USA 87:2264-2268; Altschul, (1993) J. MOL. EVOL. 36:290-300; Altschul et al., (1997) NUCLEIC ACIDS RES. 25:3389-3402, incorporated by reference herein) are tailored for sequence similarity searching. For a discussion of basic issues in searching sequence databases see Altschul et al., (1994) NATURE GENETICS 6:119-129, which is fully incorporated by reference herein. Those skilled in the art can determine appropriate parameters for measuring alignment, including any algorithms needed to achieve maximal alignment over the full length of the sequences being compared. The search parameters for histogram, descriptions, alignments, expect (i.e., the statistical significance threshold for reporting matches against database sequences), cutoff, matrix and filter are at the default settings. The default scoring matrix used by blastp, blastx, tblastn, and tblastx is the BLOSUM62 matrix (Henikoff et al., (1992) PROC. NATL. ACAD. SCI. USA 89:10915-10919, fully incorporated by reference herein). Four blastn parameters may be adjusted as follows: Q=10 (gap creation penalty); R=10 (gap extension penalty); wink=1 (generates word hits at every wink.sup.th position along the query); and gapw=16 (sets the window width within which gapped alignments are generated). The equivalent blastp parameter settings may be Q=9; R=2; wink=1; and gapw=32. Searches may also be conducted using the NCBI (National Center for Biotechnology Information) BLAST Advanced Option parameter (e.g.: −G, Cost to open gap [Integer]: default=5 for nucleotides/11 for proteins; −E, Cost to extend gap [Integer]: default=2 for nucleotides/1 for proteins; −q, Penalty for nucleotide mismatch [Integer]: default=−3; −r, reward for nucleotide match [Integer]: default=1; −e, expect value [Real]: default=10; −W, wordsize [Integer]: default=11 for nucleotides/28 for megablast/3 for proteins; −y, Dropoff (X) for blast extensions in bits: default=20 for blastn/7 for others; −X, X dropoff value for gapped alignment (in bits): default=15 for all programs, not applicable to blastn; and −Z, final X dropoff value for gapped alignment (in bits): 50 for blastn, 25 for others). ClustalW for pairwise protein alignments may also be used (default parameters may include, e.g., Blosum62 matrix and Gap Opening Penalty=10 and Gap Extension Penalty=0.1). A Bestfit comparison between sequences, available in the GCG package version 10.0, uses DNA parameters GAP=50 (gap creation penalty) and LEN=3 (gap extension penalty). The equivalent settings in Bestfit protein comparisons are GAP=8 and LEN=2.

In certain embodiments, the antibody comprises a first UAA at a first position which is indicated in a cell in TABLE 1, and a second UAA at a second position which is indicated in the same cell in TABLE 1. All positions in TABLE 1 are positions corresponding to those in SEQ ID NO: 121 (heavy chain, HC) or SEQ ID NO: 155 (light chain, LC).

TABLE 1

| | | | | |
|---|---|---|---|---|
| — | LC-K107 | LC-K107 | LC-K107 | LC-K107 |
| | LC-T109 | LC-V205 | HC-A121 | HC-A143 |
| LC-T109 | — | LC-T109 | LC-T109 | LC-T109 |
| LC-K107 | | LC-V205 | HC-A121 | HC-A143 |
| LC-V205 | LC-V205 | — | LC-V205 | LC-V205 |
| LC-K107 | LC-T109 | | HC-A121 | HC-A143 |
| HC-A121 | HC-A121 | HC-A121 | — | HC-A121 |
| LC-K107 | LC-T109 | LC-V205 | | HC-A143 |
| HC-A143 | HC-A143 | HC-A143 | HC-A143 | — |
| LC-K107 | LC-T109 | LC-V205 | HC-A121 | |
| HC-T198 | HC-T198 | HC-T198 | HC-T198 | HC-T198 |
| LC-K107 | LC-T109 | LC-V205 | HC-A121 | HC-A143 |
| HC-N204 | HC-N204 | HC-N204 | HC-N204 | HC-N204 |
| LC-K107 | LC-T109 | LC-V205 | HC-A121 | HC-A143 |
| HC-N206 | HC-N206 | HC-N206 | HC-N206 | HC-N206 |
| LC-K107 | LC-T109 | LC-V205 | HC-A121 | HC-A143 |
| HC-K213 | HC-K213 | HC-K213 | HC-K213 | HC-K213 |
| LC-K107 | LC-T109 | LC-V205 | HC-A121 | HC-A143 |
| HC-K293 | HC-K293 | HC-K293 | HC-K293 | HC-K293 |
| LC-K107 | LC-T109 | LC-V205 | HC-A121 | HC-A143 |
| LC-K107 | LC-K107 | LC-K107 | LC-K107 | LC-K107 |
| HC-T198 | HC-N204 | HC-N206 | HC-K213 | HC-K293 |
| LC-T109 | LC-T109 | LC-T109 | LC-T109 | LC-T109 |
| HC-T198 | HC-N204 | HC-N206 | HC-K213 | HC-K293 |
| LC-V205 | LC-V205 | LC-V205 | LC-V205 | LC-V205 |
| HC-T198 | HC-N204 | HC-N206 | HC-K213 | HC-K293 |
| HC-A121 | HC-A121 | HC-A121 | HC-A121 | HC-A121 |
| HC-T198 | HC-N204 | HC-N206 | HC-K213 | HC-K293 |
| HC-A143 | HC-A143 | HC-A143 | HC-A143 | HC-A143 |
| HC-T198 | HC-N204 | HC-N206 | HC-K213 | HC-K293 |
| — | HC-T198 | HC-T198 | HC-T198 | HC-T198 |
| | HC-N204 | HC-N206 | HC-K213 | HC-K293 |
| HC-N204 | — | HC-N204 | HC-N204 | HC-N204 |
| HC-T198 | | HC-N206 | HC-K213 | HC-K293 |
| HC-N206 | HC-N206 | — | HC-N206 | HC-N206 |
| HC-T198 | HC-N204 | | HC-K213 | HC-K293 |
| HC-K213 | HC-K213 | HC-K213 | — | HC-K213 |
| HC-T198 | HC-N204 | HC-N206 | | HC-K293 |
| HC-K293 | HC-K293 | HC-K293 | HC-K293 | — |
| HC-T198 | HC-N204 | HC-N206 | HC-K213 | |

The term antibody includes variants having one or more mutations (e.g., amino acid substitutions, deletions, or insertions) relative to a wild-type antibody sequence or an antibody sequence disclosed herein. In certain embodiments, an antibody variant may comprise, consist, or consist essentially of, a single mutation, or a combination of 2, 3, 4, 5, 6, 7, 8, 9, 10, 11, 12, 13, 14, 15 or more than 15 mutations relative to a wild-type antibody sequence or an antibody sequence disclosed herein. It is contemplated that an antibody variant may comprise, consist, or consist essentially 1-15, 1-10, 1-7, 1-6, 1-5, 1-4, 1-3, 1-2, 2-15, 2-10, 2-7, 2-6, 2-5, 2-4, 2-3, 3-15, 3-10, 3-7, 3-6, 3-5, or 4-10, 4-7, 4-6, 4-5, 5-10, 5-7, 5-6, 6-10, 6-7, 7-10, 7-8, or 8-10 mutations relative to a wild-type antibody sequence or an antibody sequence disclosed herein. An antibody variant may comprise a conservative substitution relative to a wild-type sequence or a sequence disclosed herein. As used herein, the term “conservative substitution” refers to a substitution with a structurally similar amino acid. For example, conservative substitutions may include those within the following groups: Ser and Cys; Leu, Ile, and Val; Glu and Asp; Lys and Arg; Phe, Tyr, and Trp; and Gln, Asn, Glu, Asp, and His. Conservative substitutions may also be defined by the BLAST (Basic Local Alignment Search Tool) algorithm, the BLOSUM substitution matrix (e.g., BLOSUM 62 matrix), or the PAM substitution:p matrix (e.g., the PAM 250 matrix).

The antibody may be selected from, or may be derived from an antibody selected from, adecatumumab, ascrinvacumab, cixutumumab, conatumumab, daratumumab, drozitumab, duligotumab, durvalumab, dusigitumab, enfortumab, enoticumab, epratuxumab, figitumumab, ganitumab, glembatumumab, intetumumab, ipilimumab, iratumumab, icrucumab, lexatumumab, lucatumumab, mapatumumab, narnatumab, necitumumab, nesvacumab, ofatumumab, olaratumab, panitumumab, patritumab, pritumumab, radretumab, ramucirumab, rilotumumab, robatumumab, seribantumab, tarextumab, teprotumumab, tovetumab, vantictumab, vesencumab, votumumab, zalutumumab, flanvotumab, altumomab, anatumomab, arcitumomab, bectumomab, blinatumomab, detumomab, ibritumomab, minretumomab, mitumomab, moxetumomab, naptumomab, nofetumomab, pemtumomab, pintumomab, racotumomab, satumomab, solitomab, taplitumomab, tenatumomab, tositumomab, tremelimumab, abagovomab, atezolizumab, durvalumab, avelumab, igovomab, oregovomab, capromab, edrecolomab, nacolomab, amatuximab, bavituximab, brentuximab, cetuximab, derlotuximab, dinutuximab, ensituximab, futuximab, girentuximab, indatuximab, isatuximab, margetuximab, rituximab, siltuximab, ublituximab, ecromeximab, abituzumab, alemtuzumab, bevacizumab, bivatuzumab, brontictuzumab, cantuzumab, cantuzumab, citatuzumab, clivatuzumab, dacetuzumab, demcizumab, dalotuzumab, denintuzumab, elotuzumab, emactuzumab, emibetuzumab, enoblituzumab, etaracizumab, farletuzumab, ficlatuzumab, gemtuzumab, imgatuzumab, inotuzumab, labetuzumab, lifastuzumab, lintuzumab, lirilumab, lorvotuzumab, lumretuzumab, matuzumab, milatuzumab, moxetumomab, nimotuzumab, obinutuzumab, ocaratuzumab, otlertuzumab, onartuzumab, oportuzumab, parsatuzumab, pertuzumab, pidilizumab, pinatuzumab, polatuzumab, sibrotuzumab, simtuzumab, tacatuzumab, tigatuzumab, trastuzumab, tucotuzumab, urelumab, vandortuzumab, vanucizumab, veltuzumab, vorsetuzumab, sofituzumab, catumaxomab, ertumaxomab, depatuxizumab, ontuxizumab, blontuvetmab, tamtuvetmab, nivolumab, pembrolizumab, epratuzumab, MEDI9447, urelumab, utomilumab, hu3F8, hu14.18-IL-2, 3F8/OKT3BsAb, lirilumab, BMS-986016 pidilizumab, AMP-224, AMP-514, BMS-936559, atezolizumab, and avelumab.

The antibody may bind an antigen selected from, for example, adenosine A2a receptor (A2aR), A kinase anchor protein 4 (AKAP4), B melanoma antigen (BAGE), brother of the regulator of imprinted sites (BORIS), breakpoint cluster region Abelson tyrosine kinase (BCR/ABL), CA125, CAIX, CD19, CD20, CD22, CD30, CD33, CD52, CD73, CD137, carcinoembryonic antigen (CEA), a claudin (e.g. a claudin 18, e.g., claudin 18.2), CS1, cytotoxic T-lymphocyte-associated antigen 4 (CTLA-4), estrogen receptor binding site associated antigen 9 (EBAG9), epidermal growth factor (EGF), epidermal growth factor receptor (EGFR), EGF-like module receptor 2 (EMR2), epithelial cell adhesion molecule (EpCAM) (17-1A), FR-alpha, G antigen (GAGE), disialoganglioside GD2 (GD2), glycoprotein 100 (gp100), human epidermal growth factor receptor 2 (HER2), hepatocyte growth factor (HGF), human papillomavirus 16 (HPV-16), heat-shock protein 105 (HSP105), isocitrate dehydrogenase type 1 (IDH1), idiotype (NeuGcGM3), indoleamine-2,3-dioxygenase 1 (IDO1), IGF-1, IGF1R, IGG1K, killer cell immunoglobulin-like receptor (KIR), lymphocyte activation gene 3 (LAG-3), lymphocyte antigen 6 complex K (LY6K), Matrix-metalloproteinase-16 (MMP16), melanotransferrin (MFI2), melanoma antigen 3 (MAGE-A3), melanoma antigen C2 (MAGE-C2), melanoma antigen D4 (MAGE-D4), melanoma antigen recognized by T-cells 1 (Melan-A/MART-1), N-methyl-N′-nitroso-guanidine human osteosarcoma transforming gene (MET), mucin 1 (MUC1), mucin 4 (MUC4), mucin 16 (MUC16), New York esophageal squamous cell carcinoma 1 (NY-ESO-1), prostatic acid phosphatase (PAP), programmed cell death receptor 1 (PD-1), programmed cell death receptor ligand 1 (PD-L1), phosphatidylserine, preferentially expressed antigen of melanoma (PRAME), prostate specific antigen (PSA), protein tyrosine kinase 7 (PTK7, also known as colon carcinoma kinase 4 (CCK4)), receptor tyrosine kinase orphan receptor 1 (ROR1), scatter factor receptor kinase, sialyl-Tn, sperm-associated antigen 9 (SPAG-9), synovial sarcoma X-chromosome breakpoint 1 (SSX1), survivin, telomerase, T-cell immunoglobulin domain and mucin domain-3 (TIM-3), vascular endothelial growth factor (VEGF) (e.g., VEGF-A), vascular endothelial growth factor Receptor 2 (VEGFR2), V-domain immunoglobulin-containing suppressor of T-cell activation (VISTA), Wilms' Tumor-1 (WT1), X chromosome antigen 1b (XAGE-1b), 5T4, Mesothelin, Glypican 3 (GPC3), Folate Receptor α (FRα), Prostate Specific Membrane Antigen (PSMA), cMET, CD38, B Cell Maturation Antigen (BCMA), CD123, CLDN6, CLDN9, LRRC15, PRLR (Prolactin Receptor), RING finger protein 43 (RNF43), Uroplakin-1 B (UPK1 B), tumor necrosis factor superfamily member 9 (TNFSF9), tumor necrosis factor receptor superfamily member 21 (TNFSRF21), bone morphogenetic protein receptor type-1B (BMPR1B), Kringle domain-containing transmembrane protein 2 (KREMEN2), Delta-like protein 3 (DLL3), Siglec7 and Siglec9. Additional exemplary cancer antigens include those found on cancer stem cells, e.g., SSEA3, SSEA4, TRA-1-60, TRA-1-81, SSEA1, CD133 (AC133), CD90 (Thy-1), CD326 (EpCAM), Cripto-1 (TDGF1), PODXL-1 (Podocalyxin-like protein 1), ABCG2, CD24, CD49f (Integrin α6), Notch2, CD146 (MCAM), CD10 (Neprilysin), CD117 (c-KIT), CD26 (DPP-4), CXCR4, CD34, CD271, CD13 (Alanine aminopeptidase), CD56 (NCAM), CD105 (Endoglin), LGR5, CD114 (CSF3R), CD54 (ICAM-1), CXCR1, 2, TIM-3 (HAVCR2), CD55 (DAF), DLL4 (Delta-like ligand 4), CD20 (MS4A1), and CD96.

TABLE 2 shows antibodies and antibody-drug conjugates suitable for use in accordance with the present invention, the antigen bound by the antibody or antibody-drug conjugate, and for certain antibodies, the type of cancer targeted by the antibody or antibody-drug conjugate.

TABLE 2

| Antibody or antibody- | | |
|---|---|---|
| drug conjugate | Cancer Antigen | Cancer Type |
| oregovomab | CA125 | |
| girentuximab | CAIX | |
| obinutuzumab | CD20 | |
| ofatumumab | CD20 | |
| rituximab | CD20 | |
| alemtuzumab | CD52 | |
| Ipilimumab | cytotoxic T-lymphocyte-associated | |
| | antigen 4 (CTLA-4) | |
| tremelimumab | CTLA-4 | |
| Cetuximab | epidermal growth factor receptor | |
| | (EGFR) | |
| necitumumab | EGFR | |
| panitumumab | EGFR | |
| zalutumumab | EGFR | |
| edrecolomab | epithelial cell adhesion molecule | |
| | (EpCAM) (17-1A) | |
| farletuzumab | FR-alpha | |
| Pertuzumab | human epidermal growth factor | |
| | receptor 2 (HER2) | |
| trastuzumab | HER2 | |
| rilotumumab | HGF | |
| figitumumab | IGF-1 | |
| Ganitumab | IGF1R | |
| durvalumab | IGG1K | |
| bavituximab | Phosphatidylserine | |
| onartuzumab | scatter factor receptor kinase | |
| bevacizumab | vascular endothelial growth factor-A | |
| | (VEGF-A) | |
| ramucirumab | vascular endothelial growth factor | |
| | Receptor 2 (VEGFR2) | |
| blinatumomab | CD19 | acute lymphoblastic leukemia |
| | | (ALL) |
| Rituximab; | CD20 | non-Hodgkin's lymphoma |
| ofatumumab; | | (NHL), chronic lymphocytic |
| ibritumomab (e.g., ⁹⁰Y- | | leukemia (CLL) |
| ibritumomab; | | B-cell NHL |
| tositumomab (e.g., ¹³¹I- | | pre-B ALL |
| tositumomab | | |
| brentuximab (e.g., | CD30 | Hodgkin's lymphoma |
| brentuximab vedotin | | |
| gemtuzumab (e.g., | CD33 | acute myelogenous leukemia |
| gemtuzumab ozogamicin | | (AML) |
| Alemtuzumab | CD52 | CLL |
| Antibody or antibody- | | Cancer Type |
| drug conjugate | | |
| Ipilimumab | cytotoxic T-lymphocyte-associated | Unresectable or metastatic |
| | antigen 4 (CTLA-4) | melanoma |
| cetuximab; | epidermal growth factor receptor | colorectal cancer (CRC) |
| panitumumab | (EGFR) | Head and Neck |
| Catumaxomab | epithelial cell adhesion molecule | Malignant ascites |
| | (EpCAM) | |
| trastuzumab; | human epidermal growth factor | Breast |
| pertuzumab | receptor 2 (HER2) | |
| nivolumab; | programmed cell death receptor 1 | Metastatic melanoma, non- |
| pembrolizumab | (PD-1) | small cell lung cancer |
| | | (NSCLC) |
| Bevacizumab | vascular endothelial growth factor | Breast, Cervical |
| | (VEGF) | CRC, NSCLC |
| | | renal cell carcinoma (RCC), |
| | | Ovarian |
| | | Glioblastoma |
| Ramucirumab | vascular endothelial growth factor | Gastric |
| | receptor 2 (VEGF-R2) | NSCLC |
| Epratuzumab; | CD22 | acute lymphoblastic leukemia |
| moxetumomab; | | (ALL) |
| inotuzumab (e.g., | | |
| inotuzumab ozogamicin) | | |
| MEDI9447 | CD73 | Advanced solid tumors |
| Urelumab; | CD137 | Advanced solid tumors |
| utomilumab (PF- | | |
| 05082566) | | |
| Elotuzumab | CD2 subset 1 (CS1) | Multiple myeloma |
| Tremelimumab | cytotoxic T-lymphocyte-associated | Malignant mesothelioma |
| | antigen 4 (CTLA-4) | |
| Necitumumab | epidermal growth factor receptor | non-small cell lung cancer |
| | (EGFR) | NSCLC) |
| dinutuximab; hu3F8; | disialoganglioside GD2 (GD2) | Neuroblastoma |
| hu14.18-IL-2; | | Retinoblastoma |
| 3F8/OKT3BsAb | | Melanoma other solid tumors |
| Racotumomab | Idiotype (NeuGcGM3) | NSCLC, Breast |
| | | Melanoma |
| Lirilumab | killer cell immunoglobulin-like | Lymphoma |
| | receptor (KIR) | |
| BMS-986016 | lymphocyte activation gene 3 (LAG- | Breast, Hematological, |
| | 3) | Advanced solid tumors |
| Onartuzumab | N-methyl-N′-nitroso-guanidine | NSCLC |
| | human osteosarcoma transforming | |
| | gene (MET) | |
| abagovomab; | mucin 16 (MUC16) | Ovarian |
| oregovomab | | |
| Antibody or antibody- | Cancer Antigen | Cancer Type |
| drug conjugate | | |
| pidilizumab; | programmed cell death receptor 1 | B-cell lymphoma |
| AMP-224; AMP-514 | (PD-1) | Melanoma, CRC |
| BMS-936559; | programmed cell death receptor | NSCLC, renal cell carcinoma |
| atezolizumab; | ligand 1 (PD-L1) | (RCC) |
| durvalumab; avelumab | | Bladder, Breast |
| | | Melanoma, squamous cell |
| | | carcinoma of the head and |
| | | neck (SCCHN) |
| naptumomab (e.g., | 5T4 | RCC, CRC |
| naptumomab estafenatox) | | Prostate |

In certain embodiments, the antibody is, or is derived from, trastuzumab (e.g., comprising a heavy chain amino acid sequence of SEQ ID NO: 121 and a light chain amino acid sequence of SEQ ID NO: 155).

In certain embodiments, the antibody has a binding affinity (K_{D}) for a target antigen of at least 20 nM, 15 nM, 10 nM, 9 nM, 8 nM, 7 nM, 6 nM, 5 nM, 4 nM, 3 nM, 2 nM, 1 nM, 0.75 nM, 0.5 nM, 0.1 nM, 0.075 nM, or 0.05 nM or lower, as measured using standard binding assays, for example, ELISA (e.g., as described in Example 3 herein), surface plasmon resonance or bio-layer interferometry. In certain embodiments, the antibody binds a target antigen with a K_{D} of from about 20 nM to about 0.05 nM, from about 20 nM to about 0.075 nM, from about 20 nM to about 0.1 nM, from about 20 nM to about 0.5 nM, from about 20 nM to about 1 nM, from about 10 nM to about 0.05 nM, from about 10 nM to about 0.075 nM, from about 10 nM to about 0.1 nM, from about 10 nM to about 0.5 nM, from about 10 nM to about 1 nM, from about 5 nM to about 0.05 nM, from about 5 nM to about 0.075 nM, from about 5 nM to about 0.1 nM, from about 5 nM to about 0.5 nM, from about 5 nM to about 1 nM, from about 3 nM to about 0.05 nM, from about 3 nM to about 0.075 nM, from about 3 nM to about 0.1 nM, from about 3 nM to about 0.5 nM, from about 3 nM to about 1 nM, from about 3 nM to about 2 nM, from about 2 nM to about 0.05 nM, from about 2 nM to about 0.075 nM, from about 2 nM to about 0.1 nM, from about 2 nM to about 0.5 nM, from about 2 nM to about 1 nM, from about 1 nM to about 0.05 nM, from about 1 nM to about 0.075 nM, from about 1 nM to about 0.1 nM, from about 1 nM to about 0.5 nM, from about 0.5 nM to about 0.05 nM, from about 0.5 nM to about 0.075 nM, from about 0.5 nM to about 0.1 nM, from about 0.1 nM to about 0.05 nM, from about 0.1 nM to about 0.075 nM, or from about 0.075 nM to about 0.05 nM, or from about 0.05 nM to about 0.035 nM, as measured using standard binding assays, for example, ELISA (e.g., as described in Example 3 herein), surface plasmon resonance or bio-layer interferometry.

In certain embodiments, the antibody has a binding affinity (K_{D}) for a target antigen that is within 0.1 fold, 0.2 fold, 0.3 fold, 0.4 fold, 0.5 fold, 1.0 fold, 1.5 fold, 2.0 fold, 3.0 fold, 4.0 fold, 5.0 fold, 6.0 fold, 8.0 fold, or 10.0 fold of the binding affinity for the target antigen of a reference antibody, wherein the reference antibody is an otherwise identical antibody that does not comprise the UAA, as measured using standard binding assays, for example, ELISA, surface plasmon resonance or bio-layer interferometry.

It is contemplated that the antibody (including an antibody conjugate) may have comparable or even improved stability relative a reference antibody, wherein the reference antibody is an otherwise identical antibody that does not comprise the UAA or a molecule conjugated to the UAA. For example, in certain embodiments, at least 40%, 50%, 60%, 70%, 80%, or 90% of the antibody remains following incubation in human plasma for 72 hours at 37° C. In certain embodiments, at least 40%, 50%, 60%, 70%, 80%, or 90% of the antibody remains following incubation with Cathepsin B for 240 minutes at 37° C. Cathepsin B stability may be assayed as described in Example 3 herein, including, for example, in 25 mM sodium acetate and 1 mM EDTA at pH 5.0.

In certain embodiments, the antibody has off-target binding or activity that is within 0.1 fold, 0.2 fold, 0.3 fold, 0.4 fold, 0.5 fold, 1.0 fold, 1.5 fold, 2.0 fold, 3.0 fold, 4.0 fold, 5.0 fold, 6.0 fold, 8.0 fold, or 10.0 fold of the off-target binding or activity of a reference antibody, wherein the reference antibody is an otherwise identical antibody that does not comprise any UAA, does not comprise the same UAA, does not comprise the same UAA at the same position, and/or does not comprise the same molecule conjugated to the UAA/antibody. In certain embodiments, the antibody has off-target binding or activity that is 0.1 fold, 0.2 fold, 0.3 fold, 0.4 fold, 0.5 fold, 1.0 fold, 1.5 fold, 2.0 fold, 3.0 fold, 4.0 fold, 5.0 fold, 6.0 fold, 8.0 fold, or 10.0 fold less than the off-target binding or activity of a reference antibody, wherein the reference antibody is an otherwise identical antibody that does not comprise any UAA, does not comprise the same UAA, does not comprise the same UAA at the same position, and/or does not comprise the same molecule conjugated to the UAA/antibody. Off-target binding or activity may be measured by any assays known in the art

In certain embodiments, the antibody has an efficacy or therapeutic activity (e.g., IC50) that is within 0.1 fold, 0.2 fold, 0.3 fold, 0.4 fold, 0.5 fold, 1.0 fold, 1.5 fold, 2.0 fold, 3.0 fold, 4.0 fold, 5.0 fold, 6.0 fold, 8.0 fold, or 10.0 fold of the efficacy or therapeutic activity of a reference antibody, wherein the reference antibody is an otherwise identical antibody that does not comprise any UAA, does not comprise the same UAA, does not comprise the same UAA at the same position, and/or does not comprise the same molecule conjugated to the UAA/antibody. In certain embodiments, the antibody has an efficacy or therapeutic activity (e.g., IC50) that is 0.1 fold, 0.2 fold, 0.3 fold, 0.4 fold, 0.5 fold, 1.0 fold, 1.5 fold, 2.0 fold, 3.0 fold, 4.0 fold, 5.0 fold, 6.0 fold, 8.0 fold, or 10.0 fold greater than the efficacy or therapeutic activity of a reference antibody, wherein the reference antibody is an otherwise identical antibody that does not comprise any UAA, does not comprise the same UAA, does not comprise the same UAA at the same position, and/or does not comprise the same molecule conjugated to the UAA/antibody. Efficacy or therapeutic activity may be measured by any assays known in the art, including, e.g., in vitro cytotoxicity assays as described in Example 4 herein.

### II. Unnatural Amino Acids (UAAs)

The invention relates to unnatural amino acids (UAAs) and their incorporation into proteins (e.g. antibodies).

As used herein, an unnatural amino acid refers to any amino acid, modified amino acid, or amino acid analogue other than the following twenty genetically encoded alpha-amino acids: alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, valine. See, e.g., Biochemistry by L. Stryer, 3rd ed. 1988, Freeman and Company, New York, for structures of the twenty natural amino acids. The term unnatural amino acid also includes amino acids that occur by modification (e.g. post-translational modifications) of a natural amino acid but are not themselves naturally incorporated into a growing polypeptide chain by the translation complex.

Because unnatural amino acids typically differ from natural amino acids only in the structure of the side chain, unnatural amino acids may, for example, form amide bonds with other amino acids in the same manner in which they are formed in naturally occurring proteins. However, the unnatural amino acids have side chain groups that distinguish them from the natural amino acids. For example, the side chain may comprise an alkyl-, aryl-, acyl-, keto-, azido-, hydroxyl-, hydrazine, cyano-, halo-, hydrazide, alkenyl, alkyl, ether, thiol, seleno-, sulfonyl-, borate, boronate, phospho, phosphono, phosphine, heterocyclic, enone, imine, aldehyde, ester, thioacid, hydroxylamine, amine, and the like, or any combination thereof. Other non-naturally occurring amino acids include, but are not limited to, amino acids comprising a photoactivatable cross-linker, spin-labeled amino acids, fluorescent amino acids, metal binding amino acids, metal-containing amino acids, radioactive amino acids, amino acids with novel functional groups, amino acids that covalently or noncovalently interact with other molecules, photocaged and/or photoisomerizable amino acids, amino acids comprising biotin or a biotin analogue, glycosylated amino acids such as a sugar substituted serine, other carbohydrate modified amino acids, keto-containing amino acids, amino acids comprising polyethylene glycol or polyether, heavy atom substituted amino acids, chemically cleavable and/or photocleavable amino acids, amino acids with an elongated side chains as compared to natural amino acids, including but not limited to, polyethers or long chain hydrocarbons, including but not limited to, greater than about 5 or greater than about 10 carbons, carbon-linked sugar-containing amino acids, redox-active amino acids, amino thioacid containing amino acids, and amino acids comprising one or more toxic moiety.

In addition to unnatural amino acids that contain novel side chains, unnatural amino acids also optionally comprise modified backbone structures.

Many unnatural amino acids are based on natural amino acids, such as tyrosine, glutamine, phenylalanine, and the like. Tyrosine analogs include para-substituted tyrosines, ortho-substituted tyrosines, and meta substituted tyrosines, wherein the substituted tyrosine comprises a keto group (including but not limited to, an acetyl group), a benzoyl group, an amino group, a hydrazine, an hydroxyamine, a thiol group, a carboxy group, an isopropyl group, a methyl group, a C₆-C₂₀ straight chain or branched hydrocarbon, a saturated or unsaturated hydrocarbon, an O-methyl group, a polyether group, a nitro group, or the like. In addition, multiply substituted aryl rings are also contemplated. Glutamine analogs include, but are not limited to, α-hydroxy derivatives, γ-substituted derivatives, cyclic derivatives, and amide substituted glutamine derivatives. Exemplary phenylalanine analogs include, but are not limited to, para-substituted phenylalanines, ortho-substituted phenylalanines, and meta-substituted phenylalanines, wherein the substituent comprises a hydroxy group, a methoxy group, a methyl group, an allyl group, an aldehyde, an azido, an iodo, a bromo, a keto group (including but not limited to, an acetyl group), or the like. Specific examples of unnatural amino acids include, but are not limited to, a p-acetyl-L-phenylalanine, a p-propargyl-phenylalanine, O-methyl-L-tyrosine, an L-3-(2-naphthyl)alanine, a 3-methyl-phenylalanine, an O-4-allyl-L-tyrosine, a 4-propyl-L-tyrosine, a tri-O-acetyl-GlcNAcβ-serine, an L-Dopa, a fluorinated phenylalanine, an isopropyl-L-phenylalanine, a p-azido-L-phenylalanine, a p-acyl-L-phenylalanine, a p-benzoyl-L-phenylalanine, an L-phosphoserine, a phosphonoserine, a phosphonotyrosine, a p-iodo-phenylalanine, a p-bromophenylalanine, a p-amino-L-phenylalanine, an isopropyl-L-phenylalanine, and a p-propargyloxy-phenylalanine, and the like.

Examples of structures of a variety of unnatural amino acids are provided in U.S. Patent Application Publication Nos. 2003/0082575 and 2003/0108885, PCT Publication No. WO 2002/085923, and Kiick et al. (2002) PROC. NATL. ACAD. SCI. USA 99:19-24.

Any suitable unnatural amino acid can be used with the methods described herein for incorporation into a protein (e.g., an antibody) of interest.

The unnatural amino acid may be a leucine analog (also referred to herein as a derivative). In certain embodiments, the leucine analog is a non-naturally occurring leucine analog. The invention provides a leucine analog depicted in FIG. 6A, or a composition comprising the leucine analog. For example, Formula A in FIG. 6A depicts an amino acid analog containing a side chain including a carbon containing chain n units (0-20 units) long. An O, S, CH₂, or NH is present in at position X, and another carbon containing chain of n units (0-20 units) long can follow. A functional group Y is attached to the terminal carbon of second carbon containing chain (for example, functional groups 1-12 as depicted in FIG. 6A, where R represents a linkage to the terminal carbon atom the second carbon containing side chain). In one example, these functional groups can be used for bioconjugation of any amenable ligand to any protein of interest that is amenable to site-specific UAA incorporation. Formula B in FIG. 6A depicts a similar amino acid analog containing an side chains denoted as either Z—Y₂ or Z—Y₃ attached to the second carbon containing chain or the first carbon containing chain, respectively. Z represents a carbon chain comprising (CH₂)n units, where n is any integer from 0-20. Y₂ or Y₃, independently, can be the same or different groups as those of Y₁. The invention also provides a leucine analog depicted in FIG. 6C (LCA, LKET, or ACA), or a composition comprising the leucine analog depicted in FIG. 6C. Additional exemplary leucine analogs include those selected from linear alkyl halides and linear aliphatic chains comprising a functional group, for example, an alkyne, azide, cyclopropene, alkene, ketone, aldehyde, diazirine, or tetrazine functional group, as well as structures 1-6 shown in FIG. 6D. However, it is contemplated that the amino and carboxylate groups both attached to the first carbon of any amino acid shown in FIG. 6A, 6C, or 6D would constitute portions of peptide bonds when the leucine analog is incorporated into a protein or polypeptide chain.

In addition, the leucine analogs set forth in FIG. 6E, referred to as C5AzMe and LCA can be used in the practice of the invention. Methods for preparing leucine analogs, e.g., C5AzMe or LCA, are described in International (PCT) Publication No. WO2021026506.

In certain embodiments, the unnatural amino acid is a tryptophan analog (also referred to herein as a derivative). In certain embodiments, the tryptophan analog is a non-naturally occurring tryptophan analog. Exemplary tryptophan analogs include 5-azidotryptophan, 5-propargyloxytryptophan, 5-aminotryptophan, 5-methoxytryptophan, 5-O-allyltryptophan or 5-bromotryptophan. Additional exemplary tryptophan analogs are depicted in FIG. 6B. However, it is contemplated that the amino and carboxylate groups both attached to the first carbon of the tryptophan analogs in FIG. 6B would constitute portions of peptide bonds when the tryptophan analog is incorporated into a protein or polypeptide chain.

In addition, the tryptophan analog set forth in FIG. 6E, referred to as AzW, can be used in the practice of the invention. Methods for preparing tryptophan analogs, e.g., AzW, are described in International (PCT) Publication No. WO2021026506.

In certain embodiments, the unnatural amino acid is a tyrosine analog (also referred to herein as a derivative). In certain embodiments, the tyrosine analog is a non-naturally occurring tyrosine analog. Exemplary tyrosine analogs include o-methyltyrosine (OmeY), p-azidophenylalanine (AzF), o-propargyltyrosine (OpropY or PrY), and p-acetylphenylalanine (AcF). Exemplary tryptophan analogs are depicted in FIG. 6F.

In certain embodiments, the unnatural amino acid is a pyrrolysine analog (also referred to herein as a derivative). In certain embodiments, the pyrrolysine analog is a non-naturally occurring pyrrolysine analog. Exemplary pyrrolysine analogs include aminocaprylic acid (Cap), H-Lys(Boc)-OH (Boc-Lysine, BocK), azidolysine (AzK), H-propargyl-lysine (hPrK), and cyclopropenelysine (CpK). Exemplary pyrrolysine analogs are depicted in FIG. 6G.

Many unnatural amino acids are commercially available, e.g., from Sigma-Aldrich (St. Louis, Mo., USA), Novabiochem (Darmstadt, Germany), or Peptech (Burlington, Mass., USA). Those that are not commercially available can be synthesized using standard methods known to those of ordinary skill in the art. For organic synthesis techniques, see, e.g., Organic Chemistry by Fessendon and Fessendon, (1982, Second Edition, Willard Grant Press, Boston Mass.); Advanced Organic Chemistry by March (Third Edition, 1985, Wiley and Sons, New York); and Advanced Organic Chemistry by Carey and Sundberg (Third Edition, Parts A and B, 1990, Plenum Press, New York). Additional exemplary publications describing the synthesis of unnatural amino acids appear in PCT Publication No. WO2002/085923, U.S. Patent Application Publication No. 2004/0198637, Matsoukas et al. (1995) J. MED. CHEM. 38:4660-4669, King et al. (1949) J. CHEM. SOC. 3315-3319, Friedman et al. (1959) J. AM. CHEM. SOC. 81:3750-3752, Craig et al. (1988) J. ORG. CHEM. 53:1167-1170, Azoulay et al. (1991) EUR. J. MED. CHEM. 26:201-5, Koskinen et al. (1989) J. ORG. CHEM. 54:1859-1866, Christie et al. (1985) J. ORG. CHEM. 50:1239-1246, Barton et al. (1987) TETRAHEDRON 43:4297-4308, and Subasinghe et al. (1992) J. MED. CHEM. 35:4602-7.

In certain embodiments, where the antibody comprises two or more than two UAAs, the antibody comprises a first unnatural amino acid (UAA) that is a tryptophan analog (e.g., a non-naturally occurring tryptophan analog) and a second UAA that is a leucine analog (e.g., a non-naturally occurring leucine analog). In certain embodiments, the tryptophan analog is selected from 5-HTP and 5-AzW and/or the leucine analog is selected from LCA and Cys-5-N3.

In certain embodiments, where the antibody comprises two or more than two UAAs, the antibody comprises a first unnatural amino acid (UAA) that is a tryptophan analog (e.g., a non-naturally occurring tryptophan analog) and a second UAA that is a tyrosine analog (e.g., a non-naturally occurring tyrosine analog). In certain embodiments, the tryptophan analog is selected from 5-HTP and 5-AzW and/or the tyrosine analog is selected from OmeY, AzF, and OpropY UAA.

In certain embodiments, where the antibody comprises two or more than two UAAs, the antibody comprises a first unnatural amino acid (UAA) that is a tryptophan analog (e.g., a non-naturally occurring tryptophan analog) and a second UAA that is a pyrrolysine analog (e.g., a non-naturally occurring pyrrolysine analog). In certain embodiments, the tryptophan analog is selected from 5-HTP and 5-AzW and/or the pyrrolysine analog is selected from Bock, CpK, AzK, and CpK.

### III. Antibody Conjugates

An unnatural amino acid in a polypeptide (e.g., an antibody) may be used to attach another molecule to the polypeptide. For example, in certain embodiments, a disclosed antibody comprises a chemical modification of an unnatural amino acid (UAA), e.g., a conjugation to a molecule. It is contemplated that an antibody may comprise one or more UAAs (e.g., one, two, three, four, five, six, seven, eight, nine, ten, or more than ten UAAs, each of which may be the same or different), and similarly, may be conjugated to one or more molecules (e.g., one, two, three, four, five, six, seven, eight, nine, ten, or more than ten molecules, each of which may be the same or different).

Exemplary molecules for conjugation include a label, a dye, a polymer, a water-soluble polymer, a stabilizing agent (e.g., a derivative of polyethylene glycol), a photoactivatable crosslinker, a radionuclide, a cytotoxic compound, a drug, an affinity label, a photoaffinity label, a reactive compound, a resin, a second protein or polypeptide or polypeptide analog (e.g., a therapeutic peptide or polypeptide), an antibody or antibody fragment (e.g., an anti-CD3 antibody or antibody fragment), a metal chelator, a cofactor, a fatty acid, a carbohydrate, a polynucleotide, a DNA (e.g., a DNA oligonucleotide), a RNA (e.g., a DNA oligonucleotide), a LNA (e.g., a LNA oligonucleotide), an antisense polynucleotide, a saccharide, a water-soluble dendrimer, a cyclodextrin, an inhibitory ribonucleic acid (e.g., a small interfering RNA (siRNA), a small nuclear RNA (snRNA), or a non-coding RNA), a biomaterial, a nanoparticle, a spin label, a fluorophore, a metal-containing moiety, a radioactive moiety, a novel functional group, a group that covalently or noncovalently interacts with other molecules, a photocaged moiety, an actinic radiation excitable moiety, a photoisomerizable moiety, biotin, a derivative of biotin, a biotin analogue, a moiety incorporating a heavy atom, a chemically cleavable group, a photocleavable group, an elongated side chain, a carbon-linked sugar, a redox-active agent, an amino thioacid, a toxic moiety, an isotopically labeled moiety, a biophysical probe or biochemical probe (e.g., a PET probe, a fluorescent probe or an EPR probe), a phosphorescent group, a chemiluminescent group, an electron dense group, a magnetic group, an intercalating group, a chromophore, an energy transfer agent, a biologically active agent, a detectable label (e.g., for analysis of uptake in viable cells versus non-viable cells), a small molecule (e.g. a therapeutic small molecule), a quantum dot, a nanotransmitter, an immunomodulatory molecule, a targeting agent, a lipid based structure (e.g., a lipid-based nanoparticle), a microsphere, or any combination of the above.

Additional exemplary molecules for conjugation include any cytotoxic, cytostatic or immunomodulatory drug. Useful classes of cytotoxic or immunomodulatory agents include, for example, antitubulin agents, auristatins, DNA minor groove binders, DNA replication inhibitors, alkylating agents (e.g., platinum complexes such as cis-platin, mono(platinum), bis(platinum) and tri-nuclear platinum complexes and carboplatin), anthracyclines, antibiotics, antifolates, antimetabolites, calmodulin inhibitors, chemotherapy sensitizers, duocarmycins, etoposides, fluorinated pyrimidines, ionophores, lexitropsins, maytansinoids, nitrosoureas, platinols, pore-forming compounds, purine antimetabolites, puromycins, radiation sensitizers, rapamycins, steroids, taxanes, topoisomerase inhibitors, vinca alkaloids, or the like.

Individual cytotoxic or immunomodulatory agents include, for example, an androgen, anthramycin (AMC), asparaginase, 5-azacytidine, azathioprine, bleomycin, busulfan, buthionine sulfoximine, calicheamicin, calicheamicin derivatives, camptothecin, carboplatin, carmustine (BSNU), CC-1065, chlorambucil, cisplatin, colchicine, cyclophosphamide, cytarabine, cytidine arabinoside, cytochalasin B, dacarbazine, dactinomycin (formerly actinomycin), daunorubicin, decarbazine, DM1, DM4, docetaxel, doxorubicin, etoposide, an estrogen, 5-fluordeoxyuridine, 5-fluorouracil, gemcitabine, gramicidin D, hydroxyurea, idarubicin, ifosfamide, irinotecan, lomustine (CCNU), maytansine, mechlorethamine, melphalan, 6-mercaptopurine, methotrexate, mithramycin, mitomycin C, mitoxantrone, nitroimidazole, paclitaxel, palytoxin, plicamycin, procarbizine, a pyrrolobenzodiazepine, rhizoxin, streptozotocin, tenoposide, 6-thioguanine, thioTEPA, topotecan, vinblastine, vincristine, vinorelbine, VP-16 and VM-26.

In certain embodiments, suitable cytotoxic agents include, for example, DNA minor groove binders (e.g., enediynes and lexitropsins, a CBI compound), duocarmycins, taxanes (e.g., paclitaxel and docetaxel), puromycins, vinca alkaloids, CC-1065, SN-38, topotecan, morpholino-doxorubicin, rhizoxin, cyanomorpholino-doxorubicin, echinomycin, combretastatin, netropsin, epothilone A and B, estramustine, cryptophycins, cemadotin, maytansinoids, discodermolide, eleutherobin, and mitoxantrone.

In certain embodiments, the molecule is an anti-tubulin agent. Examples of anti-tubulin agents include taxanes (e.g., Taxol® (paclitaxel), Taxotere® (docetaxel)), T67 (Tularik) and vinca alkyloids (e.g., vincristine, vinblastine, vindesine, and vinorelbine). Other antitubulin agents include, for example, baccatin derivatives, taxane analogs, epothilones (e.g., epothilone A and B), nocodazole, colchicine and colcimid, estramustine, cryptophycins, cemadotin, maytansinoids, combretastatins, discodermolide, and eleutherobin.

In certain embodiments, the cytotoxic agent is a maytansinoid, another group of anti-tubulin agents. For example, in specific examples, the maytansinoid can be maytansine or DM1.

In certain embodiments, the molecule is an auristatin, such as auristatin E or a derivative thereof. For example, the auristatin E derivative can be an ester formed between auristatin E and a keto acid. For example, auristatin E can be reacted with paraacetyl benzoic acid or benzoylvaleric acid to produce AEB and AEVB, respectively. Other typical auristatin derivatives include AFP, MMAF, and MMAE.

In certain embodiments, the molecule is an antimetabolite. The antimetabolite can be, for example, a purine antagonist (e.g., azothioprine or mycophenolate mofetil), a dihydrofolate reductase inhibitor (e.g., methotrexate), acyclovir, ganciclovir, zidovudine, vidarabine, ribavarin, azidothymidine, cytidine arabinoside, amantadine, dideoxyuridine, iododeoxyuridine, poscarnet, or trifluridine.

In certain embodiments, the payload is tacrolimus, cyclosporine, FU506 or rapamycin. In further examples, the molecule is aldesleukin, alemtuzumab, alitretinoin, allopurinol, altretamine, amifostine, anastrozole, arsenic trioxide, bexarotene, bexarotene, calusterone, capecitabine, celecoxib, cladribine, Darbepoetin alfa, Denileukin diftitox, dexrazoxane, dromostanolone propionate, epirubicin, Epoetin alfa, estramustine, exemestane, Filgrastim, floxuridine, fludarabine, fulvestrant, gemcitabine, gemtuzumab ozogamicin (MYLOTARG), goserelin, idarubicin, ifosfamide, imatinib mesylate, Interferon alfa-2a, irinotecan, letrozole, leucovorin, levamisole, meclorethamine or nitrogen mustard, megestrol, mesna, methotrexate, methoxsalen, mitomycin C, mitotane, nandrolone phenpropionate, oprelvekin, oxaliplatin, pamidronate, pegademase, pegaspargase, pegfilgrastim, pentostatin, pipobroman, plicamycin, porfimer sodium, procarbazine, quinacrine, rasburicase, Rituximab, Sargramostim, streptozocin, tamoxifen, temozolomide, teniposide, testolactone, thioguanine, toremifene, tositumomab, trastuzumab, tretinoin, uracil mustard, valrubicin, vinblastine, vincristine, vinorelbine, or zoledronate.

In certain embodiments, the molecule is an immunomodulatory agent. The immunomodulatory agent can be, for example, ganciclovir, etanercept, tacrolimus, cyclosporine, rapamycin, cyclophosphamide, azathioprine, mycophenolate mofetil or methotrexate. Alternatively, the immunomodulatory agent can be, for example, a glucocorticoid (e.g., cortisol or aldosterone) or a glucocorticoid analogue (e.g., prednisone or dexamethasone). Alternatively, the immunomodulatory agent can be, for example, a Toll-like receptor (TLR) agonist, e.g., a TLR7 or TLR8 agonist, e.g., imiquimod, 852A, hiltonol, resiquimod, 3M-052, CpG oligodeoxynucleotides (CpG ODN), 1V270, or SD-101.

In certain embodiments, the immunomodulatory agent is an anti-inflammatory agent, such as arylcarboxylic derivatives, pyrazole-containing derivatives, oxicam derivatives and nicotinic acid derivatives. Classes of anti-inflammatory agents include, for example, cyclooxygenase inhibitors, 5-lipoxygenase inhibitors, and leukotriene receptor antagonists.

Suitable cyclooxygenase inhibitors include meclofenamic acid, mefenamic acid, carprofen, diclofenac, diflunisal, fenbufen, fenoprofen, indomethacin, ketoprofen, nabumetone, sulindac, tenoxicam and tolmetin. Leukotriene receptor antagonists include calcitriol, and ontazolast.

Suitable lipoxygenase inhibitors include redox inhibitors (e.g., catechol butane derivatives, nordihydroguaiaretic acid (NDGA), masoprocol, phenidone, Ianopalen, indazolinones, naphazatrom, benzofuranol, alkylhydroxylamine), and non-redox inhibitors (e.g., hydroxythiazoles, methoxyalkylthiazoles, benzopyrans and derivatives thereof, methoxytetrahydropyran, boswellic acids and acetylated derivatives of boswellic acids, and quinolinemethoxyphenylacetic acids substituted with cycloalkyl radicals), and precursors of redox inhibitors. Other suitable lipoxygenase inhibitors include antioxidants (e.g., phenols, propyl gallate, flavonoids and/or naturally occurring substrates containing flavonoids, hydroxylated derivatives of the flavones, flavonol, dihydroquercetin, luteolin, galangin, orobol, derivatives of chalcone, 4,2′,4′-trihydroxychalcone, ortho-aminophenols, N-hydroxyureas, benzofuranols, ebselen and species that increase the activity of the reducing selenoenzymes), iron chelating agents (e.g., hydroxamic acids and derivatives thereof, N-hydroxyureas, 2-benzyl-1-naphthol, catechols, hydroxylamines, carnosol trolox C, catechol, naphthol, sulfasalazine, zyleuton, 5-hydroxyanthranilic acid and 4-(omega-arylalkyl)phenylalkanoic acids), imidazole-containing compounds (e.g., ketoconazole and itraconazole), phenothiazines, and benzopyran derivatives. Yet other suitable lipoxygenase inhibitors include inhibitors of eicosanoids (e.g., octadecatetraenoic, eicosatetraenoic, docosapentaenoic, eicosahexaenoic and docosahexaenoic acids and esters thereof, PGE1 (prostaglandin E1), PGA2 (prostaglandin A2), viprostol, 15-monohydroxyeicosatetraenoic, 15-monohydroxy-eicosatrienoic and 15-monohydroxyeicosapentaenoic acids, and leukotrienes B5, C5 and D5), compounds interfering with calcium flows, phenothiazines, diphenylbutylamines, verapamil, fuscoside, curcumin, chlorogenic acid, caffeic acid, 5,8,11,14-eicosatetrayenoic acid (ETYA), hydroxyphenylretinamide, Ionapalen, esculin, diethylcarbamazine, phenantroline, baicalein, proxicromil, thioethers, diallyl sulfide and di-(1-propenyl) sulfide.

Other useful molecules include chemical compounds used in the treatment of cancer. Examples of chemotherapeutic agents include Erlotinib (TARCEVA®, Genentech/OSI Pharm.), Bortezomib (VELCADE®, Millennium Pharm.), Fulvestrant (FASLODEX®, AstraZeneca), Sutent (SU11248, Pfizer), Letrozole (FEMARA®, Novartis), Imatinib mesylate (GLEEVEC®, Novartis), PTK787/ZK 222584 (Novartis), Oxaliplatin (Eloxatin®, Sanofi), 5-FU (5-fluorouracil), Leucovorin, Rapamycin (Sirolimus, RAPAMUNE®, Wyeth), Lapatinib (TYKERB®, GSK572016, Glaxo Smith Kline), Lonafarnib (SCH 66336), Sorafenib (BAY43-9006, Bayer Labs), and Gefitinib (IRESSA®, AstraZeneca), AG1478, AG1571 (SU 5271; Sugen). Further examples of chemotherapeutic agents include alkylating agents such as thiotepa and CYTOXAN® (cyclosphosphamide); alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (e.g., bullatacin and bullatacinone); a camptothecin (including the synthetic analog topotecan); bryostatin; callystatin; CC-1065 (including its adozelesin, carzelesin and bizelesin synthetic analogs); cryptophycins (e.g., cryptophycin 1 and cryptophycin 8); dolastatin; duocarmycin (including the synthetic analogs, KW-2189 and CB1-TM1); eleutherobin; pancratistatin; a sarcodictyin; spongistatin; nitrogen mustards such as chlorambucil, chlomaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin γ11 and calicheamicin omegall); dynemicin, including dynemicin A; bisphosphonates, such as clodronate; an esperamicin; as well as neocarzinostatin chromophore and related chromoprotein enediyne antibiotic chromophores). Further anti-cancer agents include aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® (doxorubicin), morpholino-doxorubicin, cyanomorpholino-doxorubicin, 2-pyrrolino-doxorubicin, deoxydoxorubicin, epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, and zorubicin. Further anti-cancer agents include anti-metabolites such as methotrexate and 5-fluorouracil (5-FU); folic acid analogs such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (e.g., T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL® (paclitaxel; Bristol-Myers Squibb Oncology, Princeton, N.J.), ABRAXANE® (Cremophor-free), albumin-engineered nanoparticle formulations of paclitaxel (American Pharmaceutical Partners, Schaumberg, I11.), and TAXOTERE® (doxetaxel; Rhone-Poulenc Rorer, Antony, France); chloranmbucil; GEMZAR® (gemcitabine); 6-thioguanine; mercaptopurine; methotrexate; platinum analogs such as cisplatin and carboplatin; vinblastine; etoposide (VP-16); ifosfamide; mitoxantrone; vincristine; NAVELBINE® (vinorelbine); novantrone; teniposide; edatrexate; daunomycin; aminopterin; capecitabine (XELODA®); ibandronate; CPT-11; topoisomerase inhibitor RFS 2000; difluoromethylornithine (DMFO); retinoids such as retinoic acid; and pharmaceutically acceptable salts, acids and derivatives of any of the above.

Other useful molecules include: (i) anti-hormonal agents that act to regulate or inhibit hormone action on tumors such as anti-estrogens and selective estrogen receptor modulators (SERMs), including, for example, tamoxifen (including NOLVADEX®; tamoxifen citrate), raloxifene, droloxifene, 4-hydroxytamoxifen, trioxifene, keoxifene, LY117018, onapristone, and FARESTON® (toremifene citrate); (ii) aromatase inhibitors that inhibit the enzyme aromatase, which regulates estrogen production in the adrenal glands, such as, for example, 4(5)-imidazoles, aminoglutethimide, MEGASE® (megestrol acetate), AROMASIN® (exemestane; Pfizer), formestanie, fadrozole, RIVISOR® (vorozole), FEMARA® (letrozole; Novartis), and ARIMIDEX® (anastrozole; AstraZeneca); (iii) antiandrogens such as flutamide, nilutamide, bicalutamide, leuprolide, and goserelin; as well as troxacitabine (a 1,3-dioxolane nucleoside cytosine analog); (iv) protein kinase inhibitors; (v) lipid kinase inhibitors; (vi) antisense oligonucleotides, particularly those which inhibit expression of genes in signaling pathways implicated in aberrant cell proliferation, such as, for example, PKC-α, Ralf and H-Ras; (vii) ribozymes such as VEGF expression inhibitors (e.g., ANGIOZYME®) and HER2 expression inhibitors; (viii) vaccines such as gene therapy vaccines, for example, ALLOVECTIN®, LEUVECTIN®, and VAXID®; PROLEUKIN® rIL-2; a topoisomerase 1 inhibitor such as LURTOTECAN®; ABARELIX® rmRH; (ix) anti-angiogenic agents such as bevacizumab (AVASTIN®, Genentech); and (x) pharmaceutically acceptable salts, acids and derivatives of any of the above. Other anti-angiogenic agents include MMP-2 (matrix-metalloproteinase 2) inhibitors, MMP-9 (matrix-metalloproteinase 9) inhibitors, COX-II (cyclooxygenase II) inhibitors, and VEGF receptor tyrosine kinase inhibitors. Examples of VEGF receptor tyrosine kinase inhibitors include 4-(4-bromo-2-fluoroanilino)-6-methoxy-7-(1-methylpiperidin-4-ylmethoxy)quinazoline (ZD6474), 4-(4-fluoro-2-methylindol-5-yloxy)-6-methoxy-7-(3-pyrrolidin-1-ylpropoxy)-quinazoline (AZD2171), vatalanib (PTK787;) and SU11248 (sunitinib).

Additional exemplary molecules for conjugation include an amatoxin, chalicheamicin, DUBA, FAM, MMAD, PBD, and a toxoid.

In certain embodiments, an unnatural amino acid (UAA) comprises a bioconjugation handle to facilitate conjugation to another molecule. In certain embodiments, a method disclosed herein can be used to site-specifically incorporate two different UAAs, each with a different bioconjugation handle, into a single protein (e.g., a single antibody). In certain embodiments, the two bioconjugation handles can be chosen such that they each can be chemoselectively conjugated to two different labels using mutually orthogonal conjugation chemistries. Such pairs of bioconjugation handles include, for example: azide and alkyne, azide and ketone/aldehyde, azide and cyclopropene, ketone/aldehyde and cyclopropene, 5-hydroxyindole and azide, 5-hydroxyindole and cyclopropene, and 5-hydroxyindole and ketone/aldehyde.

The molecule can be conjugated through a variety of linking groups (linkers). The linker may be a cleavable linker or a non-cleavable linker. Optionally or in addition, the linker may be a flexible linker or an inflexible linker. The linker should be a length sufficiently long to allow the molecule and the antibody to be linked without steric hindrance from one another and sufficiently short to retain the intended activity of the antibody. The linker preferably is sufficiently hydrophilic to avoid or minimize instability or insolubility of the antibody. The linker should be sufficiently stable in vivo (e.g., it is not cleaved by serum, enzymes, etc.) to permit the antibody to be operative (e.g., selectively operative) in vivo.

The linker may be from about 1 angstroms (Å) to about 150 Å in length, or from about 1 Å to about 120 Å in length, or from about 5 Å to about 110 Å in length, or from about 10 Å to about 100 Å in length. The linker may be greater than about 2, 3, 4, 5, 6, 7, 8, 9, 10, 11, 12, 13, 14, 15, 16, 17, 18, 19, 20, 25, 27, 30 or greater angstroms in length and/or less than about 110, 100, 90, 85, 80, 75, 70, 65, 60, 55, 50, 45, 43, 42, 41, 40, 39, 38, 37, 36, 35, 34, 33, 32, 31, or fewer Å in length. Furthermore, the linker may be about 5, 10, 15, 20, 25, 30, 35, 40, 45, 50, 55, 60, 65, 70, 75, 80, 85, 90, 95, 100, 110, and 120 Å in length.

In certain embodiments, the linker is any divalent or multivalent linker known to those of skill in the art. Generally, the linker is capable of forming covalent bonds to the molecule and the UAA. Useful divalent linkers include alkylene, substituted alkylene, heteroalkylene, substituted heteroalkylene, arylene, substituted arylene, heteroarylene and substituted heteroarylene linkers. In certain examples, the linker is C1-10 alkylene or C1-10 heteroalkylene.

The linker may include a water soluble polymer. The water soluble polymer may be any structural form including but not limited to linear, forked or branched. Typically, the water soluble polymer is a poly(alkylene glycol), such as poly(ethylene glycol) (PEG), but other water soluble polymers can also be employed. The term “PEG” is used broadly to encompass any polyethylene glycol molecule, without regard to size or to modification at an end of the PEG.

In some cases, a PEG used in the disclosure terminates on one end with hydroxy or methoxy. Alternatively, the PEG can terminate with a reactive group, thereby forming a bifunctional polymer. Typical reactive groups can include those reactive groups that are commonly used to react with the functional groups found in the 20 common amino acids (including but not limited to, maleimide groups, activated carbonates (including but not limited to, p-nitrophenyl ester), activated esters (including but not limited to, N-hydroxysuccinimide, p-nitrophenyl ester) and aldehydes) as well as functional groups that are inert to the 20 common amino acids but that react specifically with complementary functional groups present in UAAs (including but not limited to, azide groups, and alkyne groups).

Any molecular mass for a PEG can be used as practically desired, including but not limited to, from about 50 Daltons (Da) to 100,000 Da or more as desired (including but not limited to, sometimes 100 Da to 100,000 Da, 0.1-50 kDa, or 10-40 kDa). Branched chain PEGs, including but not limited to, PEG molecules with each chain having a MW ranging from 1-100 kDa (including but not limited to, 1-50 kDa or 5-20 kDa) can also be used. A contemplated linker may include any appropriate number of PEG units, e.g., from 2 to 24 PEG units, e.g., PEG2, PEG4, PEG6, PEG8, PEG10, PEG12, or PEG24. A wide range of PEG molecules are described in, including but not limited to, the Shearwater Polymers, Inc. catalog, Nektar Therapeutics catalog.

Generally, at least one terminus of the PEG molecule is available for reaction with the UAA. For example, PEG derivatives bearing alkyne and azide moieties for reaction with amino acid side chains can be used to attach PEG to UAAs as described herein. If the UAA comprises an azide, then the PEG will typically contain either an alkyne moiety to effect formation of the [3+2] cycloaddition product or an activated PEG species (i.e., ester, carbonate) containing a phosphine group to effect formation of the amide linkage. Alternatively, if the UAA comprises an alkyne, then the PEG will typically contain an azide moiety to effect formation of the [3+2] Huisgen cycloaddition product. Similarly, if the UAA comprises a tetrazine, then the PEG will typically contain a strained alkene. Alternatively, if the UAA comprises a strained alkene, then the PEG will typically contain a tetrazine. If the UAA comprises a carbonyl group, the PEG will typically comprise a potent nucleophile (including but not limited to, a hydrazide, hydrazine, hydroxylamine, or semicarbazide functionality) in order to effect formation of corresponding hydrazone, oxime, and semicarbazone linkages, respectively. In other alternatives, a reverse of the orientation of the reactive groups described above can be used, i.e., an azide moiety in the UAA can be reacted with a PEG derivative containing an alkyne.

Many other polymers are also suitable for use in the present disclosure. In some examples, polymer backbones that are water-soluble, with from 2 to about 300 termini, are particularly useful. Examples of suitable polymers include, but are not limited to, other poly(alkylene glycols), such as poly(propylene glycol) (“PPG”), copolymers thereof (including but not limited to copolymers of ethylene glycol and propylene glycol), terpolymers thereof, mixtures thereof, and the like. Although the molecular weight of each chain of the polymer backbone can vary, it is typically in the range of from about 800 Da to about 100,000 Da, often from about 6,000 Da to about 80,000 Da.

As understood in the art, PEG and related polymers may include degradable linkages in the polymer backbone or in the linker group between the polymer backbone and one or more of the terminal functional groups of the polymer molecule. For example, ester linkages formed by the reaction of PEG carboxylic acids or activated PEG carboxylic acids with alcohol groups on a biologically active agent generally hydrolyze under physiological conditions to release the agent. Other hydrolytically degradable linkages include, but are not limited to, carbonate linkages; imine linkages resulted from reaction of an amine and an aldehyde; phosphate ester linkages formed by reacting an alcohol with a phosphate group; hydrazone linkages which are reaction product of a hydrazide and an aldehyde; acetal linkages that are the reaction product of an aldehyde and an alcohol; orthoester linkages that are the reaction product of a formate and an alcohol; peptide linkages formed by an amine group, including but not limited to, at an end of a polymer such as PEG, and a carboxyl group of a peptide; and oligonucleotide linkages formed by a phosphoramidite group, including but not limited to, at the end of a polymer, and a 5′ hydroxyl group of an oligonucleotide. Branched linkers may be used in antibodies of the disclosure. A number of different cleavable linkers are known to those of skill in the art. The mechanisms for release of an agent from these linker groups include, for example, irradiation of a photolabile bond and acid-catalyzed hydrolysis. The length of the linker may be predetermined or selected depending upon a desired spatial relationship between the antibody and the molecule linked to it. In view of the large number of methods that have been reported for attaching a variety of radiodiagnostic compounds, radiotherapeutic compounds, drugs, toxins, and other agents to antibodies one skilled in the art will be able to determine a suitable method for attaching a given agent to an antibody.

Any hetero- or homo-bifunctional linker can be used to link the conjugates. The linker may have a wide range of molecular weight or molecular length. Larger or smaller molecular weight linkers may be used to provide a desired spatial relationship or conformation between the antibody and the linked entity. Linkers having longer or shorter molecular length may also be used to provide a desired space or flexibility between the antibody and the linked entity. Similarly, a linker having a particular shape or conformation may be utilized to impart a particular shape or conformation to the antibody or the linked entity, either before or after the antibody reaches its target. The functional groups present on each end of the linker may be selected to modulate the release of an antibody or a payload under desired conditions.

Some examples of water-soluble bifunctional linkers have a dumbbell structure that includes: a) an azide, an alkyne, a hydrazine, a hydrazide, a hydroxylamine, a carbonyl, a tetrazine, or a strained alkene-containing moiety on at least a first end of a polymer backbone; and b) at least a second functional group on a second end of the polymer backbone. The second functional group can be the same or different as the first functional group. The second functional group, in some examples, is not reactive with the first functional group. Provided, in some examples, are water-soluble compounds that comprise at least one arm of a branched molecular structure. For example, the branched molecular structure can be dendritic.

Further illustrative linkers include, for example, malC, thioether, AcBut, valine-citrulline peptide, malC-valine-citrulline peptide, hydrazone, and disulfide.

In certain embodiments, coupling of antibody and molecule can be accomplished via a crosslinking agent. There are several intermolecular crosslinking agents which can be utilized, see for example, Means and Feeney, CHEMICAL MODIFICATION OF PROTEINS, Holden-Day, 1974, pp. 39-43. Among these reagents are, for example, N-succinimidyl3-(2-pyridyldithio) propionate (SPDP) or N, N′-(1,3-phenylene) bismaleimide (both of which are highly specific for sulfhydryl groups and form irreversible linkages); N, N′-ethylene-bis-(iodoacetamide) or other such reagent having 6 to 11 carbon methylene bridges (which are relatively specific for sulfhydryl groups); and 1, 5-difluoro-2,4-dinitrobenzene (which forms irreversible linkages with amino and tyrosine groups). Other crosslinking agents useful for this purpose include: p,p′-difluoro-N,N′-dinitrodiphenylsulfone (which forms irreversible crosslinkages with amino and phenolic groups); dimethyl adipimidate (which is specific for amino groups); phenol-1,4-disulfonylchloride (which reacts principally with amino groups); hexamethylenediisocyanate or diisothiocyanate, or azophenyl-p-diisocyanate (which reacts principally with amino groups); glutaraldehyde (which reacts with several different side chains) and disdiazobenzidine (which reacts primarily with tyrosine and histidine); N-3-Maleimidopropanoic acid; N-6-Maleimidocaproic acid; N-11-Maleimidoundecanoic acid, 4-(N-maleimidomethyl)cyclohexane-1-carboxy-6-amidocaproic acid; 4-[(Nmaleimidoethyl)carboxamidoethyl(Peg)4 carboxamidomethyl]cyclohexanecarboxylic acid.

The crosslinking agent may be homobifunctional, i.e., having two functional groups that undergo the same reaction. An example of a homobifunctional crosslinking agent is bismaleimidohexane (“BMH”). BMH contains two maleimide functional groups, which react specifically with sulfhydryl-containing compounds under mild conditions (pH 6.5-7.7). The two maleimide groups are connected by a hydrocarbon chain. Therefore, BMH is useful for irreversible crosslinking of polypeptides that contain cysteine residues. Additional commercially available homobifunctional crosslinking agents include: BSOCOES (Bis(2 [Succinimidooxycarbonyloxy]ethyl) sulfone; DPDPB (1,4-Di-(3′-[2pyridyldithio]-propionamido) butane; DSS (disuccinimidyl suberate); DST (disuccinimidyl tartrate); Sulfo DST (sulfodisuccinimidyl tartrate); DSP (dithiobis(succinimidyl propionate); DTSSP (3,3′-Dithiobis(sulfosuccinimidyl propionate); EGS (ethylene glycol bis(succinimidyl succinate)); BASED (Bis(β-[4-azidosalicylamido]-ethyl)disulfide iodinatable); homobifunctional NHS crosslinking reagents (e.g., Bis(NHS)PEO-5 (bis N-succinimidyl-[pentaethylene glycol] ester); and homobifunctional isothiocyanate derivatives of PEG or dextran polymers.

Heterobifunctional crosslinking agents have two different functional groups, for example an amine-reactive group and a thiol-reactive group, that will crosslink two moieties having free amines and thiols, respectively. The most common commercially available heterobifunctional crosslinking agents have an amine reactive N-hydroxysuccinimide ester as one functional group, and a sulfhydryl reactive group as the second functional group. The most common sulfhydryl reactive groups are maleimides, pyridyl disulfides and active halogens. One of the functional groups can be a photoactive aryl nitrene, which upon irradiation reacts with a variety of groups. Exemplary heterobifunctional crosslinking agents include succinimidyl 4-(N maleimidomethyl) cyclohexane-1-carboxylate (“SMCC”), Succinimidyl-4-(N maleimidomethyl)-cyclohexane-1-carboxy(6-amidocaproate) (“LC-SMCC”), N maleimidobenzoyl-N-hydroxysuccinimide ester (“MBS”), and succinimide 4-(p-maleimidophenyl) butyrate (“SMPB”), an extended chain analog of MBS. The succinimidyl group of these crosslinking agents reacts with a primary amine forming an amide bond, and the thiol-reactive maleimide forms a covalent thioether bond with the thiol group (e.g., of a cysteine).

Additional exemplary crosslinking agents include: BS3 ([Bis(sulfosuccinimidyl)suberate], which is a homobifunctional N-hydroxysuccinimide ester that targets accessible primary amines; NHS/EDC (N-hydroxy-succinimide and N-ethyl-‘(dimethylaminopropyl)carbodimide, which allows for the conjugation of primary amine groups with carboxyl groups); sulfoEMCS ([N-e-Maleimido-caproic acid]hydrazide, which includes heterobifunctional reactive groups (a maleimide and an NHS-ester) that are reactive toward sulfhydryl and amino groups; hydrazide, which is useful for useful for linking carboxyl groups on exposed carbohydrates to primary amines; SATA (N-succinimidyl-S-acetylthioacetate), which is reactive towards amines and adds protected sulfhydryl groups; monofluoro cyclooctyne (MFCO); bicyclo[6.1.0]nonyne (BCN); N succinimidyl-S-acetylthiopropionate (SATP); maleimido and dibenzocyclooctyne ester (a DBCO ester); and EDC (1-Ethyl-3-[3-dimethylaminopropyl]carbodiimide hydrochloride).

The length of these crosslinking agents can be varied by the use of polymeric regions between the two reactive groups, which typically take the form of chemical linkers such as polymeric ethylene glycol or simple carbon chains, but can also include sugars, amino acids or peptides, or oligonucleotides. Polymer chain lengths of from 5 to 50 nm are typical, but can be shorter or longer as needed. For example, the crosslinking agent may comprise a <2 carbon chain arm, a 2-5 carbon chain arm, or a 3-6 carbon chain arm.

Crosslinking agents often have low solubility in water. A hydrophilic moiety, such as a sulfonate group, may be added to the crosslinking agent to improve its water solubility. Sulfo-MBS and sulfo-SMCC are examples of crosslinking agents modified for water solubility.

Many crosslinking agents yield a conjugate that is essentially non-cleavable under cellular conditions. However, some crosslinking agents contain a covalent bond, such as a disulfide, that is cleavable under cellular conditions. For example, Traut's reagent, dithiobis(succinimidylpropionate) (“DSP”), and N-succinimidyl 3-(2-pyridyldithio) propionate (“SPDP”) are well-known cleavable crosslinking agents. Direct disulfide linkage may also be useful.

Numerous crosslinking agents, including the ones discussed above, are commercially available. Detailed instructions for their use are readily available from the commercial suppliers. A general reference on protein cross-linking and conjugate preparation is: Wong, CHEMISTRY OF PROTEIN CONJUGATION AND CROSS-LINKING, CRC Press (1991).

In certain embodiments, the linker comprises a polypeptide linker that connects or fuses the molecule to the antibody. When a polypeptide linker is employed, the linker may comprise hydrophilic amino acid residues, such as Gln, Ser, Gly, Glu, Pro, His and Arg. In certain embodiments, the linker is a peptide containing 1-25 amino acid residues, 1-20 amino acid residues, 2-15 amino acid residues, 3-10 amino acid residues, 3-7 amino acid residues, 4-25 amino acid residues, 4-20 amino acid residues, 4-15 amino acid residues, 4-10 amino acid residues, 5-25 amino acid residues, 5-20 amino acid residues, 5-15 amino acid residues, or 5-10 amino acid residues. Exemplary linkers include glycine and serine-rich linkers, e.g., (GlyGlyPro)ₙ, or (GlyGlyGlyGlySer)ₙ, where n is 1-5. In certain embodiments, the linker comprises, consists, or consists essentially of GGGGS (SEQ ID NO: 176). In certain embodiments, the linker comprises, consists, or consists essentially of GGGGSGGGGS (SEQ ID NO: 177). Additional exemplary linker sequences are disclosed, e.g., in George et al. (2003) PROTEIN ENGINEERING 15:871-879, and U.S. Pat. Nos. 5,482,858 and 5,525,491.

In certain embodiments, the UAA comprises a non-natural aromatic chemical moiety (e.g., a hydroxyl-indole group; an amino-indole group; an aminophenol group; or a hydroxyl-phenol group, e.g., the UAA is 5-hydroxytryptophan (5-HTP), or an analog thereof), and/or the linker comprises a diazonium group (e.g., the linker comprises 4-nitorbenzenediazonium (4NDz); 4-carboxybenzenediazonium (4NeDz) or 4-methoxybenzenediazonium (4MCDz). The UAA and linker may react under conditions suitable to form an azo-linkage via an azo-coupling reaction between the aromatic chemical moiety and the diazonium group. Further methods for conjugation of molecules to UAAs are described, for example, in U.S. Patent Application Publication No. 2018/0360984.

In certain embodiments, when a molecule is conjugated to an antibody, the antibody has an average drug antibody ratio (DAR) of at least 0.5, 0.6, 0.7, 0.8, 0.9, 1.0, 1.5, 1.6, 1.7, 1.8, 1.9, 2.0, 3.5, 3.6, 3.7, 3.8, 3.9, or 4.0, as measured by hydrophobic interaction chromatography (HIC). In this context, it is understood that drug antibody ratio (DAR) may refer to the ratio of any conjugated molecule to antibody (e.g., a detectable label as well as a drug). In certain embodiments, the antibody has an average drug antibody ratio (DAR) that is within 5%, 10%, 15%, 20%, 25%, 30%, 35%, or 40% of the number of UAAs in the antibody.

### IV. Methods of Making Antibodies

tRNAS, aminoacyl-tRNA synthetases, and/or unnatural amino acids disclosed herein may be used to incorporate an unnatural amino acid into an antibody of interest using any appropriate translation system.

The term “translation system” refers to a system including components necessary to incorporate an amino acid into a growing polypeptide chain (protein). Components of a translation system can include, e.g., ribosomes, tRNA's, synthetases, mRNA and the like. Translation systems may be cellular or cell-free, and may be prokaryotic or eukaryotic. For example, translation systems may include, or be derived from, a non-eukaryotic cell, e.g., a bacterium (such as E. coli), a eukaryotic cell, e.g., a yeast cell, a mammalian cell, a plant cell, an algae cell, a fungus cell, or an insect cell.

Translation systems include host cells or cell lines, e.g., host cells or cell lines contemplated herein. To express a polypeptide of interest with an unnatural amino acid in a host cell, one may clone a polynucleotide encoding the polypeptide into an expression vector that contains, for example, a promoter to direct transcription, a transcription/translation terminator, and if for a nucleic acid encoding a protein, a ribosome binding site for translational initiation.

Translation systems also include whole cell preparations such as permeabilized cells or cell cultures wherein a desired nucleic acid sequence can be transcribed to mRNA and the mRNA translated. Cell-free translation systems are commercially available and many different types and systems are well-known. Examples of cell-free systems include, but are not limited to, prokaryotic lysates such as Escherichia coli lysates, and eukaryotic lysates such as wheat germ extracts, insect cell lysates, rabbit reticulocyte lysates, rabbit oocyte lysates and human cell lysates. Reconstituted translation systems may also be used. Reconstituted translation systems may include mixtures of purified translation factors as well as combinations of lysates or lysates supplemented with purified translation factors such as initiation factor-1 (IF-1), IF-2, IF-3 (α or β), elongation factor T (EF-Tu), or termination factors. Cell-free systems may also be coupled transcription/translation systems wherein DNA is introduced to the system, transcribed into mRNA and the mRNA is translated.

The invention provides methods of expressing an antibody containing an unnatural amino acid and methods of producing an antibody with one, or more, unnatural amino acids at specified positions in the antibody. The methods comprise incubating a translation system (e.g., culturing or growing a host cell or cell line, e.g., a host cell or cell line disclosed herein) under conditions that permit incorporation of the unnatural amino acid into the antibody being expressed in the cell. The translation system may be contacted with (e.g. the cell culture medium may be contacted with) one, or more, unnatural amino acids (e.g., leucyl or tryptophanyl analogs) under conditions suitable for incorporation of the one, or more, unnatural amino acids into the antibody.

In certain embodiments, the antibody is expressed from a nucleic acid sequence comprising a premature stop codon. The translation system (e.g., host cell or cell line) may, for example, contain a leucyl-tRNA synthetase mutein (e.g., a leucyl-tRNA synthetase mutein disclosed herein) capable of charging a suppressor leucyl tRNA (e.g., a suppressor leucyl tRNA disclosed herein) with an unnatural amino acid (e.g., a leucyl analog) which is incorporated into the antibody at a position corresponding to the premature stop codon. In certain embodiments, the leucyl suppressor tRNA comprises an anticodon sequence that hybridizes to the premature stop codon and permits the unnatural amino to be incorporated into the antibody at the position corresponding to the premature stop codon.

In certain embodiments, the antibody is expressed from a nucleic acid sequence comprising a premature stop codon. The translation system (e.g., host cell or cell line) may, for example, contain a tryptophanyl-tRNA synthetase mutein (e.g., a tryptophanyl-tRNA synthetase mutein disclosed herein) capable of charging a suppressor tryptophanyl tRNA (e.g., a suppressor tryptophanyl tRNA disclosed herein) with an unnatural amino acid (e.g., a tryptophan analog) which is incorporated into the antibody at a position corresponding to the premature stop codon. In certain embodiments, the tryptophanyl suppressor tRNA comprises an anticodon sequence that hybridizes to the premature stop codon and permits the unnatural amino to be incorporated into the antibody at the position corresponding to the premature stop codon.

In certain embodiments, a protein (e.g., an antibody containing a UAA) is expressed or produced in a eukaryotic cell (e.g., a mammalian cell). Features may distinguish proteins produced in prokaryotic cells (e.g., bacteria) from those produced in eukaryotic cells (e.g., mammalian cells). For example, proteins produced in mammalian cells may undergo post-translational modifications, e.g., modifications that are dependent upon enzymes located in organelles, e.g., the endoplasmic reticulum or Golgi apparatus. For example, disulfide bond formation in the endoplasmic reticulum may influence protein conformation and/or stabilization. Additional examples of such post-translational modifications include, without limitation, sulfation, amidation, palmitation, and glycosylation (e.g., N-linked glycosylation and O-linked glycosylation). Accordingly, in certain embodiments, a protein (e.g., an antibody containing a UAA) comprises one or more post-translational modifications selected from sulfation, amidation, palmitation, and glycosylation (e.g., N-linked glycosylation and O-linked glycosylation).

In certain embodiments, the expression yield of an antibody comprising the UAA, for example, when expressed by a host cell or cell line, is at least 10%, 20%, 30%, 40%, 50%, 60%, 70%, 80%, or 90% of the expression yield of a reference antibody. For example, in certain embodiments, the amount of antibody comprising the UAA expressed by the host cell or cell line is at least 10%, 20%, 30%, 40%, 50%, 60%, 70%, 80%, or 90% of the amount of a reference antibody expressed by the same cell or a similar cell. In certain embodiments, the reference antibody is an antibody that does not comprise the UAA but is otherwise identical to the antibody comprising the UAA. For example, the reference antibody may comprise a wild-type amino acid sequence, or comprise a wild-type amino acid residue at the position corresponding to the UAA. Antibody expression may be measured by any method known in the art, including for example, Western blot or ELISA. Expression may be measured by measuring protein concentration (e.g., by ultraviolet (UV) absorption at 280 nm or Bradford assay) in a solution of defined volume and purity following purification of the antibody.

In certain embodiments, a disclosed method further comprises purifying the antibody. Specific expression and purification conditions will vary depending upon the expression system employed. Purification techniques known in the art include, e.g., those employing affinity tags such as glutathione-S-transferase (GST) or histidine tags. In certain embodiments, an antibody may be purified by contacting the antibody with protein A and/or protein G. In certain embodiments, following protein G purification (e.g., following only protein G purification) less than 10%, 9%, 8%, 7%, 6%, 5%, 4%, 3%, 2%, or 1% of the antibody is aggregated, as measured by size exclusion chromatography (SEC).

In certain embodiments, a disclosed method further comprises conjugating a molecule or payload to a UAA in the antibody. In certain embodiments, the method comprises conjugating the molecule or payload to the UAA within 5 minutes to 48 hours at room temperature (e.g., for less than 48 hours, less than 36 hours, less than 24 hours, less than 12 hours, less than 6 hours, less than 1 hour, less than 30 minutes, less than 15 minutes, or less than 10 minutes).

### V. Aminoacyl-tRNA Synthetases

The invention relates to engineered aminoacyl-tRNA synthetases (or aaRSs) capable of charging a tRNA with an unnatural amino acid for incorporation into a protein (e.g., an antibody). As used herein, the term “aminoacyl-tRNA synthetase” refers to any enzyme, or a functional fragment thereof, that charges, or is capable of charging, a tRNA with an amino acid (e.g., an unnatural amino acid) for incorporation into a protein. As used herein, the term “functional fragment” of an aminoacyl-tRNA synthetase refers to fragment of a full-length aminoacyl-tRNA synthetase that retains, for example, at least 10%, at least 20%, at least 30%, at least 40%, at least 50%, at least 60%, at least 70%, at least 80%, at least 90%, at least 95%, or 100% of the enzymatic activity of the corresponding full-length tRNA synthetase (e.g., a naturally occurring tRNA synthetase). Aminoacyl-tRNA synthetase enzymatic activity may be assayed by any method known in the art. For example, in vitro aminoacylation assays are described in Hoben et al. (1985) METHODS ENZYMOL. 113:55-59 and in U.S. Patent Application Publication No. 2003/0228593 and cell-based aminoacylation assays are described in U.S. Patent Application Publication Nos. 2003/0082575 and 2005/0009049. In certain embodiments, the functional fragment comprises at least 100, 150, 200, 250, 300, 350, 400, 450, 500, 550, 600, 650, 700, 750, or 800 consecutive amino acids present in a full-length tRNA synthetase (e.g., a naturally occurring aminoacyl-tRNA synthetase).

The term aminoacyl-tRNA synthetase includes variants (i.e., muteins) having one or more mutations (e.g., amino acid substitutions, deletions, or insertions) relative to a wild-type aminoacyl-tRNA synthetase sequence. In certain embodiments, an aminoacyl-tRNA synthetase mutein may comprise, consist, or consist essentially of, a single mutation (e.g., a mutation contemplated herein), or a combination of 2, 3, 4, 5, 6, 7, 8, 9, 10, 11, 12, 13, 14, 15 or more than 15 mutations (e.g., mutations contemplated herein). It is contemplated that an aminoacyl-tRNA synthetase mutein may comprise, consist, or consist essentially 1-15, 1-10, 1-7, 1-6, 1-5, 1-4, 1-3, 1-2, 2-15, 2-10, 2-7, 2-6, 2-5, 2-4, 2-3, 3-15, 3-10, 3-7, 3-6, 3-5, or 4-10, 4-7, 4-6, 4-5, 5-10, 5-7, 5-6, 6-10, 6-7, 7-10, 7-8, or 8-10 mutations (e.g., mutations contemplated herein). An aminoacyl-tRNA synthetase mutein may comprise a conservative substitution relative to a wild-type sequence or a sequence disclosed herein.

In certain embodiments, the substrate specificity of the aminoacyl-tRNA synthetase mutein is altered relative to a corresponding (or template) wild-type aminoacyl-tRNA synthetase such that only a desired unnatural amino acid, but not any of the common 20 amino acids, is charged to the substrate tRNA.

An aminoacyl-tRNA synthetase may be derived from a bacterial source, e.g., Escherichia coli, Thermus thermophilus, or Bacillus stearothermophilus. An aminoacyl-tRNA synthetase may also be derived from an archaeal source, e.g, from the Methanosarcinacaea or Desulfitobacterium families, any of the M. barkeri (Mb), M. alvus (Ma), M. mazei (Mm) or D. hafnisense (Dh) families, Methanobacterium thermoautotrophicum, Haloferax volcanii, Halobacterium species NRC-1, or Archaeoglobus fulgidus. In other embodiments, eukaryotic sources can also be used, for example, plants, algae, protists, fungi, yeasts, or animals (e.g., mammals, insects, arthropods, etc.). As used herein, the terms “derivative” or “derived from” refer to a component that is isolated from or made using information from a specified molecule or organism. As used herein, the term “analog” refers to a component (e.g., a tRNA, tRNA synthetase, or unnatural amino acid) that is derived from or analogous with (in terms of structure and/or function) a reference component (e.g., a wild-type tRNA, a wild-type tRNA synthetase, or a natural amino acid). In certain embodiments, derivatives or analogs have at least 40%, 50%, 60%, 70%, 80%, 90%, 100% or more of a given activity as a reference or originator component (e.g., wild type component).

It is contemplated that the aminoacyl-tRNA synthetase may aminoacylate a substrate tRNA in vitro or in vivo, and can be provided to a translation system (e.g., an in vitro translation system or a cell) as a polypeptide or protein, or as a polynucleotide that encodes the aminoacyl-tRNA synthetase.

In certain embodiments, the aminoacyl-tRNA synthetase is derived from an E. coli leucyl-tRNA synthetase and, for example, the aminoacyl-tRNA synthetase preferentially aminoacylates an E. coli leucyl tRNA (or a variant thereof) with a leucine analog over the naturally-occurring leucine amino acid.

For example, the aminoacyl-tRNA synthetase may comprise SEQ ID NO: 1, or an amino acid sequence that has at least 85%, 90%, 95%, 96%, 97%, 98%, or 99% sequence identity to SEQ ID NO: 1. In certain embodiments, the aminoacyl-tRNA synthetase comprises SEQ ID NO: 1, or a functional fragment or variant thereof, and with one, two, three, four, five or more of the following mutations: (i) a substitution of a glutamine residue at a position corresponding to position 2 of SEQ ID NO: 1, e.g., a substitution by glutamic acid (Q2E); (ii) a substitution of a glutamic acid residue at a position corresponding to position 20 of SEQ ID NO: 1, e.g., a substitution by lysine (E20K), methionine (E20M), or valine (E20V); (iii) a substitution of a methionine residue at a position corresponding to position 40 of SEQ ID NO: 1, e.g., a substitution by isoleucine (M40I) or valine (M40V); (iv) a substitution of a leucine residue at a position corresponding to position 41 of SEQ ID NO: 1, e.g., a substitution by serine (L41S), valine (L41V), or alanine (L41A); (v) a substitution of a threonine residue at a position corresponding to position 252 of SEQ ID NO: 1, e.g., a substitution by alanine (T252A) or arginine (T252R); (vi) a substitution of a tyrosine residue at a position corresponding to position 499 of SEQ ID NO: 1, e.g., a substitution by isoleucine (Y499I), serine (Y499S), alanine (Y499A), or histidine (Y499H); (vii) a substitution of a tyrosine residue at a position corresponding to position 527 of SEQ ID NO: 1, e.g., a substitution by alanine (Y527A), leucine (Y527L), isoleucine (Y527I), valine (Y527V), or glycine (Y527G); or (viii) a substitution of a histidine residue at a position corresponding to position 537 of SEQ ID NO: 1, e.g., a substitution by glycine (H537G), or any combination of the foregoing.

In certain embodiments, the aminoacyl-tRNA synthetase comprises (i) at least one substitution (e.g., a substitution with a hydrophobic amino acid) at a position corresponding to His537 of SEQ ID NO: 1, (ii) at least one amino acid substitution selected from E20V, E20M, L41V, L41A, Y499H, Y499A, Y527I, Y527V, Y527G, and any combination thereof, (iii) at least one amino acid substitution selected from E20K and L41S and any combination thereof and at least one amino acid substitution selected from M40I, T252A, Y499I, and Y527A, and any combination thereof, or (iv) a combination of two or more of (i), (ii) and (iii), for example, (i) and (ii), (i) and (iii), (ii) and (iii) and (i), (ii) and (iii).

In certain embodiments, the aminoacyl-tRNA synthetase comprises a substitution of a glutamic acid residue at a position corresponding to position 20 of SEQ ID NO: 1, e.g., a substitution with an amino acid other than a Glu or Lys, e.g., a substitution with a hydrophobic amino acid (e.g., Leu, Val, or Met). In certain embodiments, the aminoacyl-tRNA synthetase comprises a substitution of a leucine residue at a position corresponding to position 41 of SEQ ID NO: 1, e.g., a substitution with an amino acid other than a Leu or Ser, e.g., a substitution with a hydrophobic amino acid other than Leu (e.g., Gly, Ala, Val, or Met). In certain embodiments, the aminoacyl-tRNA synthetase comprises a substitution of a tyrosine residue at a position corresponding to position 499 of SEQ ID NO: 1, e.g., a substitution with a small hydrophobic amino acid (e.g., Gly, Ala, or Val) or a substitution with a positively charged amino acid (e.g., Lys, Arg, or His). In certain embodiments, the aminoacyl-tRNA synthetase comprises a substitution of a tyrosine residue at a position corresponding to position 527 of SEQ ID NO: 1, e.g., a substitution with a hydrophobic amino acid other than Ala or Leu (e.g., Gly, Ile, Met, or Val). In certain embodiments, the tRNA synthetase mutein comprises L41V.

In certain embodiments, the aminoacyl-tRNA synthetase comprises a combination of mutations selected from: (i) Q2E, E20K, M40I, L41S, T252A, Y499I, Y527A, and H537G; (ii) Q2E, E20K, M40V, L41S, T252R, Y499S, Y527L, and H537G; (iii) Q2E, M40I, T252A, Y499I, Y527A, and H537G; (iv) Q2E, E20M, M40I, L41S, T252A, Y499I, Y527A, and H537G; (v) Q2E, E20V, M40I, L41S, T252A, Y499I, Y527A, and H537G; (vi) Q2E, E20K, M40I, L41V, T252A, Y499I, Y527A, and H537G; (vii) Q2E, E20K, M40I, L41A, T252A, Y499I, Y527A, and H537G; (viii) Q2E, E20K, M40I, L41S, T252A, Y499A, Y527A, and H537G; (ix) Q2E, E20K, M40I, L41S, T252A, Y499H, Y527A, and H537G; (x) Q2E, E20K, M40I, L41S, T252A, Y499I, Y527I, and H537G; (xi) Q2E, E20K, M40I, L41S, T252A, Y499I, Y527V, and H537G; (xii) Q2E, E20K, M40I, L41S, T252A, Y499I, Y527G, and H537G; (xiii) E20K, M40I, L41S, T252A, Y499I, Y527A, and H537G; (xiv) E20M, M40I, L41S, T252A, Y499I, Y527A, and H537G; (xv) E20V, M40I, L41S, T252A, Y499I, Y527A, and H537G; (xvi) E20K, M40I, L41V, T252A, Y499I, Y527A, and H537G; (vii) E20K, M40I, L41A, T252A, Y499I, Y527A, and H537G; (xviii) E20K, M40I, L41S, T252A, Y499A, Y527A, and H537G; (xix) E20K, M40I, L41S, T252A, Y499H, Y527A, and H537G; (xx) E20K, M40I, L41S, T252A, Y499I, Y527I, and H537G; (xxi) E20K, M40I, L41S, T252A, Y499I, Y527V, and H537G; and (xxii) E20K, M40I, L41S, T252A, Y499I, Y527G, and H537G.

In certain embodiments, the aminoacyl-tRNA synthetase comprises the amino acid sequence of any one of SEQ ID NOs: 2-13, or an amino acid sequence that has at least 85%, 90%, 95%, 96%, 97%, 98%, or 99% sequence identity to any one of SEQ ID NOs: 2-13.

In certain embodiments, the tRNA synthetase mutein comprises the amino acid sequence of SEQ ID NO: 14, wherein X₂ is Q or E, X₂₀ is E, K, V or M, X₄₀ is M, I, or V, X₄₁ is L, S, V, or A, X₂₅₂ is T, A, or R, X₄₉₉ is Y, A, I, H, or S, X₅₂₇ is Y, A, I, L, or V, and X₅₃₇ is H or G, and the tRNA synthetase mutein comprises at least one mutation (for example, 2, 3, 4, 5, 6, 7, 8, 9, or more mutations) relative to SEQ ID NO: 1. In certain embodiments, the tRNA synthetase mutein comprises the amino acid sequence of SEQ ID NO: 15, wherein X₂₀ is K, V or M, X₄₁ is S, V, or A, X₄₉₉ is A, I, or H, and X₅₂₇ is A, I, or V, and the tRNA synthetase mutein comprises at least one mutation relative to SEQ ID NO: 1.

In certain embodiments, the aminoacyl-tRNA synthetase is derived from an E. coli tryptophanyl-tRNA synthetase and, for example, the aminoacyl-tRNA synthetase preferentially aminoacylates an E. coli tryptophanyl tRNA (or a variant thereof) with a tryptophan analog over the naturally-occurring tryptophan amino acid.

For example, the aminoacyl-tRNA synthetase may comprise SEQ ID NO: 44, or an amino acid sequence that has at least 85%, 90%, 95%, 96%, 97%, 98%, or 99% sequence identity to SEQ ID NO: 44. In certain embodiments, the aminoacyl-tRNA synthetase comprises SEQ ID NO: 44, or a functional fragment or variant thereof, but with one or more of the following mutations: (i) a substitution of a serine residue at a position corresponding to position 8 of SEQ ID NO: 44, e.g., a substitution by alanine (S8A); (ii) a substitution of a valine residue at a position corresponding to position 144 of SEQ ID NO: 44, e.g., a substitution by serine (V144S), glycine (V144G) or alanine (V144A); (iii) a substitution of a valine residue at a position corresponding to position 146 of SEQ ID NO: 44, e.g., a substitution by alanine (V146A), isoleucine (V146I), or cysteine (V146C). In certain embodiments, the aminoacyl-tRNA synthetase comprises a combination of mutations selected from: (i) S8A, V144S, and V146A, (ii) S8A, V144G, and V146I, (iii) S8A, V144A, and V146A, and (iv) S8A, V144G, and V146C.

In certain embodiments, the aminoacyl-tRNA synthetase comprises the amino acid sequence of any one of SEQ ID NOs: 45-48, or an amino acid sequence that has at least 85%, 90%, 95%, 96%, 97%, 98%, or 99% sequence identity to any one of SEQ ID NOs: 45-48.

In certain embodiments, the aminoacyl-tRNA synthetase is derived from an E. coli tyrosyl-tRNA synthetase and, for example, the aminoacyl-tRNA synthetase preferentially aminoacylates an E. coli tyrosyl tRNA (or a variant thereof) with a tyrosine analog over the naturally-occurring tryptophan amino acid. For example, the aminoacyl-tRNA synthetase may comprise SEQ ID NO: 70, or an amino acid sequence that has at least 85%, 90%, 95%, 96%, 97%, 98%, or 99% sequence identity to SEQ ID NO: 70, or a functional fragment or variant thereof.

In certain embodiments, the aminoacyl-tRNA synthetase is derived from an M. barkeri pyrrolysyl-tRNA synthetase and, for example, the aminoacyl-tRNA synthetase preferentially aminoacylates an M. barkeri pyrrolysyl tRNA (or a variant thereof) with a pyrrolysine analog over the naturally-occurring pyrrolysine amino acid. For example, the aminoacyl-tRNA synthetase may comprise SEQ ID NO: 101, or an amino acid sequence that has at least 85%, 90%, 95%, 96%, 97%, 98%, or 99% sequence identity to SEQ ID NO: 101, or a functional fragment or variant thereof.

Methods for producing proteins, e.g., aminoacyl-tRNA synthetases, are known in the art. For example, DNA molecules encoding a protein of interest can be synthesized chemically or by recombinant DNA methodologies. The resulting DNA molecules encoding the protein interest can be ligated to other appropriate nucleotide sequences, including, for example, expression control sequences, to produce conventional gene expression constructs (i.e., expression vectors) encoding the desired protein. Production of defined gene constructs is within routine skill in the art.

Nucleic acids encoding desired proteins (e.g, aminoacyl-tRNA synthetases) can be incorporated (ligated) into expression vectors, which can be introduced into host cells through conventional transfection or transformation techniques. Exemplary host cells are E. coli cells, Chinese hamster ovary (CHO) cells, human embryonic kidney 293 (HEK 293) cells, HeLa cells, baby hamster kidney (BHK) cells, monkey kidney cells (COS), human hepatocellular carcinoma cells (e.g., Hep G2), and myeloma cells. Transformed host cells can be grown under conditions that permit the host cells to express the desired protein.

Specific expression and purification conditions will vary depending upon the expression system employed. For example, if a gene is to be expressed in E. coli, it is first cloned into an expression vector by positioning the engineered gene downstream from a suitable bacterial promoter, e.g., Trp or Tac, and a prokaryotic signal sequence. The expressed protein may be secreted. The expressed protein may accumulate in refractile or inclusion bodies, which can be harvested after disruption of the cells by French press or sonication. The refractile bodies then are solubilized, and the protein may be refolded and/or cleaved by methods known in the art.

If the engineered gene is to be expressed in eukaryotic host cells, e.g., CHO cells, it is first inserted into an expression vector containing a suitable eukaryotic promoter, a secretion signal, a poly A sequence, and a stop codon. Optionally, the vector or gene construct may contain enhancers and introns. The gene construct can be introduced into eukaryotic host cells using conventional techniques.

A protein of interest (e.g, an aminoacyl-tRNA synthetase) can be produced by growing (culturing) a host cell transfected with an expression vector encoding such a protein under conditions that permit expression of the protein. Following expression, the protein can be harvested and purified or isolated using techniques known in the art, e.g., affinity tags such as glutathione-S-transferase (GST) or histidine tags.

Additional methods for producing aminoacyl-tRNA synthetases, and for altering the substrate specificity of the synthetase can be found in U.S. Patent Application Publication Nos. 2003/0108885 and 2005/0009049, Hamano-Takaku et al. (2000) JOURNAL OF BIOL. CHEM. 275(51):40324-40328, Kiga et al. (2002) PROC. NATL. ACAD. SCI. USA 99(15): 9715-9723, and Francklyn et al. (2002) RNA, 8:1363-1372.

The invention also encompasses nucleic acids encoding aminoacyl-tRNA synthetases disclosed herein. For example, nucleotide sequences encoding leucyl-tRNA synthetase muteins disclosed herein are depicted in SEQ ID NOs: 55-67. Accordingly, the invention provides a nucleic acid comprising the nucleotide sequence of any one of SEQ ID NOs: 55-67, or a nucleotide sequence that has at least 85%, 90%, 95%, 96%, 97%, 98%, or 99% sequence identity to any one of SEQ ID NOs: 55-67. The invention also provides a nucleic acid comprising a nucleotide sequence encoding the amino acid sequence encoded by any one of SEQ ID NOs: 55-67, or a nucleotide sequence that has at least 85%, 90%, 95%, 96%, 97%, 98%, or 99% sequence identity to a nucleotide sequence encoding the amino acid sequence encoded by any one of SEQ ID NOs: 55-67.

A nucleotide sequence encoding a tryptophanyl-tRNA synthetase disclosed herein is depicted in SEQ ID NO: 103. Accordingly, the invention provides a nucleic acid comprising the nucleotide sequence of SEQ ID NO: 103, or a nucleotide sequence that has at least 85%, 90%, 95%, 96%, 97%, 98%, or 99% sequence identity to SEQ ID NO: 103. The invention also provides a nucleic acid comprising a nucleotide sequence encoding the amino acid sequence encoded by SEQ ID NO: 103, or a nucleotide sequence that has at least 85%, 90%, 95%, 96%, 97%, 98%, or 99% sequence identity to a nucleotide sequence encoding the amino acid sequence encoded by SEQ ID NO: 103.

A nucleotide sequence encoding a tyrosyl-tRNA synthetase disclosed herein is depicted in SEQ ID NO: 71. Accordingly, the invention provides a nucleic acid comprising the nucleotide sequence of SEQ ID NO: 71, or a nucleotide sequence that has at least 85%, 90%, 95%, 96%, 97%, 98%, or 99% sequence identity to SEQ ID NO: 71. The invention also provides a nucleic acid comprising a nucleotide sequence encoding the amino acid sequence encoded by SEQ ID NO: 71, or a nucleotide sequence that has at least 85%, 90%, 95%, 96%, 97%, 98%, or 99% sequence identity to a nucleotide sequence encoding the amino acid sequence encoded by SEQ ID NO: 71.

A nucleotide sequence encoding a pyrrolysyl-tRNA synthetase disclosed herein is depicted in SEQ ID NO: 102. Accordingly, the invention provides a nucleic acid comprising the nucleotide sequence of SEQ ID NO: 102, or a nucleotide sequence that has at least 85%, 90%, 95%, 96%, 97%, 98%, or 99% sequence identity to SEQ ID NO: 102. The invention also provides a nucleic acid comprising a nucleotide sequence encoding the amino acid sequence encoded by SEQ ID NO: 102, or a nucleotide sequence that has at least 85%, 90%, 95%, 96%, 97%, 98%, or 99% sequence identity to a nucleotide sequence encoding the amino acid sequence encoded by SEQ ID NO: 102.

VI. tRNAs

The invention relates to transfer RNAs (tRNAs) that mediate the incorporation of unnatural amino acids into proteins (e.g., antibodies).

During protein synthesis, a tRNA molecule delivers an amino acid to a ribosome for incorporation into a growing protein (polypeptide) chain. tRNAs typically are about 70 to 100 nucleotides in length. Active tRNAs contain a 3′ CCA sequence that may be transcribed into the tRNA during its synthesis or may be added later during post-transcriptional processing. During aminoacylation, the amino acid that is attached to a given tRNA molecule is covalently attached to the 2′ or 3′ hydroxyl group of the 3′-terminal ribose to form an aminoacyl-tRNA (aa-tRNA). It is understood that an amino acid can spontaneously migrate from the 2′-hydroxyl group to the 3′-hydroxyl group and vice versa, but it is incorporated into a growing protein chain at the ribosome from the 3′-OH position. A loop at the other end of the folded aa-tRNA molecule contains a sequence of three bases known as the anticodon. When this anticodon sequence hybridizes or base-pairs with a complementary three-base codon sequence in a ribosome-bound mRNA, the aa-tRNA binds to the ribosome and its amino acid is incorporated into the polypeptide chain being synthesized by the ribosome. Because all tRNAs that base-pair with a specific codon are aminoacylated with a single specific amino acid, the translation of the genetic code is affected by tRNAs. Each of the 61 non-termination codons in an mRNA directs the binding of its cognate aa-tRNA and the addition of a single specific amino acid to the growing polypeptide chain being synthesized by the ribosome. The term “cognate” refers to components that function together, e.g., a tRNA and an aminoacyl-tRNA synthetase.

Suppressor tRNAs are modified tRNAs that alter the reading of a mRNA in a given translation system. For example, a suppressor tRNA may read through a codon such as a stop codon, a four base codon, or a rare codon. The use of the word in suppressor is based on the fact, that under certain circumstance, the modified tRNA “suppresses” the typical phenotypic effect of the codon in the mRNA. Suppressor tRNAs typically contain a mutation (modification) in either the anticodon, changing codon specificity, or at some position that alters the aminoacylation identity of the tRNA. The term “suppression activity” refers to the ability of a tRNA, e.g., a suppressor tRNA, to read through a codon (e.g., a premature stop codon) that would not be read through by the endogenous translation machinery in a system of interest.

In certain embodiments, a tRNA (e.g., a suppressor tRNA) contains a modified anticodon region, such that the modified anticodon hybridizes with a different codon than the corresponding naturally occurring anticodon.

In certain embodiments, a tRNA comprises an anticodon that hybridizes to a codon selected from UAG (i.e., an “amber” termination codon), UGA (i.e., an “opal” termination codon), and UAA (i.e., an “ochre” termination codon).

In certain embodiments, a tRNA comprises an anticodon that hybridizes to a non-standard codon, e.g., a 4- or 5-nucleotide codon. Examples of four base codons include AGGA, CUAG, UAGA, and CCCU. Examples of five base codons include AGGAC, CCCCU, CCCUC, CUAGA, CUACU, and UAGGC. tRNAs comprising an anticodon that hybridizes to a non-standard codon, e.g., a 4- or 5-nucleotide codon, and methods of using such tRNAs to incorporate unnatural amino acids into proteins are described, for example, in Moore et al. (2000) J. MOL. BIOL. 298: 195; Hohsaka et al. (1999) J. AM. CHEM. SOC. 121:12194; Anderson et al. (2002) CHEMISTRY AND BIOLOGY 9:237-244; Magliery (2001) J. MOL. BIOL. 307: 755-769; and PCT Publication No. WO2005/007870.

As used herein, the term “tRNA” includes variants having one or more mutations (e.g., nucleotide substitutions, deletions, or insertions) relative to a reference (e.g., a wild-type) tRNA sequence. In certain embodiments, a tRNA may comprise, consist, or consist essentially of, a single mutation (e.g., a mutation contemplated herein), or a combination of 2, 3, 4, 5, 6, 7, 8, 9, 10, 11, 12, 13, 14, 15 or more than 15 mutations (e.g., mutations contemplated herein). It is contemplated that a tRNA may comprise, consist, or consist essentially 1-15, 1-10, 1-7, 1-6, 1-5, 1-4, 1-3, 1-2, 2-15, 2-10, 2-7, 2-6, 2-5, 2-4, 2-3, 3-15, 3-10, 3-7, 3-6, 3-5, or 3-4 mutations (e.g., mutations contemplated herein).

In certain embodiments, a variant suppressor tRNA has increased activity to incorporate an unnatural amino acid (e.g., an unnatural amino acid contemplated herein) into a mammalian protein relative to a counterpart wild-type suppressor tRNA (in this context, a wild-type suppressor tRNA refers to a suppressor tRNA that corresponds to a wild-type tRNA molecule but for any modifications to the anti-codon region to impart suppression activity). The activity of the variant suppressor tRNA may be increased relative to the wild type suppressor tRNA, for example, by about 2.5 to about 200 fold, about 2.5 to about 150 fold, about 2.5 to about 100 fold about 2.5 to about 80 fold, about 2.5 to about 60 fold, about 2.5 to about 40 fold, about 2.5 to about 20 fold, about 2.5 to about 10 fold, about 2.5 to about 5 fold, about 5 to about 200 fold, about 5 to about 150 fold, about 5 to about 100 fold, about 5 to about 80 fold, about 5 to about 60 fold, about 5 to about 40 fold, about 5 to about 20 fold, about 5 to about 10 fold, about 10 to about 200 fold, about 10 to about 150 fold, about 10 to about 100 fold, about 10 to about 80 fold, about 10 to about 60 fold, about 10 to about 40 fold, about 10 to about 20 fold, about 20 to about 200 fold, about 20 to about 150 fold, about 20 to about 100 fold, about 20 to about 80 fold, about 20 to about 60 fold, about 20 to about 40 fold, about 40 to about 200 fold, about 40 to about 150 fold, about 40 to about 100 fold, about 40 to about 80 fold, about 40 to about 60 fold, about 60 to about 200 fold, about 60 to about 150 fold, about 60 to about 100 fold, about 60 to about 80 fold, about 80 to about 200 fold, about 80 to about 150 fold, about 80 to about 100 fold, about 100 to about 200 fold, about 100 to about 150 fold, or about 150 to about 200 fold.

It is contemplated that the tRNA may function in vitro or in vivo and can be provided to a translation system (e.g., an in vitro translation system or a cell) as a mature tRNA (e.g., an aminoacylated tRNA), or as a polynucleotide that encodes the tRNA.

A tRNA may be derived from a bacterial source, e.g., Escherichia coli, Thermus thermophilus, or Bacillus stearothermophilus. A tRNA may also be derived from an archaeal source, e.g, from the Methanosarcinacaea or Desulfitobacterium families, any of the M. barkeri (Mb), M. alvus (Ma), M. mazei (Mm) or D. hafnisense (Dh) families, Methanobacterium thermoautotrophicum, Haloferax volcanii, Halobacterium species NRC-1, or Archaeoglobus fulgidus. In other embodiments, eukaryotic sources can also be used, for example, plants, algae, protists, fungi, yeasts, or animals (e.g., mammals, insects, arthropods, etc.).

In certain embodiments, the tRNA is derived from an E. coli leucyl tRNA and, for example, is preferentially charged with a leucine analog over the naturally-occurring leucine amino acid by an aminoacyl-tRNA synthetase derived from an E. coli leucyl-tRNA synthetase, e.g., an aminoacyl-tRNA synthetase contemplated herein.

For example, the tRNA may comprise, consist essentially of, or consist of the nucleotide sequence of any one of SEQ ID NOs: 16-43, or a nucleotide sequence that has at least 85%, 90%, 95%, 96%, 97%, 98%, or 99% sequence identity to any one of SEQ ID NOs: 16-43.

In certain embodiments, the tRNA is derived from an E. coli tryptophanyl tRNA and, for example, is preferentially charged with a tryptophan analog over the naturally-occurring tryptophan amino acid by an aminoacyl-tRNA synthetase derived from an E. coli tryptophanyl-tRNA synthetase, e.g., an aminoacyl-tRNA synthetase contemplated herein.

For example, the tRNA may comprise, consist essentially of, or consist of the nucleotide sequence of any one of SEQ ID NOs: 49-54 or 108-113, or a nucleotide sequence that has at least 85%, 90%, 95%, 96%, 97%, 98%, or 99% sequence identity to any one of SEQ ID NOs: 49-54 or 108-113.

In certain embodiments, the tRNA is derived from an E. coli tyrosyl tRNA and, for example, is preferentially charged with a tyrosine analog over the naturally-occurring tyrosine amino acid by an aminoacyl-tRNA synthetase derived from an E. coli tyrosyl-tRNA synthetase, e.g., an aminoacyl-tRNA synthetase contemplated herein.

For example, the tRNA may comprise, consist essentially of, or consist of the nucleotide sequence of any one of SEQ ID NOs: 68-69 or 104-105, or a nucleotide sequence that has at least 85%, 90%, 95%, 96%, 97%, 98%, or 99% sequence identity to any one of SEQ ID NOs: 68-69 or 104-105.

In certain embodiments, the tRNA is derived from a M. barkeri pyrrolysyl tRNA and, for example, is preferentially charged with a pyrrolysine analog over the naturally-occurring pyrrolysine amino acid by an aminoacyl-tRNA synthetase derived from a M. barkeri pyrrolysyl-tRNA synthetase, e.g., an aminoacyl-tRNA synthetase contemplated herein.

For example, the tRNA may comprise, consist essentially of, or consist of the nucleotide sequence of any one of SEQ ID NOs: 72-100 or 106-107, or a nucleotide sequence that has at least 85%, 90%, 95%, 96%, 97%, 98%, or 99% sequence identity to any one of SEQ ID NOs: 72-100 or 106-107.

It is understood that, throughout the description, in each instance where a tRNA comprises, consists essentially of, or consists of a nucleotide sequence including one or more thymines (T), a tRNA is also contemplated that comprises, consists essentially of, or consists of the same nucleotide sequence including a uracil (U) in place of one or more of the thymines (T), or a uracil (U) in place of all the thymines (T). Similarly, in each instance where a tRNA comprises, consists essentially of, or consists of a nucleotide sequence including one or more uracils (U), a tRNA is also contemplated that comprises, consists essentially of, or consists of a nucleotide sequence including a thymine (T) in place of the one or more of the uracils (U), or a thymine (T) in place of all the uracils (U). In addition, additional modifications to the bases can be present.

Methods for producing recombinant tRNA are described in U.S. Patent Application Publication Nos. 2003/0108885 and 2005/0009049, Forster et al. (2003) PROC. NATL. ACAD. SCI. USA 100(11):6353-6357, and Feng et al. (2003), PROC. NATL. ACAD. SCI. USA 100(10): 5676-5681.

A tRNA may be aminoacylated (i.e., charged) with a desired unnatural amino acid (UAA) by any method, including enzymatic or chemical methods.

Enzymatic molecules capable of charging a tRNA include aminoacyl-tRNA synthetases, e.g., aminoacyl-tRNA synthetases disclosed herein. Additional enzymatic molecules capable of charging tRNA include ribozymes, for example, as described in Illangakekare et al. (1995) SCIENCE 267:643-647, Lohse et al. (1996) NATURE 381:442-444, Murakami et al. (2003) CHEMISTRY AND BIOLOGY 10:1077-1084, U.S. Patent Application Publication No. 2003/0228593,

Chemical aminoacylation methods include those described in Hecht (1992) ACC. CHEM. RES. 25:545, Heckler et al. (1988) BIOCHEM. 1988, 27:7254, Hecht et al. (1978) J. BIOL. CHEM. 253:4517, Cornish et al. (1995) ANGEW. CHEM. INT. ED. ENGL. 34:621, Robertson et al. (1991) J. AM. CHEM. SOC. 113:2722, Noren et al. (1989) SCIENCE 244:182, Bain et al. (1989) J. AM. CHEM. SOC. 111:8013, Bain et al. (1992) NATURE 356:537, Gallivan et al. (1997) CHEM. BIOL. 4:740, Turcatti et al. (1996) J. BIOL. CHEM. 271:19991, Nowak et al. (1995) SCIENCE 268:439, Saks et al. (1996) J. BIOL. CHEM. 271:23169, and Hohsaka et al. (1999) J. AM. CHEM. SOC. 121:34.

### VII. Vectors

tRNAs, aminoacyl-tRNA synthetases, or any other molecules of interest may be expressed in a cell of interest by incorporating a gene encoding the molecule into an appropriate expression vector. As used herein, “expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system.

tRNAs, aminoacyl-tRNA synthetases, or any other molecules of interest may be introduced to a cell of interest by incorporating a gene encoding the molecule into an appropriate transfer vector. The term “transfer vector” refers to a vector comprising a recombinant polynucleotide which can be used to deliver the polynucleotide to the interior of a cell. It is understood that a vector may be both an expression vector and a transfer vector.

Vectors (e.g., expression vectors or transfer vectors) include all those known in the art, such as cosmids, plasmids (e.g., naked or contained in liposomes), retrotransposons (e.g. piggyback, sleeping beauty), and viruses (e.g., lentiviruses, retroviruses, adenoviruses, and adeno-associated viruses) that incorporate the recombinant polynucleotide of interest.

Typical vectors contain transcription and translation terminators, transcription and translation initiation sequences, and promoters useful for regulation of the expression of the particular target nucleic acid. The vectors optionally comprise generic expression cassettes containing at least one independent terminator sequence, sequences permitting replication of the cassette in eukaryotes, or prokaryotes, or both (including but not limited to, shuttle vectors) and selection markers for both prokaryotic and eukaryotic systems.

In certain embodiments, the vector comprises a regulatory sequence or promoter operably linked to the nucleotide sequence encoding the suppressor tRNA and/or the tRNA synthetase. The term “operably linked” refers to a linkage of polynucleotide elements in a functional relationship. A nucleic acid sequence is “operably linked” when it is placed into a functional relationship with another nucleic acid sequence. For instance, a promoter or enhancer is operably linked to a gene if it affects the transcription of the gene. Operably linked nucleotide sequences are typically contiguous. However, as enhancers generally function when separated from the promoter by several kilobases and intronic sequences may be of variable lengths, some polynucleotide elements may be operably linked but not directly flanked and may even function in trans from a different allele or chromosome.

Exemplary promoters which may be employed include, but are not limited to, the retroviral LTR, the SV40 promoter, the human cytomegalovirus (CMV) promoter, the U6 promoter, the EF1α promoter, the CAG promoter, the H1 promoter, the UbiC promoter, the PGK promoter, the 7SK promoter, a pol II promoter, a pol III promoter, or any other promoter (e.g., cellular promoters such as eukaryotic cellular promoters including, but not limited to, the histone, pol III, and β-actin promoters). Other viral promoters which may be employed include, but are not limited to, adenovirus promoters, TK promoters, and B19 parvovirus promoters. The selection of a suitable promoter will be apparent to those skilled in the art from the teachings contained herein. In certain embodiments, a vector comprises a nucleotide sequence encoding an aminoacyl-tRNA synthetase operably linked to a CMV or an EF1α promoter and/or a nucleotide sequence encoding a suppressor tRNA operably linked to a U6 or an H1 promoter.

In certain embodiments, the vector is a viral vector. The term “virus” is used herein to refer to an obligate intracellular parasite having no protein-synthesizing or energy-generating mechanism. Exemplary viral vectors include retroviral vectors (e.g., lentiviral vectors), adenoviral vectors, adeno-associated viral vectors, herpesviruses vectors, epstein-barr virus (EBV) vectors, polyomavirus vectors (e.g., simian vacuolating virus 40 (SV40) vectors), poxvirus vectors, and pseudotype virus vectors.

The virus may be a RNA virus (having a genome that is composed of RNA) or a DNA virus (having a genome composed of DNA). In certain embodiments, the viral vector is a DNA virus vector. Exemplary DNA viruses include parvoviruses (e.g., adeno-associated viruses), adenoviruses, asfarviruses, herpesviruses (e.g., herpes simplex virus 1 and 2 (HSV-1 and HSV-2), epstein-barr virus (EBV), cytomegalovirus (CMV)), papillomoviruses (e.g., HPV), polyomaviruses (e.g., simian vacuolating virus 40 (SV40), and poxviruses (e.g., vaccinia virus, cowpox virus, smallpox virus, fowlpox virus, sheeppox virus, myxoma virus). In certain embodiments, the viral vector is a RNA virus vector. Exemplary RNA viruses include bunyaviruses (e.g., hantavirus), coronaviruses, flaviviruses (e.g., yellow fever virus, west nile virus, dengue virus), hepatitis viruses (e.g., hepatitis A virus, hepatitis C virus, hepatitis E virus), influenza viruses (e.g., influenza virus type A, influenza virus type B, influenza virus type C), measles virus, mumps virus, noroviruses (e.g., Norwalk virus), poliovirus, respiratory syncytial virus (RSV), retroviruses (e.g., human immunodeficiency virus-1 (HIV-1)) and toroviruses.

### VIII. Host Cells and Cell Lines

Also encompassed by the invention are host cells or cell lines (e.g., prokaryotic or eukaryotic host cells or cell lines) that include a tRNA, aminoacyl-tRNA synthetase, unnatural amino acid, nucleic acid, and/or vector disclosed herein. The nucleic acid encoding the engineered tRNA and aminoacyl-tRNA synthetase can be expressed in an expression host cell either as an autonomously replicating vector within the expression host cell (e.g., a plasmid, or viral particle) or via a stable integrated element or series of stable integrated elements in the genome of the expression host cell, e.g., a mammalian host cell.

Host cells are genetically engineered (including but not limited to, transformed, transduced or transfected), for example, using nucleic acids or vectors disclosed herein. For example, in certain embodiments, one or more vectors include coding regions for an orthogonal tRNA, an orthogonal aminoacyl-tRNA synthetase, and, optionally, a protein (e.g., an antibody) to be modified by the inclusion of one or more UAAs, which are operably linked to gene expression control elements that are functional in the desired host cell or cell line. For example, the genes encoding tRNA synthetase and tRNA and an optional selectable marker (e.g., an antibiotic resistance gene, e.g., a puromycin resistance cassette) can be integrated in a transfer vector (e.g., a plasmid, which can be linearized prior to transfection), where for example, the genes encoding the tRNA synthetase can be under the control of a polymerase II promoter (e.g., CMV, EF1α, UbiC, or PGK, e.g., CMV or EF1α) and the genes encoding the tRNA can be under the control of a polymerase III promoter (e.g., U6, 7SK, or H1, e.g., U6). The vectors are transfected into cells and/or microorganisms by standard methods including electroporation or infection by viral vectors, and clones can be selected via expression of the selectable marker (for example, by antibiotic resistance).

Exemplary prokaryotic host cells or cell lines include cells derived from a bacteria, e.g., Escherichia coli, Thermus thermophilus, Bacillus stearothermophilus, Pseudomonas fluorescens, Pseudomonas aeruginosa, and Pseudomonas putida. Exemplary eukaryotic host cells or cell lines include cells derived from a plant (e.g., a complex plant such as a monocot or dicot), an algae, a protist, a fungus, a yeast (including Saccharomyces cerevisiae), or an animal (including a mammal, an insect, an arthropod, etc.). Additional exemplary host cells or cell lines include HEK293, HEK293T, Expi293, CHO, CHOK1, Sf9, Sf21, HeLa, U20S, A549, HT1080, CAD, P19, NIH 3T3, L929, N2a, MCF-7, Y79, SO-RB50, HepG2, DUKX-X11, J558L, BHK, COS, Vero, NS0, or ESCs. It is understood that a host cell or cell line can include individual colonies, isolated populations (monoclonal), or a heterogeneous mixture of cells.

A contemplated cell or cell line includes, for example, one or multiple copies of an orthogonal tRNA/aminoacyl-tRNA synthetase pair, optionally stably maintained in the cell's genome or another piece of DNA maintained by the cell. For example, the cell or cell line may contain one or more copies of (i) a tryptophanyl tRNA/aminoacyl-tRNA synthetase pair (wild type or engineered) stably maintained by the cell, and/or (ii) a leucyl tRNA/aminoacyl-tRNA synthetase pair (wild-type or engineered) stably maintained by the cell.

For example, in certain embodiments, the cell line is a stable cell line and the cell line comprises a genome having stably integrated therein (i) a nucleic acid sequence encoding an aminoacyl-tRNA synthetase (e.g., a prokaryotic tryptophanyl-tRNA synthetase mutein capable of charging a tRNA with an unnatural amino acid or a prokaryotic leucyl-tRNA synthetase mutein capable of charging a tRNA with an unnatural amino acid, e.g., a tRNA synthetase mutein disclosed herein); and/or (ii) a nucleic acid sequence encoding a suppressor tRNA (e.g., prokaryotic suppressor tryptophanyl-tRNA capable of being charged with an unnatural amino acid or prokaryotic suppressor leucyl-tRNA capable of being charged with an unnatural amino acid, e.g., a suppressor tRNA disclosed herein).

Methods to introduce a nucleic acid encoding a tRNA and/or an aminoacyl-tRNA synthetase into the genome of a cell of interest, or to stably maintain the nucleic acid in DNA replicated by the cell that is outside of the genome, are well known in the art.

The nucleic acid encoding the tRNA and/or an aminoacyl-tRNA synthetase can be provided to the cell in an expression vector, transfer vector, or DNA cassette, e.g., an expression vector, transfer vector, or DNA cassette disclosed herein. The expression vector transfer vector, or DNA cassette encoding the tRNA and/or aminoacyl-tRNA synthetase can contain one or more copies of the tRNA and/or aminoacyl-tRNA synthetase optionally under the control of an inducible or constitutively active promoter. The expression vector, transfer vector, or DNA cassette may, for example, contain other standard components (enhancers, terminators, etc.). It is contemplated that the nucleic acid encoding the tRNA and the nucleic acid encoding the aminoacyl-tRNA synthetase may be on the same or different vector, may be present in the same or different ratios, and may be introduced into the cell, or stably integrated in the cellular genome, at the same time or sequentially.

One or multiple copies of a DNA cassette encoding the tRNA and/or aminoacyl-tRNA synthetase can be integrated into a host cell genome or stably maintained in the cell using a transposon system (e.g., PiggyBac), a viral vector (e.g., a lentiviral vector or other retroviral vector), CRISPR/Cas9 based recombination, electroporation and natural recombination, a BxB1 recombinase system, or using a replicating/maintained piece of DNA (such as one derived from Epstein-Barr virus).

In order to select for cell lines which stably maintain the nucleic acid encoding the tRNA and/or aminoacyl-tRNA synthetase and/or are efficient at incorporating UAAs into a protein (e.g., an antibody) of interest, a selectable marker can be used. Exemplary selectable markers include zeocin, puromycin, neomycin, dihydrofolate reductase (DHFR), glutamine synthetase (GS), mCherry-EGFP fusion, or other fluorescent proteins. In certain embodiments, a gene encoding a selectable marker protein (or a gene encoding a protein required for a detectable function, e.g., viability, in the presence of the selectable marker) may include a premature stop codon, such that the protein will only be expressed if the cell line is capable of incorporating a UAA at the site of the premature stop codon.

In certain embodiments, to develop a host cell or cell line including two or more tRNA/aminoacyl-tRNA synthetase pairs, one can use multiple identical or distinct UAA directing codons in order to identify host cells or cell lines which have incorporated multiple copies of the two or more tRNA/aminoacyl-tRNA synthetase pairs through iterative rounds of genomic integration and selection. Host cells or cell lines which contain enhanced UAA incorporation efficiency, low background, and decreased toxicity can first be isolated via a selectable marker containing one or more stop codons. Subsequently, the host cells or cell lines can be subjected to a selection scheme to identify host cells or cell lines which contain the desired copies of tRNA/aminoacyl-tRNA synthetase pairs and express a gene of interest (either genomically integrated or not) containing one or more stop codons. Protein expression may be assayed using any method known in the art, including for example, Western blot using an antibody that binds the protein of interest or a C-terminal tag.

The host cells or cell lines be cultured in conventional nutrient media modified as appropriate for such activities as, for example, screening steps, activating promoters or selecting transformants. These cells can optionally be cultured into transgenic organisms. Other useful references, e.g. for cell isolation and culture (e.g., for subsequent nucleic acid isolation) include Freshney (1994) Culture of Animal Cells, a Manual of Basic Technique, third edition, Wiley-Liss, New York; Payne et al. (1992) Plant Cell and Tissue Culture in Liquid Systems John Wiley & Sons, Inc. New York, N.Y.; Gamborg and Phillips (eds.) (1995) Plant Cell, Tissue and Organ Culture; Fundamental Methods Springer Lab Manual, Springer-Verlag (Berlin Heidelberg N.Y.) and Atlas and Parks (eds.) The Handbook of Microbiological Media (1993) CRC Press, Boca Raton, Fla.

The production of an exemplary cell line capable of producing antibodies incorporating a UAA is described in Roy et al. (2020) MABS 12(1), e1684749).

Throughout the description, where compositions are described as having, including, or comprising specific components, or where processes and methods are described as having, including, or comprising specific steps, it is contemplated that, additionally, there are compositions of the present invention that consist essentially of, or consist of, the recited components, and that there are processes and methods according to the present invention that consist essentially of, or consist of, the recited processing steps.

In the application, where an element or component is said to be included in and/or selected from a list of recited elements or components, it should be understood that the element or component can be any one of the recited elements or components, or the element or component can be selected from a group consisting of two or more of the recited elements or components.

Further, it should be understood that elements and/or features of a composition or a method described herein can be combined in a variety of ways without departing from the spirit and scope of the present invention, whether explicit or implicit herein. For example, where reference is made to a particular compound, that compound can be used in various embodiments of compositions of the present invention and/or in methods of the present invention, unless otherwise understood from the context. In other words, within this application, embodiments have been described and depicted in a way that enables a clear and concise application to be written and drawn, but it is intended and will be appreciated that embodiments may be variously combined or separated without parting from the present teachings and invention(s). For example, it will be appreciated that all features described and depicted herein can be applicable to all aspects of the invention(s) described and depicted herein.

It should be understood that the expression “at least one of” includes individually each of the recited objects after the expression and the various combinations of two or more of the recited objects unless otherwise understood from the context and use. The expression “and/or” in connection with three or more recited objects should be understood to have the same meaning unless otherwise understood from the context.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

Where the use of the term “about” is before a quantitative value, the present invention also includes the specific quantitative value itself, unless specifically stated otherwise. As used herein, the term “about” refers to a ±10% variation from the nominal value unless otherwise indicated or inferred.

It should be understood that the order of steps or order for performing certain actions is immaterial so long as the present invention remain operable. Moreover, two or more steps or actions may be conducted simultaneously.

The use of any and all examples, or exemplary language herein, for example, “such as” or “including,” is intended merely to illustrate better the present invention and does not pose a limitation on the scope of the invention unless claimed. No language in the specification should be construed as indicating any non-claimed element as essential to the practice of the present invention.

### EXAMPLES

The following Examples are merely illustrative and are not intended to limit the scope or content of the invention in any way.

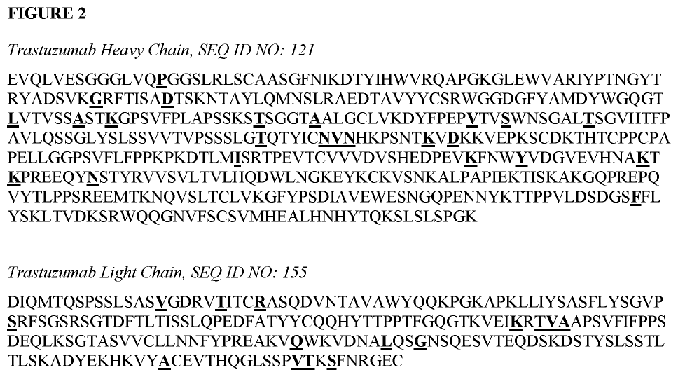

This work describes the generation of antibodies containing unnatural amino acids (for example, amino acids depicted in FIGS. 6A-6G) at specified sites. As outlined in FIG. 7, four different antibodies (Trastuzumab tested twice (Trastuzumab A and Trastuzumab B), a non-Trastuzumab IgG1, a non-Trastuzumab bispecific antibody, and a second non-Trastuzumab IgG1) were mutated to contain either a TAG or TGA stop codon at one or multiple sites in order to introduce unnatural amino acids as previously described, using transient transfection screening methods. Sites of consideration for the present studies are shown in FIG. 2. These antibodies were subsequently conjugated with an exemplary partner payload, identified from the list provided in FIG. 7. Antibodies and antibody drug conjugates (ADCs) were characterized with the parameters and assays outlined in FIG. 7.

### Example 1—Generation of UAA-Containing Antibodies

### Antibodies Containing the Unnatural Amino Acid LCA

The following protocol was used for expression of antibodies containing LCA, and conjugation of molecules to the antibodies with a target drug-antibody ratio of 2 to 4 (DAR2-4).

All monoclonal antibody expression was performed using the Expi293 Expression System according to the manufacturer's instructions. Briefly, before transfection, cells were split to a density of 2.7×10⁶ to 3.0×10⁶ cells/ml. An unnatural amino acid (UAA), LCA, was added to achieve a final concentration of 0.25 to 0.5 mM. A total of 1 mg of plasmid mix (equal parts suppressor plasmid, heavy chain plasmid, and light chain plasmid) was used for transfection into 1 L cell culture. Suppressor plasmid contained anywhere from 1 to 20 copies of leucyl tRNA (Leu-tRNA.h1; SEQ ID NO: 19) or leucyl synthetase (LeuRS.v1; SEQ ID NO: 2). Heavy chain or light chain plasmids were mutated to contain a TAG (LCA) stop codon for the incorporation of the UAA. Plasmids were diluted in 50 ml Opti-MEM medium. A PEI stock solution was incubated at room temperature for 3 minutes, subsequently diluted to achieve a 6:1 PEI:DNA final ratio, and incubated for 15 minutes at room temperature. The Plasmid: PEI complex was then added dropwise to the culture. At the time of transfection, 0.25 to 1 mM LCA was added to the cells. Cells were incubated on an orbital shaking platform at 37° C. with 8% CO₂ at a speed of 80 to 125 rpm for 5 to 8 days. Protein was purified using a PrismA column (Cytiva: 17549801) for use in characterization assays described herein.

The same expression and purification protocol described above was used for multi-DAR (e.g., DAR4) species containing the same UAA, with plasmids modified to contain the specified mutagenesis sites in order to incorporate the desired number and location of UAAs.

Expression levels of LCA-containing antibodies at single sites of incorporation are depicted in FIG. 12 and FIG. 13. As shown in FIG. 7, sites were selected for screening on Trastuzumab (TzmAb), IgG1a, or IgG1b. While incorporation of UAA at many sites resulted in moderate yields of more than 10% of wild-type (WT) protein expression, sites on the light chain such as LC-K107, LC-T109, and sites on the heavy chain such as HC-A121, HC-A143, HC-T198, HC-1256, and HC-K291, showed robust expression on multiple antibody scaffolds. Furthermore, expression yields obtained for the antibodies were without optimization of transient transfection conditions. In some examples, as demonstrated in FIG. 32A and FIG. 32B, multiple LCA amino acids were introduced and later conjugated. Together, these results suggests that the identified sites can be used to introduce UAAs on multiple, different antibodies.

### Antibodies Containing Unnatural Amino Acid HTP

Transfection and expression of HTP-containing antibodies was performed as described above for LCA-containing antibodies; however, the suppressor plasmid used contained the tryptophanyl tRNA (Trp-tRNA-UCA; SEQ ID NO: 51)/aaRS (trpRS.h14; SEQ ID NO: 45) pair rather than leucyl, as well as a TGA stop codon rather than a TAG stop codon, in order to direct the incorporation of the UAA into the antibody site of interest. At the time of transfection, HTP was added to the cells for a final concentration of 0.25 to 1 mM. Yields of HTP-containing proteins are outlined in FIG. 31. Screening yields exceeded about 24% for most sites. This data highlights the ability to use the sites A121 and T198 for efficient HTP incorporation.

### Antibodies Containing Unnatural Amino Acids LCA and HTP

Transfection and UAA addition were performed as described above with modifications to the plasmid ratios. Equal parts leucyl suppressor, tryptophanyl suppressor, heavy chain, and light chain plasmid were transfected. Mutagenesis to introduce a TAG or TGA site as appropriate was performed using standard cloning techniques. Antibodies were purified as described above. An exemplary antibody including UAAs at both T109 and A121 is described in FIG. 32A and FIG. 32B. High expression yields were obtained.

Antibodies with Site Specific Cysteines

Site-specific cysteine mutants were prepared according to techniques known in the art, to allow for direct comparison to present UAA sites of interest, such as T198. The heavy chain of trastuzumab was mutated to contain a T198-cysteine mutation, and was transfected and expressed in Expi293 cells, without suppressor plasmid or UAA addition, as described above.

As a comparison, cysteine sites were conjugated by substitution of cysteine at a site of interest using molecular biology techniques known in the art. The mAb was buffer exchanged to 50 mM HEPES, pH 7.2 with 2 mM EDTA. Forty equivalents of tris(2-carboxyethyl)phosphine (TCEP) were added and the resultant mAb composition was incubated at room temperature for 2 hours. The reduced mAb was again buffer exchanged to 50 mM HEPES, pH 7.2 with 2 mM EDTA. The reduced mAb was then re-oxidized with 12 to 20 equivalents of Dehydroabietic acid (DHAA) and incubated at room temperature for 3 hours. A solution of 5% of DMSO, followed by 4 to 6 equivalents of linker-payload, were added to the mAb, and the mixture was incubated for 1 hour at room temperature to obtain the ADC.

For SDS-PAGE, Invitrogen Novex NuPAGE 4-12% Bis-Tris Protein gels were used according to the manufacturer's instructions. Samples of 1-2 μg of antibody were mixed with 6×SDS loading buffer and loaded into the SDS-PAGE gel. SDS-PAGE were resolved at constant 180 V for about 40 minutes and Coomassie solution was used for detection of protein bands.

### Example 2—Conjugation and Biophysical Characterization of Antibodies

This Example describes the conjugation of LCA-containing antibodies with payloads shown in FIGS. 8-10. This Example demonstrates the ability to use the LCA-containing antibodies disclosed herein to introduce payloads such as PEG4/PEG8 linkers, valine-citrulline (VC) linkers, small hydrophobic payloads, peptide-based payloads, nucleic acids (such as locked nucleic acids) and other hydrophobic payloads.

### Click Chemistry Conjugation Protocol and HIC Characterization

LCA-containing antibodies were diluted to a final concentration of 1 mg/ml to 5 mg/ml in PBS. 10 mM to 50 mM of payload stock solutions were made in buffer or organic solvent (DMSO, DMF, etc.) as appropriate. Payload was added at a molar excess of 2 to 75 fold and incubated at room temperature for 1 hour to 16 hours.

FIG. 14 and FIG. 15 depict the average DAR post conjugation of LCA-containing antibodies with a variety of payloads (exemplary payloads are shown in FIG. 8, FIG. 9, FIG. 10, and FIG. 11). To obtain the HIC metrics as shown in FIG. 14 and FIG. 15, all samples were analyzed with a MabPac Butyl column. A mobile phase A was 1.5 M ammonium sulfate, 25 mM sodium phosphate dibasic, pH 7 and a mobile phase B was 25 mM sodium phosphate, pH 7. The samples were eluted with a flow rate of 0.8 mL/min and at a column temperature of 25° C. The average DAR and distribution profile were determined by peak area percentage of each species. In FIG. 14, for example, the theoretical DAR is 2.0, with an acceptable DAR (per industry standards) being as close to 2.0 as possible. In many instances, the industry standard DAR is greater than 1.5, and, importantly, the majority of the sites identified in the present study achieved this threshold for any payload, demonstrating the versatility of these sites to attach any biologically or chemically acceptable payload.

In some antibody compositions, multiple LCAs were introduced into the antibody scaffold at sites specified in FIG. 32A and FIG. 32B. These antibodies were conjugated with their corresponding payloads and their target DAR of 4 was analyzed. In all cases, a DAR greater than 3.7 was achieved, demonstrating the high efficiency of the presently described methodologies.

FIG. 4 depicts raw data for a number of the Monomethyl auristatin D (MMAD)-payload-based LCA conjugates. HIC and SEC results are shown for Trastuzumab-HC-T198-LCA (FIG. 4A, FIG. 4B, and FIG. 4C), Trastuzumab HC-A143-LCA (FIG. 4D and FIG. 4E), Trastuzumab HC-A121-LCA (FIG. 4F and FIG. 4G), Trastuzumab LC-K107-LCA (FIG. 4H and FIG. 4I), and Trastuzumab LC-T109-LCA (FIG. 4J and FIG. 4K). FIG. 5 depicts mass spectrometry results. Deconvoluted mass spectrums and a liquid chromatography mass spectrometry (LC-MS) chromatogram are shown for Trastuzumab HC-T198-LCA (FIG. 5A, FIG. 5C, and FIG. 5B, respectively), Trastuzumab HC-T198-BCN-PEG4-MMAD (FIG. 5D and FIG. 5E, respectively), Trastuzumab HC-T198-HTP (FIG. 5F, FIG. 5H, and FIG. 5G, respectively), Trastuzumab HC-T198-HTP+Diazonium-Biotin (FIG. 5I, FIG. 5K, and FIG. 5J, respectively), Trastuzumab HC-A143-LCA (FIG. 5L, FIG. 5N, and FIG. 5M, respectively), Trastuzumab HC-A143-BCN-PEG4-MMAD (FIG. 5O, FIG. 5Q, and FIG. 5P, respectively), Trastuzumab HC-A121-LCA (FIG. 5R, FIG. 5T, and FIG. 5S, respectively), Trastuzumab HC-A121-BCN-PEG8-MMAD (FIG. 5U and FIG. 5V, respectively), Trastuzumab LC-K107-LCA (FIG. 5W and FIG. 5X, respectively), Trastuzumab LC-T109-LCA (FIG. 5Y, FIG. 5AA, and FIG. 5Z, respectively), Trastuzumab LC-T109-BCN-PEG8-MMAD (FIG. 5BB, FIG. 5DD, and FIG. 5CC, respectively). For the mass spectrometry analysis, proteins were denatured, reduced, and PNGase treated prior to analysis via electrospray ionization mass spectrometry (ESI-MS). Results, as shown in FIG. 5, demonstrate successful conjugation and homogenous product, with the DAR corroborated by HIC. Each figure indicates the antibody that was analyzed.

### Chemoselective Rapid Azo-Coupling Reaction (CRACR) Conjugation Protocol and HIC Characterization

For a sampling of sites outlined in FIG. 31, the UAA HTP was introduced instead of LCA. The HTP-containing antibody was diluted to a final concentration of 1 mg/ml to 5 mg/ml in PBS. Payload stock solution (Diazo-Conjugate) was freshly made at 10 mM to 50 mM concentration in suitable buffer or organic solvent (DMSO, DMF, etc.) as appropriate. The diazonium payload was added slowly while stirring to an excess of 2 to 75 fold and incubated at room temperature for 1 hour to 3 hours. FIG. 31 provides results for both IgG1a antibodies with HTP-conjugates and bispecific antibodies containing HTP-conjugates. In each instance, the target DAR was achieved, demonstrating the versatility of the sites listed in FIG. 31 for the incorporation and conjugation of HTP-containing UAAs. Surprisingly, T198 gave an average DAR of 1 instead of the theoretical DAR of 2, demonstrating potential differentiability of sites based on the conjugation chemistry used, and suggesting the present methodologies may provide for more flexible and modular control of the final DAR of payloads.

### Multi-Drug Conjugation

For introduction of multiple distinct UAAs into the same protein (e.g., introduction of LCA and HTP, as shown in FIG. 32A and FIG. 32B), a conjugation was performed in a one pot reaction. For these proteins containing both HTP (tryptophan analogs) and LCA (leucyl analog), the CRACR or HTP protocol was performed first, followed by the azide alkyne conjugation in order to generate final antibody conjugates containing multiple, distinct payloads conjugated to their corresponding UAA-handles.

### Size-Exclusion Chromatography (SEC) Analysis

In addition to HIC as described above, conjugates were analyzed by SEC to determine protein purity and assess aggregation. Samples were diluted with the mobile phase composition (20 mM sodium phosphate, pH 6.8) at a concentration of 2 μM and injected onto a SEC Yarra-3000 column at 0.4 mL/min for 20 minutes. The aggregate and monomer species were reported as the percent of the total area for all protein-related peaks. FIG. 16 and FIG. 17 depict aggregation, as measured by SEC, for LCA-based conjugates. As shown in FIGS. 16-17, most sites resulted in little to no protein aggregation (e.g., less than or equal to 1% aggregation). Locked-nucleic-acid (LNA)-containing mAbs contained slightly more aggregation, peaking at about 7%; it is believed this aggregation value could be significantly decreased with the introduction of wash and/or additional clean-up steps. Additionally, the K291 site specifically seems to have a higher propensity for aggregation relative to other sites following conjugation to the DBCO-PEG8-VC-SM payload. The data demonstrate that incorporation of LCA at most sites using the methods described herein resulted in a low propensity for aggregation across a broad spectrum of payloads.

FIG. 31 demonstrates aggregation data for HTP-containing antibodies and their conjugates. For these antibodies, aggregation was slightly higher than that observed with LCA (about 5% instead of about 1%), demonstrating the impact of incorporating different UAAs at different sites. HTP at T198 resulted in the least amount of aggregation, 1%, which was similar to the LCA aggregation propensities described above. The majority of these aggregation propensities pass industry thresholds and can be further improved with additional cleanup steps.

Lastly, SEC of multisite conjugates (single and multi-drug) were analyzed, as shown in FIG. 32A (Trastuzumab) and FIG. 32B (non-Trastuzumab IgG1 and bispecific antibody). All combinations of the single site LCA conjugates (first nine) that demonstrated the least aggregation resulted in little to no aggregation when combined for DAR4+ or multiple distinct UAAs. Conjugates containing multiple, distinct UAAs and conjugation of LNA/Diazo-Payload resulted in minor aggregation, but surpassed the threshold of 90% monomer, a common industry standard for aggregation.

### Example 3—Predictive Functionality Assays

This example describes characterization of the presently described antibodies and antibody conjugates in preclinical assays chosen based on their ability to predict efficacy in in vivo models. These assays included human plasma stability assays, cathepsin B cleavage assays, and in vitro cytotoxicity assays.

### Human Plasma Stability Assay

Human plasma (Molecular Innovations, Cat. No.: HPLA-SER-GF-100ML) was spiked with a final antibody drug conjugate (ADC) concentration of 0.2 mg/mL and subsequently incubated at 37° C. in 100 μL aliquots for up to 72 hours; aliquots were immediately flash frozen and stored at −80° C. until further analysis. For analysis, plasma-incubated ADCs were thawed and subjected to affinity capture with Protein G magnetic beads. The eluted samples containing purified incubated ADC were then analyzed by HIC as described above. Stability was measured by calculating the percentage of ADC remaining post-human plasma incubation. As represented in FIG. 18 and FIG. 19, particular LCA incorporation sites, such as T198 and A121, have robust stability in plasma with a variety of cleavable and non-cleavable payloads. However, some sites, such as HC-T158, showed a lack of stability, even with non-cleavable payloads in human plasma, highlighting the importance of analysis across multiple sites of incorporation.

### Cathepsin B Assay:

To measure the stability of cleavable linkers based on site of incorporation, activated Cathepsin B was incubated with LCA-conjugated ADCs in reaction buffer (25 mM sodium acetate and 1 mM EDTA at pH 5.0). The samples were incubated at 37° C. for up to 240 minutes. Samples were quenched by adding E-64 protease inhibitor to a final concentration of 10 μM and stored at −80° C. prior to HIC analysis. Samples were thawed individually and analyzed by HIC analysis as described above. The percentage of cleavage is reported for each ADC; for example, 100% cleavage signifies that an ADC payload was completely liberated during the incubation period by cathepsin. Variability was again observed between sites, as seen in FIG. 20 and FIG. 21. Both the Monomethyl auristatin E (MMAE) and Non-toxic SM payloads were linked via cleavable linkers, but some sites such as L154 and K213 were found not cleavable in this assay. In particular, T109, A121, and T198 represent robust sites that can accommodate a variety of payloads which require cathepsin based cleavage. These data again suggest the variability of payload and linker can have a significant biological impact based on the site of incorporation, as seen with N206.

### HER2 Elisa Binding Assay

An ELISA binding assay was employed to compare the effect of UAA incorporation and conjugation of anti-HER2 ADCs. 5 μg of human ErbB2 receptor Fc conjugate was captured on a clear 96-well plate for 1 hour at room temperature. After blocking with 3% milk, ADC samples were added at dilutions ranging from 0 to 100 nM and incubated for 1 hour at room temperature. After washing with 0.05% Tween-20 buffer, goat anti-human κ-HRP conjugate was added at 1:1,000 dilution in blocking buffer and incubated for 1 hour at room temperature. QuantaBlu Fluorogenic Peroxidase Substrate was used for the binding quantification.

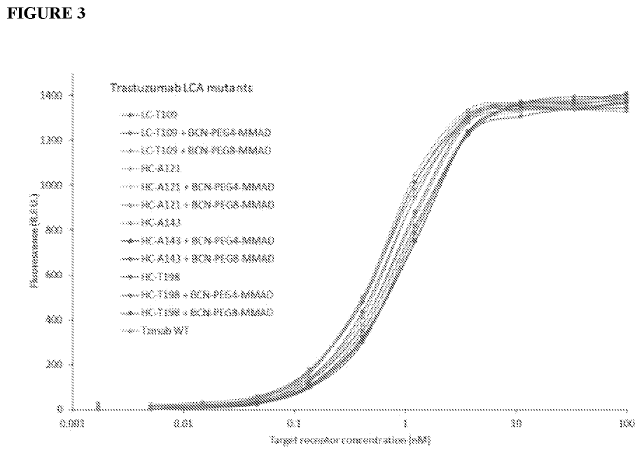

As shown in FIG. 3, FIG. 22A and FIG. 22B, the incorporation of UAAs at sites T109, A121, A143, and T198, and conjugation of these resides with DBCO-PEG4/PEG8-MMAD, maintained sub-nanomolar binding affinities. These data demonstrate that at the particular sites tested there is minimal perturbation of the antibody's antigen-binding ability.

### Example 4—In Vitro Cytotoxicity

This example describes the in vitro cytotoxicity of anti-HER2-MMAD conjugates, which are used to depict the effect of site of incorporation and linker conjugation on the overall activity of the ADC conjugates of the present disclosure.

HER2+ (NCI-N87) and HER2− (NCI-H520) cell lines were selected for testing of ADCs. 8-point serial dilutions of each test sample were tested on the selected cell line(s). After 96 hours incubation in the presence of ADCs, cell viability was measured using a CellTiter-Glo® assay according to the manufacturer's instructions. FIG. 23 depicts a table of the IC50 for all tested sites containing DBCO-PEG4/PEG8-MMAD conjugates and FIG. 24A and FIG. 24B together depict all cytotoxicity traces. FIG. 25, FIG. 26, FIG. 27, and FIG. 28 depict T198, A143, A121, and T109 curves in comparison to a T198 MC-MMAD cysteine site-specific mutant as described in Example 1 above, in NCI-N87 cells (FIG. 25A, FIG. 26A, FIG. 27A, and FIG. 28A) and NCI-H520 cells (FIG. 25B, FIG. 26B, FIG. 27B, and FIG. 28B). Overall, PEG8 payload outcompeted PEG4 payload, with slight variations observed between sites (FIG. 29A and FIG. 29B, and FIG. 30A and FIG. 30B). Some off target toxicity was observed for the particular PEG4-MMAD conjugates shown in FIG. 30A and FIG. 30B as well as the PEG8 conjugates in FIG. 29A and FIG. 29B.

The data shown and described in these Examples demonstrate that all tested antibody drug conjugates of the present studies, designed using the methodologies of the present disclosure, were efficacious and displayed superior safety profiles to ADCs obtained using alternative methods.

### INCORPORATION BY REFERENCE

The entire disclosure of each of the patent and scientific documents referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes that come within the meaning and range of equivalency of the claims are intended to be embraced therein.

What is claimed is:

1. An antibody,
wherein the antibody comprises a heavy chain, or a fragment thereof, comprising:
(i) the amino acid sequence of SEQ ID NO: 118, with an unnatural amino acid (UAA) at a position corresponding to P6 of SEQ ID NO: 118; the amino acid sequence of SEQ ID NO: 119, with an unnatural amino acid (UAA) at a position corresponding to P14 of SEQ ID NO: 119; the amino acid sequence of SEQ ID NO: 120, with an unnatural amino acid (UAA) at a position corresponding to P14 of SEQ ID NO: 120; or the amino acid sequence of SEQ ID NO: 121, with an unnatural amino acid (UAA) at a position corresponding to P14 of SEQ ID NO: 121;
(ii) the amino acid sequence of SEQ ID NO: 122, with an unnatural amino acid (UAA) at a position corresponding to G6 of SEQ ID NO: 122; the amino acid sequence of SEQ ID NO: 123, with an unnatural amino acid (UAA) at a position corresponding to G17 of SEQ ID NO: 123; the amino acid sequence of SEQ ID NO: 120, with an unnatural amino acid (UAA) at a position corresponding to G66 of SEQ ID NO: 120; or the amino acid sequence of SEQ ID NO: 121, with an unnatural amino acid (UAA) at a position corresponding to G66 of SEQ ID NO: 121;
(iii) the amino acid sequence of SEQ ID NO: 124, with an unnatural amino acid (UAA) at a position corresponding to D6 of SEQ ID NO: 124; the amino acid sequence of SEQ ID NO: 125, with an unnatural amino acid (UAA) at a position corresponding to D7 of SEQ ID NO: 125; the amino acid sequence of SEQ ID NO: 120, with an unnatural amino acid (UAA) at a position corresponding to D73 of SEQ ID NO: 120; or the amino acid sequence of SEQ ID NO: 121, with an unnatural amino acid (UAA) at a position corresponding to D73 of SEQ ID NO: 121;
(iv) the amino acid sequence of SEQ ID NO: 126, with an unnatural amino acid (UAA) at a position corresponding to L6 of SEQ ID NO: 126; the amino acid sequence of SEQ ID NO: 127, with an unnatural amino acid (UAA) at a position corresponding to L6 of SEQ ID NO: 127; the amino acid sequence of SEQ ID NO: 120, with an unnatural amino acid (UAA) at a position corresponding to L115 of SEQ ID NO: 120; or the amino acid sequence of SEQ ID NO: 121, with an unnatural amino acid (UAA) at a position corresponding to L115 of SEQ ID NO: 121;
(v) the amino acid sequence of SEQ ID NO: 131, with an unnatural amino acid (UAA) at a position corresponding to K6 of SEQ ID NO: 131; the amino acid sequence of SEQ ID NO: 129, with an unnatural amino acid (UAA) at a position corresponding to K3 of SEQ ID NO: 129; the amino acid sequence of SEQ ID NO: 130, with an unnatural amino acid (UAA) at a position corresponding to K3 of SEQ ID NO: 130; or the amino acid sequence of SEQ ID NO: 121, with an unnatural amino acid (UAA) at a position corresponding to K124 of SEQ ID NO: 121;
(vi) the amino acid sequence of SEQ ID NO: 132, with an unnatural amino acid (UAA) at a position corresponding to T6 of SEQ ID NO: 132; the amino acid sequence of SEQ ID NO: 129, with an unnatural amino acid (UAA) at a position corresponding to T18 of SEQ ID NO: 129; the amino acid sequence of SEQ ID NO: 130, with an unnatural amino acid (UAA) at a position corresponding to T18 of SEQ ID NO: 130; or the amino acid sequence of SEQ ID NO: 121, with an unnatural amino acid (UAA) at a position corresponding to T138 of SEQ ID NO: 121;
(vii) the amino acid sequence of SEQ ID NO: 133, with an unnatural amino acid (UAA) at a position corresponding to A6 of SEQ ID NO: 133; the amino acid sequence of SEQ ID NO: 129, with an unnatural amino acid (UAA) at a position corresponding to A23 of SEQ ID NO: 129; the amino acid sequence of SEQ ID NO: 130, with an unnatural amino acid (UAA) at a position corresponding to A23 of SEQ ID NO: 130; or the amino acid sequence of SEQ ID NO: 121, with an unnatural amino acid (UAA) at a position corresponding to A143 of SEQ ID NO: 121;
(viii) the amino acid sequence of SEQ ID NO: 134, with an unnatural amino acid (UAA) at a position corresponding to V6 of SEQ ID NO: 134; the amino acid sequence of SEQ ID NO: 129, with an unnatural amino acid (UAA) at a position corresponding to V37 of SEQ ID NO: 129; the amino acid sequence of SEQ ID NO: 130, with an unnatural amino acid (UAA) at a position corresponding to V37 of SEQ ID NO: 130; or the amino acid sequence of SEQ ID NO: 121, with an unnatural amino acid (UAA) at a position corresponding to V157 of SEQ ID NO: 121;
(ix) the amino acid sequence of SEQ ID NO: 174, with an unnatural amino acid (UAA) at a position corresponding to T6 of SEQ ID NO: 174; the amino acid sequence of SEQ ID NO: 129, with an unnatural amino acid (UAA) at a position corresponding to T38 of SEQ ID NO: 129; the amino acid sequence of SEQ ID NO: 130, with an unnatural amino acid (UAA) at a position corresponding to T38 of SEQ ID NO: 130; or the amino acid sequence of SEQ ID NO: 121, with an unnatural amino acid (UAA) at a position corresponding to T158 of SEQ ID NO: 121;
(x) the amino acid sequence of SEQ ID NO: 135, with an unnatural amino acid (UAA) at a position corresponding to S6 of SEQ ID NO: 135; the amino acid sequence of SEQ ID NO: 129, with an unnatural amino acid (UAA) at a position corresponding to S40 of SEQ ID NO: 129; the amino acid sequence of SEQ ID NO: 130, with an unnatural amino acid (UAA) at a position corresponding to S40 of SEQ ID NO: 130; or the amino acid sequence of SEQ ID NO: 121, with an unnatural amino acid (UAA) at a position corresponding to S160 of SEQ ID NO: 121;
(xi) the amino acid sequence of SEQ ID NO: 136, with an unnatural amino acid (UAA) at a position corresponding to T6 of SEQ ID NO: 136; the amino acid sequence of SEQ ID NO: 129, with an unnatural amino acid (UAA) at a position corresponding to T47 of SEQ ID NO: 129; the amino acid sequence of SEQ ID NO: 130, with an unnatural amino acid (UAA) at a position corresponding to T47 of SEQ ID NO: 130; or the amino acid sequence of SEQ ID NO: 121, with an unnatural amino acid (UAA) at a position corresponding to T167 of SEQ ID NO: 121;
(xii) the amino acid sequence of SEQ ID NO: 137, with an unnatural amino acid (UAA) at a position corresponding to T6 of SEQ ID NO: 137; the amino acid sequence of SEQ ID NO: 129, with an unnatural amino acid (UAA) at a position corresponding to T78 of SEQ ID NO: 129; the amino acid sequence of SEQ ID NO: 130, with an unnatural amino acid (UAA) at a position corresponding to T78 of SEQ ID NO: 130; or the amino acid sequence of SEQ ID NO: 121, with an unnatural amino acid (UAA) at a position corresponding to T198 of SEQ ID NO: 121;
(xiii) the amino acid sequence of SEQ ID NO: 138, with an unnatural amino acid (UAA) at a position corresponding to N6 of SEQ ID NO: 138; the amino acid sequence of SEQ ID NO: 129, with an unnatural amino acid (UAA) at a position corresponding to N84 of SEQ ID NO: 129; the amino acid sequence of SEQ ID NO: 130, with an unnatural amino acid (UAA) at a position corresponding to N84 of SEQ ID NO: 130; or the amino acid sequence of SEQ ID NO: 121, with an unnatural amino acid (UAA) at a position corresponding to N204 of SEQ ID NO: 121;
(xiv) the amino acid sequence of SEQ ID NO: 139, with an unnatural amino acid (UAA) at a position corresponding to V6 of SEQ ID NO: 139; the amino acid sequence of SEQ ID NO: 129, with an unnatural amino acid (UAA) at a position corresponding to V85 of SEQ ID NO: 129; the amino acid sequence of SEQ ID NO: 130, with an unnatural amino acid (UAA) at a position corresponding to V85 of SEQ ID NO: 130; or the amino acid sequence of SEQ ID NO: 121, with an unnatural amino acid (UAA) at a position corresponding to V205 of SEQ ID NO: 121;
(xv) the amino acid sequence of SEQ ID NO: 140, with an unnatural amino acid (UAA) at a position corresponding to N6 of SEQ ID NO: 140; the amino acid sequence of SEQ ID NO: 129, with an unnatural amino acid (UAA) at a position corresponding to N86 of SEQ ID NO: 129; the amino acid sequence of SEQ ID NO: 130, with an unnatural amino acid (UAA) at a position corresponding to N86 of SEQ ID NO: 130; or the amino acid sequence of SEQ ID NO: 121, with an unnatural amino acid (UAA) at a position corresponding to N206 of SEQ ID NO: 121;
(xvi) the amino acid sequence of SEQ ID NO: 141, with an unnatural amino acid (UAA) at a position corresponding to K6 of SEQ ID NO: 141; the amino acid sequence of SEQ ID NO: 129, with an unnatural amino acid (UAA) at a position corresponding to K93 of SEQ ID NO: 129; the amino acid sequence of SEQ ID NO: 130, with an unnatural amino acid (UAA) at a position corresponding to K93 of SEQ ID NO: 130; or the amino acid sequence of SEQ ID NO: 121, with an unnatural amino acid (UAA) at a position corresponding to K213 of SEQ ID NO: 121;
(xvii) the amino acid sequence of SEQ ID NO: 142, with an unnatural amino acid (UAA) at a position corresponding to D6 of SEQ ID NO: 142; the amino acid sequence of SEQ ID NO: 129, with an unnatural amino acid (UAA) at a position corresponding to D95 of SEQ ID NO: 129; the amino acid sequence of SEQ ID NO: 130, with an unnatural amino acid (UAA) at a position corresponding to D95 of SEQ ID NO: 130; or the amino acid sequence of SEQ ID NO: 121, with an unnatural amino acid (UAA) at a position corresponding to D215 of SEQ ID NO: 121;
(xviii) the amino acid sequence of SEQ ID NO: 143, with an unnatural amino acid (UAA) at a position corresponding to 16 of SEQ ID NO: 143; the amino acid sequence of SEQ ID NO: 144, with an unnatural amino acid (UAA) at a position corresponding to 123 of SEQ ID NO: 144; the amino acid sequence of SEQ ID NO: 130, with an unnatural amino acid (UAA) at a position corresponding to 1136 of SEQ ID NO: 130; or the amino acid sequence of SEQ ID NO: 121, with an unnatural amino acid (UAA) at a position corresponding to 1256 of SEQ ID NO: 121;
(xix) the amino acid sequence of SEQ ID NO: 145, with an unnatural amino acid (UAA) at a position corresponding to K6 of SEQ ID NO: 145; the amino acid sequence of SEQ ID NO: 144, with an unnatural amino acid (UAA) at a position corresponding to K44 of SEQ ID NO: 144; the amino acid sequence of SEQ ID NO: 130, with an unnatural amino acid (UAA) at a position corresponding to K157 of SEQ ID NO: 130; or the amino acid sequence of SEQ ID NO: 121, with an unnatural amino acid (UAA) at a position corresponding to K277 of SEQ ID NO: 121;
(xx) the amino acid sequence of SEQ ID NO: 146, with an unnatural amino acid (UAA) at a position corresponding to Y6 of SEQ ID NO: 146; the amino acid sequence of SEQ ID NO: 144, with an unnatural amino acid (UAA) at a position corresponding to Y48 of SEQ ID NO: 144; the amino acid sequence of SEQ ID NO: 130, with an unnatural amino acid (UAA) at a position corresponding to Y161 of SEQ ID NO: 130; or the amino acid sequence of SEQ ID NO: 121, with an unnatural amino acid (UAA) at a position corresponding to Y281 of SEQ ID NO: 121;
(xxi) the amino acid sequence of SEQ ID NO: 147, with an unnatural amino acid (UAA) at a position corresponding to K6 of SEQ ID NO: 147; the amino acid sequence of SEQ ID NO: 144, with an unnatural amino acid (UAA) at a position corresponding to K58 of SEQ ID NO: 144; the amino acid sequence of SEQ ID NO: 130, with an unnatural amino acid (UAA) at a position corresponding to K171 of SEQ ID NO: 130; or the amino acid sequence of SEQ ID NO: 121, with an unnatural amino acid (UAA) at a position corresponding to K291 of SEQ ID NO: 121;
(xxii) the amino acid sequence of SEQ ID NO: 148, with an unnatural amino acid (UAA) at a position corresponding to K6 of SEQ ID NO: 148; the amino acid sequence of SEQ ID NO: 144, with an unnatural amino acid (UAA) at a position corresponding to K60 of SEQ ID NO: 144; the amino acid sequence of SEQ ID NO: 130, with an unnatural amino acid (UAA) at a position corresponding to K173 of SEQ ID NO: 130; or the amino acid sequence of SEQ ID NO: 121, with an unnatural amino acid (UAA) at a position corresponding to K293 of SEQ ID NO: 121;
(xxiii) the amino acid sequence of SEQ ID NO: 149, with an unnatural amino acid (UAA) at a position corresponding to N6 of SEQ ID NO: 149; the amino acid sequence of SEQ ID NO: 144, with an unnatural amino acid (UAA) at a position corresponding to N67 of SEQ ID NO: 144; the amino acid sequence of SEQ ID NO: 130, with an unnatural amino acid (UAA) at a position corresponding to N180 of SEQ ID NO: 130; or the amino acid sequence of SEQ ID NO: 121, with an unnatural amino acid (UAA) at a position corresponding to N300 of SEQ ID NO: 121;
(xxiv) the amino acid sequence of SEQ ID NO: 150, with an unnatural amino acid (UAA) at a position corresponding to F6 of SEQ ID NO: 150; the amino acid sequence of SEQ ID NO: 151, with an unnatural amino acid (UAA) at a position corresponding to F64 of SEQ ID NO: 151; the amino acid sequence of SEQ ID NO: 130, with an unnatural amino acid (UAA) at a position corresponding to F287 of SEQ ID NO: 130; or the amino acid sequence of SEQ ID NO: 121, with an unnatural amino acid (UAA) at a position corresponding to F407 of SEQ ID NO: 121; and/or
(xxv) the amino acid sequence of SEQ ID NO: 191, with an unnatural amino acid (UAA) at a position corresponding to K6 of SEQ ID NO: 191; the amino acid sequence of SEQ ID NO: 129, with an unnatural amino acid (UAA) at a position corresponding to K16 of SEQ ID NO: 129; the amino acid sequence of SEQ ID NO: 130, with an unnatural amino acid (UAA) at a position corresponding to K16 of SEQ ID NO: 130; or the amino acid sequence of SEQ ID NO: 121, with an unnatural amino acid (UAA) at a position corresponding to K136 of SEQ ID NO: 121; and/or
wherein the antibody comprises a light chain, or a fragment thereof, comprising:
(i) the amino acid sequence of SEQ ID NO: 152, with an unnatural amino acid (UAA) at a position corresponding to V6 of SEQ ID NO: 152; the amino acid sequence of SEQ ID NO: 153, with an unnatural amino acid (UAA) at a position corresponding to V15 of SEQ ID NO: 153; the amino acid sequence of SEQ ID NO: 154, with an unnatural amino acid (UAA) at a position corresponding to V15 of SEQ ID NO: 154; or the amino acid sequence of SEQ ID NO: 155, with an unnatural amino acid (UAA) at a position corresponding to V15 of SEQ ID NO: 155;
(ii) the amino acid sequence of SEQ ID NO: 156, with an unnatural amino acid (UAA) at a position corresponding to T6 of SEQ ID NO: 156; the amino acid sequence of SEQ ID NO: 153, with an unnatural amino acid (UAA) at a position corresponding to T20 of SEQ ID NO: 153; the amino acid sequence of SEQ ID NO: 154, with an unnatural amino acid (UAA) at a position corresponding to T20 of SEQ ID NO: 154; or the amino acid sequence of SEQ ID NO: 155, with an unnatural amino acid (UAA) at a position corresponding to T20 of SEQ ID NO: 155;
(iii) the amino acid sequence of SEQ ID NO: 157, with an unnatural amino acid (UAA) at a position corresponding to R6 of SEQ ID NO: 157; the amino acid sequence of SEQ ID NO: 158, with an unnatural amino acid (UAA) at a position corresponding to R1 of SEQ ID NO: 158; the amino acid sequence of SEQ ID NO: 154, with an unnatural amino acid (UAA) at a position corresponding to R24 of SEQ ID NO: 154; or the amino acid sequence of SEQ ID NO: 155, with an unnatural amino acid (UAA) at a position corresponding to R24 of SEQ ID NO: 155;
(iv) the amino acid sequence of SEQ ID NO: 159, with an unnatural amino acid (UAA) at a position corresponding to S6 of SEQ ID NO: 159; the amino acid sequence of SEQ ID NO: 160, with an unnatural amino acid (UAA) at a position corresponding to S4 of SEQ ID NO: 160; the amino acid sequence of SEQ ID NO: 154, with an unnatural amino acid (UAA) at a position corresponding to S60 of SEQ ID NO: 154; or the amino acid sequence of SEQ ID NO: 155, with an unnatural amino acid (UAA) at a position corresponding to S60 of SEQ ID NO: 155;
(v) the amino acid sequence of SEQ ID NO: 161, with an unnatural amino acid (UAA) at a position corresponding to K6 of SEQ ID NO: 161; the amino acid sequence of SEQ ID NO: 162, with an unnatural amino acid (UAA) at a position corresponding to K10 of SEQ ID NO: 162; the amino acid sequence of SEQ ID NO: 154, with an unnatural amino acid (UAA) at a position corresponding to K107 of SEQ ID NO: 154; or the amino acid sequence of SEQ ID NO: 155, with an unnatural amino acid (UAA) at a position corresponding to K107 of SEQ ID NO: 155;
(vi) the amino acid sequence of SEQ ID NO: 163, with an unnatural amino acid (UAA) at a position corresponding to T6 of SEQ ID NO: 163; the amino acid sequence of SEQ ID NO: 162, with an unnatural amino acid (UAA) at a position corresponding to T12 of SEQ ID NO: 162; the amino acid sequence of SEQ ID NO: 154, with an unnatural amino acid (UAA) at a position corresponding to T109 of SEQ ID NO: 154; or the amino acid sequence of SEQ ID NO: 155, with an unnatural amino acid (UAA) at a position corresponding to T109 of SEQ ID NO: 155;
(vii) the amino acid sequence of SEQ ID NO: 164, with an unnatural amino acid (UAA) at a position corresponding to V6 of SEQ ID NO: 164; the amino acid sequence of SEQ ID NO: 162, with an unnatural amino acid (UAA) at a position corresponding to V13 of SEQ ID NO: 162; the amino acid sequence of SEQ ID NO: 154, with an unnatural amino acid (UAA) at a position corresponding to V110 of SEQ ID NO: 154; or the amino acid sequence of SEQ ID NO: 155, with an unnatural amino acid (UAA) at a position corresponding to V110 of SEQ ID NO: 155;
(viii) the amino acid sequence of SEQ ID NO: 165, with an unnatural amino acid (UAA) at a position corresponding to A6 of SEQ ID NO: 165; the amino acid sequence of SEQ ID NO: 166, with an unnatural amino acid (UAA) at a position corresponding to A1 of SEQ ID NO: 166; or the amino acid sequence of SEQ ID NO: 155, with an unnatural amino acid (UAA) at a position corresponding to A111 of SEQ ID NO: 155;
(ix) the amino acid sequence of SEQ ID NO: 167, with an unnatural amino acid (UAA) at a position corresponding to Q6 of SEQ ID NO: 167; the amino acid sequence of SEQ ID NO: 166, with an unnatural amino acid (UAA) at a position corresponding to Q37 of SEQ ID NO: 166; or the amino acid sequence of SEQ ID NO: 155, with an unnatural amino acid (UAA) at a position corresponding to Q147 of SEQ ID NO: 155;
(x) the amino acid sequence of SEQ ID NO: 168, with an unnatural amino acid (UAA) at a position corresponding to L6 of SEQ ID NO: 168; the amino acid sequence of SEQ ID NO: 166, with an unnatural amino acid (UAA) at a position corresponding to L44 of SEQ ID NO: 166; or the amino acid sequence of SEQ ID NO: 155, with an unnatural amino acid (UAA) at a position corresponding to L154 of SEQ ID NO: 155;
(xi) the amino acid sequence of SEQ ID NO: 169, with an unnatural amino acid (UAA) at a position corresponding to G6 of SEQ ID NO: 169; the amino acid sequence of SEQ ID NO: 166, with an unnatural amino acid (UAA) at a position corresponding to G47 of SEQ ID NO: 166; or the amino acid sequence of SEQ ID NO: 155, with an unnatural amino acid (UAA) at a position corresponding to G157 of SEQ ID NO: 155;
(xii) the amino acid sequence of SEQ ID NO: 175, with an unnatural amino acid (UAA) at a position corresponding to K6 of SEQ ID NO: 175; the amino acid sequence of SEQ ID NO: 166, with an unnatural amino acid (UAA) at a position corresponding to K59 of SEQ ID NO: 166; or the amino acid sequence of SEQ ID NO: 155, with an unnatural amino acid (UAA) at a position corresponding to K169 of SEQ ID NO: 155;
(xiii) the amino acid sequence of SEQ ID NO: 170, with an unnatural amino acid (UAA) at a position corresponding to A6 of SEQ ID NO: 170; the amino acid sequence of SEQ ID NO: 166, with an unnatural amino acid (UAA) at a position corresponding to A83 of SEQ ID NO: 166; or the amino acid sequence of SEQ ID NO: 155, with an unnatural amino acid (UAA) at a position corresponding to A193 of SEQ ID NO: 155;
(xiv) the amino acid sequence of SEQ ID NO: 171, with an unnatural amino acid (UAA) at a position corresponding to V6 of SEQ ID NO: 171; the amino acid sequence of SEQ ID NO: 166, with an unnatural amino acid (UAA) at a position corresponding to V95 of SEQ ID NO: 166; or the amino acid sequence of SEQ ID NO: 155, with an unnatural amino acid (UAA) at a position corresponding to V205 of SEQ ID NO: 155;
(xv) the amino acid sequence of SEQ ID NO: 172, with an unnatural amino acid (UAA) at a position corresponding to T6 of SEQ ID NO: 172; the amino acid sequence of SEQ ID NO: 166, with an unnatural amino acid (UAA) at a position corresponding to T96 of SEQ ID NO: 166; or the amino acid sequence of SEQ ID NO: 155, with an unnatural amino acid (UAA) at a position corresponding to T206 of SEQ ID NO: 155; and/or
(xvi) the amino acid sequence of SEQ ID NO: 173, with an unnatural amino acid (UAA) at a position corresponding to S6 of SEQ ID NO: 173; the amino acid sequence of SEQ ID NO: 166, with an unnatural amino acid (UAA) at a position corresponding to $98 of SEQ ID NO: 166; or the amino acid sequence of SEQ ID NO: 155, with an unnatural amino acid (UAA) at a position corresponding to S208 of SEQ ID NO: 155.

2. The antibody of claim 1, wherein the antibody comprises one, two, three, four, or more than four unnatural amino acids (UAAs).

3. The antibody of claim 1 or 2, wherein the antibody heavy chain further comprises: the amino acid sequence of SEQ ID NO: 128, with an unnatural amino acid (UAA) at a position corresponding to A6 of SEQ ID NO: 128; the amino acid sequence of SEQ ID NO: 129, with an unnatural amino acid (UAA) at a position corresponding to A1 of SEQ ID NO: 129; the amino acid sequence of SEQ ID NO: 130, with an unnatural amino acid (UAA) at a position corresponding to A1 of SEQ ID NO: 130; or the amino acid sequence of SEQ ID NO: 121, with an unnatural amino acid (UAA) at a position corresponding to A121 of SEQ ID NO: 121.

4. The antibody of any one of claims 1-3, wherein the unnatural amino acid (UAA) is a tryptophan analog.

5. The antibody of claim 4, wherein the tryptophan analog is selected from 5-HTP and 5-AzW.

6. The antibody of any one of claims 1-3, wherein the unnatural amino acid (UAA) is a leucine analog.

7. The method of claim 6, wherein the leucine analog is selected from LCA and Cys-5-N3.

8. The antibody of any one of claims 1-3, wherein the unnatural amino acid (UAA) is a tyrosine analog.

9. The antibody of claim 8, wherein the tyrosine analog is selected from OmeY, AzF, and OpropY.

10. The antibody of any one of claims 1-3, wherein the unnatural amino acid (UAA) is a pyrrolysine analog.

11. The antibody of claim 10, wherein the pyrrolysine analog is selected from Bock, CpK, and AzK.

12. An antibody,
wherein the antibody heavy chain comprises: the amino acid sequence of SEQ ID NO: 128, with an unnatural amino acid (UAA) at a position corresponding to A6 of SEQ ID NO: 128; the amino acid sequence of SEQ ID NO: 129, with an unnatural amino acid (UAA) at a position corresponding to A1 of SEQ ID NO: 129; the amino acid sequence of SEQ ID NO: 130, with an unnatural amino acid (UAA) at a position corresponding to A1 of SEQ ID NO: 130; or the amino acid sequence of SEQ ID NO: 121, with an unnatural amino acid (UAA) at a position corresponding to A121 of SEQ ID NO: 121;
and wherein the UAA is: (i) a tryptophan analog (e.g., 5-HTP and 5-AzW); (ii) a leucine analog (e.g., LCA and Cys-5-N3); (iii) a tyrosine analog (e.g., OmeY, AzF, and OpropY); or (iv) a pyrrolysine analog (e.g., Bock, CpK, and AzK).

13. The antibody of any one of claims 1-12, further comprising a chemical modification of the unnatural amino acid (UAA).

14. The antibody of claim 13, wherein the chemical modification comprises conjugation to a molecule.

15. The antibody of claim 14, wherein the molecule is a detectable label.

16. The antibody of claim 14, wherein the molecule is a drug (e.g., a small molecule drug).

17. The antibody of any one of claims 14-16, wherein the molecule is: AEB, AEVB, AFP, an amatoxin, an auristatin (e.g., auristatin E), a calicheamicin, CC-1065 or a CC-1065 analog, chalicheamicin, combretastatin, DM1, DM4, docetaxel, dolastatin-10, DUBA, a duocarmycin, echinomycin, FAM, maytansine, a maytansinoid, MMAD, MMAE, MMAF, a morpholino-doxorubicin (e.g., cyanomorpholino-doxorubicin), netropsin, an oligonucleotide (e.g., a DNA, RNA, or LNA oligonucleotide), paclitaxel, PBD, a peptide (e.g., a therapeutic peptide), rhizoxin, a small molecule (e.g., a therapeutic small molecule) SN-38, topotecan, a topoisomerase inhibitor, or a toxoid.

18. The antibody of any one of claims 14-17, wherein the molecule is conjugated to the antibody by a linker.

19. The antibody of claim 18, wherein the linker is a cleavable linker, a non-cleavable linker, a peptide-based linker, or a PEG-based linker.

20. The antibody of any one of claims 14-19, wherein
(a) the antibody has an average drug antibody ratio (DAR) of at least 0.5, 0.6, 0.7, 0.8, 0.9, 1.0, 1.5, 1.6, 1.7, 1.8, 1.9, 2.0, 3.5, 3.6, 3.7, 3.8, 3.9, or 4.0, as measured by hydrophobic interaction chromatography (HIC);
(b) the antibody has an average drug antibody ratio (DAR) that is within 5%, 10%, 15%, 20%, 25%, 30%, 35%, or 40% of the number of UAAs in the antibody;
(c) at least 40%, 50%, 60%, 70%, 80%, or 90% of the antibody remains following incubation in human plasma for 72 hours at 37° C.;
(d) at least 40%, 50%, 60%, 70%, 80%, or 90% of the antibody remains following incubation with Cathepsin B for 240 minutes at 37° C.;
(e) the antibody has a binding affinity for a target antigen of 20 nM, 15 nM, 10 nM, 9 nM, 8 nM, 7 nM, 6 nM, 5 nM, 4 nM, 3 nM, 2 nM, 1 nM, 0.75 nM, 0.5 nM, 0.1 nM, 0.075 nM, or 0.05 nM or lower, as measured by enzyme-linked immunosorbent assay (ELISA); and/or
(f) the antibody has a binding affinity for a target antigen that is within 0.1 fold, 0.2 fold, 0.3 fold, 0.4 fold, 0.5 fold, 1.0 fold, 1.5 fold, 2.0 fold, 3.0 fold, 4.0 fold, 5.0 fold, 6.0 fold, 8.0 fold, or 10.0 fold of the binding affinity for the target antigen of a reference antibody, wherein the reference antibody is an otherwise identical antibody that does not comprise the UAA, as measured by ELISA.

21. The antibody of any one of claims 1-20, wherein the antibody comprises an antigen-binding fragment.

22. The antibody of any one of claims 1-21, wherein the antibody is an IgG1, IgG2, IgG3, or IgG4 antibody.

23. The antibody of claim 22, wherein the antibody is an IgG1 antibody.

24. A method of producing the antibody of any one of claims 1-23, the method comprising culturing a cell with:
(i) a nucleic acid comprising a nucleotide sequence encoding a tRNA comprising an anticodon that hybridizes to a codon selected from UAG, UGA, and UAA, and is capable of being charged with the unnatural amino acid (UAA);
(ii) a nucleic acid comprising a nucleotide sequence encoding an aminoacyl-tRNA synthetase capable of charging the tRNA with the unnatural amino acid (UAA); and
(iii) a nucleic acid comprising a nucleotide sequence encoding a heavy chain, a light chain, or a combination of a heavy chain and light chain of the antibody and comprising the codon selected from UAG, UGA, and UAA;
under conditions that permit the tRNA, when expressed in the cell and charged with the unnatural amino acid (UAA), to hybridize to the codon and direct the incorporation of the unnatural amino acid (UAA) into the antibody.

25. The method of claim 24, wherein
(a) the amount of the antibody comprising the unnatural amino acid (UAA) expressed by the cell is at least 10%, 20%, 30%, 40%, 50%, 60%, 70%, 80%, or 90% of the amount of a reference antibody expressed by the same cell or a similar cell, wherein the reference antibody is an otherwise identical antibody that does not comprise the UAA; and/or
(b) following protein G purification, less than 10%, 9%, 8%, 7%, 6%, 5%, 4%, 3%, 2%, or 1% of the antibody is aggregated, as measured by size exclusion chromatography (SEC).

26. A method of producing an antibody comprising an unnatural amino acid (UAA), the method comprising culturing a cell with:
(i) a nucleic acid comprising a nucleotide sequence encoding a tRNA comprising an anticodon that hybridizes to a codon selected from UAG, UGA, and UAA, and is capable of being charged with the unnatural amino acid (UAA);
(ii) a nucleic acid comprising a nucleotide sequence encoding an aminoacyl-tRNA synthetase capable of charging the tRNA with the unnatural amino acid (UAA); and
(iii) a nucleic acid comprising a nucleotide sequence encoding a heavy chain, light chain or a combination of a heavy chain and light chain of the antibody and comprising the codon selected from UAG, UGA, and UAA;
under conditions that permit the tRNA, when expressed in the cell and charged with the unnatural amino acid (UAA), to hybridize to the codon and direct the incorporation of the unnatural amino acid (UAA) into the antibody;
wherein the antibody heavy chain, or a fragment thereof, comprises the amino acid sequence of SEQ ID NO: 128, with an unnatural amino acid (UAA) at a position corresponding to A6 of SEQ ID NO: 128; the amino acid sequence of SEQ ID NO: 129, with an unnatural amino acid (UAA) at a position corresponding to A1 of SEQ ID NO: 129; the amino acid sequence of SEQ ID NO: 130, with an unnatural amino acid (UAA) at a position corresponding to A1 of SEQ ID NO: 130; or the amino acid sequence of SEQ ID NO: 121, with an unnatural amino acid (UAA) at a position corresponding to A121 of SEQ ID NO: 121;
and wherein
(a) the amount of the antibody comprising the unnatural amino acid (UAA) expressed by the cell is at least 10%, 20%, 30%, 40%, 50%, 60%, 70%, 80%, or 90% of the amount of a reference antibody expressed by the same cell or a similar cell, wherein the reference antibody is an otherwise identical antibody that does not comprise the UAA; and/or
(b) following protein G purification, less than 10%, 9%, 8%, 7%, 6%, 5%, 4%, 3%, 2%, or 1% of the antibody is aggregated, as measured by size exclusion chromatography (SEC).

27. The method of any one of claims 24-26, wherein the tRNA is an analog or derivative of a prokaryotic tryptophanyl-tRNA.

28. The method of claim 27, wherein the prokaryotic tryptophanyl-tRNA is an E. coli tryptophanyl-tRNA.

29. The method of claim 28, wherein the tRNA comprises a nucleotide sequence selected from any one of SEQ ID NOs: 49-54 or 108-113.

30. The method of any one of claims 24-29, wherein the aminoacyl-tRNA synthetase is an analog or derivative of a prokaryotic tryptophanyl-tRNA synthetase.

31. The method of claim 30, wherein the prokaryotic tryptophanyl-tRNA synthetase is an E. coli tryptophanyl-tRNA synthetase.

32. The method of claim 31, wherein the aminoacyl-tRNA synthetase comprises an amino acid sequence selected from any one of SEQ ID NOs: 44-48.

33. The method of any one of claims 24-32, wherein the tRNA is an analog or derivative of a prokaryotic leucyl-tRNA.

34. The method of claim 33, wherein the prokaryotic leucyl-tRNA is an E. coli leucyl-tRNA.

35. The method of claim 34, wherein the tRNA comprises a nucleotide sequence selected from any one of SEQ ID NOs: 16-43.

36. The method of any one of claims 24-35, wherein the aminoacyl-tRNA synthetase is an analog or derivative of a prokaryotic leucyl-tRNA synthetase.

37. The method of claim 36, wherein the prokaryotic leucyl-tRNA synthetase is an E. coli leucyl-tRNA synthetase.

38. The method of claim 37, wherein the aminoacyl-tRNA synthetase comprises an amino acid sequence selected from any one of SEQ ID NOs: 1-15.

39. The method of any one of claims 24-38, wherein the tRNA is an analog or derivative of a prokaryotic tyrosyl-tRNA.

40. The method of claim 39, wherein the prokaryotic tyrosyl-tRNA is an E. coli tyrosyl-tRNA.

41. The method of claim 40, wherein the tRNA comprises a nucleotide sequence selected from any one of SEQ ID NOs: 68-69 or 104-105.

42. The method of any one of claims 24-41, wherein the aminoacyl-tRNA synthetase is an analog or derivative of a prokaryotic tyrosyl-tRNA synthetase.

43. The method of claim 42, wherein the prokaryotic tyrosyl-tRNA synthetase is an E. coli tyrosyl-tRNA synthetase.

44. The method of claim 43, wherein the aminoacyl-tRNA synthetase comprises the amino acid sequence of SEQ ID NO: 70.

45. The method of any one of claims 24-44, wherein the tRNA is an analog or derivative of an archael pyrrolysyl-tRNA.

46. The method of claim 45, wherein the archael pyrrolysyl-tRNA is an M. barkeri pyrrolysyl-tRNA.

47. The method of claim 46, wherein the tRNA comprises a nucleotide sequence selected from any one of SEQ ID NOs: 72-100 or 106-107.

48. The method of any one of claims 24-47, wherein the aminoacyl-tRNA synthetase is an analog or derivative of an archael pyrrolysyl-tRNA synthetase.

49. The method of claim 48, wherein the archael pyrrolysyl-tRNA synthetase is an M. barkeri pyrrolysyl-tRNA synthetase.

50. The method of claim 49, wherein the aminoacyl-tRNA synthetase comprises the amino acid sequence of SEQ ID NO: 101.

51. The method of any one of claims 24-50, wherein the reference antibody comprises a wild-type amino acid residue at the position corresponding to the unnatural amino acid (UAA).

52. The method of any one of claims 24-51, wherein the cell is a mammalian cell.

53. The method of claim 52, wherein the cell is a human cell.

54. The method of claim 53, wherein the cell is a human embryonic kidney (HEK) or a Chinese hamster ovary (CHO) cell.

55. The method of any one of claims 24-54, further comprising purifying the antibody.

56. The method of any one of claims 24-55, further comprising chemically modifying the unnatural amino acid (UAA).

57. The method of claim 56, wherein the chemical modification comprises conjugation to a molecule.

58. The method of claim 57, wherein molecule is a detectable label.

59. The method of claim 57, wherein the molecule is a drug (e.g., a small molecule drug).

60. The method of any one of claims 57-59, wherein the molecule is: AEB, AEVB, AFP, an amatoxin, an auristatin (e.g., auristatin E), a calicheamicin, CC-1065 or a CC-1065 analog, chalicheamicin, combretastatin, DM1, DM4, docetaxel, dolastatin-10, DUBA, a duocarmycin, echinomycin, FAM, maytansine, a maytansinoid, MMAD, MMAE, MMAF, a morpholino-doxorubicin (e.g., cyanomorpholino-doxorubicin), netropsin, an oligonucleotide (e.g., a DNA, RNA, or LNA oligonucleotide), paclitaxel, PBD, a peptide (e.g., a therapeutic peptide), rhizoxin, a small molecule (e.g., a therapeutic small molecule) SN-38, topotecan, a topoisomerase inhibitor, or a toxoid.

61. The method of any one of claims 57-60, wherein the molecule is conjugated to the antibody by a linker.

62. The method of claim 61, wherein the linker is a cleavable linker, a non-cleavable linker, a peptide-based linker, or a PEG-based linker.


SEQUENCE LISTING (196 sequences)

SEQ ID NO: 1 - protein, 863 aa, Escherichia coli
MQEQYRPEEIESKVQLHWDEKRTFEVTEDESKEKYYCLSMLPYPSGRLHMGHVRNYTIGDVIARYQRMLGKNVLQPIGWDAFGLPAEGAAVKNNTAPAPWTYDNIAYMKNQLKMLGFGYDWSRELATCTPEYYRWEQKFFTELYKKGLVYKKTSAVNWCPNDQTVLANEQVIDGCCWRCDTKVERKEIPQWFIKITAYADELLNDLDKLDHWPDTVKTMQRNWIGRSEGVEITFNVNDYDNTLTVYTTRPDTFMGCTYLAVAAGHPLAQKAAENNPELAAFIDECRNTKVAEAEMATMEKKGVDTGFKAVHPLTGEEIPVWAANFVLMEYGTGAVMAVPGHDQRDYEFASKYGLNIKPVILAADGSEPDLSQQALTEKGVLFNSGEFNGLDHEAAFNAIADKLTAMGVGERKVNYRLRDWGVSRQRYWGAPIPMVTLEDGTVMPTPDDQLPVILPEDVVMDGITSPIKADPEWAKTTVNGMPALRETDTFDTFMESSWYYARYTCPQYKEGMLDSEAANYWLPVDIYIGGIEHAIMHLLYFRFFHKLMRDAGMVNSDEPAKQLLCQGMVLADAFYYVGENGERNWVSPVDAIVERDEKGRIVKAKDAAGHELVYTGMSKMSKSKNNGIDPQVMVERYGADTVRLFMMFASPADMTLEWQESGVEGANRFLKRVWKLVYEHTAKGDVAALNVDALTENQKALRRDVHKTIAKVTDDIGRRQTFNTAIAAIMELMNKLAKAPTDGEQDRALMQEALLAVVRMLNPFTPHICFTLWQELKGEGDIDNAPWPVADEKAMVEDSTLVVVQVNGKVRAKITVPVDATEEQVRERAGQEHLVAKYLDGVTVRKVIYVPGKLLNLVVGGPV

SEQ ID NO: 2 - protein, 863 aa, Artificial Sequence
  Description of Artificial Sequence Synthetic polypeptide
MEEQYRPEEIESKVQLHWDKKRTFEVTEDESKEKYYCLSISPYPSGRLHMGHVRNYTIGDVIARYQRMLGKNVLQPIGWDAFGLPAEGAAVKNNTAPAPWTYDNIAYMKNQLKMLGFGYDWSRELATCTPEYYRWEQKFFTELYKKGLVYKKTSAVNWCPNDQTVLANEQVIDGCCWRCDTKVERKEIPQWFIKITAYADELLNDLDKLDHWPDTVKTMQRNWIGRSEGVEITFNVNDYDNTLTVYTTRPDAFMGCTYLAVAAGHPLAQKAAENNPELAAFIDECRNTKVAEAEMATMEKKGVDTGFKAVHPLTGEEIPVWAANFVLMEYGTGAVMAVPGHDQRDYEFASKYGLNIKPVILAADGSEPDLSQQALTEKGVLFNSGEFNGLDHEAAFNAIADKLTAMGVGERKVNYRLRDWGVSRQRYWGAPIPMVTLEDGTVMPTPDDQLPVILPEDVVMDGITSPIKADPEWAKTTVNGMPALRETDTFDTFMESSWIYARYTCPQYKEGMLDSEAANYWLPVDIAIGGIEHAIMGLLYFRFFHKLMRDAGMVNSDEPAKQLLCQGMVLADAFYYVGENGERNWVSPVDAIVERDEKGRIVKAKDAAGHELVYTGMSKMSKSKNNGIDPQVMVERYGADTVRLFMMFASPADMTLEWQESGVEGANRFLKRVWKLVYEHTAKGDVAALNVDALTENQKALRRDVHKTIAKVTDDIGRRQTFNTAIAAIMELMNKLAKAPTDGEQDRALMQEALLAVVRMLNPFTPHICFTLWQELKGEGDIDNAPWPVADEKAMVEDSTLVVVQVNGKVRAKITVPVDATEEQVRERAGQEHLVAKYLDGVTVRKVIYVPGKLLNLVVGGPV

SEQ ID NO: 3 - protein, 863 aa, Artificial Sequence
  Description of Artificial Sequence Synthetic polypeptide
MEEQYRPEEIESKVQLHWDKKRTFEVTEDESKEKYYCLSVSPYPSGRLHMGHVRNYTIGDVIARYQRMLGKNVLQPIGWDAFGLPAEGAAVKNNTAPAPWTYDNIAYMKNQLKMLGFGYDWSRELATCTPEYYRWEQKFFTELYKKGLVYKKTSAVNWCPNDQTVLANEQVIDGCCWRCDTKVERKEIPQWFIKITAYADELLNDLDKLDHWPDTVKTMQRNWIGRSEGVEITFNVNDYDNTLTVYTTRPDRFMGCTYLAVAAGHPLAQKAAENNPELAAFIDECRNTKVAEAEMATMEKKGVDTGFKAVHPLTGEEIPVWAANFVLMEYGTGAVMAVPGHDQRDYEFASKYGLNIKPVILAADGSEPDLSQQALTEKGVLFNSGEFNGLDHEAAFNAIADKLTAMGVGERKVNYRLRDWGVSRQRYWGAPIPMVTLEDGTVMPTPDDQLPVILPEDVVMDGITSPIKADPEWAKTTVNGMPALRETDTFDTFMESSWSYARYTCPQYKEGMLDSEAANYWLPVDILIGGIEHAIMGLLYFRFFHKLMRDAGMVNSDEPAKQLLCQGMVLADAFYYVGENGERNWVSPVDAIVERDEKGRIVKAKDAAGHELVYTGMSKMSKSKNNGIDPQVMVERYGADTVRLFMMFASPADMTLEWQESGVEGANRFLKRVWKLVYEHTAKGDVAALNVDALTENQKALRRDVHKTIAKVTDDIGRRQTFNTAIAAIMELMNKLAKAPTDGEQDRALMQEALLAVVRMLNPFTPHICFTLWQELKGEGDIDNAPWPVADEKAMVEDSTLVVVQVNGKVRAKITVPVDATEEQVRERAGQEHLVAKYLDGVTVRKVIYVPGKLLNLVVGGPV

SEQ ID NO: 4 - protein, 863 aa, Artificial Sequence
  Description of Artificial Sequence Synthetic polypeptide
MEEQYRPEEIESKVQLHWDEKRTFEVTEDESKEKYYCLSILPYPSGRLHMGHVRNYTIGDVIARYQRMLGKNVLQPIGWDAFGLPAEGAAVKNNTAPAPWTYDNIAYMKNQLKMLGFGYDWSRELATCTPEYYRWEQKFFTELYKKGLVYKKTSAVNWCPNDQTVLANEQVIDGCCWRCDTKVERKEIPQWFIKITAYADELLNDLDKLDHWPDTVKTMQRNWIGRSEGVEITFNVNDYDNTLTVYTTRPDAFMGCTYLAVAAGHPLAQKAAENNPELAAFIDECRNTKVAEAEMATMEKKGVDTGFKAVHPLTGEEIPVWAANFVLMEYGTGAVMAVPGHDQRDYEFASKYGLNIKPVILAADGSEPDLSQQALTEKGVLFNSGEFNGLDHEAAFNAIADKLTAMGVGERKVNYRLRDWGVSRQRYWGAPIPMVTLEDGTVMPTPDDQLPVILPEDVVMDGITSPIKADPEWAKTTVNGMPALRETDTFDTFMESSWIYARYTCPQYKEGMLDSEAANYWLPVDIAIGGIEHAIMGLLYFRFFHKLMRDAGMVNSDEPAKQLLCQGMVLADAFYYVGENGERNWVSPVDAIVERDEKGRIVKAKDAAGHELVYTGMSKMSKSKNNGIDPQVMVERYGADTVRLFMMFASPADMTLEWQESGVEGANRFLKRVWKLVYEHTAKGDVAALNVDALTENQKALRRDVHKTIAKVTDDIGRRQTFNTAIAAIMELMNKLAKAPTDGEQDRALMQEALLAVVRMLNPFTPHICFTLWQELKGEGDIDNAPWPVADEKAMVEDSTLVVVQVNGKVRAKITVPVDATEEQVRERAGQEHLVAKYLDGVTVRKVIYVPGKLLNLVVGGPV

SEQ ID NO: 5 - protein, 863 aa, Artificial Sequence
  Description of Artificial Sequence Synthetic polypeptide
MEEQYRPEEIESKVQLHWDMKRTFEVTEDESKEKYYCLSISPYPSGRLHMGHVRNYTIGDVIARYQRMLGKNVLQPIGWDAFGLPAEGAAVKNNTAPAPWTYDNIAYMKNQLKMLGFGYDWSRELATCTPEYYRWEQKFFTELYKKGLVYKKTSAVNWCPNDQTVLANEQVIDGCCWRCDTKVERKEIPQWFIKITAYADELLNDLDKLDHWPDTVKTMQRNWIGRSEGVEITFNVNDYDNTLTVYTTRPDAFMGCTYLAVAAGHPLAQKAAENNPELAAFIDECRNTKVAEAEMATMEKKGVDTGFKAVHPLTGEEIPVWAANFVLMEYGTGAVMAVPGHDQRDYEFASKYGLNIKPVILAADGSEPDLSQQALTEKGVLFNSGEFNGLDHEAAFNAIADKLTAMGVGERKVNYRLRDWGVSRQRYWGAPIPMVTLEDGTVMPTPDDQLPVILPEDVVMDGITSPIKADPEWAKTTVNGMPALRETDTFDTFMESSWIYARYTCPQYKEGMLDSEAANYWLPVDIAIGGIEHAIMGLLYFRFFHKLMRDAGMVNSDEPAKQLLCQGMVLADAFYYVGENGERNWVSPVDAIVERDEKGRIVKAKDAAGHELVYTGMSKMSKSKNNGIDPQVMVERYGADTVRLFMMFASPADMTLEWQESGVEGANRFLKRVWKLVYEHTAKGDVAALNVDALTENQKALRRDVHKTIAKVTDDIGRRQTFNTAIAAIMELMNKLAKAPTDGEQDRALMQEALLAVVRMLNPFTPHICFTLWQELKGEGDIDNAPWPVADEKAMVEDSTLVVVQVNGKVRAKITVPVDATEEQVRERAGQEHLVAKYLDGVTVRKVIYVPGKLLNLVVGGPV

SEQ ID NO: 6 - protein, 863 aa, Artificial Sequence
  Description of Artificial Sequence Synthetic polypeptide
MEEQYRPEEIESKVQLHWDVKRTFEVTEDESKEKYYCLSISPYPSGRLHMGHVRNYTIGDVIARYQRMLGKNVLQPIGWDAFGLPAEGAAVKNNTAPAPWTYDNIAYMKNQLKMLGFGYDWSRELATCTPEYYRWEQKFFTELYKKGLVYKKTSAVNWCPNDQTVLANEQVIDGCCWRCDTKVERKEIPQWFIKITAYADELLNDLDKLDHWPDTVKTMQRNWIGRSEGVEITFNVNDYDNTLTVYTTRPDAFMGCTYLAVAAGHPLAQKAAENNPELAAFIDECRNTKVAEAEMATMEKKGVDTGFKAVHPLTGEEIPVWAANFVLMEYGTGAVMAVPGHDQRDYEFASKYGLNIKPVILAADGSEPDLSQQALTEKGVLFNSGEFNGLDHEAAFNAIADKLTAMGVGERKVNYRLRDWGVSRQRYWGAPIPMVTLEDGTVMPTPDDQLPVILPEDVVMDGITSPIKADPEWAKTTVNGMPALRETDTFDTFMESSWIYARYTCPQYKEGMLDSEAANYWLPVDIAIGGIEHAIMGLLYFRFFHKLMRDAGMVNSDEPAKQLLCQGMVLADAFYYVGENGERNWVSPVDAIVERDEKGRIVKAKDAAGHELVYTGMSKMSKSKNNGIDPQVMVERYGADTVRLFMMFASPADMTLEWQESGVEGANRFLKRVWKLVYEHTAKGDVAALNVDALTENQKALRRDVHKTIAKVTDDIGRRQTFNTAIAAIMELMNKLAKAPTDGEQDRALMQEALLAVVRMLNPFTPHICFTLWQELKGEGDIDNAPWPVADEKAMVEDSTLVVVQVNGKVRAKITVPVDATEEQVRERAGQEHLVAKYLDGVTVRKVIYVPGKLLNLVVGGPV

SEQ ID NO: 7 - protein, 863 aa, Artificial Sequence
  Description of Artificial Sequence Synthetic polypeptide
MEEQYRPEEIESKVQLHWDKKRTFEVTEDESKEKYYCLSIVPYPSGRLHMGHVRNYTIGDVIARYQRMLGKNVLQPIGWDAFGLPAEGAAVKNNTAPAPWTYDNIAYMKNQLKMLGFGYDWSRELATCTPEYYRWEQKFFTELYKKGLVYKKTSAVNWCPNDQTVLANEQVIDGCCWRCDTKVERKEIPQWFIKITAYADELLNDLDKLDHWPDTVKTMQRNWIGRSEGVEITFNVNDYDNTLTVYTTRPDAFMGCTYLAVAAGHPLAQKAAENNPELAAFIDECRNTKVAEAEMATMEKKGVDTGFKAVHPLTGEEIPVWAANFVLMEYGTGAVMAVPGHDQRDYEFASKYGLNIKPVILAADGSEPDLSQQALTEKGVLFNSGEFNGLDHEAAFNAIADKLTAMGVGERKVNYRLRDWGVSRQRYWGAPIPMVTLEDGTVMPTPDDQLPVILPEDVVMDGITSPIKADPEWAKTTVNGMPALRETDTFDTFMESSWIYARYTCPQYKEGMLDSEAANYWLPVDIAIGGIEHAIMGLLYFRFFHKLMRDAGMVNSDEPAKQLLCQGMVLADAFYYVGENGERNWVSPVDAIVERDEKGRIVKAKDAAGHELVYTGMSKMSKSKNNGIDPQVMVERYGADTVRLFMMFASPADMTLEWQESGVEGANRFLKRVWKLVYEHTAKGDVAALNVDALTENQKALRRDVHKTIAKVTDDIGRRQTFNTAIAAIMELMNKLAKAPTDGEQDRALMQEALLAVVRMLNPFTPHICFTLWQELKGEGDIDNAPWPVADEKAMVEDSTLVVVQVNGKVRAKITVPVDATEEQVRERAGQEHLVAKYLDGVTVRKVIYVPGKLLNLVVGGPV

SEQ ID NO: 8 - protein, 863 aa, Artificial Sequence
  Description of Artificial Sequence Synthetic polypeptide
MEEQYRPEEIESKVQLHWDKKRTFEVTEDESKEKYYCLSIAPYPSGRLHMGHVRNYTIGDVIARYQRMLGKNVLQPIGWDAFGLPAEGAAVKNNTAPAPWTYDNIAYMKNQLKMLGFGYDWSRELATCTPEYYRWEQKFFTELYKKGLVYKKTSAVNWCPNDQTVLANEQVIDGCCWRCDTKVERKEIPQWFIKITAYADELLNDLDKLDHWPDTVKTMQRNWIGRSEGVEITFNVNDYDNTLTVYTTRPDAFMGCTYLAVAAGHPLAQKAAENNPELAAFIDECRNTKVAEAEMATMEKKGVDTGFKAVHPLTGEEIPVWAANFVLMEYGTGAVMAVPGHDQRDYEFASKYGLNIKPVILAADGSEPDLSQQALTEKGVLFNSGEFNGLDHEAAFNAIADKLTAMGVGERKVNYRLRDWGVSRQRYWGAPIPMVTLEDGTVMPTPDDQLPVILPEDVVMDGITSPIKADPEWAKTTVNGMPALRETDTFDTFMESSWIYARYTCPQYKEGMLDSEAANYWLPVDIAIGGIEHAIMGLLYFRFFHKLMRDAGMVNSDEPAKQLLCQGMVLADAFYYVGENGERNWVSPVDAIVERDEKGRIVKAKDAAGHELVYTGMSKMSKSKNNGIDPQVMVERYGADTVRLFMMFASPADMTLEWQESGVEGANRFLKRVWKLVYEHTAKGDVAALNVDALTENQKALRRDVHKTIAKVTDDIGRRQTFNTAIAAIMELMNKLAKAPTDGEQDRALMQEALLAVVRMLNPFTPHICFTLWQELKGEGDIDNAPWPVADEKAMVEDSTLVVVQVNGKVRAKITVPVDATEEQVRERAGQEHLVAKYLDGVTVRKVIYVPGKLLNLVVGGPV

SEQ ID NO: 9 - protein, 862 aa, Artificial Sequence
  Description of Artificial Sequence Synthetic polypeptide
MEEQYRPEEIESKVQLHWDKKRTFEVTEDESKEKYYCLSISPYPSGRLHMGHVRNYTIGDVIARYQRMLGKNVLQPIGWDAFGLPAEGAAVKNNTAPAPWTYDNIAYMKNQLKMLGFGYDWSRELATCTPEYYRWEQKFFTELYKKGLVYKKTSAVNWCPNDQTVLANEQVIDGCCWRCDTKVERKEIPQWFIKITAYADELLNDLDKLDHWPDTVKTMQRNWIGRSEGVEITFNVNDYDNTLTVYTTRPDAFMGCTYLAVAAGHPLAQKAAENNPELAAFIDECRNTKVAEAEMATMEKKGVDTGFKAVHPLTGEEIPVWAANFVLMEYGTGAVMAVPGHDQRDYEFASKYGLNIKPVILAADGSEPDLSQQALTEKGVLFNSGEFNGLDHEAAFNAIADKLTAMGVGERKVNYRLRDWGVSRQRYWGAPIPMVTLEDGTVMPTPDDQLPVILPEDVVMDGITSPIKADPEWAKTTVNGMPALRETDTFDTFMESSWAYARYTCPQYKEGMLDSEAANYWLPVDIAIGGIEHAIMGLLYFRFFHKLMRDAGMVNSDEPAKQLLCQGMVLADAFYYVGENGERNWVSPVDAIVERDEKGRIVKAKDAAGHELVYTGMSKMSKSKNNGIDPQVMVERYGADTVRLFMMFASPADMTLEWQESGVEGANRFKRVWKLVYEHTAKGDVAALNVDALTENQKALRRDVHKTIAKVTDDIGRRQTFNTAIAAIMELMNKLAKAPTDGEQDRALMQEALLAVVRMLNPFTPHICFTLWQELKGEGDIDNAPWPVADEKAMVEDSTLVVVQVNGKVRAKITVPVDATEEQVRERAGQEHLVAKYLDGVTVRKVIYVPGKLLNLVVGGPV

SEQ ID NO: 10 - protein, 863 aa, Artificial Sequence
  Description of Artificial Sequence Synthetic polypeptide
MEEQYRPEEIESKVQLHWDKKRTFEVTEDESKEKYYCLSISPYPSGRLHMGHVRNYTIGDVIARYQRMLGKNVLQPIGWDAFGLPAEGAAVKNNTAPAPWTYDNIAYMKNQLKMLGFGYDWSRELATCTPEYYRWEQKFFTELYKKGLVYKKTSAVNWCPNDQTVLANEQVIDGCCWRCDTKVERKEIPQWFIKITAYADELLNDLDKLDHWPDTVKTMQRNWIGRSEGVEITFNVNDYDNTLTVYTTRPDAFMGCTYLAVAAGHPLAQKAAENNPELAAFIDECRNTKVAEAEMATMEKKGVDTGFKAVHPLTGEEIPVWAANFVLMEYGTGAVMAVPGHDQRDYEFASKYGLNIKPVILAADGSEPDLSQQALTEKGVLFNSGEFNGLDHEAAFNAIADKLTAMGVGERKVNYRLRDWGVSRQRYWGAPIPMVTLEDGTVMPTPDDQLPVILPEDVVMDGITSPIKADPEWAKTTVNGMPALRETDTFDTFMESSWHYARYTCPQYKEGMLDSEAANYWLPVDIAIGGIEHAIMGLLYFRFFHKLMRDAGMVNSDEPAKQLLCQGMVLADAFYYVGENGERNWVSPVDAIVERDEKGRIVKAKDAAGHELVYTGMSKMSKSKNNGIDPQVMVERYGADTVRLFMMFASPADMTLEWQESGVEGANRFLKRVWKLVYEHTAKGDVAALNVDALTENQKALRRDVHKTIAKVTDDIGRRQTFNTAIAAIMELMNKLAKAPTDGEQDRALMQEALLAVVRMLNPFTPHICFTLWQELKGEGDIDNAPWPVADEKAMVEDSTLVVVQVNGKVRAKITVPVDATEEQVRERAGQEHLVAKYLDGVTVRKVIYVPGKLLNLVVGGPV

SEQ ID NO: 11 - protein, 863 aa, Artificial Sequence
  Description of Artificial Sequence Synthetic polypeptide
MEEQYRPEEIESKVQLHWDKKRTFEVTEDESKEKYYCLSISPYPSGRLHMGHVRNYTIGDVIARYQRMLGKNVLQPIGWDAFGLPAEGAAVKNNTAPAPWTYDNIAYMKNQLKMLGFGYDWSRELATCTPEYYRWEQKFFTELYKKGLVYKKTSAVNWCPNDQTVLANEQVIDGCCWRCDTKVERKEIPQWFIKITAYADELLNDLDKLDHWPDTVKTMQRNWIGRSEGVEITFNVNDYDNTLTVYTTRPDAFMGCTYLAVAAGHPLAQKAAENNPELAAFIDECRNTKVAEAEMATMEKKGVDTGFKAVHPLTGEEIPVWAANFVLMEYGTGAVMAVPGHDQRDYEFASKYGLNIKPVILAADGSEPDLSQQALTEKGVLFNSGEFNGLDHEAAFNAIADKLTAMGVGERKVNYRLRDWGVSRQRYWGAPIPMVTLEDGTVMPTPDDQLPVILPEDVVMDGITSPIKADPEWAKTTVNGMPALRETDTFDTFMESSWIYARYTCPQYKEGMLDSEAANYWLPVDIIIGGIEHAIMGLLYFRFFHKLMRDAGMVNSDEPAKQLLCQGMVLADAFYYVGENGERNWVSPVDAIVERDEKGRIVKAKDAAGHELVYTGMSKMSKSKNNGIDPQVMVERYGADTVRLFMMFASPADMTLEWQESGVEGANRFLKRVWKLVYEHTAKGDVAALNVDALTENQKALRRDVHKTIAKVTDDIGRRQTFNTAIAAIMELMNKLAKAPTDGEQDRALMQEALLAVVRMLNPFTPHICFTLWQELKGEGDIDNAPWPVADEKAMVEDSTLVVVQVNGKVRAKITVPVDATEEQVRERAGQEHLVAKYLDGVTVRKVIYVPGKLLNLVVGGPV

SEQ ID NO: 12 - protein, 863 aa, Artificial Sequence
  Description of Artificial Sequence Synthetic polypeptide
MEEQYRPEEIESKVQLHWDKKRTFEVTEDESKEKYYCLSISPYPSGRLHMGHVRNYTIGDVIARYQRMLGKNVLQPIGWDAFGLPAEGAAVKNNTAPAPWTYDNIAYMKNQLKMLGFGYDWSRELATCTPEYYRWEQKFFTELYKKGLVYKKTSAVNWCPNDQTVLANEQVIDGCCWRCDTKVERKEIPQWFIKITAYADELLNDLDKLDHWPDTVKTMQRNWIGRSEGVEITFNVNDYDNTLTVYTTRPDAFMGCTYLAVAAGHPLAQKAAENNPELAAFIDECRNTKVAEAEMATMEKKGVDTGFKAVHPLTGEEIPVWAANFVLMEYGTGAVMAVPGHDQRDYEFASKYGLNIKPVILAADGSEPDLSQQALTEKGVLFNSGEFNGLDHEAAFNAIADKLTAMGVGERKVNYRLRDWGVSRQRYWGAPIPMVTLEDGTVMPTPDDQLPVILPEDVVMDGITSPIKADPEWAKTTVNGMPALRETDTFDTFMESSWIYARYTCPQYKEGMLDSEAANYWLPVDIVIGGIEHAIMGLLYFRFFHKLMRDAGMVNSDEPAKQLLCQGMVLADAFYYVGENGERNWVSPVDAIVERDEKGRIVKAKDAAGHELVYTGMSKMSKSKNNGIDPQVMVERYGADTVRLFMMFASPADMTLEWQESGVEGANRFLKRVWKLVYEHTAKGDVAALNVDALTENQKALRRDVHKTIAKVTDDIGRRQTFNTAIAAIMELMNKLAKAPTDGEQDRALMQEALLAVVRMLNPFTPHICFTLWQELKGEGDIDNAPWPVADEKAMVEDSTLVVVQVNGKVRAKITVPVDATEEQVRERAGQEHLVAKYLDGVTVRKVIYVPGKLLNLVVGGPV

SEQ ID NO: 13 - protein, 863 aa, Artificial Sequence
  Description of Artificial Sequence Synthetic polypeptide
MEEQYRPEEIESKVQLHWDKKRTFEVTEDESKEKYYCLSISPYPSGRLHMGHVRNYTIGDVIARYQRMLGKNVLQPIGWDAFGLPAEGAAVKNNTAPAPWTYDNIAYMKNQLKMLGFGYDWSRELATCTPEYYRWEQKFFTELYKKGLVYKKTSAVNWCPNDQTVLANEQVIDGCCWRCDTKVERKEIPQWFIKITAYADELLNDLDKLDHWPDTVKTMQRNWIGRSEGVEITFNVNDYDNTLTVYTTRPDAFMGCTYLAVAAGHPLAQKAAENNPELAAFIDECRNTKVAEAEMATMEKKGVDTGFKAVHPLTGEEIPVWAANFVLMEYGTGAVMAVPGHDQRDYEFASKYGLNIKPVILAADGSEPDLSQQALTEKGVLFNSGEFNGLDHEAAFNAIADKLTAMGVGERKVNYRLRDWGVSRQRYWGAPIPMVTLEDGTVMPTPDDQLPVILPEDVVMDGITSPIKADPEWAKTTVNGMPALRETDTFDTFMESSWIYARYTCPQYKEGMLDSEAANYWLPVDIGIGGIEHAIMGLLYFRFFHKLMRDAGMVNSDEPAKQLLCQGMVLADAFYYVGENGERNWVSPVDAIVERDEKGRIVKAKDAAGHELVYTGMSKMSKSKNNGIDPQVMVERYGADTVRLFMMFASPADMTLEWQESGVEGANRFLKRVWKLVYEHTAKGDVAALNVDALTENQKALRRDVHKTIAKVTDDIGRRQTFNTAIAAIMELMNKLAKAPTDGEQDRALMQEALLAVVRMLNPFTPHICFTLWQELKGEGDIDNAPWPVADEKAMVEDSTLVVVQVNGKVRAKITVPVDATEEQVRERAGQEHLVAKYLDGVTVRKVIYVPGKLLNLVVGGPV

SEQ ID NO: 14 - protein, 863 aa, Artificial Sequence
  Description of Artificial Sequence Synthetic polypeptide
  MOD_RES (2)..(2): Q or E
  MOD_RES (20)..(20): E, K, V, or M
  MOD_RES (40)..(40): M, I, or V
  MOD_RES (41)..(41): L, S, V, or A
  MOD_RES (252)..(252): T, A, or R
  MOD_RES (499)..(499): Y, A, I, H, or S
  MOD_RES (527)..(527): Y, A, I, L, or V
  MOD_RES (537)..(537): H or G
MXEQYRPEEIESKVQLHWDXKRTFEVTEDESKEKYYCLSXXPYPSGRLHMGHVRNYTIGDVIARYQRMLGKNVLQPIGWDAFGLPAEGAAVKNNTAPAPWTYDNIAYMKNQLKMLGFGYDWSRELATCTPEYYRWEQKFFTELYKKGLVYKKTSAVNWCPNDQTVLANEQVIDGCCWRCDTKVERKEIPQWFIKITAYADELLNDLDKLDHWPDTVKTMQRNWIGRSEGVEITFNVNDYDNTLTVYTTRPDXFMGCTYLAVAAGHPLAQKAAENNPELAAFIDECRNTKVAEAEMATMEKKGVDTGFKAVHPLTGEEIPVWAANFVLMEYGTGAVMAVPGHDQRDYEFASKYGLNIKPVILAADGSEPDLSQQALTEKGVLFNSGEFNGLDHEAAFNAIADKLTAMGVGERKVNYRLRDWGVSRQRYWGAPIPMVTLEDGTVMPTPDDQLPVILPEDVVMDGITSPIKADPEWAKTTVNGMPALRETDTFDTFMESSWXYARYTCPQYKEGMLDSEAANYWLPVDIXIGGIEHAIMXLLYFRFFHKLMRDAGMVNSDEPAKQLLCQGMVLADAFYYVGENGERNWVSPVDAIVERDEKGRIVKAKDAAGHELVYTGMSKMSKSKNNGIDPQVMVERYGADTVRLFMMFASPADMTLEWQESGVEGANRFLKRVWKLVYEHTAKGDVAALNVDALTENQKALRRDVHKTIAKVTDDIGRRQTFNTAIAAIMELMNKLAKAPTDGEQDRALMQEALLAVVRMLNPFTPHICFTLWQELKGEGDIDNAPWPVADEKAMVEDSTLVVVQVNGKVRAKITVPVDATEEQVRERAGQEHLVAKYLDGVTVRKVIYVPGKLLNLVVGGPV

SEQ ID NO: 15 - protein, 863 aa, Artificial Sequence
  Description of Artificial Sequence Synthetic polypeptide
  MOD_RES (20)..(20): K, V, or M
  MOD_RES (41)..(41): S, V, or A
  MOD_RES (499)..(499): A, I, or H
  MOD_RES (527)..(527): A, I, or V
MEEQYRPEEIESKVQLHWDXKRTFEVTEDESKEKYYCLSIXPYPSGRLHMGHVRNYTIGDVIARYQRMLGKNVLQPIGWDAFGLPAEGAAVKNNTAPAPWTYDNIAYMKNQLKMLGFGYDWSRELATCTPEYYRWEQKFFTELYKKGLVYKKTSAVNWCPNDQTVLANEQVIDGCCWRCDTKVERKEIPQWFIKITAYADELLNDLDKLDHWPDTVKTMQRNWIGRSEGVEITFNVNDYDNTLTVYTTRPDAFMGCTYLAVAAGHPLAQKAAENNPELAAFIDECRNTKVAEAEMATMEKKGVDTGFKAVHPLTGEEIPVWAANFVLMEYGTGAVMAVPGHDQRDYEFASKYGLNIKPVILAADGSEPDLSQQALTEKGVLFNSGEFNGLDHEAAFNAIADKLTAMGVGERKVNYRLRDWGVSRQRYWGAPIPMVTLEDGTVMPTPDDQLPVILPEDVVMDGITSPIKADPEWAKTTVNGMPALRETDTFDTFMESSWXYARYTCPQYKEGMLDSEAANYWLPVDIXIGGIEHAIMGLLYFRFFHKLMRDAGMVNSDEPAKQLLCQGMVLADAFYYVGENGERNWVSPVDAIVERDEKGRIVKAKDAAGHELVYTGMSKMSKSKNNGIDPQVMVERYGADTVRLFMMFASPADMTLEWQESGVEGANRFLKRVWKLVYEHTAKGDVAALNVDALTENQKALRRDVHKTIAKVTDDIGRRQTFNTAIAAIMELMNKLAKAPTDGEQDRALMQEALLAVVRMLNPFTPHICFTLWQELKGEGDIDNAPWPVADEKAMVEDSTLVVVQVNGKVRAKITVPVDATEEQVRERAGQEHLVAKYLDGVTVRKVIYVPGKLLNLVVGGPV

SEQ ID NO: 16 - DNA, 84 bp, Artificial Sequence
  Description of Artificial Sequence Synthetic oligonucleotide
gcccggatggtggaatcggtagacacaagggattctaaatccctcggcgttcgcgctgtgcgggttcaagtcccgctccgggta

SEQ ID NO: 17 - DNA, 84 bp, Artificial Sequence
  Description of Artificial Sequence Synthetic oligonucleotide
gcccggatggtggaatcggtagacacaagggatttcaaatccctcggcgttcgcgctgtgcgggttcaagtcccgctccgggta

SEQ ID NO: 18 - DNA, 84 bp, Artificial Sequence
  Description of Artificial Sequence Synthetic oligonucleotide
gcccggatggtggaatcggtagacacaagggattttaaatccctcggcgttcgcgctgtgcgggttcaagtcccgctccgggta

SEQ ID NO: 19 - DNA, 84 bp, Artificial Sequence
  Description of Artificial Sequence Synthetic oligonucleotide
gcccggatggtggaatcggtagacacaagggactctaaatccctcggcgttcgcgctgtgcgggttcaagtcccgctccgggca

SEQ ID NO: 20 - DNA, 84 bp, Artificial Sequence
  Description of Artificial Sequence Synthetic oligonucleotide
gcccggatggtggaatcggtagacacaagggacttcaaatccctcggcgttcgcgctgtgcgggttcaagtcccgctccgggca

SEQ ID NO: 21 - DNA, 84 bp, Artificial Sequence
  Description of Artificial Sequence Synthetic oligonucleotide
gcccggatggtggaatcggtagacacaagggactttaaatccctcggcgttcgcgctgtgcgggttcaagtcccgctccgggca

SEQ ID NO: 22 - DNA, 87 bp, Artificial Sequence
  Description of Artificial Sequence Synthetic oligonucleotide
gcccggatggtggaatcggtagacacaagggattctaaatccctcggcgttcgcgctgtgcgggttcaagtcccgctccgggtacca

SEQ ID NO: 23 - DNA, 87 bp, Artificial Sequence
  Description of Artificial Sequence Synthetic oligonucleotide
gcccggatggtggaatcggtagacacaagggatttcaaatccctcggcgttcgcgctgtgcgggttcaagtcccgctccgggtacca

SEQ ID NO: 24 - DNA, 87 bp, Artificial Sequence
  Description of Artificial Sequence Synthetic oligonucleotide
gcccggatggtggaatcggtagacacaagggattttaaatccctcggcgttcgcgctgtgcgggttcaagtcccgctccgggtacca

SEQ ID NO: 25 - DNA, 87 bp, Artificial Sequence
  Description of Artificial Sequence Synthetic oligonucleotide
gcccggatggtggaatcggtagacacaagggactctaaatccctcggcgttcgcgctgtgcgggttcaagtcccgctccgggcacca

SEQ ID NO: 26 - DNA, 87 bp, Artificial Sequence
  Description of Artificial Sequence Synthetic oligonucleotide
gcccggatggtggaatcggtagacacaagggacttcaaatccctcggcgttcgcgctgtgcgggttcaagtcccgctccgggcacca

SEQ ID NO: 27 - DNA, 87 bp, Artificial Sequence
  Description of Artificial Sequence Synthetic oligonucleotide
gcccggatggtggaatcggtagacacaagggactttaaatccctcggcgttcgcgctgtgcgggttcaagtcccgctccgggcacca

SEQ ID NO: 28 - DNA, 87 bp, Artificial Sequence
  Description of Artificial Sequence Synthetic oligonucleotide
  modified_base (35)..(37): cta, tca, or tta
gcccggatggtggaatcggtagacacaagggactnnnaatccctcggcgttcgcgctgtgcgggttcaagtcccgctccgggcacca

SEQ ID NO: 29 - DNA, 87 bp, Artificial Sequence
  Description of Artificial Sequence Synthetic oligonucleotide
  modified_base (35)..(37): cta, tca, or tta
gggcgtgtggtggaatcggtagacacaagggattnnnaatccctcggcgttcgcgctgtgcgggttcaagtcccgccgcgcccacca

SEQ ID NO: 30 - DNA, 87 bp, Artificial Sequence
  Description of Artificial Sequence Synthetic oligonucleotide
  modified_base (35)..(37): cta, tca, or tta
gggcgcgtggtggaatcggtagacacaagggattnnnaatccctcggcgttcgcgctgtgcgggttcaagtcccgccgcgcccacca

SEQ ID NO: 31 - DNA, 87 bp, Artificial Sequence
  Description of Artificial Sequence Synthetic oligonucleotide
  modified_base (35)..(37): cta, tca, or tta
gggcatgtggtggaatcggtagacacaagggattnnnaatccctcggcgttcgcgctgtgcgggttcaagtcccgccatgcccacca

SEQ ID NO: 32 - DNA, 87 bp, Artificial Sequence
  Description of Artificial Sequence Synthetic oligonucleotide
  modified_base (35)..(37): cta, tca, or tta
gggcacgtggtggaatcggtagacacaagggattnnnaatccctcggcgttcgcgctgtgcgggttcaagtcccgccgtgcccacca

SEQ ID NO: 33 - DNA, 87 bp, Artificial Sequence
  Description of Artificial Sequence Synthetic oligonucleotide
  modified_base (35)..(37): cta, tca, or tta
gggggtgtggtggaatcggtagacacaagggattnnnaatccctcggcgttcgcgctgtgcgggttcaagtcccgccgcccccacca

SEQ ID NO: 34 - DNA, 87 bp, Artificial Sequence
  Description of Artificial Sequence Synthetic oligonucleotide
  modified_base (35)..(37): cta, tca, or tta
gggggcgtggtggaatcggtagacacaagggattnnnaatccctcggcgttcgcgctgtgcgggttcaagtcccgccgtccccacca

SEQ ID NO: 35 - DNA, 87 bp, Artificial Sequence
  Description of Artificial Sequence Synthetic oligonucleotide
  modified_base (35)..(37): cta, tca, or tta
ggggatgtggtggaatcggtagacacaagggattnnnaatccctcggcgttcgcgctgtgcgggttcaagtcccgccgtccccacca

SEQ ID NO: 36 - DNA, 87 bp, Artificial Sequence
  Description of Artificial Sequence Synthetic oligonucleotide
  modified_base (35)..(37): cta, tca, or tta
ggggacgtggtggaatcggtagacacaagggattnnnaatccctcggcgttcgcgctgtgcgggttcaagtcccgccgtccccacca

SEQ ID NO: 37 - DNA, 87 bp, Artificial Sequence
  Description of Artificial Sequence Synthetic oligonucleotide
  modified_base (35)..(37): cta, tca, or tta
gcccgtatggtggaatcggtagacacaagggattnnnaatccctcggcgttcgcgctgtgcgggttcaagtcccgctgcgggcacca

SEQ ID NO: 38 - DNA, 87 bp, Artificial Sequence
  Description of Artificial Sequence Synthetic oligonucleotide
  modified_base (35)..(37): cta, tca, or tta
gggatagtggtggaatcggtagacacaagggattnnnaatccctcggcgttcgcgctgtgcgggttcaagtcccgcctatcccacca

SEQ ID NO: 39 - DNA, 87 bp, Artificial Sequence
  Description of Artificial Sequence Synthetic oligonucleotide
  modified_base (35)..(37): cta, tca, or tta
gggcatgtggtggaatcggtagacacaagggattnnnaatccctcggcgttcgcgctgtgcgggttcaagtcccgccgtgcccacca

SEQ ID NO: 40 - DNA, 87 bp, Artificial Sequence
  Description of Artificial Sequence Synthetic oligonucleotide
  modified_base (35)..(37): cta, tca, or tta
gggcagatggtggaatcggtagacacaagggattnnnaatccctcggcgttcgcgctgtgcgggttcaagtcccgctctgcccacca

SEQ ID NO: 41 - DNA, 87 bp, Artificial Sequence
  Description of Artificial Sequence Synthetic oligonucleotide
  modified_base (35)..(37): cta, tca, or tta
gggcgtatggtggaatcggtagacacaagggattnnnaatccctcggcgttcgcgctgtgcgggttcaagtcccgctgcgcccacca

SEQ ID NO: 42 - DNA, 87 bp, Artificial Sequence
  Description of Artificial Sequence Synthetic oligonucleotide
  modified_base (35)..(37): cta, tca, or tta
gggcaagtggtggaatcggtagacacaagggattnnnaatccctcggcgttcgcgctgtgcgggttcaagtcccgccttgcccacca

SEQ ID NO: 43 - DNA, 87 bp, Artificial Sequence
  Description of Artificial Sequence Synthetic oligonucleotide
  modified_base (35)..(37): cta, tca, or tta
gcacacatggtggaatcggtagacacaagggattnnnaatccctcggcgttcgcgctgtgcgggttcaagtcccgctgtgtgcacca

SEQ ID NO: 44 - protein, 334 aa, Escherichia coli
MTKPIVFSGAQPSGELTIGNYMGALRQWVNMQDDYHCIYCIVDQHAITVRQDAQKLRKATLDTLALYLACGIDPEKSTIFVQSHVPEHAQLGWALNCYTYFGELSRMTQFKDKSARYAENINAGLFDYPVLMAADILLYQTNLVPVGEDQKQHLELSRDIAQRFNALYGEIFKVPEPFIPKSGARVMSLLEPTKKMSKSDDNRNNVIGLLEDPKSVVKKIKRAVTDSDEPPVVRYDVQNKAGVSNLLDILSAVTGQSIPELEKQFEGKMYGHLKGEVADAVSGMLTELQERYHRFRNDEAFLQQVMKDGAEKASAHASRTLKAVYEAIGFVAKP

SEQ ID NO: 45 - protein, 334 aa, Artificial Sequence
  Description of Artificial Sequence Synthetic polypeptide
MTKPIVFAGAQPSGELTIGNYMGALRQWVNMQDDYHCIYCIVDQHAITVRQDAQKLRKATLDTLALYLACGIDPEKSTIFVQSHVPEHAQLGWALNCYTYFGELSRMTQFKDKSARYAENINAGLFDYPVLMAADILLYQTNLGPCGEDQKQHLELSRDIAQRFNALYGEIFKVPEPFIPKSGARVMSLLEPTKKMSKSDDNRNNVIGLLEDPKSVVKKIKRAVTDSDEPPVVRYDVQNKAGVSNLLDILSAVTGQSIPELEKQFEGKMYGHLKGEVADAVSGMLTELQERYHRFRNDEAFLQQVMKDGAEKASAHASRTLKAVYEAIGFVAKP

SEQ ID NO: 46 - protein, 334 aa, Artificial Sequence
  Description of Artificial Sequence Synthetic polypeptide
MTKPIVFAGAQPSGELTIGNYMGALRQWVNMQDDYHCIYCIVDQHAITVRQDAQKLRKATLDTLALYLACGIDPEKSTIFVQSHVPEHAQLGWALNCYTYFGELSRMTQFKDKSARYAENINAGLFDYPVLMAADILLYQTNLAPAGEDQKQHLELSRDIAQRFNALYGEIFKVPEPFIPKSGARVMSLLEPTKKMSKSDDNRNNVIGLLEDPKSVVKKIKRAVTDSDEPPVVRYDVQNKAGVSNLLDILSAVTGQSIPELEKQFEGKMYGHLKGEVADAVSGMLTELQERYHRFRNDEAFLQQVMKDGAEKASAHASRTLKAVYEAIGFVAKP

SEQ ID NO: 47 - protein, 334 aa, Artificial Sequence
  Description of Artificial Sequence Synthetic polypeptide
MTKPIVFAGAQPSGELTIGNYMGALRQWVNMQDDYHCIYCIVDQHAITVRQDAQKLRKATLDTLALYLACGIDPEKSTIFVQSHVPEHAQLGWALNCYTYFGELSRMTQFKDKSARYAENINAGLFDYPVLMAADILLYQTNLSPAGEDQKQHLELSRDIAQRFNALYGEIFKVPEPFIPKSGARVMSLLEPTKKMSKSDDNRNNVIGLLEDPKSVVKKIKRAVTDSDEPPVVRYDVQNKAGVSNLLDILSAVTGQSIPELEKQFEGKMYGHLKGEVADAVSGMLTELQERYHRFRNDEAFLQQVMKDGAEKASAHASRTLKAVYEAIGFVAKP

SEQ ID NO: 48 - protein, 334 aa, Artificial Sequence
  Description of Artificial Sequence Synthetic polypeptide
MTKPIVFAGAQPSGELTIGNYMGALRQWVNMQDDYHCIYCIVDQHAITVRQDAQKLRKATLDTLALYLACGIDPEKSTIFVQSHVPEHAQLGWALNCYTYFGELSRMTQFKDKSARYAENINAGLFDYPVLMAADILLYQTNLGPAGEDQKQHLELSRDIAQRFNALYGEIFKVPEPFIPKSGARVMSLLEPTKKMSKSDDNRNNVIGLLEDPKSVVKKIKRAVTDSDEPPVVRYDVQNKAGVSNLLDILSAVTGQSIPELEKQFEGKMYGHLKGEVADAVSGMLTELQERYHRFRNDEAFLQQVMKDGAEKASAHASRTLKAVYEAIGFVAKP

SEQ ID NO: 49 - DNA, 76 bp, Escherichia coli
aggggcgtagttcaattggtagagcaccggtctccaaaaccgggtgttgggagttcgagtctctccgcccctgcca

SEQ ID NO: 50 - DNA, 76 bp, Artificial Sequence
  Description of Artificial Sequence Synthetic oligonucleotide
aggggcgtagttcaattggtagagcaccggtctctaaaaccgggtgttgggagttcgagtctctccgcccctgcca

SEQ ID NO: 51 - DNA, 76 bp, Artificial Sequence
  Description of Artificial Sequence Synthetic oligonucleotide
aggggcgtagttcaattggtagagcaccggtcttcaaaaccgggtgttgggagttcgagtctctccgcccctgcca

SEQ ID NO: 52 - DNA, 76 bp, Artificial Sequence
  Description of Artificial Sequence Synthetic oligonucleotide
aggggcgtagttcaattggtagagcaccggtctttaaaaccgggtgttgggagttcgagtctctccgcccctgcca

SEQ ID NO: 53 - DNA, 75 bp, Artificial Sequence
  Description of Artificial Sequence Synthetic oligonucleotide
  modified_base (34)..(36): cta, tca, or tta
tggggtatcgccaagcggtaaggcaccggattcnnnttccggcattccgaggttcgaatcctcgtaccccagcca

SEQ ID NO: 54 - DNA, 76 bp, Artificial Sequence
  Description of Artificial Sequence Synthetic oligonucleotide
  modified_base (34)..(36): cta, tca, or tta
aggggcatagctcaagcggtaaagcaccggactnnnaaaccggcagtccgaagttcgaatccccccaccccagcca

SEQ ID NO: 55 - DNA, 2589 bp, Escherichia coli
atgcaggaacagtatcgcccggaagaaattgaaagcaaagtgcagctgcattgggatgaaaaacgcacctttgaagtgaccgaagatgaaagcaaagaaaaatattattgcctgagcatgctgccgtatccgagcggccgcctgcatatgggccatgtgcgcaactataccattggcgatgtgattgcgcgctatcagcgcatgctgggcaaaaacgtgctgcagccgattggctgggatgcgtttggcctgccggcggaaggcgcggcggtgaaaaacaacaccgcgccggcgccgtggacctatgataacattgcgtatatgaaaaaccagctgaaaatgctgggctttggctatgattggagccgcgaactggcgacctgcaccccggaatattatcgctgggaacagaaattttttaccgaactgtataaaaaaggcctggtgtataaaaaaaccagcgcggtgaactggtgcccgaacgatcagaccgtgctggcgaacgaacaggtgattgatggctgctgctggcgctgcgataccaaagtggaacgcaaagaaattccgcagtggtttattaaaattaccgcgtatgcggatgaactgctgaacgatctggataaactggatcattggccggataccgtgaaaaccatgcagcgcaactggattggccgcagcgaaggcgtggaaattacctttaacgtgaacgattatgataacaccctgaccgtgtataccacccgcccggatacctttatgggctgcacctatctggcggtggcggcgggccatccgctggcgcagaaagcggcggaaaacaacccggaactggcggcgtttattgatgaatgccgcaacaccaaagtggcggaagcggaaatggcgaccatggaaaaaaaaggcgtggataccggctttaaagcggtgcatccgctgaccggcgaagaaattccggtgtgggcggcgaactttgtgctgatggaatatggcaccggcgcggtgatggcggtgccgggccatgatcagcgcgattatgaatttgcgagcaaatatggcctgaacattaaaccggtgattctggcggcggatggcagcgaaccggatctgagccagcaggcgctgaccgaaaaaggcgtgctgtttaacagcggcgaatttaacggcctggatcatgaagcggcgtttaacgcgattgcggataaactgaccgcgatgggcgtgggcgaacgcaaagtgaactatcgcctgcgcgattggggcgtgagccgccagcgctattggggcgcgccgattccgatggtgaccctggaagatggcaccgtgatgccgaccccggatgatcagctgccggtgattctgccggaagatgtggtgatggatggcattaccagcccgattaaagcggatccggaatgggcgaaaaccaccgtgaacggcatgccggcgctgcgcgaaaccgatacctttgatacctttatggaaagcagctggtattatgcgcgctatacctgcccgcagtataaagaaggcatgctggatagcgaagcggcgaactattggctgccggtggatatttatattggcggcattgaacatgcgattatgcatctgctgtattttcgcttttttcataaactgatgcgcgatgcgggcatggtgaacagcgatgaaccggcgaaacagctgctgtgccagggcatggtgctggcggatgcgttttattatgtgggcgaaaacggcgaacgcaactgggtgagcccggtggatgcgattgtggaacgcgatgaaaaaggccgcattgtgaaagcgaaagatgcggcgggccatgaactggtgtataccggcatgagcaaaatgagcaaaagcaaaaacaacggcattgatccgcaggtgatggtggaacgctatggcgcggataccgtgcgcctgtttatgatgtttgcgagcccggcggatatgaccctggaatggcaggaaagcggcgtggaaggcgcgaaccgctttctgaaacgcgtgtggaaactggtgtatgaacataccgcgaaaggcgatgtggcggcgctgaacgtggatgcgctgaccgaaaaccagaaagcgctgcgccgcgatgtgcataaaaccattgcgaaagtgaccgatgatattggccgccgccagacctttaacaccgcgattgcggcgattatggaactgatgaacaaactggcgaaagcgccgaccgatggcgaacaggatcgcgcgctgatgcaggaagcgctgctggcggtggtgcgcatgctgaacccgtttaccccgcatatttgctttaccctgtggcaggaactgaaaggcgaaggcgatattgataacgcgccgtggccggtggcggatgaaaaagcgatggtggaagatagcaccctggtggtggtgcaggtgaacggcaaagtgcgcgcgaaaattaccgtgccggtggatgcgaccgaagaacaggtgcgcgaacgcgcgggccaggaacatctggtggcgaaatatctggatggcgtgaccgtgcgcaaagtgatttatgtgccgggcaaactgctgaacctggtggtgggcggcccggtg

SEQ ID NO: 56 - DNA, 2589 bp, Artificial Sequence
  Description of Artificial Sequence Synthetic polynucleotide
atggaagaacagtatcgcccggaagaaattgaaagcaaagtgcagctgcattgggataaaaaacgcacctttgaagtgaccgaagatgaaagcaaagaaaaatattattgcctgagcattagcccgtatccgagcggccgcctgcatatgggccatgtgcgcaactataccattggcgatgtgattgcgcgctatcagcgcatgctgggcaaaaacgtgctgcagccgattggctgggatgcgtttggcctgccggcggaaggcgcggcggtgaaaaacaacaccgcgccggcgccgtggacctatgataacattgcgtatatgaaaaaccagctgaaaatgctgggctttggctatgattggagccgcgaactggcgacctgcaccccggaatattatcgctgggaacagaaattttttaccgaactgtataaaaaaggcctggtgtataaaaaaaccagcgcggtgaactggtgcccgaacgatcagaccgtgctggcgaacgaacaggtgattgatggctgctgctggcgctgcgataccaaagtggaacgcaaagaaattccgcagtggtttattaaaattaccgcgtatgcggatgaactgctgaacgatctggataaactggatcattggccggataccgtgaaaaccatgcagcgcaactggattggccgcagcgaaggcgtggaaattacctttaacgtgaacgattatgataacaccctgaccgtgtataccacccgcccggatgcgtttatgggctgcacctatctggcggtggcggcgggccatccgctggcgcagaaagcggcggaaaacaacccggaactggcggcgtttattgatgaatgccgcaacaccaaagtggcggaagcggaaatggcgaccatggaaaaaaaaggcgtggataccggctttaaagcggtgcatccgctgaccggcgaagaaattccggtgtgggcggcgaactttgtgctgatggaatatggcaccggcgcggtgatggcggtgccgggccatgatcagcgcgattatgaatttgcgagcaaatatggcctgaacattaaaccggtgattctggcggcggatggcagcgaaccggatctgagccagcaggcgctgaccgaaaaaggcgtgctgtttaacagcggcgaatttaacggcctggatcatgaagcggcgtttaacgcgattgcggataaactgaccgcgatgggcgtgggcgaacgcaaagtgaactatcgcctgcgcgattggggcgtgagccgccagcgctattggggcgcgccgattccgatggtgaccctggaagatggcaccgtgatgccgaccccggatgatcagctgccggtgattctgccggaagatgtggtgatggatggcattaccagcccgattaaagcggatccggaatgggcgaaaaccaccgtgaacggcatgccggcgctgcgcgaaaccgatacctttgatacctttatggaaagcagctggatttatgcgcgctatacctgcccgcagtataaagaaggcatgctggatagcgaagcggcgaactattggctgccggtggatattgcgattggcggcattgaacatgcgattatgggcctgctgtattttcgcttttttcataaactgatgcgcgatgcgggcatggtgaacagcgatgaaccggcgaaacagctgctgtgccagggcatggtgctggcggatgcgttttattatgtgggcgaaaacggcgaacgcaactgggtgagcccggtggatgcgattgtggaacgcgatgaaaaaggccgcattgtgaaagcgaaagatgcggcgggccatgaactggtgtataccggcatgagcaaaatgagcaaaagcaaaaacaacggcattgatccgcaggtgatggtggaacgctatggcgcggataccgtgcgcctgtttatgatgtttgcgagcccggcggatatgaccctggaatggcaggaaagcggcgtggaaggcgcgaaccgctttctgaaacgcgtgtggaaactggtgtatgaacataccgcgaaaggcgatgtggcggcgctgaacgtggatgcgctgaccgaaaaccagaaagcgctgcgccgcgatgtgcataaaaccattgcgaaagtgaccgatgatattggccgccgccagacctttaacaccgcgattgcggcgattatggaactgatgaacaaactggcgaaagcgccgaccgatggcgaacaggatcgcgcgctgatgcaggaagcgctgctggcggtggtgcgcatgctgaacccgtttaccccgcatatttgctttaccctgtggcaggaactgaaaggcgaaggcgatattgataacgcgccgtggccggtggcggatgaaaaagcgatggtggaagatagcaccctggtggtggtgcaggtgaacggcaaagtgcgcgcgaaaattaccgtgccggtggatgcgaccgaagaacaggtgcgcgaacgcgcgggccaggaacatctggtggcgaaatatctggatggcgtgaccgtgcgcaaagtgatttatgtgccgggcaaactgctgaacctggtggtgggcggcccggtg

SEQ ID NO: 57 - DNA, 2589 bp, Artificial Sequence
  Description of Artificial Sequence Synthetic polynucleotide
atggaagaacagtatcgcccggaagaaattgaaagcaaagtgcagctgcattgggataaaaaacgcacctttgaagtgaccgaagatgaaagcaaagaaaaatattattgcctgagcgtgagcccgtatccgagcggccgcctgcatatgggccatgtgcgcaactataccattggcgatgtgattgcgcgctatcagcgcatgctgggcaaaaacgtgctgcagccgattggctgggatgcgtttggcctgccggcggaaggcgcggcggtgaaaaacaacaccgcgccggcgccgtggacctatgataacattgcgtatatgaaaaaccagctgaaaatgctgggctttggctatgattggagccgcgaactggcgacctgcaccccggaatattatcgctgggaacagaaattttttaccgaactgtataaaaaaggcctggtgtataaaaaaaccagcgcggtgaactggtgcccgaacgatcagaccgtgctggcgaacgaacaggtgattgatggctgctgctggcgctgcgataccaaagtggaacgcaaagaaattccgcagtggtttattaaaattaccgcgtatgcggatgaactgctgaacgatctggataaactggatcattggccggataccgtgaaaaccatgcagcgcaactggattggccgcagcgaaggcgtggaaattacctttaacgtgaacgattatgataacaccctgaccgtgtataccacccgcccggatcgctttatgggctgcacctatctggcggtggcggcgggccatccgctggcgcagaaagcggcggaaaacaacccggaactggcggcgtttattgatgaatgccgcaacaccaaagtggcggaagcggaaatggcgaccatggaaaaaaaaggcgtggataccggctttaaagcggtgcatccgctgaccggcgaagaaattccggtgtgggcggcgaactttgtgctgatggaatatggcaccggcgcggtgatggcggtgccgggccatgatcagcgcgattatgaatttgcgagcaaatatggcctgaacattaaaccggtgattctggcggcggatggcagcgaaccggatctgagccagcaggcgctgaccgaaaaaggcgtgctgtttaacagcggcgaatttaacggcctggatcatgaagcggcgtttaacgcgattgcggataaactgaccgcgatgggcgtgggcgaacgcaaagtgaactatcgcctgcgcgattggggcgtgagccgccagcgctattggggcgcgccgattccgatggtgaccctggaagatggcaccgtgatgccgaccccggatgatcagctgccggtgattctgccggaagatgtggtgatggatggcattaccagcccgattaaagcggatccggaatgggcgaaaaccaccgtgaacggcatgccggcgctgcgcgaaaccgatacctttgatacctttatggaaagcagctggagctatgcgcgctatacctgcccgcagtataaagaaggcatgctggatagcgaagcggcgaactattggctgccggtggatattctgattggcggcattgaacatgcgattatgggcctgctgtattttcgcttttttcataaactgatgcgcgatgcgggcatggtgaacagcgatgaaccggcgaaacagctgctgtgccagggcatggtgctggcggatgcgttttattatgtgggcgaaaacggcgaacgcaactgggtgagcccggtggatgcgattgtggaacgcgatgaaaaaggccgcattgtgaaagcgaaagatgcggcgggccatgaactggtgtataccggcatgagcaaaatgagcaaaagcaaaaacaacggcattgatccgcaggtgatggtggaacgctatggcgcggataccgtgcgcctgtttatgatgtttgcgagcccggcggatatgaccctggaatggcaggaaagcggcgtggaaggcgcgaaccgctttctgaaacgcgtgtggaaactggtgtatgaacataccgcgaaaggcgatgtggcggcgctgaacgtggatgcgctgaccgaaaaccagaaagcgctgcgccgcgatgtgcataaaaccattgcgaaagtgaccgatgatattggccgccgccagacctttaacaccgcgattgcggcgattatggaactgatgaacaaactggcgaaagcgccgaccgatggcgaacaggatcgcgcgctgatgcaggaagcgctgctggcggtggtgcgcatgctgaacccgtttaccccgcatatttgctttaccctgtggcaggaactgaaaggcgaaggcgatattgataacgcgccgtggccggtggcggatgaaaaagcgatggtggaagatagcaccctggtggtggtgcaggtgaacggcaaagtgcgcgcgaaaattaccgtgccggtggatgcgaccgaagaacaggtgcgcgaacgcgcgggccaggaacatctggtggcgaaatatctggatggcgtgaccgtgcgcaaagtgatttatgtgccgggcaaactgctgaacctggtggtgggcggcccggtg

SEQ ID NO: 58 - DNA, 2589 bp, Artificial Sequence
  Description of Artificial Sequence Synthetic polynucleotide
atggaagaacagtatcgcccggaagaaattgaaagcaaagtgcagctgcattgggatgaaaaacgcacctttgaagtgaccgaagatgaaagcaaagaaaaatattattgcctgagcattctgccgtatccgagcggccgcctgcatatgggccatgtgcgcaactataccattggcgatgtgattgcgcgctatcagcgcatgctgggcaaaaacgtgctgcagccgattggctgggatgcgtttggcctgccggcggaaggcgcggcggtgaaaaacaacaccgcgccggcgccgtggacctatgataacattgcgtatatgaaaaaccagctgaaaatgctgggctttggctatgattggagccgcgaactggcgacctgcaccccggaatattatcgctgggaacagaaattttttaccgaactgtataaaaaaggcctggtgtataaaaaaaccagcgcggtgaactggtgcccgaacgatcagaccgtgctggcgaacgaacaggtgattgatggctgctgctggcgctgcgataccaaagtggaacgcaaagaaattccgcagtggtttattaaaattaccgcgtatgcggatgaactgctgaacgatctggataaactggatcattggccggataccgtgaaaaccatgcagcgcaactggattggccgcagcgaaggcgtggaaattacctttaacgtgaacgattatgataacaccctgaccgtgtataccacccgcccggatgcgtttatgggctgcacctatctggcggtggcggcgggccatccgctggcgcagaaagcggcggaaaacaacccggaactggcggcgtttattgatgaatgccgcaacaccaaagtggcggaagcggaaatggcgaccatggaaaaaaaaggcgtggataccggctttaaagcggtgcatccgctgaccggcgaagaaattccggtgtgggcggcgaactttgtgctgatggaatatggcaccggcgcggtgatggcggtgccgggccatgatcagcgcgattatgaatttgcgagcaaatatggcctgaacattaaaccggtgattctggcggcggatggcagcgaaccggatctgagccagcaggcgctgaccgaaaaaggcgtgctgtttaacagcggcgaatttaacggcctggatcatgaagcggcgtttaacgcgattgcggataaactgaccgcgatgggcgtgggcgaacgcaaagtgaactatcgcctgcgcgattggggcgtgagccgccagcgctattggggcgcgccgattccgatggtgaccctggaagatggcaccgtgatgccgaccccggatgatcagctgccggtgattctgccggaagatgtggtgatggatggcattaccagcccgattaaagcggatccggaatgggcgaaaaccaccgtgaacggcatgccggcgctgcgcgaaaccgatacctttgatacctttatggaaagcagctggatttatgcgcgctatacctgcccgcagtataaagaaggcatgctggatagcgaagcggcgaactattggctgccggtggatattgcgattggcggcattgaacatgcgattatgggcctgctgtattttcgcttttttcataaactgatgcgcgatgcgggcatggtgaacagcgatgaaccggcgaaacagctgctgtgccagggcatggtgctggcggatgcgttttattatgtgggcgaaaacggcgaacgcaactgggtgagcccggtggatgcgattgtggaacgcgatgaaaaaggccgcattgtgaaagcgaaagatgcggcgggccatgaactggtgtataccggcatgagcaaaatgagcaaaagcaaaaacaacggcattgatccgcaggtgatggtggaacgctatggcgcggataccgtgcgcctgtttatgatgtttgcgagcccggcggatatgaccctggaatggcaggaaagcggcgtggaaggcgcgaaccgctttctgaaacgcgtgtggaaactggtgtatgaacataccgcgaaaggcgatgtggcggcgctgaacgtggatgcgctgaccgaaaaccagaaagcgctgcgccgcgatgtgcataaaaccattgcgaaagtgaccgatgatattggccgccgccagacctttaacaccgcgattgcggcgattatggaactgatgaacaaactggcgaaagcgccgaccgatggcgaacaggatcgcgcgctgatgcaggaagcgctgctggcggtggtgcgcatgctgaacccgtttaccccgcatatttgctttaccctgtggcaggaactgaaaggcgaaggcgatattgataacgcgccgtggccggtggcggatgaaaaagcgatggtggaagatagcaccctggtggtggtgcaggtgaacggcaaagtgcgcgcgaaaattaccgtgccggtggatgcgaccgaagaacaggtgcgcgaacgcgcgggccaggaacatctggtggcgaaatatctggatggcgtgaccgtgcgcaaagtgatttatgtgccgggcaaactgctgaacctggtggtgggcggcccggtg

SEQ ID NO: 59 - DNA, 2589 bp, Artificial Sequence
  Description of Artificial Sequence Synthetic polynucleotide
atggaagaacagtatcgcccggaagaaattgaaagcaaagtgcagctgcattgggatatgaaacgcacctttgaagtgaccgaagatgaaagcaaagaaaaatattattgcctgagcattagcccgtatccgagcggccgcctgcatatgggccatgtgcgcaactataccattggcgatgtgattgcgcgctatcagcgcatgctgggcaaaaacgtgctgcagccgattggctgggatgcgtttggcctgccggcggaaggcgcggcggtgaaaaacaacaccgcgccggcgccgtggacctatgataacattgcgtatatgaaaaaccagctgaaaatgctgggctttggctatgattggagccgcgaactggcgacctgcaccccggaatattatcgctgggaacagaaattttttaccgaactgtataaaaaaggcctggtgtataaaaaaaccagcgcggtgaactggtgcccgaacgatcagaccgtgctggcgaacgaacaggtgattgatggctgctgctggcgctgcgataccaaagtggaacgcaaagaaattccgcagtggtttattaaaattaccgcgtatgcggatgaactgctgaacgatctggataaactggatcattggccggataccgtgaaaaccatgcagcgcaactggattggccgcagcgaaggcgtggaaattacctttaacgtgaacgattatgataacaccctgaccgtgtataccacccgcccggatgcgtttatgggctgcacctatctggcggtggcggcgggccatccgctggcgcagaaagcggcggaaaacaacccggaactggcggcgtttattgatgaatgccgcaacaccaaagtggcggaagcggaaatggcgaccatggaaaaaaaaggcgtggataccggctttaaagcggtgcatccgctgaccggcgaagaaattccggtgtgggcggcgaactttgtgctgatggaatatggcaccggcgcggtgatggcggtgccgggccatgatcagcgcgattatgaatttgcgagcaaatatggcctgaacattaaaccggtgattctggcggcggatggcagcgaaccggatctgagccagcaggcgctgaccgaaaaaggcgtgctgtttaacagcggcgaatttaacggcctggatcatgaagcggcgtttaacgcgattgcggataaactgaccgcgatgggcgtgggcgaacgcaaagtgaactatcgcctgcgcgattggggcgtgagccgccagcgctattggggcgcgccgattccgatggtgaccctggaagatggcaccgtgatgccgaccccggatgatcagctgccggtgattctgccggaagatgtggtgatggatggcattaccagcccgattaaagcggatccggaatgggcgaaaaccaccgtgaacggcatgccggcgctgcgcgaaaccgatacctttgatacctttatggaaagcagctggatttatgcgcgctatacctgcccgcagtataaagaaggcatgctggatagcgaagcggcgaactattggctgccggtggatattgcgattggcggcattgaacatgcgattatgggcctgctgtattttcgcttttttcataaactgatgcgcgatgcgggcatggtgaacagcgatgaaccggcgaaacagctgctgtgccagggcatggtgctggcggatgcgttttattatgtgggcgaaaacggcgaacgcaactgggtgagcccggtggatgcgattgtggaacgcgatgaaaaaggccgcattgtgaaagcgaaagatgcggcgggccatgaactggtgtataccggcatgagcaaaatgagcaaaagcaaaaacaacggcattgatccgcaggtgatggtggaacgctatggcgcggataccgtgcgcctgtttatgatgtttgcgagcccggcggatatgaccctggaatggcaggaaagcggcgtggaaggcgcgaaccgctttctgaaacgcgtgtggaaactggtgtatgaacataccgcgaaaggcgatgtggcggcgctgaacgtggatgcgctgaccgaaaaccagaaagcgctgcgccgcgatgtgcataaaaccattgcgaaagtgaccgatgatattggccgccgccagacctttaacaccgcgattgcggcgattatggaactgatgaacaaactggcgaaagcgccgaccgatggcgaacaggatcgcgcgctgatgcaggaagcgctgctggcggtggtgcgcatgctgaacccgtttaccccgcatatttgctttaccctgtggcaggaactgaaaggcgaaggcgatattgataacgcgccgtggccggtggcggatgaaaaagcgatggtggaagatagcaccctggtggtggtgcaggtgaacggcaaagtgcgcgcgaaaattaccgtgccggtggatgcgaccgaagaacaggtgcgcgaacgcgcgggccaggaacatctggtggcgaaatatctggatggcgtgaccgtgcgcaaagtgatttatgtgccgggcaaactgctgaacctggtggtgggcggcccggtg

SEQ ID NO: 60 - DNA, 2589 bp, Artificial Sequence
  Description of Artificial Sequence Synthetic polynucleotide
atggaagaacagtatcgcccggaagaaattgaaagcaaagtgcagctgcattgggatgtgaaacgcacctttgaagtgaccgaagatgaaagcaaagaaaaatattattgcctgagcattagcccgtatccgagcggccgcctgcatatgggccatgtgcgcaactataccattggcgatgtgattgcgcgctatcagcgcatgctgggcaaaaacgtgctgcagccgattggctgggatgcgtttggcctgccggcggaaggcgcggcggtgaaaaacaacaccgcgccggcgccgtggacctatgataacattgcgtatatgaaaaaccagctgaaaatgctgggctttggctatgattggagccgcgaactggcgacctgcaccccggaatattatcgctgggaacagaaattttttaccgaactgtataaaaaaggcctggtgtataaaaaaaccagcgcggtgaactggtgcccgaacgatcagaccgtgctggcgaacgaacaggtgattgatggctgctgctggcgctgcgataccaaagtggaacgcaaagaaattccgcagtggtttattaaaattaccgcgtatgcggatgaactgctgaacgatctggataaactggatcattggccggataccgtgaaaaccatgcagcgcaactggattggccgcagcgaaggcgtggaaattacctttaacgtgaacgattatgataacaccctgaccgtgtataccacccgcccggatgcgtttatgggctgcacctatctggcggtggcggcgggccatccgctggcgcagaaagcggcggaaaacaacccggaactggcggcgtttattgatgaatgccgcaacaccaaagtggcggaagcggaaatggcgaccatggaaaaaaaaggcgtggataccggctttaaagcggtgcatccgctgaccggcgaagaaattccggtgtgggcggcgaactttgtgctgatggaatatggcaccggcgcggtgatggcggtgccgggccatgatcagcgcgattatgaatttgcgagcaaatatggcctgaacattaaaccggtgattctggcggcggatggcagcgaaccggatctgagccagcaggcgctgaccgaaaaaggcgtgctgtttaacagcggcgaatttaacggcctggatcatgaagcggcgtttaacgcgattgcggataaactgaccgcgatgggcgtgggcgaacgcaaagtgaactatcgcctgcgcgattggggcgtgagccgccagcgctattggggcgcgccgattccgatggtgaccctggaagatggcaccgtgatgccgaccccggatgatcagctgccggtgattctgccggaagatgtggtgatggatggcattaccagcccgattaaagcggatccggaatgggcgaaaaccaccgtgaacggcatgccggcgctgcgcgaaaccgatacctttgatacctttatggaaagcagctggatttatgcgcgctatacctgcccgcagtataaagaaggcatgctggatagcgaagcggcgaactattggctgccggtggatattgcgattggcggcattgaacatgcgattatgggcctgctgtattttcgcttttttcataaactgatgcgcgatgcgggcatggtgaacagcgatgaaccggcgaaacagctgctgtgccagggcatggtgctggcggatgcgttttattatgtgggcgaaaacggcgaacgcaactgggtgagcccggtggatgcgattgtggaacgcgatgaaaaaggccgcattgtgaaagcgaaagatgcggcgggccatgaactggtgtataccggcatgagcaaaatgagcaaaagcaaaaacaacggcattgatccgcaggtgatggtggaacgctatggcgcggataccgtgcgcctgtttatgatgtttgcgagcccggcggatatgaccctggaatggcaggaaagcggcgtggaaggcgcgaaccgctttctgaaacgcgtgtggaaactggtgtatgaacataccgcgaaaggcgatgtggcggcgctgaacgtggatgcgctgaccgaaaaccagaaagcgctgcgccgcgatgtgcataaaaccattgcgaaagtgaccgatgatattggccgccgccagacctttaacaccgcgattgcggcgattatggaactgatgaacaaactggcgaaagcgccgaccgatggcgaacaggatcgcgcgctgatgcaggaagcgctgctggcggtggtgcgcatgctgaacccgtttaccccgcatatttgctttaccctgtggcaggaactgaaaggcgaaggcgatattgataacgcgccgtggccggtggcggatgaaaaagcgatggtggaagatagcaccctggtggtggtgcaggtgaacggcaaagtgcgcgcgaaaattaccgtgccggtggatgcgaccgaagaacaggtgcgcgaacgcgcgggccaggaacatctggtggcgaaatatctggatggcgtgaccgtgcgcaaagtgatttatgtgccgggcaaactgctgaacctggtggtgggcggcccggtg

SEQ ID NO: 61 - DNA, 2589 bp, Artificial Sequence
  Description of Artificial Sequence Synthetic polynucleotide
atggaagaacagtatcgcccggaagaaattgaaagcaaagtgcagctgcattgggataaaaaacgcacctttgaagtgaccgaagatgaaagcaaagaaaaatattattgcctgagcattgtgccgtatccgagcggccgcctgcatatgggccatgtgcgcaactataccattggcgatgtgattgcgcgctatcagcgcatgctgggcaaaaacgtgctgcagccgattggctgggatgcgtttggcctgccggcggaaggcgcggcggtgaaaaacaacaccgcgccggcgccgtggacctatgataacattgcgtatatgaaaaaccagctgaaaatgctgggctttggctatgattggagccgcgaactggcgacctgcaccccggaatattatcgctgggaacagaaattttttaccgaactgtataaaaaaggcctggtgtataaaaaaaccagcgcggtgaactggtgcccgaacgatcagaccgtgctggcgaacgaacaggtgattgatggctgctgctggcgctgcgataccaaagtggaacgcaaagaaattccgcagtggtttattaaaattaccgcgtatgcggatgaactgctgaacgatctggataaactggatcattggccggataccgtgaaaaccatgcagcgcaactggattggccgcagcgaaggcgtggaaattacctttaacgtgaacgattatgataacaccctgaccgtgtataccacccgcccggatgcgtttatgggctgcacctatctggcggtggcggcgggccatccgctggcgcagaaagcggcggaaaacaacccggaactggcggcgtttattgatgaatgccgcaacaccaaagtggcggaagcggaaatggcgaccatggaaaaaaaaggcgtggataccggctttaaagcggtgcatccgctgaccggcgaagaaattccggtgtgggcggcgaactttgtgctgatggaatatggcaccggcgcggtgatggcggtgccgggccatgatcagcgcgattatgaatttgcgagcaaatatggcctgaacattaaaccggtgattctggcggcggatggcagcgaaccggatctgagccagcaggcgctgaccgaaaaaggcgtgctgtttaacagcggcgaatttaacggcctggatcatgaagcggcgtttaacgcgattgcggataaactgaccgcgatgggcgtgggcgaacgcaaagtgaactatcgcctgcgcgattggggcgtgagccgccagcgctattggggcgcgccgattccgatggtgaccctggaagatggcaccgtgatgccgaccccggatgatcagctgccggtgattctgccggaagatgtggtgatggatggcattaccagcccgattaaagcggatccggaatgggcgaaaaccaccgtgaacggcatgccggcgctgcgcgaaaccgatacctttgatacctttatggaaagcagctggatttatgcgcgctatacctgcccgcagtataaagaaggcatgctggatagcgaagcggcgaactattggctgccggtggatattgcgattggcggcattgaacatgcgattatgggcctgctgtattttcgcttttttcataaactgatgcgcgatgcgggcatggtgaacagcgatgaaccggcgaaacagctgctgtgccagggcatggtgctggcggatgcgttttattatgtgggcgaaaacggcgaacgcaactgggtgagcccggtggatgcgattgtggaacgcgatgaaaaaggccgcattgtgaaagcgaaagatgcggcgggccatgaactggtgtataccggcatgagcaaaatgagcaaaagcaaaaacaacggcattgatccgcaggtgatggtggaacgctatggcgcggataccgtgcgcctgtttatgatgtttgcgagcccggcggatatgaccctggaatggcaggaaagcggcgtggaaggcgcgaaccgctttctgaaacgcgtgtggaaactggtgtatgaacataccgcgaaaggcgatgtggcggcgctgaacgtggatgcgctgaccgaaaaccagaaagcgctgcgccgcgatgtgcataaaaccattgcgaaagtgaccgatgatattggccgccgccagacctttaacaccgcgattgcggcgattatggaactgatgaacaaactggcgaaagcgccgaccgatggcgaacaggatcgcgcgctgatgcaggaagcgctgctggcggtggtgcgcatgctgaacccgtttaccccgcatatttgctttaccctgtggcaggaactgaaaggcgaaggcgatattgataacgcgccgtggccggtggcggatgaaaaagcgatggtggaagatagcaccctggtggtggtgcaggtgaacggcaaagtgcgcgcgaaaattaccgtgccggtggatgcgaccgaagaacaggtgcgcgaacgcgcgggccaggaacatctggtggcgaaatatctggatggcgtgaccgtgcgcaaagtgatttatgtgccgggcaaactgctgaacctggtggtgggcggcccggtg

SEQ ID NO: 62 - DNA, 2589 bp, Artificial Sequence
  Description of Artificial Sequence Synthetic polynucleotide
atggaagaacagtatcgcccggaagaaattgaaagcaaagtgcagctgcattgggataaaaaacgcacctttgaagtgaccgaagatgaaagcaaagaaaaatattattgcctgagcattgcgccgtatccgagcggccgcctgcatatgggccatgtgcgcaactataccattggcgatgtgattgcgcgctatcagcgcatgctgggcaaaaacgtgctgcagccgattggctgggatgcgtttggcctgccggcggaaggcgcggcggtgaaaaacaacaccgcgccggcgccgtggacctatgataacattgcgtatatgaaaaaccagctgaaaatgctgggctttggctatgattggagccgcgaactggcgacctgcaccccggaatattatcgctgggaacagaaattttttaccgaactgtataaaaaaggcctggtgtataaaaaaaccagcgcggtgaactggtgcccgaacgatcagaccgtgctggcgaacgaacaggtgattgatggctgctgctggcgctgcgataccaaagtggaacgcaaagaaattccgcagtggtttattaaaattaccgcgtatgcggatgaactgctgaacgatctggataaactggatcattggccggataccgtgaaaaccatgcagcgcaactggattggccgcagcgaaggcgtggaaattacctttaacgtgaacgattatgataacaccctgaccgtgtataccacccgcccggatgcgtttatgggctgcacctatctggcggtggcggcgggccatccgctggcgcagaaagcggcggaaaacaacccggaactggcggcgtttattgatgaatgccgcaacaccaaagtggcggaagcggaaatggcgaccatggaaaaaaaaggcgtggataccggctttaaagcggtgcatccgctgaccggcgaagaaattccggtgtgggcggcgaactttgtgctgatggaatatggcaccggcgcggtgatggcggtgccgggccatgatcagcgcgattatgaatttgcgagcaaatatggcctgaacattaaaccggtgattctggcggcggatggcagcgaaccggatctgagccagcaggcgctgaccgaaaaaggcgtgctgtttaacagcggcgaatttaacggcctggatcatgaagcggcgtttaacgcgattgcggataaactgaccgcgatgggcgtgggcgaacgcaaagtgaactatcgcctgcgcgattggggcgtgagccgccagcgctattggggcgcgccgattccgatggtgaccctggaagatggcaccgtgatgccgaccccggatgatcagctgccggtgattctgccggaagatgtggtgatggatggcattaccagcccgattaaagcggatccggaatgggcgaaaaccaccgtgaacggcatgccggcgctgcgcgaaaccgatacctttgatacctttatggaaagcagctggatttatgcgcgctatacctgcccgcagtataaagaaggcatgctggatagcgaagcggcgaactattggctgccggtggatattgcgattggcggcattgaacatgcgattatgggcctgctgtattttcgcttttttcataaactgatgcgcgatgcgggcatggtgaacagcgatgaaccggcgaaacagctgctgtgccagggcatggtgctggcggatgcgttttattatgtgggcgaaaacggcgaacgcaactgggtgagcccggtggatgcgattgtggaacgcgatgaaaaaggccgcattgtgaaagcgaaagatgcggcgggccatgaactggtgtataccggcatgagcaaaatgagcaaaagcaaaaacaacggcattgatccgcaggtgatggtggaacgctatggcgcggataccgtgcgcctgtttatgatgtttgcgagcccggcggatatgaccctggaatggcaggaaagcggcgtggaaggcgcgaaccgctttctgaaacgcgtgtggaaactggtgtatgaacataccgcgaaaggcgatgtggcggcgctgaacgtggatgcgctgaccgaaaaccagaaagcgctgcgccgcgatgtgcataaaaccattgcgaaagtgaccgatgatattggccgccgccagacctttaacaccgcgattgcggcgattatggaactgatgaacaaactggcgaaagcgccgaccgatggcgaacaggatcgcgcgctgatgcaggaagcgctgctggcggtggtgcgcatgctgaacccgtttaccccgcatatttgctttaccctgtggcaggaactgaaaggcgaaggcgatattgataacgcgccgtggccggtggcggatgaaaaagcgatggtggaagatagcaccctggtggtggtgcaggtgaacggcaaagtgcgcgcgaaaattaccgtgccggtggatgcgaccgaagaacaggtgcgcgaacgcgcgggccaggaacatctggtggcgaaatatctggatggcgtgaccgtgcgcaaagtgatttatgtgccgggcaaactgctgaacctggtggtgggcggcccggtg

SEQ ID NO: 63 - DNA, 2586 bp, Artificial Sequence
  Description of Artificial Sequence Synthetic polynucleotide
atggaagaacagtatcgcccggaagaaattgaaagcaaagtgcagctgcattgggataaaaaacgcacctttgaagtgaccgaagatgaaagcaaagaaaaatattattgcctgagcattagcccgtatccgagcggccgcctgcatatgggccatgtgcgcaactataccattggcgatgtgattgcgcgctatcagcgcatgctgggcaaaaacgtgctgcagccgattggctgggatgcgtttggcctgccggcggaaggcgcggcggtgaaaaacaacaccgcgccggcgccgtggacctatgataacattgcgtatatgaaaaaccagctgaaaatgctgggctttggctatgattggagccgcgaactggcgacctgcaccccggaatattatcgctgggaacagaaattttttaccgaactgtataaaaaaggcctggtgtataaaaaaaccagcgcggtgaactggtgcccgaacgatcagaccgtgctggcgaacgaacaggtgattgatggctgctgctggcgctgcgataccaaagtggaacgcaaagaaattccgcagtggtttattaaaattaccgcgtatgcggatgaactgctgaacgatctggataaactggatcattggccggataccgtgaaaaccatgcagcgcaactggattggccgcagcgaaggcgtggaaattacctttaacgtgaacgattatgataacaccctgaccgtgtataccacccgcccggatgcgtttatgggctgcacctatctggcggtggcggcgggccatccgctggcgcagaaagcggcggaaaacaacccggaactggcggcgtttattgatgaatgccgcaacaccaaagtggcggaagcggaaatggcgaccatggaaaaaaaaggcgtggataccggctttaaagcggtgcatccgctgaccggcgaagaaattccggtgtgggcggcgaactttgtgctgatggaatatggcaccggcgcggtgatggcggtgccgggccatgatcagcgcgattatgaatttgcgagcaaatatggcctgaacattaaaccggtgattctggcggcggatggcagcgaaccggatctgagccagcaggcgctgaccgaaaaaggcgtgctgtttaacagcggcgaatttaacggcctggatcatgaagcggcgtttaacgcgattgcggataaactgaccgcgatgggcgtgggcgaacgcaaagtgaactatcgcctgcgcgattggggcgtgagccgccagcgctattggggcgcgccgattccgatggtgaccctggaagatggcaccgtgatgccgaccccggatgatcagctgccggtgattctgccggaagatgtggtgatggatggcattaccagcccgattaaagcggatccggaatgggcgaaaaccaccgtgaacggcatgccggcgctgcgcgaaaccgatacctttgatacctttatggaaagcagctgggcgtatgcgcgctatacctgcccgcagtataaagaaggcatgctggatagcgaagcggcgaactattggctgccggtggatattgcgattggcggcattgaacatgcgattatgggcctgctgtattttcgcttttttcataaactgatgcgcgatgcgggcatggtgaacagcgatgaaccggcgaaacagctgctgtgccagggcatggtgctggcggatgcgttttattatgtgggcgaaaacggcgaacgcaactgggtgagcccggtggatgcgattgtggaacgcgatgaaaaaggccgcattgtgaaagcgaaagatgcggcgggccatgaactggtgtataccggcatgagcaaaatgagcaaaagcaaaaacaacggcattgatccgcaggtgatggtggaacgctatggcgcggataccgtgcgcctgtttatgatgtttgcgagcccggcggatatgaccctggaatggcaggaaagcggcgtggaaggcgcgaaccgctttaaacgcgtgtggaaactggtgtatgaacataccgcgaaaggcgatgtggcggcgctgaacgtggatgcgctgaccgaaaaccagaaagcgctgcgccgcgatgtgcataaaaccattgcgaaagtgaccgatgatattggccgccgccagacctttaacaccgcgattgcggcgattatggaactgatgaacaaactggcgaaagcgccgaccgatggcgaacaggatcgcgcgctgatgcaggaagcgctgctggcggtggtgcgcatgctgaacccgtttaccccgcatatttgctttaccctgtggcaggaactgaaaggcgaaggcgatattgataacgcgccgtggccggtggcggatgaaaaagcgatggtggaagatagcaccctggtggtggtgcaggtgaacggcaaagtgcgcgcgaaaattaccgtgccggtggatgcgaccgaagaacaggtgcgcgaacgcgcgggccaggaacatctggtggcgaaatatctggatggcgtgaccgtgcgcaaagtgatttatgtgccgggcaaactgctgaacctggtggtgggcggcccggtg

SEQ ID NO: 64 - DNA, 2589 bp, Artificial Sequence
  Description of Artificial Sequence Synthetic polynucleotide
atggaagaacagtatcgcccggaagaaattgaaagcaaagtgcagctgcattgggataaaaaacgcacctttgaagtgaccgaagatgaaagcaaagaaaaatattattgcctgagcattagcccgtatccgagcggccgcctgcatatgggccatgtgcgcaactataccattggcgatgtgattgcgcgctatcagcgcatgctgggcaaaaacgtgctgcagccgattggctgggatgcgtttggcctgccggcggaaggcgcggcggtgaaaaacaacaccgcgccggcgccgtggacctatgataacattgcgtatatgaaaaaccagctgaaaatgctgggctttggctatgattggagccgcgaactggcgacctgcaccccggaatattatcgctgggaacagaaattttttaccgaactgtataaaaaaggcctggtgtataaaaaaaccagcgcggtgaactggtgcccgaacgatcagaccgtgctggcgaacgaacaggtgattgatggctgctgctggcgctgcgataccaaagtggaacgcaaagaaattccgcagtggtttattaaaattaccgcgtatgcggatgaactgctgaacgatctggataaactggatcattggccggataccgtgaaaaccatgcagcgcaactggattggccgcagcgaaggcgtggaaattacctttaacgtgaacgattatgataacaccctgaccgtgtataccacccgcccggatgcgtttatgggctgcacctatctggcggtggcggcgggccatccgctggcgcagaaagcggcggaaaacaacccggaactggcggcgtttattgatgaatgccgcaacaccaaagtggcggaagcggaaatggcgaccatggaaaaaaaaggcgtggataccggctttaaagcggtgcatccgctgaccggcgaagaaattccggtgtgggcggcgaactttgtgctgatggaatatggcaccggcgcggtgatggcggtgccgggccatgatcagcgcgattatgaatttgcgagcaaatatggcctgaacattaaaccggtgattctggcggcggatggcagcgaaccggatctgagccagcaggcgctgaccgaaaaaggcgtgctgtttaacagcggcgaatttaacggcctggatcatgaagcggcgtttaacgcgattgcggataaactgaccgcgatgggcgtgggcgaacgcaaagtgaactatcgcctgcgcgattggggcgtgagccgccagcgctattggggcgcgccgattccgatggtgaccctggaagatggcaccgtgatgccgaccccggatgatcagctgccggtgattctgccggaagatgtggtgatggatggcattaccagcccgattaaagcggatccggaatgggcgaaaaccaccgtgaacggcatgccggcgctgcgcgaaaccgatacctttgatacctttatggaaagcagctggcattatgcgcgctatacctgcccgcagtataaagaaggcatgctggatagcgaagcggcgaactattggctgccggtggatattgcgattggcggcattgaacatgcgattatgggcctgctgtattttcgcttttttcataaactgatgcgcgatgcgggcatggtgaacagcgatgaaccggcgaaacagctgctgtgccagggcatggtgctggcggatgcgttttattatgtgggcgaaaacggcgaacgcaactgggtgagcccggtggatgcgattgtggaacgcgatgaaaaaggccgcattgtgaaagcgaaagatgcggcgggccatgaactggtgtataccggcatgagcaaaatgagcaaaagcaaaaacaacggcattgatccgcaggtgatggtggaacgctatggcgcggataccgtgcgcctgtttatgatgtttgcgagcccggcggatatgaccctggaatggcaggaaagcggcgtggaaggcgcgaaccgctttctgaaacgcgtgtggaaactggtgtatgaacataccgcgaaaggcgatgtggcggcgctgaacgtggatgcgctgaccgaaaaccagaaagcgctgcgccgcgatgtgcataaaaccattgcgaaagtgaccgatgatattggccgccgccagacctttaacaccgcgattgcggcgattatggaactgatgaacaaactggcgaaagcgccgaccgatggcgaacaggatcgcgcgctgatgcaggaagcgctgctggcggtggtgcgcatgctgaacccgtttaccccgcatatttgctttaccctgtggcaggaactgaaaggcgaaggcgatattgataacgcgccgtggccggtggcggatgaaaaagcgatggtggaagatagcaccctggtggtggtgcaggtgaacggcaaagtgcgcgcgaaaattaccgtgccggtggatgcgaccgaagaacaggtgcgcgaacgcgcgggccaggaacatctggtggcgaaatatctggatggcgtgaccgtgcgcaaagtgatttatgtgccgggcaaactgctgaacctggtggtgggcggcccggtg

SEQ ID NO: 65 - DNA, 2589 bp, Artificial Sequence
  Description of Artificial Sequence Synthetic polynucleotide
atggaagaacagtatcgcccggaagaaattgaaagcaaagtgcagctgcattgggataaaaaacgcacctttgaagtgaccgaagatgaaagcaaagaaaaatattattgcctgagcattagcccgtatccgagcggccgcctgcatatgggccatgtgcgcaactataccattggcgatgtgattgcgcgctatcagcgcatgctgggcaaaaacgtgctgcagccgattggctgggatgcgtttggcctgccggcggaaggcgcggcggtgaaaaacaacaccgcgccggcgccgtggacctatgataacattgcgtatatgaaaaaccagctgaaaatgctgggctttggctatgattggagccgcgaactggcgacctgcaccccggaatattatcgctgggaacagaaattttttaccgaactgtataaaaaaggcctggtgtataaaaaaaccagcgcggtgaactggtgcccgaacgatcagaccgtgctggcgaacgaacaggtgattgatggctgctgctggcgctgcgataccaaagtggaacgcaaagaaattccgcagtggtttattaaaattaccgcgtatgcggatgaactgctgaacgatctggataaactggatcattggccggataccgtgaaaaccatgcagcgcaactggattggccgcagcgaaggcgtggaaattacctttaacgtgaacgattatgataacaccctgaccgtgtataccacccgcccggatgcgtttatgggctgcacctatctggcggtggcggcgggccatccgctggcgcagaaagcggcggaaaacaacccggaactggcggcgtttattgatgaatgccgcaacaccaaagtggcggaagcggaaatggcgaccatggaaaaaaaaggcgtggataccggctttaaagcggtgcatccgctgaccggcgaagaaattccggtgtgggcggcgaactttgtgctgatggaatatggcaccggcgcggtgatggcggtgccgggccatgatcagcgcgattatgaatttgcgagcaaatatggcctgaacattaaaccggtgattctggcggcggatggcagcgaaccggatctgagccagcaggcgctgaccgaaaaaggcgtgctgtttaacagcggcgaatttaacggcctggatcatgaagcggcgtttaacgcgattgcggataaactgaccgcgatgggcgtgggcgaacgcaaagtgaactatcgcctgcgcgattggggcgtgagccgccagcgctattggggcgcgccgattccgatggtgaccctggaagatggcaccgtgatgccgaccccggatgatcagctgccggtgattctgccggaagatgtggtgatggatggcattaccagcccgattaaagcggatccggaatgggcgaaaaccaccgtgaacggcatgccggcgctgcgcgaaaccgatacctttgatacctttatggaaagcagctggatttatgcgcgctatacctgcccgcagtataaagaaggcatgctggatagcgaagcggcgaactattggctgccggtggatattattattggcggcattgaacatgcgattatgggcctgctgtattttcgcttttttcataaactgatgcgcgatgcgggcatggtgaacagcgatgaaccggcgaaacagctgctgtgccagggcatggtgctggcggatgcgttttattatgtgggcgaaaacggcgaacgcaactgggtgagcccggtggatgcgattgtggaacgcgatgaaaaaggccgcattgtgaaagcgaaagatgcggcgggccatgaactggtgtataccggcatgagcaaaatgagcaaaagcaaaaacaacggcattgatccgcaggtgatggtggaacgctatggcgcggataccgtgcgcctgtttatgatgtttgcgagcccggcggatatgaccctggaatggcaggaaagcggcgtggaaggcgcgaaccgctttctgaaacgcgtgtggaaactggtgtatgaacataccgcgaaaggcgatgtggcggcgctgaacgtggatgcgctgaccgaaaaccagaaagcgctgcgccgcgatgtgcataaaaccattgcgaaagtgaccgatgatattggccgccgccagacctttaacaccgcgattgcggcgattatggaactgatgaacaaactggcgaaagcgccgaccgatggcgaacaggatcgcgcgctgatgcaggaagcgctgctggcggtggtgcgcatgctgaacccgtttaccccgcatatttgctttaccctgtggcaggaactgaaaggcgaaggcgatattgataacgcgccgtggccggtggcggatgaaaaagcgatggtggaagatagcaccctggtggtggtgcaggtgaacggcaaagtgcgcgcgaaaattaccgtgccggtggatgcgaccgaagaacaggtgcgcgaacgcgcgggccaggaacatctggtggcgaaatatctggatggcgtgaccgtgcgcaaagtgatttatgtgccgggcaaactgctgaacctggtggtgggcggcccggtg

SEQ ID NO: 66 - DNA, 2589 bp, Artificial Sequence
  Description of Artificial Sequence Synthetic polynucleotide
atggaagaacagtatcgcccggaagaaattgaaagcaaagtgcagctgcattgggataaaaaacgcacctttgaagtgaccgaagatgaaagcaaagaaaaatattattgcctgagcattagcccgtatccgagcggccgcctgcatatgggccatgtgcgcaactataccattggcgatgtgattgcgcgctatcagcgcatgctgggcaaaaacgtgctgcagccgattggctgggatgcgtttggcctgccggcggaaggcgcggcggtgaaaaacaacaccgcgccggcgccgtggacctatgataacattgcgtatatgaaaaaccagctgaaaatgctgggctttggctatgattggagccgcgaactggcgacctgcaccccggaatattatcgctgggaacagaaattttttaccgaactgtataaaaaaggcctggtgtataaaaaaaccagcgcggtgaactggtgcccgaacgatcagaccgtgctggcgaacgaacaggtgattgatggctgctgctggcgctgcgataccaaagtggaacgcaaagaaattccgcagtggtttattaaaattaccgcgtatgcggatgaactgctgaacgatctggataaactggatcattggccggataccgtgaaaaccatgcagcgcaactggattggccgcagcgaaggcgtggaaattacctttaacgtgaacgattatgataacaccctgaccgtgtataccacccgcccggatgcgtttatgggctgcacctatctggcggtggcggcgggccatccgctggcgcagaaagcggcggaaaacaacccggaactggcggcgtttattgatgaatgccgcaacaccaaagtggcggaagcggaaatggcgaccatggaaaaaaaaggcgtggataccggctttaaagcggtgcatccgctgaccggcgaagaaattccggtgtgggcggcgaactttgtgctgatggaatatggcaccggcgcggtgatggcggtgccgggccatgatcagcgcgattatgaatttgcgagcaaatatggcctgaacattaaaccggtgattctggcggcggatggcagcgaaccggatctgagccagcaggcgctgaccgaaaaaggcgtgctgtttaacagcggcgaatttaacggcctggatcatgaagcggcgtttaacgcgattgcggataaactgaccgcgatgggcgtgggcgaacgcaaagtgaactatcgcctgcgcgattggggcgtgagccgccagcgctattggggcgcgccgattccgatggtgaccctggaagatggcaccgtgatgccgaccccggatgatcagctgccggtgattctgccggaagatgtggtgatggatggcattaccagcccgattaaagcggatccggaatgggcgaaaaccaccgtgaacggcatgccggcgctgcgcgaaaccgatacctttgatacctttatggaaagcagctggatttatgcgcgctatacctgcccgcagtataaagaaggcatgctggatagcgaagcggcgaactattggctgccggtggatattgtgattggcggcattgaacatgcgattatgggcctgctgtattttcgcttttttcataaactgatgcgcgatgcgggcatggtgaacagcgatgaaccggcgaaacagctgctgtgccagggcatggtgctggcggatgcgttttattatgtgggcgaaaacggcgaacgcaactgggtgagcccggtggatgcgattgtggaacgcgatgaaaaaggccgcattgtgaaagcgaaagatgcggcgggccatgaactggtgtataccggcatgagcaaaatgagcaaaagcaaaaacaacggcattgatccgcaggtgatggtggaacgctatggcgcggataccgtgcgcctgtttatgatgtttgcgagcccggcggatatgaccctggaatggcaggaaagcggcgtggaaggcgcgaaccgctttctgaaacgcgtgtggaaactggtgtatgaacataccgcgaaaggcgatgtggcggcgctgaacgtggatgcgctgaccgaaaaccagaaagcgctgcgccgcgatgtgcataaaaccattgcgaaagtgaccgatgatattggccgccgccagacctttaacaccgcgattgcggcgattatggaactgatgaacaaactggcgaaagcgccgaccgatggcgaacaggatcgcgcgctgatgcaggaagcgctgctggcggtggtgcgcatgctgaacccgtttaccccgcatatttgctttaccctgtggcaggaactgaaaggcgaaggcgatattgataacgcgccgtggccggtggcggatgaaaaagcgatggtggaagatagcaccctggtggtggtgcaggtgaacggcaaagtgcgcgcgaaaattaccgtgccggtggatgcgaccgaagaacaggtgcgcgaacgcgcgggccaggaacatctggtggcgaaatatctggatggcgtgaccgtgcgcaaagtgatttatgtgccgggcaaactgctgaacctggtggtgggcggcccggtg

SEQ ID NO: 67 - DNA, 2589 bp, Artificial Sequence
  Description of Artificial Sequence Synthetic polynucleotide
atggaagaacagtatcgcccggaagaaattgaaagcaaagtgcagctgcattgggataaaaaacgcacctttgaagtgaccgaagatgaaagcaaagaaaaatattattgcctgagcattagcccgtatccgagcggccgcctgcatatgggccatgtgcgcaactataccattggcgatgtgattgcgcgctatcagcgcatgctgggcaaaaacgtgctgcagccgattggctgggatgcgtttggcctgccggcggaaggcgcggcggtgaaaaacaacaccgcgccggcgccgtggacctatgataacattgcgtatatgaaaaaccagctgaaaatgctgggctttggctatgattggagccgcgaactggcgacctgcaccccggaatattatcgctgggaacagaaattttttaccgaactgtataaaaaaggcctggtgtataaaaaaaccagcgcggtgaactggtgcccgaacgatcagaccgtgctggcgaacgaacaggtgattgatggctgctgctggcgctgcgataccaaagtggaacgcaaagaaattccgcagtggtttattaaaattaccgcgtatgcggatgaactgctgaacgatctggataaactggatcattggccggataccgtgaaaaccatgcagcgcaactggattggccgcagcgaaggcgtggaaattacctttaacgtgaacgattatgataacaccctgaccgtgtataccacccgcccggatgcgtttatgggctgcacctatctggcggtggcggcgggccatccgctggcgcagaaagcggcggaaaacaacccggaactggcggcgtttattgatgaatgccgcaacaccaaagtggcggaagcggaaatggcgaccatggaaaaaaaaggcgtggataccggctttaaagcggtgcatccgctgaccggcgaagaaattccggtgtgggcggcgaactttgtgctgatggaatatggcaccggcgcggtgatggcggtgccgggccatgatcagcgcgattatgaatttgcgagcaaatatggcctgaacattaaaccggtgattctggcggcggatggcagcgaaccggatctgagccagcaggcgctgaccgaaaaaggcgtgctgtttaacagcggcgaatttaacggcctggatcatgaagcggcgtttaacgcgattgcggataaactgaccgcgatgggcgtgggcgaacgcaaagtgaactatcgcctgcgcgattggggcgtgagccgccagcgctattggggcgcgccgattccgatggtgaccctggaagatggcaccgtgatgccgaccccggatgatcagctgccggtgattctgccggaagatgtggtgatggatggcattaccagcccgattaaagcggatccggaatgggcgaaaaccaccgtgaacggcatgccggcgctgcgcgaaaccgatacctttgatacctttatggaaagcagctggatttatgcgcgctatacctgcccgcagtataaagaaggcatgctggatagcgaagcggcgaactattggctgccggtggatattggcattggcggcattgaacatgcgattatgggcctgctgtattttcgcttttttcataaactgatgcgcgatgcgggcatggtgaacagcgatgaaccggcgaaacagctgctgtgccagggcatggtgctggcggatgcgttttattatgtgggcgaaaacggcgaacgcaactgggtgagcccggtggatgcgattgtggaacgcgatgaaaaaggccgcattgtgaaagcgaaagatgcggcgggccatgaactggtgtataccggcatgagcaaaatgagcaaaagcaaaaacaacggcattgatccgcaggtgatggtggaacgctatggcgcggataccgtgcgcctgtttatgatgtttgcgagcccggcggatatgaccctggaatggcaggaaagcggcgtggaaggcgcgaaccgctttctgaaacgcgtgtggaaactggtgtatgaacataccgcgaaaggcgatgtggcggcgctgaacgtggatgcgctgaccgaaaaccagaaagcgctgcgccgcgatgtgcataaaaccattgcgaaagtgaccgatgatattggccgccgccagacctttaacaccgcgattgcggcgattatggaactgatgaacaaactggcgaaagcgccgaccgatggcgaacaggatcgcgcgctgatgcaggaagcgctgctggcggtggtgcgcatgctgaacccgtttaccccgcatatttgctttaccctgtggcaggaactgaaaggcgaaggcgatattgataacgcgccgtggccggtggcggatgaaaaagcgatggtggaagatagcaccctggtggtggtgcaggtgaacggcaaagtgcgcgcgaaaattaccgtgccggtggatgcgaccgaagaacaggtgcgcgaacgcgcgggccaggaacatctggtggcgaaatatctggatggcgtgaccgtgcgcaaagtgatttatgtgccgggcaaactgctgaacctggtggtgggcggcccggtg

SEQ ID NO: 68 - DNA, 81 bp, Artificial Sequence
  Description of Artificial Sequence Synthetic oligonucleotide
gtggggttcccgagcggccaaagggagcagacttcaaatctgccgtcacagacttcgaaggttcgaatccttcccccacca

SEQ ID NO: 69 - DNA, 81 bp, Artificial Sequence
  Description of Artificial Sequence Synthetic oligonucleotide
  modified_base (34)..(36): cta, tca, or tta
gtggggttcccgagcggccaaagggagcagactnnnaatctgccgtcacagacttcgaaggttcgaatccttcccccacca

SEQ ID NO: 70 - protein, 427 aa, Artificial Sequence
  Description of Artificial Sequence Synthetic polypeptide
MASSNLIKQLQERGLVAQVTDEEALAERLAQGPIALVCGFDPTADSLHLGHLVPLLCLKRFQQAGHKPVALVGGATGLIGDPSFKAAERKLNTEETVQEWVDKIRKQVAPFLDFDCGENSAIAANNYDWFGNMNVLTFLRDIGKHFSVNQMINKEAVKQRLNREDQGISFTEFSYNLLQGYSMACLNKQYGVVLQIGGSDQWGNITSGIDLTRRLHQNQVFGLTVPLITKADGTKFGKTEGGAVWLDPKKTSPYKFYQFWINTADADVYRFLKFFTFMSIEEINALEEEDKNSGKAPRAQYVLAEQVTRLVHGEEGLQAAKRITECLFSGSLSALSEADFEQLAQDGVPMVEMEKGADLMQALVDSELQPSRGQARKTIASNAITINGEKQSDPEYFFKEEDRLFGRFTLLRRGKKNYCLICWKGPV

SEQ ID NO: 71 - DNA, 1284 bp, Artificial Sequence
  Description of Artificial Sequence Synthetic polynucleotide
atggcaagcagtaacttgattaaacaattgcaagagcgggggctggtagcccaggtgacggacgaggaagcgttagcagagcgactggcgcaaggcccgatcgcactcgtgtgtggcttcgatcctaccgctgacagcttgcatttggggcatcttgttccattgttatgcctgaaacgcttccagcaggcgggccacaagccggttgcgctggtaggcggcgcgacgggtctgattggcgacccgagcttcaaagctgccgagcgtaagctgaacaccgaagaaactgttcaggagtgggtggacaaaatccgtaagcaggttgccccgttcctcgatttcgactgtggagaaaactctgctatcgcggccaataattatgactggttcggcaatatgaatgtgctgaccttcctgcgcgatattggcaaacacttctccgttaaccagatgatcaacaaagaagcggttaagcagcgtctcaaccgtgaagatcaggggatttcgttcactgagttttcctacaacctgctgcagggttatagtatggcctgtttgaacaaacagtacggtgtggtgctgcaaattggtggttctgaccaatggggtaacatcacttctggtatcgacctgacccgtcgtctgcatcagaatcaggtgtttggcctgaccgttccgctgatcactaaagcagatggcaccaaatttggtaaaactgaaggcggcgcagtctggttggacccgaagaaaaccagcccgtacaaattctaccagttctggatcaacactgcggatgccgacgtttaccgcttcctgaagttcttcacctttatgagcattgaagagatcaacgccctggaagaagaagataaaaacagcggtaaagcaccgcgcgcccagtatgtactggcggagcaggtgactcgtctggttcacggtgaagaaggtttacaggcggcaaaacgtattaccgaatgcctgttcagcggttctttgagtgcgctgagtgaagcggacttcgaacagctggcgcaggacggcgtaccgatggttgagatggaaaagggcgcagacctgatgcaggcactggtcgattctgaactgcaaccttcccgtggtcaggcacgtaaaactatcgcctccaatgccatcaccattaacggtgaaaaacagtccgatcctgaatacttctttaaagaagaagatcgtctgtttggtcgttttaccttactgcgtcgcggtaaaaagaattactgtctgatttgctggaaagggcccgtttaa

SEQ ID NO: 72 - DNA, 69 bp, Artificial Sequence
  Description of Artificial Sequence Synthetic oligonucleotide
ggaaacctgatcatgtagatcgaacggactctaaatccgttcagccgggttagattcccggggtttccg

SEQ ID NO: 73 - DNA, 69 bp, Artificial Sequence
  Description of Artificial Sequence Synthetic oligonucleotide
  modified_base (31)..(33): a, c, t, g, unknown or other
ggaaacctgatcatgtagatcgaacggactnnnaatccgttcagccgggttagattcccggggtttccg

SEQ ID NO: 74 - RNA, 72 nt, Artificial Sequence
  Description of Artificial Sequence Synthetic oligonucleotide
ggaaaccugaucauguagaucgaacggacucuaaauccguucagccggguuagauucccgggguuuccgcca

SEQ ID NO: 75 - RNA, 72 nt, Artificial Sequence
  Description of Artificial Sequence Synthetic oligonucleotide
gggcggcugaucauguagaucgaacggacucuaaauccguucagccggguuagauucccgggcugcccgcca

SEQ ID NO: 76 - RNA, 72 nt, Artificial Sequence
  Description of Artificial Sequence Synthetic oligonucleotide
gggugacugaucauguagaucgaacggacucuaaauccguucagccggguuagauucccggguugcccgcca

SEQ ID NO: 77 - RNA, 72 nt, Artificial Sequence
  Description of Artificial Sequence Synthetic oligonucleotide
ggggggcugaucauguagaucgaacggacucuaaauccguucagccggguuagauucccgggcuccccgcca

SEQ ID NO: 78 - RNA, 72 nt, Artificial Sequence
  Description of Artificial Sequence Synthetic oligonucleotide
gggcggcugaucauguagaucgaacggacucuaaauccguucagccggguuagauucccggguugcccgcca

SEQ ID NO: 79 - RNA, 72 nt, Artificial Sequence
  Description of Artificial Sequence Synthetic oligonucleotide
gggcgccugaucauguagaucgaacggacucuaaauccguucagccggguuagauucccggggcgcccgcca

SEQ ID NO: 80 - RNA, 72 nt, Artificial Sequence
  Description of Artificial Sequence Synthetic oligonucleotide
ggggaggugaucauguagaucgaacggacucuaaauccguucagccggguuagauucccggccuccccgcca

SEQ ID NO: 81 - RNA, 72 nt, Artificial Sequence
  Description of Artificial Sequence Synthetic oligonucleotide
ggggaccugaucauguagaucgaacggacucuaaauccguucagccggguuagauucccgggguccccgcca

SEQ ID NO: 82 - RNA, 72 nt, Artificial Sequence
  Description of Artificial Sequence Synthetic oligonucleotide
ggccgggugaucauguagaucgaacggacucuaaauccguucagccggguuagauucccggccuggccgcca

SEQ ID NO: 83 - RNA, 72 nt, Artificial Sequence
  Description of Artificial Sequence Synthetic oligonucleotide
ggggaccugaucauguagaucgaacggacucuaaauccguucagccggguuagauucccgggguccucgcca

SEQ ID NO: 84 - RNA, 72 nt, Artificial Sequence
  Description of Artificial Sequence Synthetic oligonucleotide
ggggcccugaucauguagaucgaacggacucuaaauccguucagccggguuagauucccgggggucccgcca

SEQ ID NO: 85 - RNA, 72 nt, Artificial Sequence
  Description of Artificial Sequence Synthetic oligonucleotide
gggggccugaucauguagaucgaacggacucuaaauccguucagccggguuagauucccggggcucccgcca

SEQ ID NO: 86 - RNA, 72 nt, Artificial Sequence
  Description of Artificial Sequence Synthetic oligonucleotide
gggguccugaucauguagaucgaacggacucuaaauccguucagccggguuagauucccggggaucccgcca

SEQ ID NO: 87 - RNA, 72 nt, Artificial Sequence
  Description of Artificial Sequence Synthetic oligonucleotide
ggggaccugaucauguagaucgaacggacucuaaauccguucagccggguuagauucccgggguucccgcca

SEQ ID NO: 88 - RNA, 72 nt, Artificial Sequence
  Description of Artificial Sequence Synthetic oligonucleotide
ggggaggugaucauguagaucgaacggacucuaaauccguucagccggguuagauucccggucuucccgcca

SEQ ID NO: 89 - RNA, 72 nt, Artificial Sequence
  Description of Artificial Sequence Synthetic oligonucleotide
gggggggugaucauguagaucgaacggacucuaaauccguucagccggguuagauucccggcccccucgcca

SEQ ID NO: 90 - RNA, 72 nt, Artificial Sequence
  Description of Artificial Sequence Synthetic oligonucleotide
gguggggugaucauguagaucgaacggacucuaaauccguucagccggguuagauucccggcccuaccgcca

SEQ ID NO: 91 - RNA, 72 nt, Artificial Sequence
  Description of Artificial Sequence Synthetic oligonucleotide
gggggucugaucauguagaucgaacggacucuaaauccguucagccggguuagauucccgggacucccgcca

SEQ ID NO: 92 - RNA, 72 nt, Artificial Sequence
  Description of Artificial Sequence Synthetic oligonucleotide
gggucccugaucauguagaucgaacggacucuaaauccguucagccggguuagauucccgggggguccgcca

SEQ ID NO: 93 - RNA, 72 nt, Artificial Sequence
  Description of Artificial Sequence Synthetic oligonucleotide
ggggaccugaucauguagaucgaacggacucuaaauccguucagccggguuagauucccgggguucucgcca

SEQ ID NO: 94 - RNA, 72 nt, Artificial Sequence
  Description of Artificial Sequence Synthetic oligonucleotide
gggcggcugaucauguagaucgaacggacucuaaauccguucagccggguuagauucccgggccgcccgcca

SEQ ID NO: 95 - RNA, 72 nt, Artificial Sequence
  Description of Artificial Sequence Synthetic oligonucleotide
gagcaccugaucauguagaucgaacggacucuaaauccguucagccggguuagauucccggggugcucgcca

SEQ ID NO: 96 - RNA, 72 nt, Artificial Sequence
  Description of Artificial Sequence Synthetic oligonucleotide
gggggggugaucauguagaucgaacggacucuaaauccguucagccggguuagauucccggcccccccgcca

SEQ ID NO: 97 - RNA, 72 nt, Artificial Sequence
  Description of Artificial Sequence Synthetic oligonucleotide
gagggggugaucauguagaucgaacggacucuaaauccguucagccggguuagauucccggcccccucgcca

SEQ ID NO: 98 - RNA, 72 nt, Artificial Sequence
  Description of Artificial Sequence Synthetic oligonucleotide
gggagccugaucauguagaucgaacggacucuaaauccguucagccggguuagauucccgggguucccgcca

SEQ ID NO: 99 - RNA, 72 nt, Artificial Sequence; identical to SEQ ID NO: 98
  Description of Artificial Sequence Synthetic oligonucleotide

SEQ ID NO: 100 - RNA, 72 nt, Artificial Sequence
  Description of Artificial Sequence Synthetic oligonucleotide
gaggaccugaucauguagaucgaacggacucuaaauccguucagccggguuagauucccgggguucucgcca

SEQ ID NO: 101 - protein, 419 aa, Artificial Sequence
  Description of Artificial Sequence Synthetic polypeptide
MDKKPLDVLISATGLWMSRTGTLHKIKHHEVSRSKIYIEMACGDHLVVNNSRSCRTARAFRHHKYRKTCKRCRVSDEDINNFLTRSTESKNSVKVRVVSAPKVKKAMPKSVSRAPKPLENSVSAKASTNTSRSVPSPAKSTPNSSVPASAPAPSLTRSQLDRVEALLSPEDKISLNMAKPFRELEPELVTRRKNDFQRLYTNDREDYLGKLERDITKFFVDRGFLEIKSPILIPAEYVERMGINNDTELSKQIFRVDKNLCLRPMLAPTLYNYLRKLDRILPGPIKIFEVGPCYRKESDGKEHLEEFTMVNFCQMGSGCTRENLEALIKEFLDYLEIDFEIVGDSCMVFGDTLDIMHGDLELSSAVVGPVSLDREWGIDKPWIGAGFGLERLLKVMHGFKNIKRASRSESYYNGISTNL

SEQ ID NO: 102 - DNA, 1260 bp, Artificial Sequence
  Description of Artificial Sequence Synthetic polynucleotide
atggataaaaaaccattagatgttttaatatctgcgaccgggctctggatgtccaggactggcacgctccacaaaatcaagcaccatgaggtctcaagaagtaaaatatacattgaaatggcgtgtggagaccatcttgttgtgaataattccaggagttgtagaacagccagagcattcagacatcataagtacagaaaaacctgcaaacgatgtagggtttcggacgaggatatcaataattttctcacaagatcaaccgaaagcaaaaacagtgtgaaagttagggtagtttctgctccaaaggtcaaaaaagctatgccgaaatcagtttcaagggctccgaagcctctggaaaattctgtttctgcaaaggcatcaacgaacacatccagatctgtaccttcgcctgcaaaatcaactccaaattcgtctgttcccgcatcggctcctgctccttcacttacaagaagccagcttgatagggttgaggctctcttaagtccagaggataaaatttctctaaatatggcaaagcctttcagggaacttgagcctgaacttgtgacaagaagaaaaaacgattttcagcggctctataccaatgatagagaagactacctcggtaaactcgaacgtgatattacgaaatttttcgtagaccggggttttctggagataaagtctcctatccttattccggcggaatacgtggagagaatgggtattaataatgatactgaactttcaaaacagatcttccgggtggataaaaatctctgcttgaggccaatgcttgccccgactctttacaactatctgcgaaaactcgataggattttaccaggcccaataaaaattttcgaagtcggaccttgttaccggaaagagtctgacggcaaagagcacctggaagaatttactatggtgaacttctgtcagatgggttcgggatgtactcgggaaaatcttgaagctctcatcaaagagtttctggactatctggaaatcgacttcgaaatcgtaggagattcctgtatggtctttggggatactcttgatataatgcacggggacctggagctttcttcggcagtcgtcgggccagtttctcttgatagagaatggggtattgacaaaccatggataggtgcaggttttggtcttgaacgcttgctcaaggttatgcacggctttaaaaacattaagagggcatcaaggtccgaatcttactataatgggatttcaaccaatctgtaa

SEQ ID NO: 103 - DNA, 1005 bp, Artificial Sequence
  Description of Artificial Sequence Synthetic polynucleotide
atgactaagcccatcgtttttgctggcgcacagccctcaggtgaattgaccattggtaactacatgggtgcgctgcgtcagtgggtaaacatgcaggatgactaccattgcatttactgtatcgttgaccaacacgcgatcaccgtgcgccaggatgcacagaagctgcgtaaagcgacgctggatacgctggccttgtatctggcttgtggtatcgatcctgagaaaagcaccatttttgttcagtcccacgtgccggaacatgcacagttaggctgggcactgaactgctatacctacttcggcgaactgagtcgcatgacgcagtttaaagataaatctgcgcgttatgccgagaacatcaacgctggtctgtttgactatccggtgctgatggcagcggacatcctgctgtatcaaactaatctgggtccttgtggtgaagaccagaaacagcacctcgaactgagccgcgatattgcccagcgtttcaacgcgctgtatggcgagatctttaaggtgccggagccgtttattccgaaatctggcgcgcgcgtaatgtcgctgctggagccgaccaagaagatgtccaagtctgacgataatcgcaataacgttatcggcctgctggaagatccgaaatcggtagtgaagaaaatcaaacgtgcggtcactgactccgacgagccgccggtagttcgctacgatgtgcagaacaaagcgggcgtttccaacctgttggatatcctttcagcggtaacgggccagagcatcccagaactggaaaaacagttcgaaggcaagatgtatggtcatctgaaaggtgaagtggctgatgccgtttccggtatgctgactgaattgcaggaacgctatcaccgtttccgcaacgatgaagccttcctgcaacaggtgatgaaagatggcgcggaaaaagccagcgcgcacgcttcccgtacgctaaaagcggtgtacgaagcgattggttttgtggcgaagccgtaa

SEQ ID NO: 104 - DNA, 84 bp, Artificial Sequence
  Description of Artificial Sequence Synthetic oligonucleotide
gtggggttcccgagcggccaaagggagcagacttcaaatctgccgtcacagacttcgaaggttcgaatccttcccccaccacca

SEQ ID NO: 105 - DNA, 84 bp, Artificial Sequence
  Description of Artificial Sequence Synthetic oligonucleotide
  modified_base (34)..(36): cta, tca, or tta
gtggggttcccgagcggccaaagggagcagactnnnaatctgccgtcacagacttcgaaggttcgaatccttcccccaccacca

SEQ ID NO: 106 - DNA, 72 bp, Artificial Sequence
  Description of Artificial Sequence Synthetic oligonucleotide
ggaaacctgatcatgtagatcgaacggactctaaatccgttcagccgggttagattcccggggtttccgcca

SEQ ID NO: 107 - DNA, 72 bp, Artificial Sequence
  Description of Artificial Sequence Synthetic oligonucleotide
  modified_base (31)..(33): a, c, t, g, unknown or other
ggaaacctgatcatgtagatcgaacggactnnnaatccgttcagccgggttagattcccggggtttccgcca

SEQ ID NO: 108 - DNA, 73 bp, Artificial Sequence
  Description of Artificial Sequence Synthetic oligonucleotide
aggggcgtagttcaattggtagagcaccggtctccaaaaccgggtgttgggagttcgagtctctccgcccctg

SEQ ID NO: 109 - DNA, 73 bp, Artificial Sequence
  Description of Artificial Sequence Synthetic oligonucleotide
aggggcgtagttcaattggtagagcaccggtctctaaaaccgggtgttgggagttcgagtctctccgcccctg

SEQ ID NO: 110 - DNA, 73 bp, Artificial Sequence
  Description of Artificial Sequence Synthetic oligonucleotide
aggggcgtagttcaattggtagagcaccggtcttcaaaaccgggtgttgggagttcgagtctctccgcccctg

SEQ ID NO: 111 - DNA, 73 bp, Artificial Sequence
  Description of Artificial Sequence Synthetic oligonucleotide
aggggcgtagttcaattggtagagcaccggtctttaaaaccgggtgttgggagttcgagtctctccgcccctg

SEQ ID NO: 112 - DNA, 72 bp, Artificial Sequence
  Description of Artificial Sequence Synthetic oligonucleotide
  modified_base (34)..(36): cta, tca, or tta
tggggtatcgccaagcggtaaggcaccggattcnnnttccggcattccgaggttcgaatcctcgtaccccag

SEQ ID NO: 113 - DNA, 73 bp, Artificial Sequence
  Description of Artificial Sequence Synthetic oligonucleotide
  modified_base (34)..(36): cta, tca, or tta
aggggcatagctcaagcggtaaagcaccggactnnnaaaccggcagtccgaagttcgaatccccccaccccag

SEQ ID NO: 114 - protein, 330 aa, Homo sapiens [sequence omitted]

SEQ ID NO: 115 - protein, 106 aa, Homo sapiens
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 116 - protein, 24 aa, Artificial Sequence
  Description of Artificial Sequence Synthetic peptide
VVTVPSSSLGTQTYICNVNHKPSN

SEQ ID NO: 117 - protein, 22 aa, Artificial Sequence
  Description of Artificial Sequence Synthetic peptide
TFGQGTKVEIKRTVAAPSVFIF

SEQ ID NO: 118 - protein, 10 aa, Artificial Sequence
  Description of Artificial Sequence Synthetic peptide
GLVQPGGSLR

SEQ ID NO: 119 - protein, 30 aa, Artificial Sequence
  Description of Artificial Sequence Synthetic polypeptide
EVQLVESGGGLVQPGGSLRLSCAASGFNIK

SEQ ID NO: 120 - protein, 120 aa, Artificial Sequence [sequence omitted]

SEQ ID NO: 121 - protein, 450 aa, Artificial Sequence
  Description of Artificial Sequence Synthetic polypeptide
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 122 - protein, 11 aa, Artificial Sequence
  Description of Artificial Sequence Synthetic peptide
ADSVKGRFTIS

SEQ ID NO: 123 - protein, 17 aa, Artificial Sequence
  Description of Artificial Sequence Synthetic peptide
RIYPTNGYTRYADSVKG

SEQ ID NO: 124 - protein, 11 aa, Artificial Sequence
  Description of Artificial Sequence Synthetic peptide
FTISADTSKNT

SEQ ID NO: 125 - protein, 32 aa, Artificial Sequence
  Description of Artificial Sequence Synthetic polypeptide
RFTISADTSKNTAYLQMNSLRAEDTAVYYCSR

SEQ ID NO: 126 - protein, 11 aa, Artificial Sequence
  Description of Artificial Sequence Synthetic peptide
WGQGTLVTVSS

SEQ ID NO: 127 - protein, 11 aa, Artificial Sequence
  Description of Artificial Sequence Synthetic peptide
WGQGTLVTVSS

SEQ ID NO: 128 - protein, 11 aa, Artificial Sequence
  Description of Artificial Sequence Synthetic peptide
VTVSSASTKGP

SEQ ID NO: 129 - protein, 97 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKK

SEQ ID NO: 130 - protein, 330 aa, Artificial Sequence
  Description of Artificial Sequence Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 131 - protein, 10 aa, Artificial Sequence
  Description of Artificial Sequence Synthetic peptide
VTVSSASTKG

SEQ ID NO: 132 - protein, 10 aa, Artificial Sequence
  Description of Artificial Sequence Synthetic peptide
SSKSTSGGTA

SEQ ID NO: 133 - protein, 11 aa, Artificial Sequence
  Description of Artificial Sequence Synthetic peptide
TSGGTAALGCL

SEQ ID NO: 134 - protein, 11 aa, Artificial Sequence
  Description of Artificial Sequence Synthetic peptide
YFPEPVTVSWN

SEQ ID NO: 135 - protein, 11 aa, Artificial Sequence
  Description of Artificial Sequence Synthetic peptide
EPVTVSWNSGA

SEQ ID NO: 136 - protein, 11 aa, Artificial Sequence
  Description of Artificial Sequence Synthetic peptide
NSGALTSGVHT

SEQ ID NO: 137 - protein, 11 aa, Artificial Sequence
  Description of Artificial Sequence Synthetic peptide
SSSLGTQTYIC

SEQ ID NO: 138 - protein, 11 aa, Artificial Sequence
  Description of Artificial Sequence Synthetic peptide
QTYICNVNHKP

SEQ ID NO: 139 - protein, 11 aa, Artificial Sequence
  Description of Artificial Sequence Synthetic peptide
TYICNVNHKPS

SEQ ID NO: 140 - protein, 11 aa, Artificial Sequence
  Description of Artificial Sequence Synthetic peptide
YICNVNHKPSN

SEQ ID NO: 141 - protein, 11 aa, Artificial Sequence
  Description of Artificial Sequence Synthetic peptide
KPSNTKVDKKV

SEQ ID NO: 142 - protein, 11 aa, Artificial Sequence
  Description of Artificial Sequence Synthetic peptide
SNTKVDKKVEP

SEQ ID NO: 143 - protein, 11 aa, Artificial Sequence
  Description of Artificial Sequence Synthetic peptide
KDTLMISRTPE

SEQ ID NO: 144 - protein, 110 aa, Homo sapiens
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 145 - protein, 11 aa, Artificial Sequence
  Description of Artificial Sequence Synthetic peptide
EDPEVKFNWYV

SEQ ID NO: 146 - protein, 11 aa, Artificial Sequence
  Description of Artificial Sequence Synthetic peptide
VKFNWYVDGVE

SEQ ID NO: 147 - protein, 11 aa, Artificial Sequence
  Description of Artificial Sequence Synthetic peptide
EVHNAKTKPRE

SEQ ID NO: 148 - protein, 11 aa, Artificial Sequence
  Description of Artificial Sequence Synthetic peptide
HNAKTKPREEQ

SEQ ID NO: 149 - protein, 11 aa, Artificial Sequence
  Description of Artificial Sequence Synthetic peptide
REEQYNSTYRV

SEQ ID NO: 150 - protein, 11 aa, Artificial Sequence
  Description of Artificial Sequence Synthetic peptide
DSDGSFFLYSK

SEQ ID NO: 151 - protein, 107 aa, Artificial Sequence
  Description of Artificial Sequence Synthetic polypeptide
GQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 152 - protein, 11 aa, Artificial Sequence
  Description of Artificial Sequence Synthetic peptide
SLSASVGDRVT

SEQ ID NO: 153 - protein, 23 aa, Artificial Sequence
  Description of Artificial Sequence Synthetic peptide
DIQMTQSPSSLSASVGDRVTITC

SEQ ID NO: 154 - protein, 110 aa, Artificial Sequence
  Description of Artificial Sequence Synthetic polypeptide
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTV

SEQ ID NO: 155 - protein, 214 aa, Artificial Sequence
  Description of Artificial Sequence Synthetic polypeptide
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 156 - protein, 11 aa, Artificial Sequence
  Description of Artificial Sequence Synthetic peptide
VGDRVTITCRA

SEQ ID NO: 157 - protein, 11 aa, Artificial Sequence
  Description of Artificial Sequence Synthetic peptide
VTITCRASQDV

SEQ ID NO: 158 - protein, 11 aa, Artificial Sequence
  Description of Artificial Sequence Synthetic peptide
RASQDVNTAVA

SEQ ID NO: 159 - protein, 11 aa, Artificial Sequence
  Description of Artificial Sequence Synthetic peptide
YSGVPSRFSGS

SEQ ID NO: 160 - protein, 32 aa, Artificial Sequence
  Description of Artificial Sequence Synthetic polypeptide
GVPSRFSGSRSGTDFTLTISSLQPEDFATYYC

SEQ ID NO: 161 - protein, 11 aa, Artificial Sequence
  Description of Artificial Sequence Synthetic peptide
TKVEIKRTVAA

SEQ ID NO: 162 - protein, 13 aa, Artificial Sequence
  Description of Artificial Sequence Synthetic peptide
FGQGTKVEIKRTV

SEQ ID NO: 163 - protein, 11 aa, Artificial Sequence
  Description of Artificial Sequence Synthetic peptide
VEIKRTVAAPS

SEQ ID NO: 164 - protein, 11 aa, Artificial Sequence
  Description of Artificial Sequence Synthetic peptide
EIKRTVAAPSV

SEQ ID NO: 165 - protein, 11 aa, Artificial Sequence
  Description of Artificial Sequence Synthetic peptide
IKRTVAAPSVF

SEQ ID NO: 166 - protein, 104 aa, Homo sapiens
AAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 167 - protein, 11 aa, Artificial Sequence
  Description of Artificial Sequence Synthetic peptide
REAKVQWKVDN

SEQ ID NO: 168 - protein, 11 aa, Artificial Sequence
  Description of Artificial Sequence Synthetic peptide
KVDNALQSGNS

SEQ ID NO: 169 - protein, 11 aa, Artificial Sequence
  Description of Artificial Sequence Synthetic peptide
NALQSGNSQES

SEQ ID NO: 170 - protein, 11 aa, Artificial Sequence
  Description of Artificial Sequence Synthetic peptide
KHKVYACEVTH

SEQ ID NO: 171 - protein, 11 aa, Artificial Sequence
  Description of Artificial Sequence Synthetic peptide
GLSSPVTKSFN

SEQ ID NO: 172 - protein, 11 aa, Artificial Sequence
  Description of Artificial Sequence Synthetic peptide
LSSPVTKSFNR

SEQ ID NO: 173 - protein, 11 aa, Artificial Sequence
  Description of Artificial Sequence Synthetic peptide
SPVTKSFNRGE

SEQ ID NO: 174 - protein, 11 aa, Artificial Sequence
  Description of Artificial Sequence Synthetic peptide
FPEPVTVSWNS

SEQ ID NO: 175 - protein, 11 aa, Artificial Sequence
  Description of Artificial Sequence Synthetic peptide
TEQDSKDSTYS

SEQ ID NO: 176 - protein, 5 aa, Artificial Sequence
  Description of Artificial Sequence Synthetic peptide
GGGGS

SEQ ID NO: 177 - protein, 10 aa, Artificial Sequence
  Description of Artificial Sequence Synthetic peptide
GGGGSGGGGS

SEQ ID NO: 178 - protein, 11 aa, Artificial Sequence
  Description of Artificial Sequence Synthetic peptide
GDRVTITCRAS

SEQ ID NO: 179 - protein, 11 aa, Artificial Sequence
  Description of Artificial Sequence Synthetic peptide
ESGVPSRFSGS

SEQ ID NO: 180 - protein, 9 aa, Artificial Sequence
  Description of Artificial Sequence Synthetic peptide
PREAKVQWK

SEQ ID NO: 181 - protein, 9 aa, Artificial Sequence
  Description of Artificial Sequence Synthetic peptide
KVDNALQSG

SEQ ID NO: 182 - protein, 9 aa, Artificial Sequence
  Description of Artificial Sequence Synthetic peptide
KHKVYACEV

SEQ ID NO: 183 - protein, 9 aa, Artificial Sequence
  Description of Artificial Sequence Synthetic peptide
LSSPVTKSF

SEQ ID NO: 184 - protein, 9 aa, Artificial Sequence
  Description of Artificial Sequence Synthetic peptide
SSPVTKSFN

SEQ ID NO: 185 - protein, 11 aa, Artificial Sequence
  Description of Artificial Sequence Synthetic peptide
VTVSSASTKGP

SEQ ID NO: 186 - protein, 8 aa, Artificial Sequence
  Description of Artificial Sequence Synthetic peptide
SKSTSGGT

SEQ ID NO: 187 - protein, 8 aa, Artificial Sequence
  Description of Artificial Sequence Synthetic peptide
PEPVTVSW

SEQ ID NO: 188 - protein, 8 aa, Artificial Sequence
  Description of Artificial Sequence Synthetic peptide
GALTSGVH

SEQ ID NO: 189 - protein, 9 aa, Artificial Sequence
  Description of Artificial Sequence Synthetic peptide
QTYICNVNH

SEQ ID NO: 190 - protein, 10 aa, Artificial Sequence
  Description of Artificial Sequence Synthetic peptide
TYICNVNHKP

SEQ ID NO: 191 - protein, 11 aa, Artificial Sequence
  Description of Artificial Sequence Synthetic peptide
LAPSSKSTSGG

SEQ ID NO: 192 - protein, 9 aa, Artificial Sequence
  Description of Artificial Sequence Synthetic peptide
PSNTKVDKK

SEQ ID NO: 193 - protein, 12 aa, Artificial Sequence
  Description of Artificial Sequence Synthetic peptide
EDPEVKFNWYVD

SEQ ID NO: 194 - protein, 13 aa, Artificial Sequence
  Description of Artificial Sequence Synthetic peptide
VHNAKTKPREEQY

SEQ ID NO: 195 - protein, 15 aa, Artificial Sequence
  Description of Artificial Sequence Synthetic peptide
  SITE (1)..(15): This sequence may encompass 1-5 “Gly Gly Pro” repeating units
GGPGGPGGPGGPGGP

SEQ ID NO: 196 - protein, 25 aa, Artificial Sequence
  Description of Artificial Sequence Synthetic peptide
  SITE (1)..(25): This sequence may encompass 1-5 “Gly Gly Gly Gly Ser” repeating units
GGGGSGGGGSGGGGSGGGGSGGGGS